(19) United States
(12) Patent Application Publication
(10) Pub. No.: US 2023/0265151 A1
(43) Pub. Date: Aug. 24, 2023

(54) GIP/GLP1 CO-AGONIST COMPOUNDS

(71) Applicant: Eli Lilly and Company, Indianapolis, IN (US)

(72) Inventors: Jorge ALSINA-FERNANDEZ, Indianapolis, IN (US); Robert Andrew BROWN, Carmel, IN (US); Robert Chadwick CUMMINS, Carmel, IN (US); Mohamed Elsayed Hamed ELSAYED, Fishers, IN (US); Andrea Renee GEISER, Greenwood, IN (US); Xianyin LAI, Carmel, IN (US); Hongchang QU, Carmel, IN (US); Brian Morgan WATSON, Carmel, IN (US)

(21) Appl. No.: 17/794,128

(22) PCT Filed: Jan. 21, 2021
(86) PCT No.: PCT/US2021/014302

§ 371 Date: Jul. 20, 2022

Related U.S. Application Data
(60) Provisional application No. 62/964,932, filed on Jan. 23, 2020.

(51) Int. Cl. C07K 14/605; C07K 14/645; A61K 38/26; A61K 38/22; A61P 3/10

(52) U.S. Cl. CPC C07K 14/605; A61K 38/26; A61K 38/2235; A61P 3/10; C07K 14/645

(57) ABSTRACT

The present invention relates to compounds having activity at both the human glucose-dependent insulinotropic polypeptide (GIP) and glucagon-like peptide-1 (GLP-1) receptors. The present invention also relates to compounds having an extended duration of action at each of these receptors. Furthermore, the present invention relates to compounds that may be administered orally. These compounds may be useful in the treatment of type 2 diabetes mellitus (“T2DM”). These compounds may be useful in the treatment of obesity.

The present invention relates to compounds having activity at both the human glucose-dependent insulinotropic polypeptide (GIP) and glucagon-like peptide-1 (GLP-1) receptors. The present invention also relates to compounds having an extended duration of action at each of these receptors. Furthermore, the present invention relates to compounds that may be administered orally. These compounds may be useful in the treatment of type 2 diabetes mellitus (“T2DM”). Also, the compounds may be useful in the treatment of obesity.

Over the past several decades, the prevalence of diabetes has continued to rise. T2DM is the most common form of diabetes accounting for approximately 90% of all diabetes. T2DM is characterized by high blood glucose levels associated mainly with insulin resistance. The current standard of care for T2DM includes diet and exercise, treatment with oral medications, and injectable glucose lowering drugs, including incretin-based therapies, such as GLP-1 receptor agonists. A variety of GLP-1 receptor agonists are currently available for treatment of T2DM, although currently marketed GLP-1 receptor agonists are generally dose-limited by gastrointestinal side effects such as nausea and vomiting. Subcutaneous injection is the typical route of administration for the available GLP-1 receptor agonists. When treatment with oral medications and incretin-based therapies are insufficient, insulin treatment is considered. Despite the advances in treatment available today, many patients with T2DM are unable to reach their glycemic control goals. Uncontrolled diabetes leads to several conditions associated with increased morbidity and mortality of patients. There is a need for a treatment to enable more patients with T2DM to reach their glycemic treatment goal.

Obesity is a complex medical disorder resulting in excessive accumulation of adipose tissue mass. Today obesity is a global public health concern that is associated with undesired health outcomes and morbidities. Desired treatments for patients with obesity strive to reduce excess body weight, improve obesity-related co-morbidities, and maintain long-term weight reduction. Available treatments for obesity are particularly unsatisfactory for patients with severe obesity. There is a need for alternative treatment options to induce therapeutic weight loss in patients in need of such treatment.

WO2016/111971 describes peptides stated to have GLP-1 and GIP activity. WO2013/164483 also discloses compounds stated to have GLP-1 and GIP activity.

There is a need for T2DM treatments capable of providing effective glucose control for a larger portion of the patients in need of such treatment. There is a further need for T2D treatments capable of providing effective glucose control and with a favorable side effect profile. There is a need for alternate treatment options to provide therapeutic weight loss in a patient in need of such treatment. There is a need for an alternate treatment option for a patient in need of treatment for severe obesity.

There is a desire for compounds having agonist activity at the GIP and GLP-1 receptors that are suitable for oral administration. Compounds with extended duration of action at each of the GIP and GLP-1 receptors are desirable to allow for less frequent dosing of the compound. Compounds with two acylation modifications in combination with an amino acid sequence to provide GLP-1 and GIP activity from a single compound are desired.

Accordingly, provided are a compound of Formula I

X₁X₂EGTX₆TSDX₁₀X₁₁X₁₂X₁₃LDX₁₆X₁₇AQX₂₀X₂₁X₂₂IX₂₄X₂₅
LIX₂₈GX₃₀(SEQ ID NO:505)

wherein
X₁ is selected from the group consisting of Y and R₁Y;
R₁ is an Ac modification of the N-terminal amino group;
X₂is Aib;
X₆ is selected from the group consisting of αMeF and αMeF(2F);
X₁₀ is selected from the group consisting of 4Pal, Y, αMeF, αMeF(2F), αMeL, αMeV, Ac4c, Ac5c, Ac6c, Bip, 1Nal, 2Nal, OMeY, hTyr, Nle, V, 4CPhe, ChG, ChA, Bzt, 2FA, 4TAA, 2TA, 3TA, and KZ₁;
X₁₁ is selected from the group consisting of S, αMeS, Aib, G, Dap, Ac5c, and Tle;
X₁₂ is selected from the group consisting of I and KZ₁;
X₁₃ is selected from the group consisting of αMeL and αMeF;
X₁₆ is Orn;
X₁₇ is selected from the group consisting of Q, I, and KZ₁;
X₂₀ is selected from the group consisting of Aib, Orn, 4Pal, αMeF, Ac5c, and Ac6c;
X₂₁ is selected from the group consisting of E, KZ₁, G, Orn, and 4Pal;
X₂₂ is selected from the group consisting of F, 2ClPhe, 3ClPhe, 2FPhe, 3FPhe, 3,5FPhe, 1Nal, 2Nal, αMeF(2F), ChA, Bzt, and αMeF;
X₂₄ is selected from the group consisting of D-Glu, E, G, and KZ₁;
X₂₅ is selected from the group consisting of Y, αMeY, αMeF, and KZ₁;
X₂₈ is selected from the group consisting of E, Orn, and KZ₁;
X₃₀ is selected from the group consisting of G, Orn, KZ₁, K(Z₁)R₆, OmR₂, and GR₂;
R₂ is selected from the group consisting of X₃₁, X₃₁SSG(SEQ ID NO:5), X₃₁SSG-R₃ (SEQ ID NO:6), X₃₁SSGX_{3S}PPPX₃₉ (SEQ ID NO:7), X₃₁SSGX₃₅PPPX₃₉R₃ (SEQ ID NO:8), X₃₁SSGX₃₅PPPX₃₉X₄₀ (SEQ ID NO:9), X₃₁SSGX_{3S}PPPX₃₉X₄₀R₃ (SEQ ID NO:10), and a modification of the c-terminal group wherein the modification is NH₂;
R₆ is selected from the group consisting of PSSG(SEQ ID NO:506), PSSG-R₃ (SEQ ID NO:507), PSSGX₃₅PPPX₃₉ (SEQ ID NO:508), PSSGX₃₅PPPX₃₉R₃ (SEQ ID NO:509), PSSGX₃₅PPPX₃₉X₄₀ (SEQ ID NO:510), PSSGX₃₅PPPX₃₉X₄₀R₃ (SEQ ID NO:511), and a modification of the c-terminal group wherein the modification is NH₂;
X₃₁ is selected from the group consisting of P and KZ₁;
X₃₅ is selected from the group consisting of A and Orn;
X₃₉ is selected from the group consisting of S and Orn;
X₄₀ is KZ₁,
R₃ is a modification of the C-terminal group, wherein the modification is NH₂; wherein two, and only two, of X₁₀, X₁₂, X₁₇, X₂₁, X₂₄, X₂₅, X₂₈, X₃₀, X₃₁, and X₄₀ are KZ₁ or K(Z₁)R₆;
Z₁ is selected from the group consisting of R₅ and -R₄R₅;
R₄ is a linker; and
R₅ is a fatty acid; or
a pharmaceutically acceptable salt thereof.

Provided is a compound of Formula II:

X₁X₂EGTX₆TSDX₁₀X₁₁X₁₂X₁₃LDX₁₆X₁₇AQX₂₀X₂₁X₂₂IX₂₄X₂₅
LIX₂₈GX₃₀ (SEQID NO:4)

wherein
X₁ is selected from the group consisting of Y and R₁Y;
R₁ is an Ac modification of the N-terminal amino group;
X₂ is Aib;
X₆ is selected from the group consisting of αMeF and αMeF(2F);
X₁₀ is selected from the group consisting of 4Pal, Y, αMeF, αMeF(2F), αMeL, αMeV, Ac4c, Ac5c, Ac6c, Bip, 1Nal, 2Nal, OMeY, hTyr, Nle, V, 4CPhe, ChG, ChA, Bzt, 2FA, 4TAA, 2TA, 3TA, and KZ₁;
X₁₁ is selected from the group consisting of Ac5c, S, αMeS, Aib, G, Dap, and Tle;
X₁₂ is selected from the group consisting of I and KZ₁;
X₁₃ is selected from the group consisting of αMeL and αMeF;
X₁₆ is Orn;
X₁₇ is selected from the group consisting of Q, I, and KZ₁;
X₂₀ is selected from the group consisting of Aib, Orn, 4Pal, αMeF, Ac5c, and Ac6c;
X₂₁ is selected from the group consisting of E, KZ₁, G, Orn, and 4Pal;
X₂₂ is selected from the group consisting of F, 2ClPhe, 3ClPhe, 2FPhe, 3FPhe, 3,5FPhe, 1Nal, 2Nal, αMeF(2F), ChA, Bzt, and αMeF;
X₂₄ is selected from the group consisting of D-Glu, E, G, and KZ₁;
X₂₅ is selected from the group consisting of Y, αMeY, αMeF, and KZ₁;
X₂₈ is selected from the group consisting of E, Orn, and KZ₁;
X₃₀ is selected from the group consisting of G, Orn, KZ₁, and GR₂;
R₂ is selected from the group consisting of X₃₁SSG (SEQ ID NO:5), X₃₁SSG-R₃ (SEQ ID NO:6), X₃₁SSGX₃₅PPPX₃₉ (SEQ ID NO:7), X₃₁SSGX₃₅PPPX₃₉R₃ (SEQ ID NO:8), X₃₁SSGX₃₅PPPX₃₉X₄₀ (SEQ ID NO:9), X₃₁SSGX_{3S}PPPX₃₉X₄₀R₃ (SEQ ID NO: 10), and a modification of the c-terminal group wherein the modification is NH₂;
X₃₁ is selected from the group consisting of P and KZ₁;
X₃₅ is selected from the group consisting of A and Orn;
X₃₉ is selected from the group consisting of S and Orn;
X₄₀ is KZ₁,
R₃ is a modification of the C-terminal group, wherein the modification is NH₂; wherein two, and only two, of X₁₀, X₁₂, X₁₇, X₂₁, X₂₄, X₂₅, X₂₈, X₃₀, X₃₁, and X₄₀ are KZ₁;
Z₁ is selected from the group consisting of R₅ and -R₄R₅; and
R₄ is a linker;
R₅ is a fatty acid; or
a pharmaceutically acceptable salt thereof.

Provided is a peptide of Formula I, or a pharmaceutically acceptable salt thereof, wherein Z₁ is selected from the group consisting of
-(2-[2-(2-Amino-ethoxy)-ethoxy]-acetyl)₂-(γ-Glu)-CO-(CH₂)₁₂-CO₂H,
-(2-[2-(2-Amino-ethoxy)-ethoxy]-acetyl)₂-(γ-Glu)-CO-(CH₂)₁₀-CO₂H,
-(2-[2-(2-Amino-ethoxy)-ethoxy]-acetyl)₂-(y-Glu)-(10-(4-carboxyphenoxy)decanoyl),
-(2-[2-(2-Amino-ethoxy)-ethoxy]-acetyl)₂-(γ-Glu)-(4-(4-iodophenyl)butanoyl),
-(2-[2-(2-Amino-ethoxy)-ethoxy]-acetyl)₂-(γ-Glu)-(4-(4-tert-butylphenyl)butanoyl),
-(2-[2-(2-Amino-ethoxy)-ethoxy]-acetyl)₂-(γ-Glu)-CO-(CH₂)₁₄-CH₃,
-(2-[2-(2-Amino-ethoxy)-ethoxy]-acetyl)₂-(γ-Glu)-CO-(CH₂)₁₂-CH₃,
-(2-[2-(2-Amino-ethoxy)-ethoxy]-acetyl)₂-(γ-Glu)-CO-(CH₂)io-CH₃,
-(2-[2-(2-Amino-ethoxy)-ethoxy]-acetyl)₂-CO-(CH₂)₁₂-CO₂H,
-(2-[2-(2-Amino-ethoxy)-ethoxy]-acetyl)₂-(10-(4-carboxyphenoxy)decanoyl),
-(2-[2-(2-Amino-ethoxy)-ethoxy]-acetyl)-(γ-Glu)-CO-(CH₂)₁₂-CO₂H,
-(2-[2-(2-Amino-ethoxy)-ethoxy]-acetyl)-(γ-Glu)-(10-(4-carboxyphenoxy)decanoyl),
-(2-[2-(2-Amino-ethoxy)-ethoxy]-acetyl)-(γ-Glu)-CO-(CH₂)₁₄-CH₃,
-(2-[2-(2-Amino-ethoxy)-ethoxy]-acetyl)-(γ-Glu)-CO-(CH₂)io-CH₃,
-(2-[2-(2-Amino-ethoxy)-ethoxy]-acetyl)₃-(γ-Glu)-CO-(CH₂)₁₂-CO₂H,
-(2-[2-(2-Amino-ethoxy)-ethoxy]-acetyl)₃-(γ-Glu)-CO-(CH₂)₁₄-CH₃,
-(2-[2-(2-Amino-ethoxy)-ethoxy]-acetyl)₂-(εK)-CO-(CH₂)₁₂-CO₂H,
-(2-[2-(2-Amino-ethoxy)-ethoxy]-acetyl)₂-(εK)-(γ-Glu)-CO-(CH₂)₁₂-CO₂H,
-(2-[2-(2-Amino-ethoxy)-ethoxy]-acetyl)-(εK)-(γ-Glu)-CO-(CH₂)₁₂-CO₂H,
-(εK)-(2-[2-(2-Amino-ethoxy)-ethoxy]-acetyl)-(γ-Glu)-CO-(CH₂)₁₂-CO₂H,
-(εK)-(2-[2-(2-Amino-ethoxy)-ethoxy]-acetyl)₂-(y-Glu)-CO-(CH₂)₁₂-CO₂H,
-(εK)-(γ-Glu)-CO-(CH₂)₁₂-CO₂H,
-(εK)-(γ-Glu)-(γ-Glu)-CO-(CH₂)₁₂-CO₂H,
-(γ-Glu)-(2-[2-(2-Amino-ethoxy)-ethoxy]-acetyl)₂-CO-(CH₂)₁₂-CO₂H,
-(εK)-(εK)-(γ-Glu)-CO-(CH₂)₁₂-CO₂H,
-(γ-Glu)-(2-[2-(2-Amino-ethoxy)-ethoxy]-acetyl)-CO-(CH₂)₁₂-CO₂H,
-(2-[2-(2-Amino-ethoxy)-ethoxy]-acetyl)-(γ-Glu)-(2-[2-(2-Amino-ethoxy)-ethoxy]-acetyl)-CO-(CH₂)₁₂-CO₂H,
-(2-[2-(2-Amino-ethoxy)-ethoxy]-acetyl)₂-(γ-Glu)-(7-(4-carboxyphenoxy)heptanoyl),
-(2-[2-(2-Amino-ethoxy)-ethoxy]-acetyl)₂-(γ-Glu)-(8-(4-carboxyphenoxy)octanoyl),
-(2-[2-(2-Amino-ethoxy)-ethoxy]-acetyl)-(γ-Glu)-(4-(4-tert-butylphenyl)butanoyl),
-(2-[2-(2-Amino-ethoxy)-ethoxy]-acetyl)₂-(γ-Glu)-(ll-(4-carboxyphenoxy)undecanoyl),
-(2-[2-(2-Amino-ethoxy)-ethoxy]-acetyl)₃-(γ-Glu)-(10-(4-carboxyphenoxy)decanoyl),
-(2-[2-(2-Amino-ethoxy)-ethoxy]-acetyl)₂-(γ-Glu)-(2-[2-(2-Amino-ethoxy)-ethoxy]-acetyl)-CO-(CH₂)₁₂-CO₂H,
-(2-[2-(2-Amino-ethoxy)-ethoxy]-acetyl)-(y-Glu)-(2-[2-(2-Amino-ethoxy)-ethoxy]-acetyl)₂-CO-(CH₂)₁₂-CO₂H,
-PEG3-(γ-Glu)-CO-(CH₂)₁₂-CO₂H,
-PEG4-(γ-Glu)-CO-(CH₂)₁₂-CO₂H,
-(2-[2-(2-Amino-ethoxy)-ethoxy]-acetyl)₃-(γ-Glu)-CO-(CH₂)₁₀-CH₃,
-(2-[2-(2-Amino-ethoxy)-ethoxy]-acetyl)₂-(γ-Glu)-(11-(4-carboxyphenoxy)undecanoyl,
-(2-[2-(2-Amino-ethoxy)-ethoxy]-acetyl)₂-(γ-Glu)-CO-(CH₂)₁₄-CO₂H,
-(2-[2-(2-Amino-ethoxy)-ethoxy]-acetyl)-Ahx-(γ-Glu)-CO-(CH₂)₁₂-CO₂H,
-(2-[2-(2-Amino-ethoxy)-ethoxy]-acetyl)-Aoc-(γ-Glu)-CO-(CH₂)₁₂-CO₂H,
-Ahx-(2-[2-(2-Amino-ethoxy)-ethoxy]-acetyl)-(γ-Glu)-CO-(CH₂)₁₂-CO₂H,
-Aoc-(2-[2-(2-Amino-ethoxy)-ethoxy]-acetyl)-(γ-Glu)-CO-(CH₂)₁₂-CO₂H,
-(2-[2-(2-Amino-ethoxy)-ethoxy]-acetyl)-Ahx-(γ-Glu)-(10-(4-carboxyphenoxy)decanoyl),
-(2-[2-(2-Amino-ethoxy)-ethoxy]-acetyl)-Aoc-(γ-Glu)-(10-(4-carboxyphenoxy)decanoyl),
-Ahx-(2-[2-(2-Amino-ethoxy)-ethoxy]-acetyl)-(γ-Glu)-(10-(4-carboxyphenoxy)decanoyl),
-Aoc-(2-[2-(2-Amino-ethoxy)-ethoxy]-acetyl)-(γ-Glu)-(10-(4-carboxyphenoxy)decanoyl),
-PEG4-(γ-Glu)-(10-(4-carboxyphenoxy)decanoyl),
-PEG3-(γ-Glu)-(10-(4-carboxyphenoxy)decanoyl),
-(2-[2-(2-Amino-ethoxy)-ethoxy]-acetyl)-Aoc-CO-(CH₂)₁₂-CO₂H,
-PEG6-(γ-Glu)-CO-(CH₂)₁₂-CO₂H,
-PEG5-(γ-Glu)-CO-(CH₂)₁₂-CO₂H,
-PEG6-(γ-Glu)-(10-(4-carboxyphenoxy)decanoyl),
-PEG5-(γ-Glu)-(10-(4-carboxyphenoxy)decanoyl),
-(2-[2-(2-Amino-ethoxy)-ethoxy]-acetyl)₂-Trx-(γ-Glu)-CO-(CH₂)₁₂-CO₂H,
-(2-[2-(2-Amino-ethoxy)-ethoxy]-acetyl)₂-Trx-(γ-Glu)-(10-(4-carboxyphenoxy)decanoyl),
-(2-[2-(2-Amino-ethoxy)-ethoxy]-acetyl)-Trx-(γ-Glu)-CO-(CH₂)₁₂-CO₂H,
-(2-[2-(2-Amino-ethoxy)-ethoxy]-acetyl)-Trx-(γ-Glu)-(10-(4-carboxyphenoxy)decanoyl),
-(2-[2-(2-Amino-ethoxy)-ethoxy]-acetyl)₂-(γ-Glu)₂-CO-(CH₂)₁₂-CO₂H,
-(2-[2-(2-Amino-ethoxy)-ethoxy]-acetyl)₂-(γ-Glu)₂-(10-(4-carboxyphenoxy)decanoyl),
-(γ-Glu)-(2-[2-(2-Amino-ethoxy)-ethoxy]-acetyl)₂-(γ-Glu)-CO-(CH₂)₁₂-CO₂H,
-(γ-Glu)-(2-[2-(2-Amino-ethoxy)-ethoxy]-acetyl)₂-(γ-Glu)-(10-(4-carboxyphenoxy)decanoyl),
-(2-[2-(2-Amino-ethoxy)-ethoxy]-acetyl)-(γ-Glu)-(2-[2-(2-Amino-ethoxy)-ethoxy]-acetyl)-(γ-Glu)-CO-(CH₂)₁₂-CO₂H, and
-(2-[2-(2-Amino-ethoxy)-ethoxy]-acetyl)-(γ-Glu)-(2-[2-(2-Amino-ethoxy)-ethoxy]-acetyl)-(γ-Glu)-(10-(4-carboxyphenoxy)decanoyl).

Provided is a peptide of Formula I, or a pharmaceutically acceptable salt thereof, wherein Z₁ is selected from the group consisting of
-(2-[2-(2-Amino-ethoxy)-ethoxy]-acetyl)₂-(γ-Glu)-CO-(CH₂)₁₂-CO₂H,
-(2-[2-(2-Amino-ethoxy)-ethoxy]-acetyl)₂-(γ-Glu)-CO-(CH₂)₁₀-CO₂H,
-(2-[2-(2-Amino-ethoxy)-ethoxy]-acetyl)₂-(γ-Glu)-(10-(4-carboxyphenoxy)decanoyl),
-(2-[2-(2-Amino-ethoxy)-ethoxy]-acetyl)₂-(γ-Glu)-(4-(4-iodophenyl)butanoyl),
-(2-[2-(2-Amino-ethoxy)-ethoxy]-acetyl)₂-(γ-Glu)-(4-(4-tert-butylphenyl)butanoyl),
-(2-[2-(2-Amino-ethoxy)-ethoxy]-acetyl)₂-(γ-Glu)-CO-(CH₂)₁₄-CH₃,
-(2-[2-(2-Amino-ethoxy)-ethoxy]-acetyl)₂-(γ-Glu)-CO-(CH₂)₁₂-CH₃,
-(2-[2-(2-Amino-ethoxy)-ethoxy]-acetyl)₂-(γ-Glu)-CO-(CH₂)io-CH₃,
-(2-[2-(2-Amino-ethoxy)-ethoxy]-acetyl)₂-CO-(CH₂)₁₂-CO₂H,
-(2-[2-(2-Amino-ethoxy)-ethoxy]-acetyl)₂-(10-(4-carboxyphenoxy)decanoyl),
-(2-[2-(2-Amino-ethoxy)-ethoxy]-acetyl)-(γ-Glu)-CO-(CH₂)₁₂-CO₂H,
-(2-[2-(2-Amino-ethoxy)-ethoxy]-acetyl)-(γ-Glu)-(10-(4-carboxyphenoxy)decanoyl),
-(2-[2-(2-Amino-ethoxy)-ethoxy]-acetyl)-(γ-Glu)-CO-(CH₂)₁₄-CH₃,
-(2-[2-(2-Amino-ethoxy)-ethoxy]-acetyl)-(γ-Glu)-CO-(CH₂)io-CH₃,
-(2-[2-(2-Amino-ethoxy)-ethoxy]-acetyl)₃-(γ-Glu)-CO-(CH₂)₁₂-CO₂H,
-(2-[2-(2-Amino-ethoxy)-ethoxy]-acetyl)₃-(γ-Glu)-CO-(CH₂)₁₄-CH₃,
-(2-[2-(2-Amino-ethoxy)-ethoxy]-acetyl)₂-(εK)-CO-(CH₂)₁₂-CO₂H,
-(2-[2-(2-Amino-ethoxy)-ethoxy]-acetyl)₂-(εK)-(γ-Glu)-CO-(CH₂)₁₂-CO₂H,
-(2-[2-(2-Amino-ethoxy)-ethoxy]-acetyl)-(εK)-(γ-Glu)-CO-(CH₂)₁₂-CO₂H,
-(εK)-(2-[2-(2-Amino-ethoxy)-ethoxy]-acetyl)-(γ-Glu)-CO-(CH₂)₁₂-CO₂H,
-(sK)-(2-[2-(2-Amino-ethoxy)-ethoxy]-acetyl)₂-(γ-Glu)-CO-(CH₂)i₂-CO₂H,
-(εK)-(γ-Glu)-CO-(CH₂)₁₂-CO₂H,
-(εK)-(γ-Glu)-(γ-Glu)-CO-(CH₂)₁₂-CO₂H,
-(γ-Glu)-(2-[2-(2-Amino-ethoxy)-ethoxy]-acetyl)₂-CO-(CH₂)₁₂-CO₂H,
-(εK)-(εK)-(γ-Glu)-CO-(CH₂)₁₂-CO₂H,
-(γ-Glu)-(2-[2-(2-Amino-ethoxy)-ethoxy]-acetyl)-CO-(CH₂)₁₂-CO₂H
-(2-[2-(2-Amino-ethoxy)-ethoxy]-acetyl)-(γ-Glu)-(2-[2-(2-Amino-ethoxy)-ethoxy]-acetyl)-CO-(CH₂)₁₂-CO₂H,
-(2-[2-(2-Amino-ethoxy)-ethoxy]-acetyl)₂-(γ-Glu)-(7-(4-carboxyphenoxy)heptanoyl),
-(2-[2-(2-Amino-ethoxy)-ethoxy]-acetyl)₂-(γ-Glu)-(8-(4-carboxyphenoxy)octanoyl), and
-(2-[2-(2-Amino-ethoxy)-ethoxy]-acetyl)-(γ-Glu)-(4-(4-tert-butylphenyl)butanoyl).

Provided is a peptide of Formula I, or a pharmaceutically acceptable salt thereof, wherein Z₁ is selected from the group consisting of
-(2-[2-(2-Amino-ethoxy)-ethoxy]-acetyl)₂-(γ-Glu)-CO-(CH₂)₁₂-CO₂H,
-(2-[2-(2-Amino-ethoxy)-ethoxy]-acetyl)₂-(γ-Glu)-CO-(CH₂)₁₀-CO₂H,
-(2-[2-(2-Amino-ethoxy)-ethoxy]-acetyl)₂-(γ-Glu)-(10-(4-carboxyphenoxy)decanoyl),
-(2-[2-(2-Amino-ethoxy)-ethoxy]-acetyl)₂-(γ-Glu)-(4-(4-iodophenyl)butanoyl),
-(2-[2-(2-Amino-ethoxy)-ethoxy]-acetyl)₂-(γ-Glu)-(4-(4-tert-butylphenyl)butanoyl),
-(2-[2-(2-Amino-ethoxy)-ethoxy]-acetyl)₂-(γ-Glu)-CO-(CH₂)₁₄-CH₃,
-(2-[2-(2-Amino-ethoxy)-ethoxy]-acetyl)₂-(γ-Glu)-CO-(CH₂)₁₂-CH₃,
-(2-[2-(2-Amino-ethoxy)-ethoxy]-acetyl)₂-(γ-Glu)-CO-(CH₂)io-CH₃,
-(2-[2-(2-Amino-ethoxy)-ethoxy]-acetyl)₂-CO-(CH₂)₁₂-CO₂H,
-(2-[2-(2-Amino-ethoxy)-ethoxy]-acetyl)₂-(10-(4-carboxyphenoxy)decanoyl),
-(2-[2-(2-Amino-ethoxy)-ethoxy]-acetyl)-(γ-Glu)-CO-(CH₂)₁₂-CO₂H,
-(2-[2-(2-Amino-ethoxy)-ethoxy]-acetyl)-(γ-Glu)-(10-(4-carboxyphenoxy)decanoyl),
-(2-[2-(2-Amino-ethoxy)-ethoxy]-acetyl)-(γ-Glu)-CO-(CH₂)₁₄-CH₃,
-(2-[2-(2-Amino-ethoxy)-ethoxy]-acetyl)-(γ-Glu)-CO-(CH₂)io-CH₃,
-(2-[2-(2-Amino-ethoxy)-ethoxy]-acetyl)₃-(γ-Glu)-CO-(CH₂)₁₂-CO₂H,
-(2-[2-(2-Amino-ethoxy)-ethoxy]-acetyl)₃-(γ-Glu)-CO-(CH₂)₁₄-CH₃,
-(2-[2-(2-Amino-ethoxy)-ethoxy]-acetyl)₂-(εK)-CO-(CH₂)₁₂-CO₂H,
-(2-[2-(2-Amino-ethoxy)-ethoxy]-acetyl)₂-(εK)-(γ-Glu)-CO-(CH₂)₁₂-CO₂H,
-(2-[2-(2-Amino-ethoxy)-ethoxy]-acetyl)-(εK)-(γ-Glu)-CO-(CH₂)₁₂-CO₂H,
-(εK)-(2-[2-(2-Amino-ethoxy)-ethoxy]-acetyl)-(γ-Glu)-CO-(CH₂)₁₂-CO₂H,
-(sK)-(2-[2-(2-Amino-ethoxy)-ethoxy]-acetyl)₂-(y-Glu)-CO-(CH₂)i₂-CO₂H,
-(εK)-(γ-Glu)-CO-(CH₂)₁₂-CO₂H,
-(εK)-(γ-Glu)-(γ-Glu)-CO-(CH₂)₁₂-CO₂H,
-(γ-Glu)-(2-[2-(2-Amino-ethoxy)-ethoxy]-acetyl)₂-CO-(CH₂)₁₂-CO₂H,
-(εK)-(εK)-(γ-Glu)-CO-(CH₂)₁₂-CO₂H,
-(γ-Glu)-(2-[2-(2-Amino-ethoxy)-ethoxy]-acetyl)-CO-(CH₂)₁₂-CO₂H,
-(2-[2-(2-Amino-ethoxy)-ethoxy]-acetyl)-(γ-Glu)-(2-[2-(2-Amino-ethoxy)-ethoxy]-acetyl)-CO-(CH₂)₁₂-CO₂H,
-(2-[2-(2-Amino-ethoxy)-ethoxy]-acetyl)₂-(γ-Glu)-(7-(4-carboxyphenoxy)heptanoyl),
-(2-[2-(2-Amino-ethoxy)-ethoxy]-acetyl)₂-(γ-Glu)-(8-(4-carboxyphenoxy)octanoyl), and
-(2-[2-(2-Amino-ethoxy)-ethoxy]-acetyl)-(γ-Glu)-(4-(4-tert-butylphenyl)butanoyl).

Provided is a compound of Formula I, or a pharmaceutically acceptable salt thereof, wherein R₅ is selected from the group consisting of —CO—(CH₂)₁₂—CO₂H, —CO—(CH₂)₁₀—CO₂H, -(10-(4-carboxyphenoxy)decanoyl), -(4-(4-iodophenyl)butanoyl), -(4-(4-tert-butylphenyl)butanoyl), —CO—(CH₂)₁₄—CH₃, —CO—(CH₂)₁₂—CH₃, —CO—(CH₂)₁₀—CH₃, -(7-(4-carboxyphenoxy)heptanoyl), -(8-(4-carboxyphenoxy)octanoyl), (11-(4-carboxyphenoxy)undecanoyl), -(12-(4-carboxyphenoxy)dodecanoyl), and CO—(CH₂)₁₁—CO₂H.

Provided is a compound of Formula I, or a pharmaceutically acceptable salt thereof, wherein R₅ is selected from the group consisting of —CO—(CH₂)₁₂—CO₂H, —CO—(CH₂)₁₀—CO₂H, -(10-(4-carboxyphenoxy)decanoyl), -(4-(4-iodophenyl)butanoyl), -(4-(4-tert-butylphenyl)butanoyl), —CO—(CH₂)₁₄—CH₃, —CO—(CH₂)₁₂—CH₃, —CO—(CHz)₁₀—CH₃, -(7-(4-carboxyphenoxy)heptanoyl), and -(8-(4-carboxyphenoxy)octanoyl).

Provided is a compound of Formula I, or a pharmaceutically acceptable salt thereof, wherein R₄ is selected from the group consisting of
-(2-[2-(2-Amino-ethoxy)-ethoxy]-acetyl)₂-(γ-Glu)-,
-(2-[2-(2-Amino-ethoxy)-ethoxy]-acetyl)₂-,
-(2-[2-(2-Amino-ethoxy)-ethoxy]-acetyl)-(γ-Glu)-,
-(2-[2-(2-Amino-ethoxy)-ethoxy]-acetyl)₂-(γ-Glu)₂)-,
-(2-[2-(2-Amino-ethoxy)-ethoxy]-acetyl)₃-(γ-Glu)-,
-(2-[2-(2-Amino-ethoxy)-ethoxy]-acetyl)₂-(εK)-,
-(2-[2-(2-Amino-ethoxy)-ethoxy]-acetyl)₂-(εK)-(γ-Glu)-,
-(2-[2-(2-Amino-ethoxy)-ethoxy]-acetyl)-(εK)-(γ-Glu)-,
-(2-[2-(2-Amino-ethoxy)-ethoxy]-acetyl)-Trx-(γ-Glu)-,
-(2-[2-(2-Amino-ethoxy)-ethoxy]-acetyl)₂-Trx-(γ-Glu)-,
-(2-[2-(2-Amino-ethoxy)-ethoxy]-acetyl)-Aoc-,
-(2-[2-(2-Amino-ethoxy)-ethoxy]-acetyl)-Aoc-(γ-Glu)-,
-(2-[2-(2-Amino-ethoxy)-ethoxy]-acetyl)-Ahx-(γ-Glu)-,
-PEG3-(γ-Glu)-,
-PEG4-(γ-Glu)-,
-PEG5-(γ-Glu)-,
-PEG6-(γ-Glu)-,
-Ahx-(2-[2-(2-Amino-ethoxy)-ethoxy]-acetyl)-(γ-Glu)-,
-Aoc-(2-[2-(2-Amino-ethoxy)-ethoxy]-acetyl)-(γ-Glu)-,
-(εK)-(2-[2-(2-Amino-ethoxy)-ethoxy]-acetyl)-(γ-Glu)-,
-(εK)-(2-[2-(2-Amino-ethoxy)-ethoxy]-acetyl)₂-(γ-Glu)-,
-(εK)-(γ-Glu)-,
-(εK)-(γ-Glu)-(γ-Glu)-,
-(γ-Glu)-(2-[2-(2-Amino-ethoxy)-ethoxy]-acetyl)₂-,
-(εK)-(eK)-(γ-Glu)-,
-(γ-Glu)-(2-[2-(2-Amino-ethoxy)-ethoxy]-acetyl)-,
-(γ-Glu)-(2-[2-(2-Amino-ethoxy)-ethoxy]-acetyl)₂-(γ-Glu)-,
-(2-[2-(2-Amino-ethoxy)-ethoxy]-acetyl)-(γ-Glu)-(2-[2-(2-Amino-ethoxy)-ethoxy]-acetyl)-,
-(2-[2-(2-Amino-ethoxy)-ethoxy]-acetyl)₂-(γ-Glu)-(2-[2-(2-Amino-ethoxy)-ethoxy]-acetyl)-,
-(2-[2-(2-Amino-ethoxy)-ethoxy]-acetyl)-(γ-Glu)-(2-[2-(2-Amino-ethoxy)-ethoxy]-acetyl)₂-, and
(2-[2-(2-Amino-ethoxy)-ethoxy]-acetyl)-(γ-Glu)-(2-[2-(2-Amino-ethoxy)-ethoxy]-acetyl)-(γ-Glu)-.

Provided is a compound of Formula I, or a pharmaceutically acceptable salt thereof, wherein R₄ is selected from the group consisting of
-(2-[2-(2-Amino-ethoxy)-ethoxy]-acetyl)₂-(γ-Glu)-.
-(2-[2-(2-Amino-ethoxy)-ethoxy]-acetyl)₂-,
-(2-[2-(2-Amino-ethoxy)-ethoxy]-acetyl)-(γ-Glu)-,
-(2-[2-(2-Amino-ethoxy)-ethoxy]-acetyl)₃-(γ-Glu)-,
-(2-[2-(2-Amino-ethoxy)-ethoxy]-acetyl)₂-(εK)-,
-(2-[2-(2-Amino-ethoxy)-ethoxy]-acetyl)₂-(εK)-(y-Glu)-,
-(2-[2-(2-Amino-ethoxy)-ethoxy]-acetyl)-(εK)-(γ-Glu)-,
-(εK)-(2-[2-(2-Amino-ethoxy)-ethoxy]-acetyl)-(γ-Glu)-,
-(εK)-(2-[2-(2-Amino-ethoxy)-ethoxy]-acetyl)₂-(γ-Glu)-,
-(εK)-(γ-Glu)-,
-(εK)-(γ-Glu)-(γ-Glu)-,
-(γ-Glu)-(2-[2-(2-Amino-ethoxy)-ethoxy]-acetyl)₂-,
-(εK)-(εK)-(γ-Glu)-,
-(y-Glu)-(2-[2-(2-Amino-ethoxy)-ethoxy]-acetyl)-, and
-(2-[2-(2-Amino-ethoxy)-ethoxy]-acetyl)-(y-Glu)-(2-[2-(2-Amino-ethoxy)-ethoxy]-acetyl)-.

Provided is a compound of Formula I, or a pharmaceutically acceptable salt thereof, wherein X₁₇ and X₃₁ are each KZ₁. Provided is a compound of Formula I, or a pharmaceutically acceptable salt thereof, wherein X₁₇ and X₂₄ are each KZ₁. Provided is a compound of Formula I, or a pharmaceutically acceptable salt thereof, wherein X₁₇ and X₂₁ are each KZ₁. Provided is a compound of Formula I, or a pharmaceutically acceptable salt thereof, wherein X₁₇ and X₂₈ are each KZ₁. Provided is a compound of Formula I, or a pharmaceutically acceptable salt thereof, wherein X₁₇ and X₄₀ are each KZ₁. Provided is a compound of Formula I, or a pharmaceutically acceptable salt thereof, wherein X₂₁ and X₄₀ are each KZ₁. Provided is a compound of Formula I, or a pharmaceutically acceptable salt thereof, wherein X₂₁ and X₂₈ are each KZ₁. Provided is a compound of Formula I, or a pharmaceutically acceptable salt thereof, wherein X₂₄ and X₂₈ are each KZ₁.

Provided is a compound of Formula I, or a pharmaceutically acceptable salt thereof, wherein X₁ is Y; X₆ is αMeF(2F);X₁₀ is selected from the group consisting of 4Pal, Y, and KZ₁; X₁₁ is selected from the group consisting of S, αMeS, and Aib; X₁₂ is I; X₁₃ is αMeL; X₁₆ is Orn; X₁₇ is selected from the group consisting of I and KZ₁; X₂₀ is Aib; X₂₁ is selected from the group consisting of KZ₁ and E; X₂₂ is selected from the group consisting of F and αMeF;X₂₄ is selected from the group consisting of D-Glu, and KZ₁; X₂₅ is αMeY; X₂₈ is selected from the group consisting of E and KZ₁; X₃₀ is selected from the group consisting of G and GR₂; R₂ is selected from the group consisting of X₃₁SSGX₃₅PPPX₃₉ (SEQ ID NO:7), X₃₁SSGX₃₅PPPX₃₉R₃ (SEQ ID NO:8), and X₃₁SSGX₃₅PPPX₃₉X₄₀ (SEQ ID NO:9), and a modification of the c-terminal group wherein the modification is NH₂; X₃₁ is selected from the group consisting of P and KZ₁; X₃₅ is selected from the group consisting of A and Orn; X₃₉ is selected from the group consisting of S, Orn; X₄₀ is selected from the group consisting of KZ₁; R₃ is a modification of the C-terminal group, wherein the modification is NH₂; wherein one, and only one, selected from the group consisting of X₁₀, X₁₇, X₂₁, X₂₄, X₂₈, and X₃₁ is KZ₁; Z₁ is -R₄R₅; R₄ is a linker; and R₅ is a fatty acid; or a pharmaceutically acceptable salt thereof.

Provided is a compound of Formula I, or a pharmaceutically acceptable salt thereof, wherein the compound is selected from the group consisting of
Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂,
Y-Aib-EGT-αMeF(2F)-TSD-4Pal-SI-αMeL-LD-Om-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIEGGK((2-[2-(2-Amino-ethoxy)-ethoxy]-acetyl)₂-(γ-Glu)-(10-(4-carboxyphenoxy)decanoyl))SSGAPPPS-NH₂,
Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(4-(4-iodophenyl)butanoyl))AQ-Aib-EFI-(D-Glu)-αMeY-LIEGGK((2-[2-(2-Amino-ethoxy)-ethoxy]-acetyl)₂-(γ-Glu)-(4-(4-iodophenyl)butanoyl))SSGAPPPS-NH₂,
Y-Aib-EGT-αMeF(2F)-TSD-4Pal-SI-aMeL-LD-Orn-K((2-[2-(2-Amino-ethoxy)-ethoxy]-acetyl)₂-(γ-Glu)-(4-(4-tert-butylphenyl)butanoyl))AQ-Aib-EFI-(D-Glu)-αMeY-LIEGGK((2-[2-(2-Amino-ethoxy)-ethoxy]-acetyl)₂-(γ-Glu)-(4-(4-tert-butylphenyl)butanoyl))SSGAPPPS-NH₂, and
Y-Aib-EGT-αMeF(2F)-TSD-4Pal-SI-αMeL-LD-Om-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGGK((2-[2-(2-Amino-ethoxy)-ethoxy]-acetyl)₂-(γ-Glu)-CO-(CH₂)₁₀-CH₃)SSGAPPPS-NH₂

In an embodiment the R₄ linker is one to two amino acids selected from the group consisting of εK and γ-Glu. In an embodiment the R₄ linker comprises from one to three (2-[2-(2-Amino-ethoxy)-ethoxy]-acetyl) moieties. In an embodiment, the R₅ fatty acid moieties are conjugated to a lysine via an R₄ linker between the lysine and the R₅ fatty acid.

In an embodiment is a compound of Formula I, or a pharmaceutically acceptable salt thereof, wherein the R₄ linker comprises from zero to four amino acids; and from zero to three (2-[2-(2-Amino-ethoxy)-ethoxy]-acetyl) moieties. In an embodiment, the R₄ linker comprises from one to three amino acids each independently selected from the group consisting of εK and γ-Glu. In an embodiment, is a compound of Formula I, or a pharmaceutically acceptable salt thereof, wherein the R₄ linker comprises from 1 to 2 amino acids each independently selected from the group consisting of εK and γ-Glu. In an embodiment is a compound of Formula I, or a pharmaceutically acceptable salt thereof, comprising two Z₁ fatty acid moieties wherein each R₅ fatty acid of the Z₁ moiety is conjugated to different lysines of the peptide via an R₄ linker wherein, the R₄ linker comprises from zero to 2 γ-Glu amino acid residues. In an embodiment is a compound of Formula I, or a pharmaceutically acceptable salt thereof, comprising two Z₁ fatty acid moieties wherein each R₅ fatty acid of the Z₁ is conjugated to different lysines of the peptide via an R₄ linker wherein R₄ comprises from one to three amino acids and from one to three (2-[2-(2-Amino-ethoxy)-ethoxy]-acetyl) moieties. In an embodiment is a compound of Formula I, or a pharmaceutically acceptable salt thereof, comprising two of the same Z₁ fatty acid moieties wherein the R₅ fatty acid of the Z₁ is each conjugated to a different lysine of the peptide via an R₄ linker wherein, R₄ comprises from one to three amino acids each independently selected from the group consisting of εK and ɤ-Glu; and from one to three (2-[2-(2-Amino-ethoxy)-ethoxy]-acetyl) moieties attached to the amino acid. In an embodiment is a Formula I compound, or a pharmaceutically acceptable salt thereof, comprising two of the same Z₁ fatty acid moieties wherein the R₅ fatty acid of the Z₁ is each conjugated to different lysines of the peptide via an R₄ linker wherein R₄ comprises up to three amino acids each independently selected from the group consisting of γ-Glu and εK attached to one or two (2-[2-(2-Amino-ethoxy)-ethoxy]-acetyl) moieties.

In an embodiment is a Formula I compound, or a pharmaceutically acceptable salt thereof, comprising two of the same Z₁ fatty acid moieties wherein the R₅ fatty acid of the Z₁ is each conjugated via an R₄ linker, wherein the R₄ linker has the following formula:
-(εK)ₐ₁-(γ-Glu)ₐ₂-(2-[2-(2-Amino-ethoxy)-ethoxy]-acetyl)ₐ₃-(εK)_{b1}-(γ-Glu)_{b2}-;
wherein a1 is selected from the group consisting of 0, 1, and 2; a2 is selected from the group consisting of 0, 1, and 2; a3 is selected from the group consisting of 0, 1, 2, and 3; b1 is 0 or 1; and b2 is 0 or 1. In an embodiment is a Formula I compound, or a pharmaceutically acceptable salt thereof, comprising two of the same Z₁ fatty acid moieties wherein the R₅ fatty acid of the Z₁ is each conjugated via an R₄ linker wherein Z₁ is of the formula:

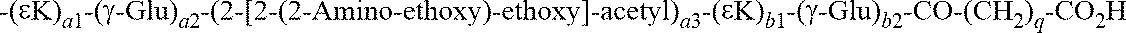

wherein wherein a1 is selected from the group consisting of 0, 1, and 2; a2 is selected from the group consisting of 0, 1, and 2; a3 is selected from the group consisting of 0, 1, 2, and 3; b1 is 0 or 1; b2 is 0 or 1; and q is selected from the group consisting of 10, 12, 14 and 16.

In an embodiment, a1 is 1, a2 is 0, a3 is 2, b1 is 0, b2 is 1, and q is 12; and the structure is:

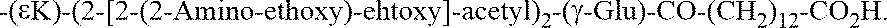

In an embodiment is a Formula I compound, or a pharmaceutically acceptable salt thereof, comprising two of the same Z₁ fatty acid moieties wherein the R₅ fatty acid of the Z₁ is each conjugated via an R₄ linker, wherein the R₄ linker and R₅ fatty acid components have the following formula:

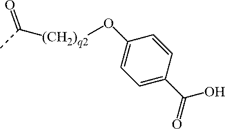

wherein q2 is selected from the group consisting of 7, 8, 10, 11, and 12.

In an embodiment is a Formula I compound, or a pharmaceutically acceptable salt thereof, comprising two of the same Z₁ fatty acid moieties wherein the R₅ fatty acid of the Z₁ is each conjugated via an R₄ linker, wherein the R₅ fatty acid is selected from the group consisting of -(7-(4-carboxyphenoxy)heptanoyl) and -(8-(4-carboxyphenoxy)octanoyl). In an embodiment is a Formula I compound, or a pharmaceutically acceptable salt thereof, comprising two of the same Z₁ fatty acid moieties each conjugated via an R₄ linker, wherein the R₅ fatty acid is selected from the group consisting of -(10-(4-carboxyphenoxy)decanoyl), -(4-(4-iodophenyl)butanoyl), and -(4-(4-tert-butylphenyl)butanoyl).

In an embodiment is a Formula I compound, or a pharmaceutically acceptable salt thereof, comprising two of the same Z₁ fatty acid moieties each conjugated via an R₄ linker, wherein the R₅ fatty acid is selected from the group consisting of —CO—(CH₂)₁₄—CH₃, —CO—(CH₂)₁₂—CH₃, and —CO—(CH₂)₁₀—CH₃.

In an embodiment is a Formula I compound, or a pharmaceutically acceptable salt thereof, comprising two of the same Z₁ fatty acid moieties each conjugated via an R₄ linker, wherein the R₅ fatty acid is selected from the group consisting of —CO—(CH₂)₁₂—CO₂H and —CO—(CH₂)₁₀—CO₂H. In an embodiment is a Formula I compound, or a pharmaceutically acceptable salt thereof, comprising two of the same Z₁ fatty acid moieties each conjugated via an R₄ linker, wherein the R₅ fatty acid is selected from the group consisting of —CO—(CH₂)₁₀—CH₃ and —CO—(CH₂)₁₂—CH₃.

In an embodiment, R₅ fatty acid is selected from the group consisting of —CO—(CH₂)₁₂—CO₂H, —CO—(CH₂)₁₀—CO₂H, -(10-(4-carboxyphenoxy)decanoyl), -(4-(4-iodophenyl)butanoyl), -(4-(4-tert-butylphenyl)butanoyl), —CO—(CH₂)₁₄—CH₃, —CO—(CH₂)₁₂—CH₃, —CO—(CH₂)₁₀—CH₃, -(7-(4-carboxyphenoxy)heptanoyl), and -(8-(4-carboxyphenoxy)octanoyl).

An embodiment provides a method of treating a condition selected from the group consisting of T2DM, obesity, nonalcoholic fatty liver disease (NAFLD), nonalcoholic steatohepatitis (NASH), dyslipidemia and metabolic syndrome, comprising administering to a subject in need thereof, an effective amount of a compound of Formula I or a pharmaceutically acceptable salt thereof. An embodiment provides a method for providing therapeutic weight loss comprising administering to a subject in need thereof, an effective amount of a compound of Formula I, or a pharmaceutically acceptable salt thereof. In one embodiment, the condition is NAFLD. In one embodiment, the condition is NASH.

An embodiment provides a compound of Formula I, or a pharmaceutically acceptable salt thereof, for use in therapy. An embodiment provides a compound of Formula I, or a pharmaceutically acceptable salt thereof, for use in the treatment of a condition selected from the group consisting of T2DM, obesity, NAFLD, NASH, dyslipidemia, and metabolic syndrome. In an embodiment, the condition is T2DM. In an embodiment, the condition is obesity. In an embodiment, the condition is NAFLD. In an embodiment, the condition is NASH. In an embodiment, the condition is metabolic syndrome.

The compounds of Formula I, or a pharmaceutically acceptable salt thereof, may be useful in the treatment of a variety of symptoms or disorders. For example, certain embodiments, provide a method for treatment of T2DM in a patient comprising administering to a subject in need of such treatment an effective amount of a compound of Formula I, or a pharmaceutically acceptable salt thereof. In an embodiment, is a method for treatment of obesity in a patient comprising administering to a subject in need of such treatment an effective amount of a compound of Formula I, or a pharmaceutically acceptable salt thereof. In an embodiment, the method is inducing non-therapeutic weight loss in a subject, comprising administering to a subject in need of such treatment an effective amount of a compound of Formula I, or a pharmaceutically acceptable salt thereof.

In certain embodiments, the present invention provides a method for treatment of metabolic syndrome in a patient comprising administering to a subject in need of such treatment an effective amount of a compound of Formula I, or a pharmaceutically acceptable salt thereof. In an embodiment, the method is treatment of NASH comprising administering to a subject in need of such treatment an effective amount of a compound of Formula I, or a pharmaceutically acceptable salt thereof.

Also provided herein is a compound of the present invention for use in simultaneous, separate and sequential combinations with one or more agents selected from metformin, a thiazolidinedione, a sulfonylurea, a dipeptidyl peptidase 4 inhibitor, a sodium glucose co-transporter, a SGLT-2 inhibitor, a growth differentiation factor 15 modulator (“GDF15”), a peptide tyrosine tyrosine modulator (“PYY”), a modified insulin, amylin, a dual amylin calcitonin receptor agonist, and oxyntomodulin agonist (“OXM”) in the treatment of a condition selected from the group consisting of T2DM, obesity, NAFLD, NASH, dyslipidemia and metabolic syndrome. In an embodiment, a compound of the present invention is provided in a fixed dose combination with one or more agents selected from metformin, a thiazolidinedione, a sulfonylurea, a dipeptidyl peptidase 4 inhibitor, a sodium glucose co-transporter, a SGLT-2 inhibitor, GDF15, PYY, a modified insulin, amylin, a dual amylin calcitonin receptor agonist, and OXM. In an embodiment is a compound of the present invention for use in simultaneous, separate and sequential combinations with one or more agents selected from metformin, a thiazolidinedione, a sulfonylurea, a dipeptidyl peptidase 4 inhibitor, a sodium glucose co-transporter, a SGLT-2 inhibitor, GDF15, PYY, a modified insulin, amylin, a dual amylin calcitonin receptor agonist, and OXM in the treatment of a condition selected from the group consisting of T2DM and obesity. In an embodiment is a compound of the present invention for use in simultaneous, separate and sequential combinations with one or more agents selected from metformin, a thiazolidinedione, a sulfonylurea, a dipeptidyl peptidase 4 inhibitor, a sodium glucose co-transporter, and a SGLT-2 inhibitor in the treatment of a condition selected from the group consisting of T2DM and obesity.

In other embodiments, the compounds, or a pharmaceutically acceptable salt thereof, may be useful to improve bone strength in subjects in need thereof. The compounds of the present invention, or a pharmaceutically acceptable salt thereof, may be useful in the treatment of other disorders such as Parkinson’s disease or Alzheimer’s disease. Incretins and incretin analogs having activity at one or more of the GIP, GLP-1 and/or glucagon receptors have been described as having the potential to have therapeutic value in a number of other diseases or conditions, including for example obesity, NAFLD and NASH, dyslipidemia, metabolic syndrome, bone related disorders, Alzheimer’s disease, and Parkinson’s disease. See, e.g., Jail S., et. al, Monomeric GLP-1/GIP/glucagon triagonism corrects obesity, hepatosteatosis, and dyslipidemia in female mice, MOL. METAB. 6(5):440-446 (March 2017); Carbone L.J., et. al., Incretin-based therapies for the treatment of non-alcoholic fatty liver disease: A systematic review and meta-analysis. J.GASTROENTEROL. HEPATOL., 31(1):23-31 (January 2016); B. Finan, et. al, Reappraisal of GIP Pharmacology for Metabolic Diseases. TRENDS MOL. MED., 22(5):359-76 (May 2016); Choi, I.Y., et al., Potent body weight loss and efficacy in a NASH animal model by a novel long-acting GLP-1/Glucagon/GIP triple-agonist (HM15211), ADA 2017 Poster 1139-P; Ding, K.H., Impact of glucose-dependent insulinotropic peptide on age-induced bone loss, J. BONE MINER. RES., 23(4):536-43 (2008); Tai, J. et. al, Neuroprotective effects of a triple GLP-⅟GIP/glucagon receptor agonist in the APP/PS1 transgenic mouse model of Alzheimer’s disease, BRAIN RES. 1678, 64-74 (2018); T.D. Müller et al., The New Biology and Pharmacology of Glucagon, PHYSIOL. REV. 97: 721-766 (2017); Finan, B. et. al, Unimolecular Dual Incretins Maximize Metabolic Benefits in Rodents, Monkeys, and Humans, SCI. TRANSL. MED., 5:209 (October 2013); Hölscher C, Insulin, incretins and other growth factors as potential novel treatments for Alzheimer’s and Parkinson’s diseases. BIOCHEM. Soc. TRANS. 42(2):593-0 (April 2014).

Another embodiment provides the use of a compound of the present invention, or a pharmaceutically acceptable salt thereof, in the manufacture of a medicament for the treatment of a condition selected from the group consisting of T2DM, obesity, NAFLD, NASH, dyslipidemia and metabolic syndrome. In an embodiment, the medicament is for the treatment of T2DM. In an embodiment, the medicament is for the treatment of obesity. In an embodiment, the medicament is for the treatment of NAFLD. In an embodiment, the medicament is for the treatment of NASH.

Another embodiment provides a pharmaceutical composition comprising a compound of Formula I, or a pharmaceutically acceptable salt thereof, and at least one selected from the group consisting of a carrier, diluent, and excipient.

As used herein, the term “treating” or “to treat” includes restraining, slowing, stopping, or reversing the progression or severity of a symptom, condition, or disorder.

Certain compounds provided herein are generally effective over a wide dosage range. For example, dosages for once weekly parenteral dosing may fall within the range of 0.05 mg to about 30 mg per person per week. Compounds provided herein are orally available and may be dosed using oral formulation techniques. Oral formulations may be formulated for periodic dosing, such as once daily.

The compounds of the present invention include novel amino acid sequences having affinity for the respective GLP-1 and GIP receptors, with desired potency at each of these receptors. GLP-1 is a 36 amino acid peptide, the major biologically active fragment of which is produced as a 30-amino acid, C-terminal amidated peptide (GLP-1₇₋₃₆) (SEQ ID NO:2).

GIP is a 42 amino acid peptide (SEQ ID NO:1), which, like GLP-1, is also known as an incretin, and plays a physiological role in glucose homeostasis by stimulating insulin secretion from pancreatic beta cells in the presence of glucose.

The compounds provide desired potency at each of the GIP and GLP-1 receptors. In an embodiment, compounds are suitable for oral administration. In an embodiment, compounds have desirable GIP and GLP receptor extended time action.

As used herein, “linker” means a group conjugating the R₅ fatty acid to a lysine of the peptide. As used herein, “fatty acid” means a hydrocarbon with a carboxyl group. As used herein “Ac” means acetyl modification. In an embodiment, a fatty acid is an albumin binding group. As used herein the term “amino acid” means both naturally occurring amino acids and unnatural amino acids. The amino acids are typically depicted using standard one letter codes (e.g., L = leucine), as well as alpha-methyl substituted residues of natural amino acids (e.g., α-methyl leucine, or αMeL and α-methyl lysine, or αMeK) and certain other unnatural amino acids, such as alpha amino isobutyric acid, or “Aib,” “4Pal,” “Orn,” and the like. The structures of non-natural amino acids and other abbreviations appear below:

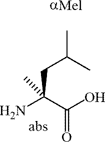

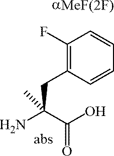

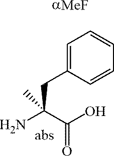

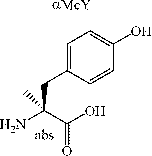

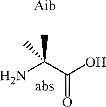

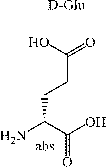

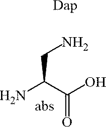

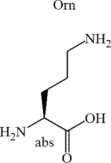

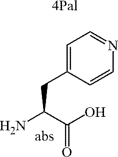

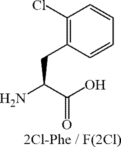

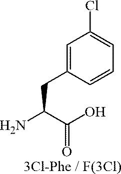

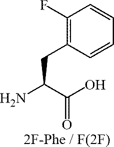

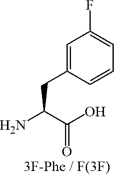

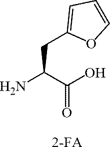

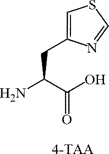

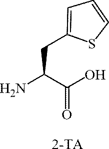

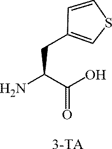

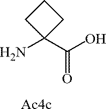

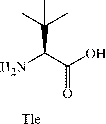

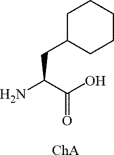

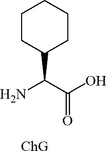 [SMILES: N[C@H](C(=O)O)C1CCCCC1 ; "ChG"]

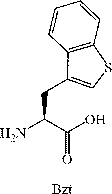

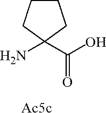

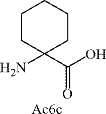

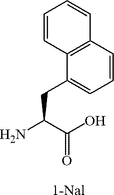

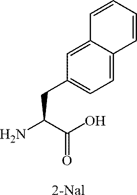

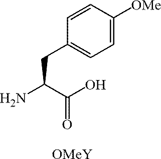 [SMILES: COc1ccc(C[C@H](N)C(=O)O)cc1 ; "OMeY"]

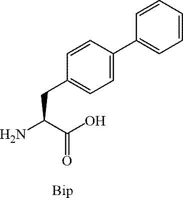

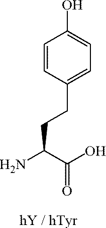

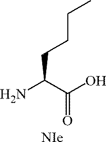

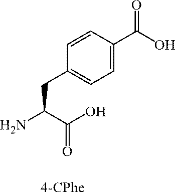

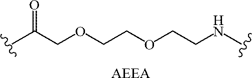

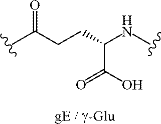

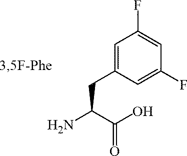

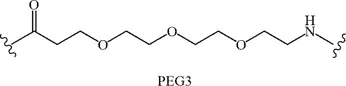

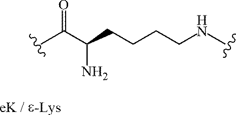

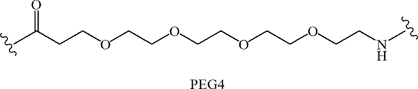

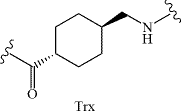

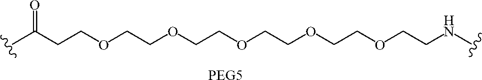

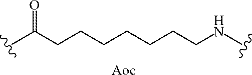

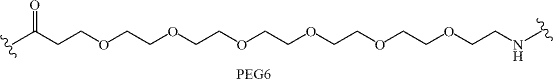

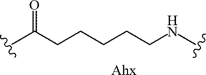

As used herein “Orn” means ornithine. As used herein “4Pal” means 3-(4-Pyridyl)-L-alanine. As used herein “αMeF(2F)” means alpha-methyl 2-F-phenylalanine, alpha-methyl-F(2F), and alpha-methyl-Phe(2F). As used herein “αMeY,” mean alpha methyl tyrosine, alpha methyl-Tyr, and alpha methyl-Y. As used herein, “αMeL” means alpha methyl leucine, alpha methyl-L, and alpha methyl-Leu. As used herein, “e” and “D-Glu” mean D-glutamic acid. As used herein, “αMeF”, means alpha-methyl-F and alpha-methyl-Phe. As used herein, “αMeS”, means alpha-methyl-serine, alpha methyl-S, and alpha-methyl-Ser. As used herein, “AEEA” means (2-[2-(2-Amino-ethoxy)-ethoxy]-acetyl), “AEEA2” means (2-[2-(2-Amino-ethoxy)-ethoxy]-acetyl)₂; and “AEEA3” means (2-[2-(2-Amino-ethoxy)-ethoxy]-acetyl)₃.

As used herein, “Ahx” is 6-Aminohexanoyl-; “Aoc” is 8-Aminooctanoyl-; “PEG3” is [3-(2-[2-(2-Amino-ethoxy)-ethoxy]-ethoxy)-propanoyl]-; “PEG4” is (3-[2-(2-[2-(2-Amino-ethoxy)-ethoxy]-ethoxy)-ethoxy]-propanoyl)-; “PEG5” is [3-(2-[2-(2-[2-(2-Amino-ethoxy)-ethoxy]-ethoxy)-ethoxy]-ethoxy)-propanoyl]-; and “PEG6” is (3-[2-(2-[2-(2-[2-(2-Amino-ethoxy)-ethoxy]-ethoxy)-ethoxy]-ethoxy)-ethoxy]-propanoyl)-. “Tle” is tert-Leucine.

As shown in the chemical structures of Examples below, in an embodiment the linker-fatty acid moieties (-R₅R₄), described above, link to the epsilon-amino group of a lysine side-chain.

When used herein in reference to one or more of the GIP or GLP-1 receptors, the terms “activity,” “activate[s]” “activat[ing]” and the like refers to the capacity of a compound, or a pharmaceutically acceptable salt thereof, to bind to and induce a response at the receptor(s), as measured using assays known in the art, such as the in vitro assays described below.

The affinity of compounds, or a pharmaceutically acceptable salt thereof, of the present invention for each of the GIP and GLP-1 receptors may be measured using techniques known for measuring receptor binding levels in the art, including, for example those described in the examples below, and is commonly expressed as a Ki value. The activity of the compounds of the present invention at each of the receptors may also be measured using techniques known in the art, including for example the in vitro activity assays described below, and is commonly expressed as an EC₅₀ value, which is the concentration of compound causing half-maximal simulation in a dose response curve.

In an embodiment, a pharmaceutical composition of a compound of Formula I is suitable for administration by a parenteral route (e.g., subcutaneous, intravenous, intraperitoneal, intramuscular, or transdermal). In an embodiment, a pharmaceutical composition of a compound of Formula I is suitable for oral administration (e.g., tablet, capsule). Some pharmaceutical compositions and processes for preparing same are well known in the art. (See, e.g., Remington: The Science and Practice of Pharmacy (D.B. Troy, Editor, 21st Edition, Lippincott, Williams & Wilkins, 2006).

Compounds of the provided herein may react with a number of inorganic and organic acids/bases to form pharmaceutically acceptable acid/base addition salts. Pharmaceutically acceptable salts and common methodology for preparing them are well known in the art. (See, e.g., P. Stahl, et al. Handbook of Pharmaceutical Salts: Properties, Selection and Use, 2nd Revised Edition (Wiley-VCH, 2011)). Pharmaceutically acceptable salts of the present invention include, but are not limited to, sodium, trifluoroacetate, hydrochloride, ammonium, and acetate salts. In an embodiment, a pharmaceutically acceptable salt of is selected from the group consisting of sodium, hydrochloride, and acetate salts.

The present invention also encompasses novel intermediates and processes useful for the synthesis of compounds of the present invention, or a pharmaceutically acceptable salt thereof. The intermediates and compounds of the present invention may be prepared by a variety of procedures known in the art. In particular, the Examples below describe a process using chemical synthesis. The specific synthetic steps for each of the routes described may be combined in different ways to prepare compounds of the present invention. The reagents and starting materials are readily available to one of ordinary skill in the art.

When used herein, the term “effective amount” refers to the amount or dose of a compound provided herein, or a pharmaceutically acceptable salt thereof, which, upon single or multiple dose administration to the patient, provides the desired effect in the patient under diagnosis or treatment. An effective amount can be determined by a person of skill in the art using known techniques and by observing results obtained under analogous circumstances. In determining the effective amount for a subject, a number of factors are considered, including, but not limited to: the species of mammal; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected, the use of concomitant medication; and other relevant circumstances.

When used herein, the term “subject in need thereof” refers to a mammal, preferably a human, with a disease or condition requiring treatment or therapy, including for example those listed in the preceding paragraphs. As used herein “EDTA” means ethylenediaminetetraacetic acid. As used herein “DMSO” means dimethyl sulfoxide. As used herein “CPM” means counts per minute. As used herein “IBMX” means 3-isobutyl-1-methylxanthine. As used herein “LC/MS” means liquid chromatography/mass spectrometry. As used herein “HTRF” means homogeneous time-resolved fluorescence. As used herein “BSA” mean bovine serum albumin.

The invention is further illustrated by the following examples, which are not to be construed as limiting.

### PEPTIDE SYNTHESIS

### Example 1

### Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ (SEQ ID NO:11).

The structure of SEQ ID NO: 11 is depicted below using the standard single letter amino acid codes with the exception of residues Aib2, αMeF(2F)6, 4Pal10, αMeL13, Orn16, K17, Aib20, D-Glu24, αMeY25, K31 and Ser39, where the structures of these amino acid residues have been expanded:

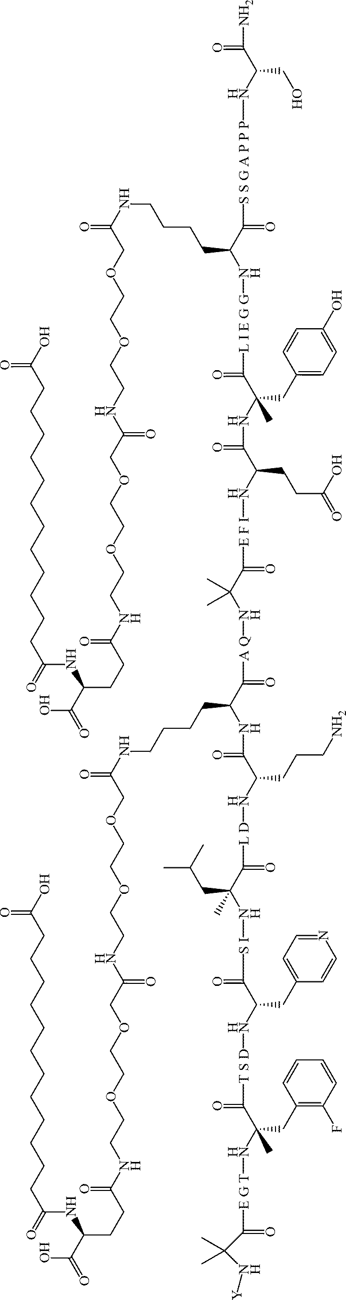

The peptide backbone of Example 1 is synthesized using Fluorenylmethyloxycarbonyl (Fmoc)/tert-Butyl (t-Bu) chemistry on a Symphony multiplex peptide synthesizer (Gyros Protein Technologies. Tucson, AZ).

The resin consists of 1% DVB cross-linked polystyrene (Fmoc-Rink-MBHA Low Loading resin, 100-200 mesh, EMD Millipore) at a substitution of 0.35 mmol/g. Standard side-chain protecting groups were used. Fmoc-Lys(Mtt)-OH is used for the lysine residues at positions 17 and 31, and Boc-Tyr(tBu)-OH was used for the tyrosine residue at position 1. Fmoc groups are removed prior to each coupling step (2 × 7 minutes) using 20% piperidine in DMF. All standard amino acid couplings are performed for 1 hour, using an equal molar ratio of Fmoc amino acid (0.3 mM in DMF), diisopropylcarbodiimide (0.9 mM in DCM) and Oxyma (0.9 mM in DMF), at a 9-fold molar excess over the theoretical peptide loading. Exceptions are couplings to Cα-methylated amino acids, which are coupled for 3 hours. After completion of the synthesis of the peptide backbone, the resin is thoroughly washed with DCM to remove residual DMF. The Mtt protecting groups on the lysine residues at positions 17 and 31 are selectively removed from the peptide resin using 30% hexafluoroisopropanol (Oakwood Chemicals) in DCM (3 × 1 hour treatments), and the resin is thoroughly washed with DCM and DMF.

Subsequent attachment of the linker moieties is accomplished by stepwise coupling of 2-[2-(2-Fmoc-amino-ethoxy)-ethoxy]-acetic acid (Fmoc-AEEA-OH, ChemPep, Inc.) and Fmoc-glutamic acid α-t-butyl ester (Fmoc-Glu-OtBu, Ark Pharm, Inc.), following the procedures described above for standard coupling and deprotection reactions. After removal of the final Fmoc protecting groups, mono-OtBu-tetradecanedioic acid (WuXi AppTec, Shanghai, China) is coupled overnight using a 4-fold excess of the fatty acid, diisopropylcarbodiimide, and Oxyma (1: 1 : 1 mol/mol/mol) in 1:1 DCM/DMF. After the synthesis is complete, the peptide-resin is washed with DCM and then thoroughly dried under vacuum.

The dry resin is treated with 10 mL of cleavage cocktail (trifluoroacetic acid: water: triisopropylsilane, 95:2.5:2.5 v/v) for 2 hours at room temperature. The resin is filtered off, washed twice each with 2 mL of neat TFA, and the combined filtrates are treated with 5-fold excess volume of cold diethyl ether (-20° C.) to precipitate the crude peptide. The peptide/ether suspension is then centrifuged at 3500 rpm for 2 min to form a solid pellet, the supernatant is decanted, and the solid pellet is triturated with ether two additional times and dried in vacuo. The crude peptide is solubilized in 20 mL of 20% acetonitrile/20%acetic acid/60%water and purified by RP- HPLC on a SymmetryPrep 7 µm C18 preparative column (19 × 300 mm, Waters) with linear gradients of 100% acetonitrile and 0.1% TFA/water buffer system (35-55% acetonitrile in 60 min). The purity of peptide is assessed using analytical RP-HPLC and pooling criteria is >95%. The main pool purity of Example 1 is found to be 96.0%. Subsequent lyophilization of the final main product pool yielded the lyophilized peptide TFA salt. The molecular weight is determined by LC- MS (obsd: M+3 =1853.9; calc M+3 = 1854.1)

### Example 2

### Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIEGGK((2-[2-(2-Amino-ethoxy)-ethoxy]-acetyl)₂-(γ-Glu)-(10-(4-carboxyphenoxy)decanoyl))SSGAPPPS-NH₂ (SEQ ID NO: 12)

The structure of SEQ ID NO: 12 is depicted below using the standard single letter amino acid codes with the exception of residues Aib2, αMeF(2F)6, 4Pal10, αMeL13, Orn16, K17, Aib20, D-Glu24, αMeY25, K31 and Ser39, where the structures of these amino acid residues have been expanded:

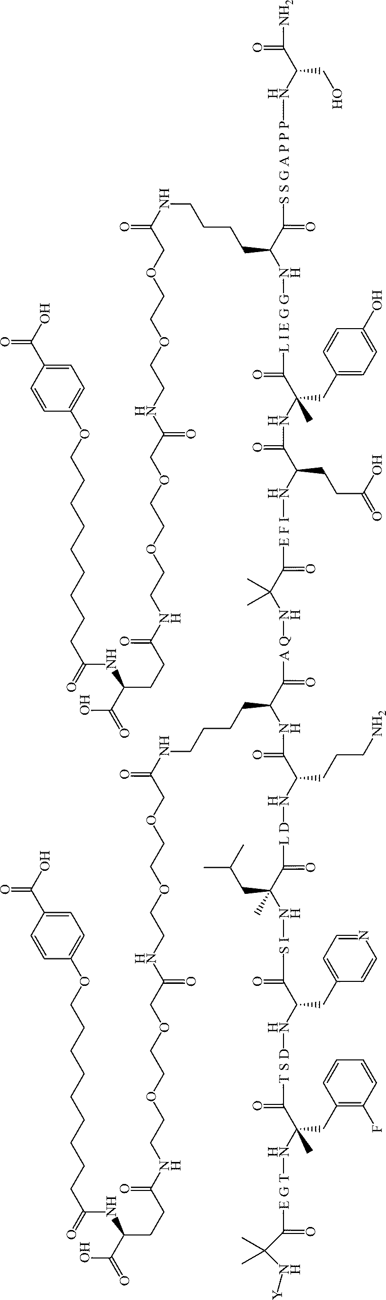

The compound according to SEQ ID NO: 12 is prepared substantially as described by the procedures of Example 1, except 4-(9-carboxy-nonyloxy)benzoic acid tert-butyl ester (WuXi AppTec, Shanghai, China) was used in the final coupling step. The molecular weight is determined by LC-MS (obsd: M+3 =1887.1; calc M+3 =1887.4).

### Example 3

### Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(4-(4-iodophenyl)butanoyl))AQ-Aib-EFI-(D-Glu)-αMeY-LIEGGK((2-[2-(2-Amino-ethoxy)-ethoxy]-acetyl)₂-(γ-Glu)-(4-(4-iodophenyl)butanoyl))SSGAPPPS-NH₂ (SEQ ID NO: 13)

The structure of SEQ ID NO: 13 is depicted below using the standard single letter amino acid codes with the exception of residues Aib2, αMeF(2F)6, 4Pal10, αMeL13, Orn16, K17, Aib20, D-Glu24, αMeY25, K31 and Ser39, where the structures of these amino acid residues have been expanded:

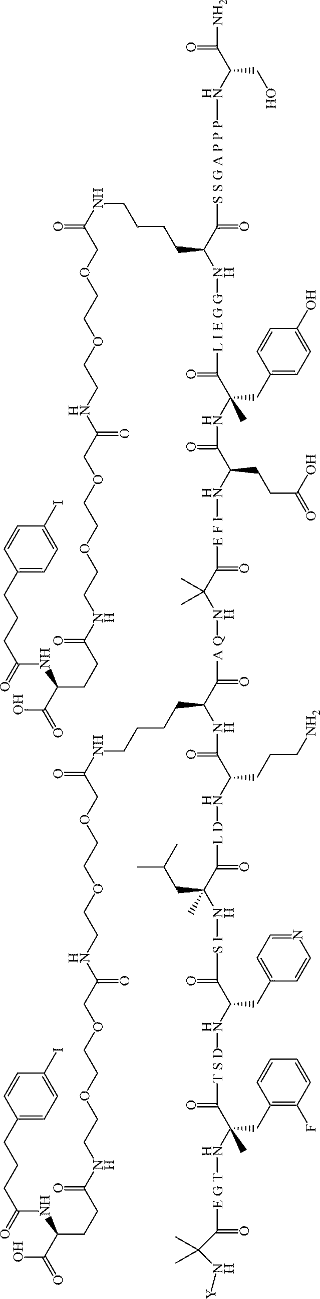

The compound according to SEQ ID NO: 13 is prepared substantially as described by the procedures of Example 1, except 4-(4-Iodophenyl)butyric acid (WuXi AppTec, Shanghai, China) was used in the final coupling step. The molecular weight is determined by LC-MS (obsd: M+3 =1875.1; calc M+3 =1875.2).

### Example 4

### Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(4-(4-tert-butylphenyl)butanoyl))AQ-Aib-EFI-(D-Glu)-αMeY-LIEGGK((2-[2-(2-Amino-ethoxy)-ethoxy]-acetyl)₂-(γ-Glu)-(4-(4-tert-butylphenyl)butanoyl))SSGAPPPS-NHz (SEQ ID NO: 14)

The structure of SEQ ID NO: 14 is depicted below using the standard single letter amino acid codes with the exception of residues Aib2, αMeF(2F)6, 4Pal10, αMeL13, Orn16, K17, Aib20, D-Glu24, αMeY25, K31 and Ser39, where the structures of these amino acid residues have been expanded:

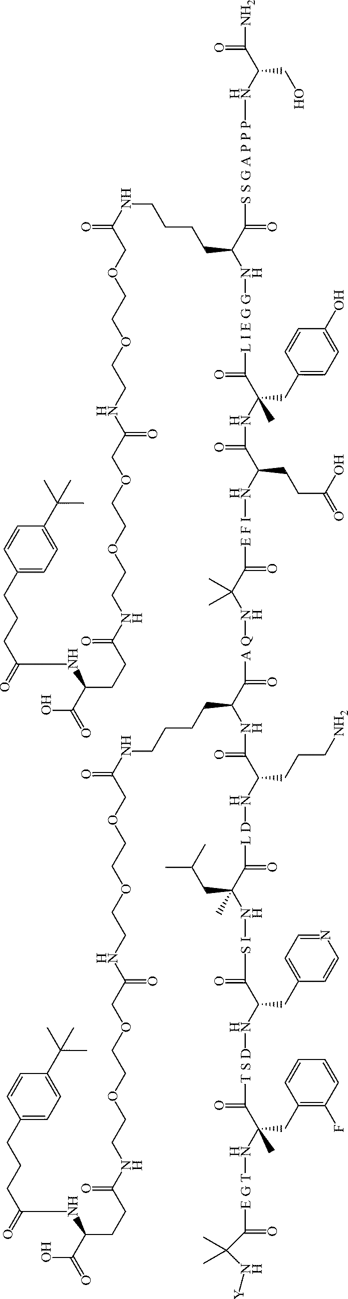

The compound according to SEQ ID NO: 14 is prepared substantially as described by the procedures of Example 1, except 4-(4-tert-Butylphenyl)butyric acid (WuXi AppTec, Shanghai, China) was used in the final coupling step. The molecular weight is determined by LC-MS (obsd: M+3 =1828.3; calc M+3 =1828.7).

### Example 5

### Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGGK((2-[2-(2-Amino-ethoxy)-ethoxy]-acetyl)₂-(γ-Glu)-CO-(CH₂)₁₀-CH₃)SSGAPPPS-NH₂ (SEQ ID NO:15).

The structure of SEQ ID NO: 15 is depicted below using the standard single letter amino acid codes with the exception of residues Aib2, αMeF(2F)6, 4Pal10, αMeL13, Orn16, K17, Aib20, D-Glu24, αMeY25, K31 and Ser39, where the structures of these amino acid residues have been expanded:

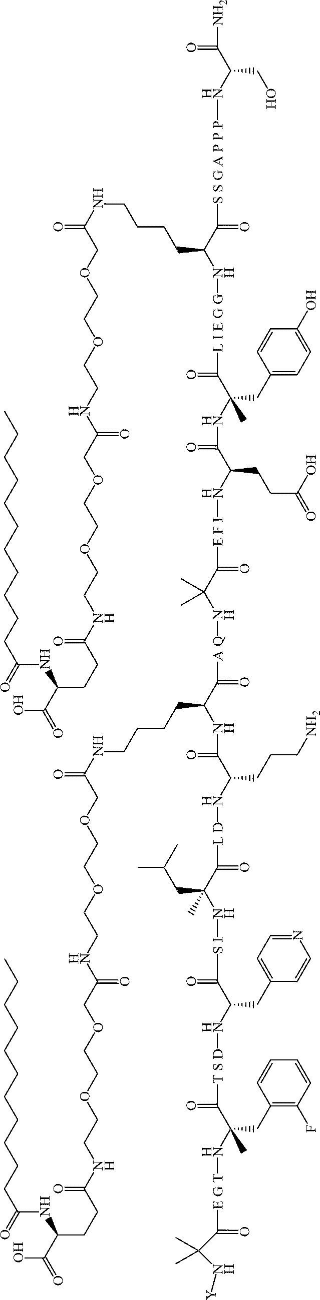

The compound according to SEQ ID NO: 15 is prepared substantially as described by the procedures of Example 1, except Lauric acid (Sigma Aldrich) was used in the final coupling step. The molecular weight is determined by LC-MS (obsd: M+3 =1815.1; calc M+3 =1815.4).

### Examples 6 Through Example 211

The compounds according to Examples 6 (SEQ ID NO: 16) through Example 494 (SEQ ID NO:504) are prepared substantially as described by the procedures of Example 1.

| Example | Name | SEQ ID NO | MW (g/mol) Calculated |
|---|---|---|---|
| 6 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₀-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₀-CO₂H)SSGAPPPS-NH₂ | 16 | 5503.09 |
| 7 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₄-CH₃)AQ-Aib-EFI-(D-Glu)-αMeY-LIEGGK((2-[2-(2-Amino-ethoxy)-ethoxy]-acetyl)₂-(γ-Glu)-CO-(CH₂)₁₄-CH₃)SSGAPPPS-NH₂ | 17 | 5555.33 |
| 8 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CH₃)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CH₃)SSGAPPPS-NH₂ | 18 | 5499.23 |
| 9 | Y-Aib-EGT-αMeF(2F)-TSD-4Pal-SI-αMeL-LD-Orn-K((2-[2-(2-Amino-ethoxy)-ethoxy]-acetyl)₂-CO-(CH₂)₁₂-CO₂H)AQ-Aib-EFI-(D-Glu)-αMeY-LIEGGK((2-[2-(2-Amino-ethoxy)-ethoxy]-acetyl)₂-CO-(CH₂)₁₂-CO₂H)SSGAPPPS-NH₂ | 19 | 5300.97 |
| 10 | Y-Aib-EGT-αMeF(2F)-TSD-4Pal-SI-αMeL-LD-Orn-K((2-[2-(2-Amino-ethoxy)-ethoxy]-acetyl)₂-(10-(4-carboxyphenoxy)decanoyl))AQ-Aib-EFI-(D-Glu)-αMeY-LIEGGK((2-[2-(2-Amino-ethoxy)-ethoxy]-acetyl)₂-(10-(4-carboxyphenoxy)decanoyl))SSGAPPPS-NH₂ | 20 | 5401 |
| 11 | Y-Aib-EGT-αMeF(2F)-TSD-4Pal-SI-αMeL-LD-Orn-K((2-[2-(2-Amino-ethoxy)-ethoxy]-acetyl)-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)-(y-Glu)-CO-(CH₂)₁₂-CO₂H)SSGAPPPS-NH₂ | 21 | 5268.88 |
| 12 | Y-Aib-EGT-αMeF(2F)-TSD-4Pal-SI-αMeL-LD-Orn-K((2-[2-(2-Amino-ethoxy)-ethoxy]-acetyl)-(γ-Glu)-(10-(4-carboxyphenoxy)decanoyl))AQ-Aib-EFI-(D-Glu)-αMeY-LIEGGK((2-[2-(2-Amino-ethoxy)-ethoxy]-acetyl)-(γ-Glu)-(10-(4-carboxyphenoxy)decanoyl))SSGAPPPS-NH₂ | 22 | 5368.91 |
| 13 | Y-Aib-EGT-αMeF(2F)-TSD-4Pal-SI-αMeL-LD-Orn-K((2-[2-(2-Amino-ethoxy)-ethoxy]-acetyl)-(γ-Glu)-CO-(CH₂)₁₄-CH₃)AQ-Aib-EFI-(D-Glu)-αMeY-LIEGGK((2-[2-(2-Amino-ethoxy)-ethoxy]-acetyl)-(γ-Glu)-CO-(CH₂)₁₄-CH₃)SSGAPPPS-NH₂ | 23 | 5265.02 |
| 14 | Y-Aib-EGT-αMeF(2F)-TSD-4Pal-SI-αMeL-LD-Orn-K((2-[2-(2-Amino-ethoxy)-ethoxy]-acetyl)-(γ-Glu)-CO-(CH₂)₁₀-CH₃)AQ-Aib-EFI-(D-Glu)-αMeY-LIEGGK((2-[2-(2-Amino-ethoxy)-ethoxy]-acetyl)-(γ-Glu)-CO-(CH₂)₁₀-CH₃)SSGAPPPS-NH₂ | 24 | 5152.81 |
| 15 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIEGGPSSGAPPPS-NH₂ | 25 | 5527.2 |
| 16 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFIK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 26 | 5527.2 |
| 17 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 27 | 5527.2 |
| 18 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGK((2-[2-(2-Amino-ethoxy)-ethoxy]-acetyl)₂-(γ-Glu)-CO-(CH₂)₁₂-CO₂H)PSSGAPPPS-NH₂ | 28 | 5599.26 |
| 19 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGP S SGAPPP SK((2-[2-(2-Amino-ethoxy)-ethoxy]-acetyl)₂-(γ-Glu)-CO-(CH₂)₁₂-CO₂H)-NH₂ | 29 | 5656.31 |
| 20 | Y-Aib-EGT-αMeF(2F)-TSD-4Pal-SI-αMeL-LD-Orn-IAQ-Aib-EFI-(D-Glu)-αMeY-LIK((2-[2-(2-Amino-ethoxy)-ethoxy]-acetyl)₂-(γ-Glu)-CO-(CH₂)₁₂-CO₂H)GGK((2-[2-(2-Amino-ethoxy)-ethoxy]-acetyl)₂-(γ-Glu)-CO-(CH₂)₁₂-CO₂H)SSGAPPPS-NH₂ | 30 | 5543.24 |
| 21 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₄-CH₃)AQ-Aib-K((2-[2-(2-Amino-ethoxy)-ethoxy]-acetyl)₂-(γ-Glu)-CO-(CHz)₁₄-CH₃)FI-(D-Glu)-αMeY-LIEGGPSSGAPPPS-NH₂ | 31 | 5583.3 |
| 22 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₄-CH₃)AQ-Aib-EFIK((2-[2-(2-Amino-ethoxy)-ethoxy]-acetyl)₂-(γ-Glu)-CO-(CH₂)₁₄-CH₃)-αMeY-LIEGGPSSGAPPPS-NH₂ | 32 | 5583.3 |
| 23 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₄-CH₃)AQ-Aib-EFI-(D-Glu)-αMeY-LIK((2-[2-(2-Amino-ethoxy)-ethoxy]-acetyl)₂-(γ-Glu)-CO-(CH₂)₁₄-CH₃ )GGPSSGAPPPS-NH₂ | 33 | 5523.34 |
| 24 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₄-CH₃)AQ-Aib-EFI-(D-Glu)-αMeY-LIEGK((2-[2-(2-Amino-ethoxy)-ethoxy]-acetyl)₂-(γ-Glu)-CO-(CH₂)₁₄-CH₃)PSSGAPPPS-NH₂ | 34 | 5595.4 |
| 25 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₄-CH₃)AQ-Aib-EFI-(D-Glu)-αMeY-LIEGGPSSGAPPPSK((2-[2-(2-Amino-ethoxy)-ethoxy]-acetyl)₂-(γ-Glu)-CO-(CH₂)₁₄-CH₃)-NH₂ | 35 | 5652.45 |
| 26 | Y-Aib-EGT-αMeF(2F)-TSD-4Pal-SI-αMeL-LD-Orn-IAQ-Aib-EFI-(D-Glu)-αMeY-LIK((2-[2-(2-Amino-ethoxy)-ethoxy]-acetyl)₂-(γ-Glu)-CO-(CH₂)₁₄-CH₃)GGK((2-[2-(2-Amino-ethoxy)-ethoxy]-acetyl)₂-(γ-Glu)-CO-(CH₂)₁₄-CH₃)SSGAPPPS-NH₂ | 36 | 5539.38 |
| 27 | Y-Aib-EGT-αMeF(2F)-TSDK((2-[2-(2-Amino-ethoxy)-ethoxy]-acetyl)₂-(γ-Glu)-CO-(CH₂)₁₂-CO₂H)SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGPSSGAPPPS-NH₂ | 37 | 5508.15 |
| 28 | Y-Aib-EGT-αMeF(2F)-TSD-4Pal-SK((2-[2-(2-Amino-ethoxy)-ethoxy]-acetyl)₂-(γ-Glu)-CO-(CH₂)₁₂-CO₂H)-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGPSSGAPPPS-NH₂ | 38 | 5543.15 |
| 29 | Y-Aib-EGT-αMeF(2F)-TSDK((2-[2-(2-Amino-ethoxy)-ethoxy]-acetyl)₂-(γ-Glu)-CO-(CH₂)₁₄-CH₃)SI-αMeL-LD-Orn-K((2-[2-(2-Amino-ethoxy)-ethoxy]-acetyl)₂-(γ-Glu)-CO-(CH₂)₁₄-CH₃)AQ-Aib-EFI-(D-Glu)-αMeY-LIEGGPSSGAPPPS-NH₂ | 39 | 5504.29 |
| 30 | Y-Aib-EGT-αMeF(2F)-TSD-4Pal-SI-αMeL-LD-Orn-K((2-[2-(2-Amino-ethoxy)-ethoxy]-acetyl)₃-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₃-(γ-Glu)-CO-(CH₂)₁₂-CO₂H)SSGAPPPS-NH₂ | 40 | 5849.51 |
| 31 | Y-Aib-EGT-αMeF(2F)-TSD-4Pal-SI-αMeL-LD-Orn-K((2-[2-(2-Amino-ethoxy)-ethoxy]-acetyl)₃-(γ-Glu)-CO-(CH₂)₁₄-CH₃)AQ-Aib-EFI-(D-Glu)-αMeY-LIEGGK((2-[2-(2-Amino-ethoxy)-ethoxy]-acetyl)₃-(γ-Glu)-CO-(CH₂)₁₄-CH₃)SSGAPPPS-NH₂ | 41 | 5845.65 |
| 32 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₄-CH₃)AQ-Aib-EFI-(D-Glu)K((2-[2-(2-Amino-ethoxy)-ethoxy]-acetyl)₂-(γ-Glu)-CO-(CH₂)₁₄-CH₃)LIEGGPSSGAPPPS-NH₂ | 42 | 5475.25 |
| 33 | Y-Aib-EGT-αMeF(2F)-TSD-4Pal-SI-αMeL-LD-Orn-K((2-[2-(2-Amino-ethoxy)-ethoxy]-acetyl)₂-(εK)-CO-(CH₂)₁₂-CO₂H)AQ-Aib-EFI-(D-Glu)-αMeY-LIEGGK((2-[2-(2-Amino-ethoxy)-ethoxy]-acetyl)₂-(εK)-CO-(CH₂)₁₂-CO₂H)SSGAPPPS-NH₂ | 43 | 5557.31 |
| 34 | Y-Aib-EGT-αMeF(2F)-TSD-4Pal-SI-αMeL-LD-Orn-K((2-[2-(2-Amino-ethoxy)-ethoxy]-acetyl)₂-(εK)-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(εK)-(γ-Glu)-CO-(CH₂)₁₂-CO₂H)SSGAPPPS-NH₂ | 44 | 5815.54 |
| 35 | Y-Aib-EGT-αMeF(2F)-TSD-4Pal-SI-αMeL-LD-Orn-K((2-[2-(2-Amino-ethoxy)-ethoxy]-acetyl)-(εK)-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)-(εK)-(γ-Glu)-CO-(CH₂)₁₂-CO₂H)SSGAPPP S-NH₂ | 45 | 5525.23 |
| 36 | Y-Aib-EGT-αMeF(2F)-TSD-4Pal-SI-αMeL-LD-Orn-K((εK)-(2-[2-(2-Amino-ethoxy)-ethoxy]-acetyl)-(γ-Glu)-CO-(CH₂)₁₂-CO₂H)AQ-Aib-EFI-(D-Glu)-αMeY-LIEGGK((εK)-(2-[2-(2-Amino-ethoxy)-ethoxy]-acetyl)-(γ-Glu)-CO-(CH₂)₁₂-CO₂H)SSGAPPPS-NH₂ | 46 | 5525.23 |
| 37 | Y-Aib-EGT-αMeF(2F)-TSD-4Pal-SI-αMeL-LD-Orn-K((εK)-(2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((εK)-(2-[2-(2-Amino-ethoxy)-ethoxy]-acetyl)₂-(γ-Glu)-CO-(CH₂)₁₂-CO₂H)SSGAPPPS-NH₂ | 47 | 5815.54 |
| 38 | Y-Aib-EGT-αMeF(2F)-TSD-4Pal-SI-αMeL-LD-Orn-K((εK)-(γ-Glu)-CO-(CH₂)₁₂-CO₂H)AQ-Aib-EFI-(D-Glu)-αMeY-LIEGGK((εK)-(γ-Glu)-CO-(CH₂)₁₂-CO₂H)SSGAPPPS-NH₂ | 48 | 5234.91 |
| 39 | Y-Aib-EGT-αMeF(2F)-TSD-4Pal-SI-αMeL-LD-Orn-K((εK)-(γ-Glu)-(γ-Glu)-CO-(CH₂)₁₂-CO₂H)AQ-Aib-EFI-(D-Glu)-αMeY-LIEGGK((εK)-(γ-Glu)-(γ-Glu)-CO-(CH₂)₁₂-CO₂H)SSGAPPPS-NH₂ | 49 | 5493.14 |
| 40 | Y-Aib-EGT-αMeF(2F)-TSD-4Pal-SI-αMeL-LD-Orn-K((εK)-(εK)-(γ-Glu)-CO-(CH₂)₁₂-CO₂H)AQ-Aib-EFI-(D-Glu)-αMeY-LIEGGK((εK)-(εK)-(γ-Glu)-CO-(CH₂)₁₂-CO₂H)SSGAPPPS-NH₂ | 50 | 5491.26 |
| 41 | Y-Aib-EGT-αMeF(2F)-TSD-4Pal-SI-αMeL-LD-Orn-K((γ-Glu)-(2-[2-(2-Amino-ethoxy)-ethoxy]-acetyl)₂-CO-(CH₂)₁₂-CO₂H)AQ-Aib-EFI-(D-Glu)-αMeY-LIEGGK((γ-Glu)-(2-[2-(2-Amino-ethoxy)-ethoxy]-acetyl)₂-CO-(CH₂)₁₂-CO₂H)SSGAPPPS-NH₂ | 51 | 5559.19 |
| 42 | Y-Aib-EGT-αMeF(2F)-TSD-4Pal-SI-αMeL-LD-Orn-K((γ-Glu)-(2-[2-(2-Amino-ethoxy)-ethoxy]-acetyl)-CO-(CH₂)₁₂-CO₂H)AQ-Aib-EFI-(D-Glu)-αMeY-LIEGGK((γ-Glu)-(2-[2-(2-Amino-ethoxy)-ethoxy]-acetyl)-CO-(CH₂)₁₂-CO₂H)SSGAPPPS-NH₂ | 52 | 5268.88 |
| 43 | Y-Aib-EGT-αMeF(2F)-TSD-4Pal-SI-αMeL-LD-Orn-K((2-[2-(2-Amino-ethoxy)-ethoxy]-acetyl)-(γ-Glu)-(2-[2-(2-Amino-ethoxy)-ethoxy]-acetyl)-CO-(CH₂)₁₂-CO₂H)AQ-Aib-EFI-(D-Glu)-αMeY-LIEGGK((2-[2-(2-Amino-ethoxy)-ethoxy]-acetyl)-(γ-Glu)-(2-[2-(2-Amino-ethoxy)-ethoxy]-acetyl)-CO-(CH₂)₁₂-CO₂H)SSGAPPPS-NH₂ | 53 | 5559.19 |
| 44 | Y-Aib-EGT-αMeF-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 54 | 5541.2 |
| 45 | Y-Aib-EGT-αMeF-TSD-αMeF-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 55 | 5554.24 |
| 46 | Y-Aib-EGT-αMeF-TSD-4Pal-SI-αMeF-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 56 | 5575.22 |
| 47 | Y-Aib-EGT-αMeF-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 57 | 5555.23 |
| 48 | Y-Aib-EGT-αMeF-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeF-LIEGGK((2-[2-(2-Amino-ethoxy)-ethoxy]-acetyl)₂-(γ-Glu)-CO-(CH₂)₁₂-CO₂H)SSGAPPPS-NH₂ | 58 | 5525.2 |
| 49 | Y-Aib-EGT-αMeF(2F)-TSDY-aMeS-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 59 | 5588.23 |
| 50 | Y-Aib-EGT-αMeF(2F)-TSDY-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 60 | 5572.23 |
| 51 | Y-Aib-EGT-αMeF(2F)-TSD-αMeL-SI-aMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 61 | 5538.22 |
| 52 | Y-Aib-EGT-αMeF(2F)-TSD-aMeV-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 62 | 5524.19 |
| 53 | Y-Aib-EGT-αMeF(2F)-TSD-Ac5c-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 63 | 5522.17 |
| 54 | Y-Aib-EGT-αMeF(2F)-TSD-Ac6c-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 64 | 5536.2 |
| 55 | Y-Aib-EGT-αMeF(2F)-TSD-Bip-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 65 | 5634.3 |
| 56 | Y-Aib-EGT-αMeF(2F)-TSD-1Nal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 66 | 5608.26 |
| 57 | Y-Aib-EGT-αMeF(2F)-TSD-2Nal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 67 | 5608.26 |
| 58 | Y-Aib-EGT-αMeF(2F)-TSD-OMeY-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 68 | 5588.23 |
| 59 | Y-Aib-EGT-αMeF(2F)-TSD-hTyr-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 69 | 5588.23 |
| 60 | Y-Aib-EGT-αMeF(2F)-TSD-Nle-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 70 | 5524.19 |
| 61 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-(2ClPhe)-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 71 | 5593.64 |
| 62 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-(3ClPhe)-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 72 | 5593.64 |
| 63 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-(2FPhe)-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 73 | 5577.18 |
| 64 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-3FPhe-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 74 | 5577.18 |
| 65 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-3,5FPhe-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 75 | 5595.17 |
| 66 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-1Nal-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 76 | 5609.25 |
| 67 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(4-(4-tert-butylphenyl)butanoyl))AQ-Aib-EFI-(D-Glu)-K((2-[2-(2-Amino-ethoxy)-ethoxy]-acetyl)₂-(γ-Glu)-(4-(4-tert-butylphenyl)butanoyl))LIEGGPSSGAPPPS-NH₂ | 77 | 5403.02 |
| 68 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(4-(4-tert-butylphenyl)butanoyl))AQ-Aib-EFIE-K((2-[2-(2-Amino-ethoxy)-ethoxy]-acetyl)₂-(γ-Glu)-(4-(4-tert-butylphenyl)butanoyl))LIEGGPSSGAPPPS-NH₂ | 78 | 5403.02 |
| 69 | Y-Aib-EGT-αMeF(2F)-TSD-4Pal-SI-αMeL-LD-Orn-IAQ-Aib-K((2-[2-(2-Amino-ethoxy)-ethoxy]-acetyl)₂-(γ-Glu)-CO-(CH₂)₁₂-CO₂H)FIK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 79 | 5511.24 |
| 70 | Y-Aib-EGT-αMeF(2F)-TSD-4Pal-SI-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 80 | 5511.24 |
| 71 | Y-Aib-EGT-αMeF(2F)-TSD-4Pal-SI-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 81 | 5543.24 |
| 72 | Y-Aib-EGT-αMeF(2F)-TSD-4Pal-SI-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIEGGPSSGAPPPSK((2-[2-(2-Amino-ethoxy)-ethoxyl-acetyl)₂-(γ-Glu)-CO-(CH₂)₁₂-CO₂H)-NH₂ | 82 | 5640.35 |
| 73 | Y-Aib-EGT-αMeF(2F)-TSD-4Pal-SI-αMeL-LD-Orn-IAQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 83 | 5511.24 |
| 74 | Y-Aib-EGT-αMeF(2F)-TSD-4Pal-SI-αMeL-LD-Orn-IAQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 84 | 5543.24 |
| 75 | Y-Aib-EGT-αMeF(2F)-TSD-4Pal-SI-αMeL-LD-Orn-IAQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIEGGPSSGAPPPSK((2-[2-(2-Amino-ethoxy)-ethoxyl-acetyl)₂-(γ-Glu)-CO-(CH₂)₁₂-CO₂H)-NH₂ | 85 | 5640.35 |
| 76 | Y-Aib-EGT-αMeF(2F)-TSD-4Pal-SI-αMeL-LD-Orn-K((2-[2-(2-Amino-ethoxy)-ethoxy]-acetyl)-(y-Glu)-CO-(CH₂)₁₂-CO₂H)AQ-Aib-K((2-[2-(2-Amino-ethoxy)-ethoxy]-acetyl)-(γ-Glu)-CO-(CH₂)₁₂-CO₂H)FI-(D-Glu)-αMeY-LIEGGPSSGAPPPS-NH₂ | 86 | 5236.88 |
| 77 | Y-Aib-EGT-αMeF(2F)-TSD-4Pal-SI-αMeL-LD-Orn-K((2-[2-(2-Amino-ethoxy)-ethoxy]-acetyl)₃-(γ-Glu)-CO-(CH₂)₁₂-CO₂H)AQ-Aib-K((2-[2-(2-Amino-ethoxy)-ethoxy]-acetyl)₃-(γ-Glu)-CO-(CH₂)₁₂-CO₂H)FI-(D-Glu)-αMeY-LIEGGPSSGAPPPS-NH₂ | 87 | 5817.51 |
| 78 | Y-Aib-EGT-αMeF(2F)-TSD-4Pal-SI-αMeL-LD-Orn-K((2-[2-(2-Amino-ethoxy)-ethoxy]-acetyl)₂-CO-(CH₂)₁₂-CO₂H)AQ-Aib-K((2-[2-(2-Amino-ethoxy)-ethoxy]-acetyl)₂-CO-(CH₂)₁₂-CO₂H)Fl-(D-Glu)-aMeY-LIEGGPSSGAPPPS-NH₂ | 88 | 5268.97 |
| 79 | Y-Aib-EGT-αMeF(2F)-TSD-4Pal-SI-αMeL-LD-Orn-K((2-[2-(2-Amino-ethoxy)-ethoxy]-acetyl)-(γ-Glu)-CO-(CH₂)₁₂-CO₂H)AQ-Aib-EFIK((2-[2-(2-Amino-ethoxy)-ethoxy]-acetyl)-(γ-Glu)-CO-(CH₂)₁₂-CO₂H)-αMeY-LIEGGPSSGAPPPS-NH₂ | 89 | 5236.88 |
| 80 | Y-Aib-EGT-αMeF(2F)-TSD-4Pal-SI-αMeL-LD-Orn-K((2-[2-(2-Amino-ethoxy)-ethoxy]-acetyl)₃-(γ-Glu)-CO-(CH₂)₁₂-CO₂H)AQ-Aib-EFIK((2-[2-(2-Amino-ethoxy)-ethoxy]-acetyl)₃-(γ-Glu)-CO-(CH₂)₁₂-CO₂H)-αMeY-LIEGGPSSGAPPPS-NH₂ | 90 | 5817.51 |
| 81 | Y-Aib-EGT-aMeF(2F)-TSD-4Pal-SI-aMeL-LD-Orn-K((2-[2-(2-Amino-ethoxy)-ethoxy]-acetyl)₂-CO-(CH₂)₁₂-CO₂H)AQ-Aib-EFIK((2-[2-(2-Amino-ethoxy)-ethoxy]-acetyl)₂-CO-(CH₂)₁₂-CO₂H)-αMeY-LIEGGPSSGAPPPS-NH₂ | 91 | 5268.97 |
| 82 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-IK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 92 | 5541.22 |
| 83 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFIK((2-[2-(2-Amino-ethoxy)-ethoxy]-acetyl)z-(γ-Glu)-CO-(CH₂)₁₂-CO₂H)-αMeF-LIEGGPSSGAPPPS-NH₂ | 93 | 5511.2 |
| 84 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-IK((2-[2-(2-Amino-ethoxy)-ethoxy]-acetyl)₂-(γ-Glu)-CO-(CH₂)₁₂-CO₂H)-αMeF-LIEGGPSSGAPPPS-NH₂ | 94 | 5525.22 |
| 85 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-IK((2-[2-(2-Amino-ethoxy)-ethoxy]-acetyl)₂-(γ-Glu)-CO-(CH₂)₁₂-CO₂H)YLIEGGPSSGAPPPS-NH₂ | 95 | 5527.2 |
| 86 | Y-Aib-EGT-αMeF(2F)-TSD-4Pal-SI-αMeL-LD-Orn-QAQ-Aib-EFI-(D-Glu)-αMeY-LIK((2-[2-(2-Amino-ethoxy)-ethoxy]-acetyl)₂-(γ-Glu)-CO-(CH₂)₁₂-CO₂H)GGK((2-[2-(2-Amino-ethoxy)-ethoxy]-acetyl)₂-(γ-Glu)-Co-(CH₂)₁₂-CO₂H)SSGAPPPS-NH₂ | 96 | 5558.21 |
| 87 | Y-Aib-EGT-αMeF(2F)-TSD-αMeF(2F)-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 97 | 5590.22 |
| 88 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF(2F)-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 98 | 5587.25 |
| 89 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 99 | 5557.22 |
| 90 | Y-Aib-EGT-αMeF(2F)-TSDVSI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 100 | 5510.16 |
| 91 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 101 | 5508.19 |
| 92 | Y-Aib-EGT-αMeF(2F)-TSDVSI-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 102 | 5524.19 |
| 93 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 103 | 5522.22 |
| 94 | Y-Aib-EGT-αMeF(2F)-TSD-Ac5c-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 104 | 5536.2 |
| 95 | Y-Aib-EGT-αMeF(2F)-TSD-Ac4c-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 105 | 5508.15 |
| 96 | Y-Aib-EGT-αMeF(2F)-TSD-4CPhe-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 106 | 5602.22 |
| 97 | Y-Aib-EGT-αMeF(2F)-TSD-ChG-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 107 | 5550.23 |
| 98 | Y-Aib-EGT-αMeF(2F)-TSD-ChA-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 108 | 5564.25 |
| 99 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-2Nal-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 109 | 5609.25 |
| 100 | Y-Aib-EGT-αMeF(2F)-TSD-4Pal-GI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 110 | 5529.17 |
| 101 | Y-Aib-EGT-αMeF(2F)-TSD-4Pal-SI-αMeL-LDGK((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 111 | 5502.1 |
| 102 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)GQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 112 | 5545.17 |
| 103 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-GFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 113 | 5487.13 |
| 104 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFIG-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 114 | 5487.13 |
| 105 | Y-Aib-EGT-αMeF(2F)-TSDK((2-[2-(2-Amino-ethoxy)-ethoxy]-acetyl)₂-(γ-Glu)-CO-(CH₂)₁₂-CO₂H)SI-αMeL-LD-Om-IAQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIEGGPSSGAPPPS-NH₂ | 115 | 5492.19 |
| 106 | Y-Aib-EGT-αMeF(2F)-TSDK((2-[2-(2-Amino-ethoxy)-ethoxy]-acetyl)₂-(γ-Glu)-CO-(CH₂)₁₂-CO₂H)SI-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 116 | 5492.19 |
| 107 | Y-Aib-EGT-αMeF(2F)-TSDK((2-[2-(2-Amino-ethoxy)-ethoxy]-acetyl)₂-(γ-Glu)-CO-(CH₂)₁₂-CO₂H)SI-αMeL-LD-Orn-IAQ-Aib-EFI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 117 | 5492.19 |
| 108 | Y-Aib-EGT-αMeF(2F)-TSDK((2-[2-(2-Amino-ethoxy)-ethoxy]-acetyl)₂-(γ-Glu)-CO-(CH₂)₁₂-CO₂H)SI-αMeL-LD-Orn-IAQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 118 | 5524.19 |
| 109 | Y-Aib-EGT-αMeF(2F)-TSDK((2-[2-(2-Amino-ethoxy)-ethoxy]-acetyl)₂-(γ-Glu)-CO-(CH₂)₁₂-CO₂H)SI-αMeL-LD-Om-IAQ-Aib-K((2-[2-(2-Amino-ethoxy)-ethoxy]-acetyl)₂-(γ-Glu)-CO-(CH₂)₁₂-CO₂H)-αMeF-I-(D-Glu)-αMeY-LIEGGPSSGAPPPS-NH₂ | 119 | 5506.22 |
| 110 | Y-Aib-EGT-αMeF(2F)-TSDK((2-[2-(2-Amino-ethoxy)-ethoxy]-acetyl)₂-(γ-Glu)-CO-(CH₂)₁₂-CO₂H)SI-αMeL-LD-Orn-IAQ-Aib-E-αMeF-IK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 120 | 5506.22 |
| 111 | Y-Aib-EGT-αMeF(2F)-TSDK((2-[2-(2-Amino-ethoxy)-ethoxy]-acetyl)₂-(γ-Glu)-CO-(CH₂)₁₂-CO₂H)SI-αMeL-LD-Orn-IAQ-Aib-E-αMeF-I-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 121 | 5506.22 |
| 112 | Y-Aib-EGT-αMeF(2F)-TSDK((2-[2-(2-Amino-ethoxy)-ethoxy]-acetyl)₂-(γ-Glu)-CO-(CH₂)₁₂-CO₂H)SI-αMeL-LD-Orn-IAQ-Aib-E-αMeF-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 122 | 5538.22 |
| 113 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFIK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 123 | 5525.22 |
| 114 | Y-Aib-EGT-αMeF(2F)-TSDVSI-αMeL-LD-Orn-K((2-[2-(2-Amino-ethoxy)-ethoxy]-acetyl)₂-(γ-Glu)-CO-(CH₂)₁₂-CO₂H)AQ-Aib-EFIK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 124 | 5478.16 |
| 115 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFIK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 125 | 5476.19 |
| 116 | Y-Aib-EGT-αMeF(2F)-TSD-Ac5c-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFIK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 126 | 5490.18 |
| 117 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-IK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 127 | 5539.25 |
| 118 | Y-Aib-EGT-αMeF(2F)-TSDVSI-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-IK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 128 | 5492.19 |
| 119 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-IK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 129 | 5490.22 |
| 120 | Y-Aib-EGT-αMeF(2F)-TSD-Ac5c-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-IK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 130 | 5504.2 |
| 121 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-IK((2-[2-(2-Amino-ethoxy)-ethoxy]-acetyl)₂-(γ-Glu)-CO-(CH₂)₁₂-CO₂H)-αMeF-LIEGGPSSGAPPPS-NH₂ | 131 | 5523.25 |
| 122 | Y-Aib-EGT-αMeF(2F)-TSDVSI-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-IK((2-[2-(2-Amino-ethoxy)-ethoxy]-acetyl)₂-(γ-Glu)-CO-(CH₂)₁₂-CO₂H)-αMeF-LIEGGPSSGAPPPS-NH₂ | 132 | 5476.19 |
| 123 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-IK((2-[2-(2-Amino-ethoxy)-ethoxy]-acetyl)₂-(γ-Glu)-CO-(CH₂)₁₂-CO₂H)-αMeF-LIEGGPSSGAPPPS-NH₂ | 133 | 5474.22 |
| 124 | Y-Aib-EGT-αMeF(2F)-TSD-Ac5c-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-αMeF-IK((2-[2-(2-Amino-ethoxy)-ethoxy]-acetyl)₂-(γ-Glu)-CO-(CH₂)₁₂-CO₂H)-αMeF-LIEGGPSSGAPPPS-NH₂ | 134 | 5488.2 |
| 125 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(7-(4-carboxyphenoxy)heptanoyl))AQ-Aib-EFI-(D-Glu)-αMeY-LIEGGK((2-[2-(2-Amino-ethoxy)-ethoxy]-acetyl)₂-(γ-Glu)-(7-(4-carboxyphenoxy)heptanoyl))SSGAPPPS-NH₂ | 135 | 5575.07 |
| 126 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(8-(4-carboxyphenoxy)octanoyl))AQ-Aib-EFI-(D-Glu)-αMeY-LIEGGK((2-[2-(2-Amino-ethoxy)-ethoxy]-acetyl)₂-(γ-Glu)-(8-(4-carboxyphenoxy)octanoyl))SSGAPPPS-NH₂ | 136 | 5603.12 |
| 127 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(10-(4-carboxyphenoxy)decanoyl))AQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIEGGPSSGAPPPS-NH₂ | 137 | 5643.27 |
| 128 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFIK((2-[2-(2-Amino-ethoxy)-ethoxy]-acetyl)₂-(γ-Glu)-(10-(4-carboxyphenoxy)decanoyl))-αMeY-LIEGGPSSGAPPPS-NH₂ | 138 | 5627.23 |
| 129 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 139 | 5627.23 |
| 130 | Y-Aib-EGT-αMeF(2F)-TSDK((2-[2-(2-Amino-ethoxy)-ethoxy]-acetyl)₂-(γ-Glu)-(4-(4-tert-butylphenyl)butanoyl))SI-αMeL-LD-Orn-K((2-[2-(2-Amino-ethoxy)-ethoxy]-acetyl)₂-(γ-Glu)-(4-(4-tert-butylphenyl)butanoyl))AQ-Aib-EFI-(D-Glu)-αMeY-LIEGGPSSGAPPPS-NH₂ | 140 | 5432.05 |
| 131 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(4-(4-tert-butylphenyl)butanoyl))AQ-Aib-K((2-[2-(2-Amino-ethoxy)-ethoxy]-acetyl)₂-(γ-Glu)-(4-(4-tert-butylphenyl)butanoyl))FI-(D-Glu)-αMeY-LIEGGPSSGAPPPS-NH₂ | 141 | 5451.1 |
| 132 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(4-(4-tert-butylphenyl)butanoyl))AQ-Aib-EFIK((2-[2-(2-Amino-ethoxy)-ethoxy]-acetyl)₂-(γ-Glu)-(4-(4-tert-butylphenyl)butanoyl))-αMeY-LIEGGPSSGAPPPS-NH₂ | 142 | 5451.1 |
| 133 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(4-(4-tert-butylphenyl)butanoyl))AQ-Aib-EFI-(D-Glu)-αMeY-LIK((2-[2-(2-Amino-ethoxy)-ethoxy]-acetyl)₂-(γ-Glu)-(4-(4-tert-butylphenyl)butanoyl))GGPSSGAPPPS-NH₂ | 143 | 5451.1 |
| 134 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(4-(4-tert-butylphenyl)butanoyl))AQ-Aib-EFI-(D-Glu)-αMeY-LIEGK((2-[2-(2-Amino-ethoxy)-ethoxy]-acetyl)₂-(γ-Glu)-(4-(4-tert- butylphenyl)butanoyl))PSSGAPPPS-NH₂ | 144 | 5523.17 |
| 135 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(4-(4-tert-butylphenyl)butanoyl))AQ-Aib-EFI-(D-Glu)-αMeY-LIEGGPSSGAPPPSK((2-[2-(2-Amino-ethoxy)-ethoxy]-acetyl)₂-(γ-Glu)-(4-(4-tert-butylphenyl)butanoyl))-NH₂ | 145 | 5580.22 |
| 136 | Y-Aib-EGT-αMeF(2F)-TSDK((2-[2-(2-Amino-ethoxy)-ethoxy]-acetyl)₂-(γ-Glu)-CO-(CH₂)₁₀-CH₃)SI-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGGPSSGAPPPS-NH₂ | 146 | 5392.08 |
| 137 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-K((2-[2-(2-Amino-ethoxy)-ethoxy]-acetyl)₂-(γ-Glu)-CO-(CH₂)₁₀-CH₃)FI-(D-Glu)-αMeY-LIEGGPSSGAPPPS-NH₂ | 147 | 5411.12 |
| 138 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-EFIK((2-[2-(2-Amino-ethoxy)-ethoxy]-acetyl)₂-(γ-Glu)-CO-(CH₂)₁₀-CH₃)-αMeY-LIEGGPSSGAPPPS-NH₂ | 148 | 5411.12 |
| 139 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIK((2-[2-(2-Amino-ethoxy)-ethoxy]-acetyl)₂-(γ-Glu)-CO-(CH₂)₁₀-CH₃)GGPSSGAPPPS-NH₂ | 149 | 5411.12 |
| 140 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGK((2-[2-(2-Amino-ethoxy)-ethoxy]-acetyl)₂-(γ-Glu)-CO-(CH₂)₁₀-CH₃)PSSGAPPPS-NH₂ | 150 | 5483.19 |
| 141 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGGP S SGAPPP SK((2-[2-(2-Amino-ethoxy)-ethoxy]-acetyl)₂-(γ-Glu)-CO-(CH₂)₁₀-CH₃)-NH₂ | 151 | 5540.24 |
| 142 | Y-Aib-EGT-αMeF(2F)-TSD-Bzt-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 152 | 5614.29 |
| 143 | Y-Aib-EGT-αMeF(2F)-TSD-2FA-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 153 | 5548.17 |
| 144 | Y-Aib-EGT-αMeF(2F)-TSD-4TAA-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 154 | 5565.22 |
| 145 | Y-Aib-EGT-αMeF(2F)-TSD-2TA-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 155 | 5564.23 |
| 146 | Y-Aib-EGT-αMeF(2F)-TSD-3TA-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 156 | 5564.23 |
| 147 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-Bzt-I-(D-Glu)-αMeY-LIEGGK((2-[2-(2-Amino-ethoxy)-ethoxy]-acetyl)₂-(γ- Glu)-CO-(CH₂)₁₂-CO₂H)SSGAPPPS-NH₂ | 157 | 5615.28 |
| 148 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-E-ChA-I-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 158 | 5565.24 |
| 149 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Orn-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 159 | 5588.24 |
| 150 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-Orn-FI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 160 | 5544.23 |
| 151 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LI-Orn-GGK((2-[2-(2-Amino-ethoxy)-ethoxy]-acetyl)₂-(γ-Glu)-CO-(CH₂)₁₂-CO₂H)SSGAPPPS-NH₂ | 161 | 5544.23 |
| 152 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEG-Orn-K((2-[2-(2-Amino-ethoxy)-ethoxy]-acetyl)₂-(γ-Glu)-CO-(CH₂)₁₂-CO₂H)SSGAPPPS-NH₂ | 162 | 5616.29 |
| 153 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSG-Orn-PPPS-NH₂ | 163 | 5602.26 |
| 154 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPP-Orn-NH₂ | 164 | 5586.26 |
| 155 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSG-Orn-PPP-Orn-NH₂ | 165 | 5629.33 |
| 156 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-4Pal-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 166 | 5622.25 |
| 157 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)ᵢ₂-CO₂H)AQ-Aib-4Pal-FI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 167 | 5578.24 |
| 158 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFIE-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 168 | 5559.19 |
| 159 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFIE-αMeF-LIEGGK((2-[2-(2-Amino-ethoxy)-ethoxy]-acetyl)₂-(γ-Glu)-CO-(CH₂)₁₂-CO₂H)SSGAPPPS-NH₂ | 169 | 5543.19 |
| 160 | Y-Aib-EGT-αMeF(2F)-TSD-4Pal-SI-αMeL-LD-Orn-K((2-[2-(2-Amino-ethoxy)-ethoxy]-acetyl)₂-(εK)-CO-(CH₂)₁₂-CO₂H)AQ-Aib-EFIK((2-[2-(2-Amino-ethoxy)-ethoxy]-acetyl)₂-(εK)-CO-(CH₂)₁₂-CO₂H)-αMeY-LIEGGPSSGAPPPS-NH₂ | 170 | 5525.31 |
| 161 | Y-Aib-EGT-aMeF(2F)-TSD-4Pal-SI-aMeL-LD-Orn-K(eK-gE-C14_OH)AQ-Aib-EFIK(eK-gE-C14_OH)-αMeY-LIEGGPSSGAPPPS-NH₂ | 171 | 5202.91 |
| 162 | Y-Aib-EGT-αMeF(2F)-TSD-4Pal-SI-αMeL-LD-Orn-K((εK)-(εK)-(γ-Glu)-CO-(CH₂)₁₂-CO₂H)AQ-Aib-EFIK((εK)-(εK)-(γ-Glu)-CO-(CH₂)₁₂-CO₂H)-αMeY-LIEGGPSSGAPPPS-NH₂ | 172 | 5459.26 |
| 163 | Y-Aib-EGT-αMeF(2F)-TSDV-Aib-I-αMeL-LD-Orn-K((2-[2-(2-Amino-ethoxy)-ethoxy]-acetyl)₂-(εK)-CO-(CH₂)₁₂-CO₂H)AQ-Aib-EFI-(D-Glu)-αMeY-LIEGGK((2-[2-(2-Amino-ethoxy)-ethoxy]-acetyl)₂-(εK)-CO-(CH₂)₁₂-CO₂H)SSGAPPPS-NH₂ | 173 | 5506.31 |
| 164 | Y-Aib-EGT-αMeF(2F)-TSDV-Aib-I-αMeL-LD-Orn-K((εK)-(γ-Glu)-CO-(CH₂)₁₂-CO₂H)AQ-Aib-EFI-(D-Glu)-αMeY-LIEGGK((εK)-(γ-Glu)-CO-(CH₂)₁₂-CO₂H)SSGAPPPS-NH₂ | 174 | 5183.91 |
| 165 | Y-Aib-EGT-αMeF(2F)-TSDV-Aib-I-αMeL-LD-Orn-K((εK)-(εK)-(γ-Glu)-CO-(CH₂)₁₂-CO₂H)AQ-Aib-EFI-(D-Glu)-αMeY-LIEGGK((εK)-(εK)-(γ-Glu)-CO-(CH₂)₁₂CO₂H)SSGAPPPS-NH₂ | 175 | 5440.25 |
| 166 | Y-Aib-EGT-αMeF(2F)-TSDV-Aib-I-αMeL-LD-Orn-K((2-[2-(2-Amino-ethoxy)-ethoxy]-acetyl)₂-(εK)-CO-(CH₂)₁₂-CO₂H)AQ-Aib-EFIK((2-[2-(2-Amino-ethoxy)-ethoxy]-acetyl)₂-(εK)-CO-(CH₂)₁₂-CO₂H)-αMeY-LIEGGPSSGAPPPS-NH₂ | 176 | 5474.31 |
| 167 | Y-Aib-EGT-αMeF(2F)-TSDV-Aib-I-αMeL-LD-Orn-K((εK)-(γ-Glu)-CO-(CH₂)₁₂-CO₂H)AQ-Aib-EFIK((εK)-(γ-Glu)-CO-(CH₂)₁₂-CO₂H)-αMeY-LIEGGPSSGAPPPS-NH₂ | 177 | 5151.91 |
| 168 | Y-Aib-EGT-αMeF(2F)-TSDV-Aib-I-αMeL-LD-Orn-K((εK)-(εK)-(γ-Glu)-CO-(CH₂)₁₂-CO₂H)AQ-Aib-EFIK((εK)-(εK)-(γ-Glu)-CO-(CH₂)₁₂-CO₂H)-αMeY-LIEGGPSSGAPPPS-NH₂ | 178 | 5408.25 |
| 169 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-aMeF-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 179 | 5635.29 |
| 170 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Ac5c-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 180 | 5585.23 |
| 171 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Ac6c-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 181 | 5599.26 |
| 172 | Y-Aib-EGT-αMeF-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGGK((2-[2-(2-Amino-ethoxy)-ethoxy]-acetyl)₂-(γ-Glu)-CO-(CH₂)₁₀-CH₃)SSGAPPPS-NH₂ | 182 | 5425.13 |
| 173 | Y-Aib-EGT-αMeF-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-E-αMeF-I-(D-Glu)-αMeY-LIEGGK((2-[2-(2-Amino-ethoxy)-ethoxy]-acetyl)₂-(γ-Glu)-CO-(CH₂)₁₀-CH₃)SSGAPPPS-NH₂ | 183 | 5439.16 |
| 174 | Y-Aib-EGT-αMeF-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-E-αMeF-I-(D-Glu)-αMeF-LIEGGK((2-[2-(2-Amino-ethoxy)-ethoxy]-acetyl)₂-(γ-Glu)-CO-(CH₂)₁₀-CH₃)SSGAPPPS-NH₂ | 184 | 5423.16 |
| 175 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGGK((2-[2-(2-Amino-ethoxy)-ethoxy]-acetyl)₂-(γ-Glu)-CO-(CH₂)₁₀-CH₃)SSGAPPPS-NH₂ | 185 | 5392.12 |
| 176 | Y-Aib-EGT-αMeF-TSDV-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGGK((2-[2-(2-Amino-ethoxy)-ethoxy]-acetyl)₂-(γ-Glu)-CO-(CH₂)₁₀-CH₃)SSGAPPPS-NH₂ | 186 | 5374.13 |
| 177 | Y-Aib-EGT-αMeF(2F)-TSD-V-Aib-I-αMeL-LD-Orn-K((2-[2-(2-Amino-ethoxy)-ethoxy]-acetyl)₂-(γ-Glu)-(4-(4-tert-butylphenyl)butanoyl))AQ-Aib-EFI-(D-Glu)-αMeY-LIEGGK((2-[2-(2-Amino-ethoxy)-ethoxy]-acetyl)₂-(γ-Glu)-(4-(4-tert-butylphenyl)butanoyl))SSGAPPPS-NH₂ | 187 | 5432.1 |
| 178 | Y-Aib-EGT-αMeF-TSD-V-Aib-I-αMeL-LD-Orn-K((2-[2-(2-Amino-ethoxy)-ethoxy]-acetyl)₂-(γ-Glu)-(4-(4-tert-butylphenyl)butanoyl))AQ-Aib-EFI-(D-Glu)-αMeY-LIEGGK((2-[2-(2-Amino-ethoxy)-ethoxy]-acetyl)₂-(γ-Glu)-(4-(4-tert-butylphenyl)butanoyl))SSGAPPPS-NH₂ | 188 | 5414.11 |
| 179 | Y-Aib-EGT-αMeF-TSDVSI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 189 | 5492.17 |
| 180 | Y-Aib-EGT-αMeF-TSDV-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 190 | 5490.2 |
| 181 | Y-Aib-EGT-αMeF-TSDVSI-αMeL-LD-Orn-K((2-[2-(2-Amino-ethoxy)-ethoxy]-acetyl)₂-(γ-Glu)-CO-(CH₂)₁₂-CO₂H)AQ-Aib-EFIK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 191 | 5460.17 |
| 182 | Y-Aib-EGT-αMeF-TSDV-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFIK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 192 | 5458.2 |
| 183 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSG-NH₂ | 193 | 5109.69 |
| 184 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)-NH₂ | 194 | 4878.49 |
| 185 | Y-Aib-EGT-αMeF(2F)-TSD-4Pal-SI-αMeL-LD-Orn-K((2-[2-(2-Amino-ethoxy)-ethoxy]-acetyl)₂-(εK)-CO-(CH₂)₁₂-CO₂H)AQ-Aib-EFI-(D-Glu)-αMeY-LIEGGK((2-[2-(2-Amino-ethoxy)-ethoxy]-acetyl)₂-(εK)-CO-(CH₂)₁₂-CO₂H)-NH₂ | 195 | 4876.6 |
| 186 | Y-Aib-EGT-αMeF(2F)-TSD-4Pal-SI-αMeL-LD-Orn-K((εK)-(γ-Glu)-CO-(CH₂)₁₂-CO₂H)AQ-Aib-EFI-(D-Glu)-αMeY-LIEGGK((εK)-(γ-Glu)-CO-(CH₂)₁₂-CO₂H)-NH₂ | 196 | 4554.21 |
| 187 | Y-Aib-EGT-αMeF(2F)-TSD-4Pal-SI-αMeL-LD-Orn-K((εK)-(εK)-(γ-Glu)-CO-(CH₂)₁₂-CO₂H)AQ-Aib-EFI-(D-Glu)-αMeY-LIEGGK((εK)-(εK)-(γ-Glu)-CO-(CH₂)₁₂-CO₂H)-NH₂ | 197 | 4810.55 |
| 188 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFIK((2-[2-(2-Amino-ethoxy)-ethoxy]-acetyl)₂-(γ-Glu)-CO-(CH₂)₁₂-CO₂H)-αMeY-LIEGGPSSG-NH₂ | 198 | 5077.69 |
| 189 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFIK((2-[2-(2-Amino-ethoxy)-ethoxy]-acetyl)₂-(γ-Glu)-CO-(CH₂)₁₂-CO₂H)-αMeY-LIEGG-NH₂ | 199 | 4749.37 |
| 190 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFIK((2-[2-(2-Amino-ethoxy)-ethoxy]-acetyl)₂-(γ-Glu)-CO-(CH₂)₁₂-CO₂H)-αMeY-LIEG-Orn-NH₂ | 200 | 4806.47 |
| 191 | Y-Aib-EGT-αMeF(2F)-TSD-4Pal-SI-αMeL-LD-Orn-IAQ-Aib-K((2-[2-(2-Amino-ethoxy)-ethoxy]-acetyl)₂-CO-(CH₂)₁₂-CO₂H)FIK((2-[2-(2-Amino-ethoxy)-ethoxy]-acetyl)₂-CO-(CH₂)₁₂-CO₂H)-αMeY-LIEGGPSSGAPPPS-NH₂ | 201 | 5253.01 |
| 192 | Y-Aib-EGT-αMeF(2F)-TSD-4Pal-SI-αMeL-LD-Orn-IAQ-Aib-EFIK((2-[2-(2-Amino-ethoxy)-ethoxy]-acetyl)₂-CO-(CH₂)₁₂-CO₂H)-αMeY-LIK((2-[2-(2-Amino-ethoxy)-ethoxy]-acetyl)₂-CO-(CH₂)₁₂-CO₂H)GGPSSGAPPPS-NH₂ | 202 | 5253.01 |
| 193 | Y-Aib-EGT-αMeF(2F)-TSD-4Pal-SI-αMeL-LD-Orn-IAQ-Aib-EFIK((2-[2-(2-Amino-ethoxy)-ethoxy]-acetyl)₂-CO-(CH₂)₁₂-CO₂H)-αMeY-LIEGGK((2-[2-(2-Amino-ethoxy)-ethoxy]-acetyl)₂-CO-(CH₂)₁₂-CO₂H)SSGAPPPS-NH₂ | 203 | 5285.01 |
| 194 | Y-Aib-EGT-αMeF(2F)-TSD-4Pal-SI-αMeL-LD-Orn-IAQ-Aib-EFIK((2-[2-(2-Amino-ethoxy)-ethoxy]-acetyl)₂-CO-(CH₂)₁₂-CO₂H)-αMeY-LIEGGPSSGAPPP SK((2-[2-(2-Amino-ethoxy)-ethoxy]-acetyl)₂-CO-(CH₂)₁₂-CO₂H)-NH₂ | 204 | 5382.13 |
| 195 | Y-Aib-EGT-αMeF(2F)-TSD-4Pal-SI-αMeL-LD-Orn-IAQ-Aib-K((2-[2-(2-Amino-ethoxy)-ethoxy]-acetyl)₂-CO-(CH₂)₁₂-CO₂H)FI-(D-Glu)-αMeY-LIEGGK((2-[2-(2-Amino-ethoxy)-ethoxy]-acetyl)₂-CO-(CH₂)₂-CO₂H)SSGAPPPS-NH₂ | 205 | 5285.01 |
| 196 | Y-Aib-EGT-αMeF(2F)-TSD-4Pal-SI-αMeL-LD-Orn-IAQ-Aib-K((2-[2-(2-Amino-ethoxy)-ethoxy]-acetyl)₂-CO-(CH₂)₁₂-CO₂H)FI-(D-Glu)-αMeY-LIEGGPSSGAPPP SK((2-[2-(2-Amino-ethoxy)-ethoxy]-acetyl)₂-CO-(CH₂)₁₂-CO₂H)-NH₂ | 206 | 5382.13 |
| 197 | Y-Aib-EGT-αMeF(2F)-TSD-4Pal-SI-αMeL-LD-Orn-IAQ-Aib-EFIK((εK)-(γ-Glu)-CO-(CH₂)₁₂-CO₂H)-αMeY-LIEGGK((εK)-(γ-Glu)-CO-(CH₂)₁₂-CO₂H)SSGAPPPS-NH₂ | 207 | 5218.96 |
| 198 | Y-Aib-EGT-αMeF(2F)-TSD-4Pal-SI-αMeL-LD-Orn-IAQ-Aib-EFIK((εK)-(εK)-(γ-Glu)-CO-(CH₂)₁₂-CO₂H)-αMeY-LIEGGK((εK)-(εK)-(γ-Glu)-CO-(CH₂)₁₂-CO₂H)SSGAPPPS-NH₂ | 208 | 5475.3 |
| 199 | Y-Aib-EGT-αMeF(2F)-TSD-4Pal-Dap-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 209 | 5558.21 |
| 200 | Y-Aib-EGT-αMeF(2F)-TSDY-Dap-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 210 | 5573.22 |
| 201 | Y-Aib-EGT-αMeF(2F)-TSDV-Dap-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFIe-αMeY-LIEGGK((2-[2-(2-Amino-ethoxy)-ethoxy]-acetyl)₂-(γ-Glu)-CO- (CH₂)₁₂-CO₂H)SSGAPPPS-NH₂ | 211 | 5509.18 |
| 202 | Y-Aib-EGT-αMeF(2F)-TSD-4Pal-Tle-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 212 | 5585.27 |
| 203 | Y-Aib-EGT-αMeF(2F)-TSD-4Pal-SI-αMeL-LD-Orn-IAQ-Aib-EFIK((2-[2-(2-Amino-ethoxy)-ethoxy]-acetyl)₂-(γ-Glu)-(4-(4-tert-butylphenyl)butanoyl))-αMeY-LIK((2-[2-(2-Amino-ethoxy)-ethoxy]-acetyl)₂-(γ-Glu)-(4-(4-tert-butylphenyl)butanoyl))GGPSSGAPPPS-NH2 | 213 | 5435.15 |
| 204 | Y-Aib-EGT-αMeF(2F)-TSD-4Pal-SI-αMeL-LD-Orn-IAQ-Aib-K((2-[2-(2-Amino-ethoxy)-ethoxy]-acetyl)₂-(γ-Glu)-(4-(4-tert-butylphenyl)butanoyl))FI-(D-Glu)-αMeY-LIK((2-[2-(2-Amino-ethoxy)-ethoxy]-acetyl)₂-(γ-Glu)-(4-(4-tert-butylphenyl)butanoyl))GGPSSGAPPPS-NH2 | 214 | 5435.15 |
| 205 | Y-Aib-EGT-αMeF(2F)-TSD-4Pal-SI-αMeL-LD-Orn-IAQ-Aib-K((2-[2-(2-Amino-ethoxy)-ethoxy]-acetyl)₂-(γ-Glu)-(4-(4-tert-butylphenyl)butanoyl))FI-(D-Glu)-αMeY-LIEGGPSSGAPPPSK((2-[2-(2-Amino-ethoxy)-ethoxy]-acetyl)₂-(γ-Glu)-(4-(4-tert-butylphenyl)butanoyl))-NH2 | 215 | 5564.26 |
| 206 | Y-Aib-EGT-αMeF(2F)-TSD-4Pal-SI-αMeL-LD-Orn-IAQ-Aib-EFI-(D-Glu)-αMeY-LIK((2-[2-(2-Amino-ethoxy)-ethoxy]-acetyl)₂-(γ-Glu)-(4-(4-tert-butylphenyl)butanoyl))GGPSSGAPPPSK((2-[2-(2-Amino-ethoxy)-ethoxy]-acetyl)₂-(γ-Glu)-(4-(4-tert-butylphenyl)butanoyl))-NH2 | 216 | 5564.26 |
| 207 | Y-Aib-EGT-αMeF(2F)-TSD-4Pal-SI-αMeL-LD-Orn-K((2-[2-(2-Amino-ethoxy)-ethoxy]-acetyl)-(γ-Glu)-(4-(4-tert-butylphenyl)butanoyl))AQ-Aib-K((2-[2-(2-Amino-ethoxy)-ethoxy]-acetyl)-(γ-Glu)-(4-(4-tert-butylphenyl)butanoyl))FI-(D-Glu)-αMeY-LIEGGPSSGAPPPS-NH2 | 217 | 5160.79 |
| 208 | Y-Aib-EGT-αMeF(2F)-TSD-4Pal-SI-αMeL-LD-Orn-K((2-[2-(2-Amino-ethoxy)-ethoxy]-acetyl)-(γ-Glu)-(4-(4-tert-butylphenyl)butanoyl))AQ-Aib-EFI-(D-Glu)-αMeY-LIK((2-[2-(2-Amino-ethoxy)-ethoxy]-acetyl)-(γ-Glu)-(4-(4-tert-butylphenyl)butanoyl))GGPSSGAPPPS-NH₂ | 218 | 5160.79 |
| 209 | Y-Aib-EGT-αMeF(2F)-TSD-4Pal-SI-αMeL-LD-Orn-K((2-[2-(2-Amino-ethoxy)-ethoxy]-acetyl)-(γ-Glu)-(4-(4-tert-butylphenyl)butanoyl))AQ-Aib-EFI-(D-Glu)-αMeY-LIEGGPSSGAPPPSK((2-[2-(2-Amino-ethoxy)-ethoxy]-acetyl)-(γ-Glu)-(4-(4-tert-butylphenyl)butanoyl))-NH₂ | 219 | 5289.9 |
| 210 | Y-Aib-EGT-αMeF(2F)-TSD-4Pal-SI-αMeL-LD-Orn-K((2-[2-(2-Amino-ethoxy)-ethoxy]-acetyl)₂-CO-(CH₂)₁₂-CO₂H)AQ-Aib-EFI-(D-Glu)-αMeY-LIK((2-[2-(2-Amino-ethoxy)-ethoxy]-acetyl)₂-CO-(CH₂)₁₂-CO₂H)GGPSSGAPPPS-NH₂ | 220 | 5398.12 |
| 211 | Y-Aib-EGT-αMeF(2F)-TSD-4Pal-Ac5c-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 221 | 5583.26 |
| 212 | Y-Aib-EGT-αMeF(2F)-TSDV-αMeS-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 222 | 5524.19 |
| 213 | Y-Aib-EGT-αMeF(2F)-TSD-4Pal-αMeS-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂ | 223 | 5573.22 |
| 214 | Y-Aib-EGT-αMeF(2F)-TSD-4Pal-αMeS-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFIK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 224 | 5541.22 |
| 215 | Y-Aib-EGT-αMeF(2F)-TSD-4Pal-αMeS-I-αMeL-LD-Orn-IAQ-Aib-K((2-[2-(2-Amino-ethoxy)-ethoxy]-acetyl)₂-(γ-Glu)-CO-(CH₂)₁₂-CO₂H)FIK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 225 | 5525.27 |
| 216 | Y-Aib-EGT-αMeF(2F)-TSD-4Pal-αMeS-I-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 226 | 5525.27 |
| 217 | Y-Aib-EGT-αMeF(2F)-TSD-4Pal-αMeS-I-αMeL-LD-Orn-IAQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 227 | 5525.27 |
| 218 | Y-Aib-EGT-αMeF(2F)-TSD-4Pal-Aib-I-αMeL-LD-Orn-IAQ-Aib-K((2-[2-(2-Amino-ethoxy)-ethoxy]-acetyl)₂-(γ-Glu)-CO-(CH₂)₁₂-CO₂H)FIK((2-[2-(2-Amino-ethoxy)-ethoxy]-acetyl)₂-(γ-Glu)-CO-(CH₂)₁₂-CO₂H)-αMeY-LIEGGPSSGAPPPS-NH₂ | 228 | 5509.27 |
| 219 | Y-Aib-EGT-αMeF(2F)-TSD-4Pal-Aib-I-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 229 | 5509.27 |
| 220 | Y-Aib-EGT-αMeF(2F)-TSD-4Pal-Aib-I-αMeL-LD-Orn-IAQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 230 | 5509.27 |
| 221 | Y-Aib-EGT-αMeF(2F)-TSD-4Pal-SI-αMeL-LD-Orn-K((2-[2-(2-Amino-ethoxy)-ethoxy]-acetyl)₂-CO-(CH₂)₁₂-CO₂H)AQ-Aib-EFI-(D-Glu)-αMeY-LIEGGPSSGAPPPSK((2-[2-(2-Amino-ethoxy)-ethoxy]-acetyl)₂-CO-(CH₂)₁₂-CO₂H)-NH₂ | 231 | 5398.08 |
| 222 | Y-Aib-EGT-αMeF(2F)-TSD-4Pal-SI-αMeL-LD-Orn-IAQ-Aib-K((2-[2-(2-Amino-ethoxy)-ethoxy]-acetyl)₂-CO-(CH₂)₁₂-CO₂H)Fl-(D-Glu)-αMeY-LIK((2-[2-(2-Amino-ethoxy)-ethoxy]-acetyl)₂-CO-(CH₂)₁₂-CO₂H)GGPSSGAPPPS-NH₂ | 232 | 5253.01 |
| 223 | Y-Aib-EGT-αMeF(2F)-TSD-4Pal-SI-αMeL-LD-Orn-IAQ-Aib-EFI-(D-Glu)-αMeY-LIK((2-[2-(2-Amino-ethoxy)-ethoxy]-acetyl)₂-CO-(CH₂)₁₂-CO₂H)GGPSSGAPPPSK((2-[2-(2-Amino-ethoxy)-ethoxy]-acetyl)₂-CO-(CH₂)₁₂-CO₂H)-NH₂ | 233 | 5382.13 |
| 224 | Y-Aib-EGT-αMeF(2F)-TSD-4Pal-SI-αMeL-LD-Orn-K((2-[2-(2-Amino-ethoxy)-ethoxy]-acetyl)₃-(γ-Glu)-CO-(CH₂)₁₂-CO₂H)AQ-Aib-EFI-(D-Glu)-αMeY-LIK((2-[2-(2-Amino-ethoxy)-ethoxy]-acetyl)₃-(γ-Glu)-CO-(CH₂)₁₂-CO₂H)GGPSSGAPPPS-NH₂ | 234 | 5817.51 |
| 225 | Y-Aib-EGT-αMeF(2F)-TSD-4Pal-SI-αMeL-LD-Orn-K((2-[2-(2-Amino-ethoxy)-ethoxy]-acetyl)₃-(γ-Glu)-CO-(CH₂)₁₂-CO₂H)AQ-Aib-EFI-(D-Glu)-αMeY-LIEGGPSSGAPPPSK((2-[2-(2-Amino-ethoxy)-ethoxy]-acetyl)₃-(γ-Glu)-CO-(CH₂)₁₂-CO₂H)-NH₂ | 235 | 5946.62 |
| 226 | Y-Aib-EGT-αMeF(2F)-TSD-4Pal-SI-αMeL-LD-Orn-IAQ-Aib-EFIK((2-[2-(2-Amino-ethoxy)-ethoxy]-acetyl)₃-(γ-Glu)-CO-(CH₂)₁₂-CO₂H)-αMeY-LIK((2-[2-(2-Amino-ethoxy)-ethoxy]-acetyl)₃-(γ-Glu)-CO-(CH₂)₁₂-CO₂H)GGPSSGAPPPS-NH₂ | 236 | 5801.55 |
| 227 | Y-Aib-EGT-αMeF(2F)-TSD-4Pal-SI-αMeL-LD-Orn-IAQ-Aib-K((2-[2-(2-Amino-ethoxy)-ethoxy]-acetyl)₃-(γ-Glu)-CO-(CH₂)₁₂-CO₂H)FI-(D-Glu)-αMeY-LIK((2-[2-(2-Amino-ethoxy)-ethoxy]-acetyl)₃-(γ-Glu)-CO-(CH₂)₁₂-CO₂H)GGPSSGAPPPS-NH₂ | 237 | 5801.55 |
| 228 | Y-Aib-EGT-αMeF(2F)-TSD-4Pal-SI-αMeL-LD-Orn-IAQ-Aib-K((2-[2-(2-Amino-ethoxy)-ethoxy]-acetyl)₃-(γ-Glu)-CO-(CH₂)₁₂-CO₂H)FI-(D-Glu)-αMeY-LIEGGPSSGAPPPSK((2-[2-(2-Amino-ethoxy)-ethoxy]-acetyl)₃-(γ-Glu)-CO-(CH₂)₁₂-CO₂H)-NH₂ | 238 | 5930.67 |
| 229 | Y-Aib-EGT-αMeF(2F)-TSD-4Pal-SI-αMeL-LD-Orn-IAQ-Aib-EFI-(D-Glu)-αMeY-LIK((2-[2-(2-Amino-ethoxy)-ethoxy]-acetyl)₃-(γ-Glu)-CO-(CH₂)₁₂-CO₂H)GGPSSGAPPPSK((2-[2-(2-Amino-ethoxy)-ethoxy]-acetyl)₃-(γ-Glu)-CO-(CH₂)₁₂-CO₂H)-NH₂ | 239 | 5930.67 |
| 230 | Y-Aib-EGT-αMeF(2F)-TSDY-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIEGGPSSGAPPPS-NH₂ | 240 | 5540.23 |
| 231 | Y-Aib-EGT-αMeF(2F)-TSDY-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 241 | 5540.23 |
| 232 | Y-Aib-EGT-αMeF(2F)-TSDY-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGPSSGAPPPSK((2-[2-(2-Amino-ethoxy)-ethoxyl-acetyl)₂-(γ-Glu)-CO-(CH₂)₁₂-CO₂H)-NH₂ | 242 | 5669.35 |
| 233 | Y-Aib-EGT-αMeF(2F)-TSDY-Aib-I-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 243 | 5524.28 |
| 234 | Y-Aib-EGT-αMeF(2F)-TSDY-Aib-I-αMeL-LD-Orn-IAQ-Aib-K((2-[2-(2-Amino-ethoxy)-ethoxy]-acetyl)₂-(γ-Glu)-CO-(CH₂)₁₂-CO₂H)Fl-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 244 | 5524.28 |
| 235 | Y-Aib-EGT-aMeF(2F)-TSDY-Aib-I-aMeL-LD-Orn-IAQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIEGGPSSGAPPPSK((2-[2-(2-Amino-ethoxy)-ethoxy]-acetyl)₂-(γ-Glu)-CO-(CH₂)₁₂-CO₂H)-NH₂ | 245 | 5653.39 |
| 236 | Y-Aib-EGT-αMeF(2F)-TSDY-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIEGGPSSGAPPPS-NH₂ | 246 | 5640.27 |
| 237 | Y-Aib-EGT-αMeF(2F)-TSDY-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 247 | 5640.27 |
| 238 | Y-Aib-EGT-αMeF(2F)-TSDY-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIEGGPSSGAPPPSK((2-[2-(2-Amino-ethoxy)-ethoxy]-acetyl)₂-(γ-Glu)-(10-(4-carboxyphenoxy)decanoyl))-NH₂ | 248 | 5769.38 |
| 239 | Y-Aib-EGT-αMeF(2F)-TSDY-Aib-I-αMeL-LD-Orn-IAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 249 | 5624.31 |
| 240 | Y-Aib-EGT-αMeF(2F)-TSDY-Aib-I-αMeL-LD-Orn-IAQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 250 | 5624.31 |
| 241 | Y-Aib-EGT-αMeF(2F)-TSDY-Aib-I-αMeL-LD-Orn-IAQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIEGGPSSGAPPPSK((2-[2-(2-Amino-ethoxy)-ethoxy]-acetyl)₂-(γ-Glu)-(10-(4-carboxyphenoxy)decanoyl))-NH₂ | 251 | 5753.42 |
| 242 | Y-Aib-EGT-αMeF(2F)-TSD-4Pal-Aib-I-αMeL-LD-Orn-IAQ-Aib-K((2-[2-(2-Amino-ethoxy)-ethoxy]-acetyl)₂-(γ-Glu)-(11-(4-carboxyphenoxy)undecanoyl)FI-(D-Glu)-αMeY-LIEGGPSSGAPPPSK((2-[2-(2-Amino-ethoxy)-ethoxy]-acetyl)₂-(γ-Glu)-(11-(4-carboxyphenoxy)undecanoyl)-NH₂ | 252 | 5766.46 |
| 243 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIEGGPSSGAPPPS-NH₂ | 253 | 5625.25 |
| 244 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 254 | 5625.25 |
| 245 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIEGGPSSGAPPPSK((2-[2-(2-Amino-ethoxy)-ethoxy]-acetyl)₂-(γ-Glu)-(10-(4-carboxyphenoxy)decanoyl))-NH₂ | 255 | 5754.37 |
| 246 | Y-Aib-EGT-αMeF(2F)-TSD-4Pal-Aib-I-αMeL-LD-Orn-IAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 256 | 5609.3 |
| 247 | Y-Aib-EGT-αMeF(2F)-TSD-4Pal-Aib-I-αMeL-LD-Orn-IAQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 257 | 5609.3 |
| 248 | Y-Aib-EGT-αMeF(2F)-TSD-4Pal-Aib-I-αMeL-LD-Orn-IAQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIEGGPSSGAPPPSK((2-[2-(2-Amino-ethoxy)-ethoxy]-acetyl)₂-(γ-Glu)-(10-(4-carboxyphenoxy)decanoyl))-NH₂ | 258 | 5738.41 |
| 249 | Y-Aib-EGT-αMeF(2F)-TSD-2Nal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIEGGPSSGAPPPS-NH₂ | 259 | 5576.27 |
| 250 | Y-Aib-EGT-αMeF(2F)-TSD-2Nal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 260 | 5576.27 |
| 251 | Y-Aib-EGT-αMeF(2F)-TSD-2Nal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGPSSGAPPPSK((2-[2-(2-Amino-ethoxy)-ethoxyl-acetyl)₂-(γ-Glu)-CO-(CH₂)₁₂-CO₂H)-NH₂ | 261 | 5705.38 |
| 252 | Y-Aib-EGT-αMeF(2F)-TSD-2Nal-SI-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 262 | 5560.31 |
| 253 | Y-Aib-EGT-αMeF(2F)-TSD-2Nal-SI-αMeL-LD-Orn-IAQ-Aib-K((2-[2-(2-Amino-ethoxy)-ethoxy]-acetyl)₂-(γ-Glu)-CO-(CH₂)₁₂-CO₂H)Fl-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 263 | 5560.31 |
| 254 | Y-Aib-EGT-αMeF(2F)-TSD-2Nal-SI-αMeL-LD-Orn-IAQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIEGGPSSGAPPPSK((2-[2-(2-Amino-ethoxy)-ethoxyl-acetyl)₂-(γ-Glu)-CO-(CH₂)₁₂-CO₂H)-NH₂ | 264 | 5689.42 |
| 255 | Y-Aib-EGT-αMeF(2F)-TSD-2Nal-SI-αMeL-LD-Orn-K((2-[2-(2-Amino-ethoxy)-ethoxy]-acetyl)₂-CO-(CH₂)₁₂-CO₂H)AQ-Aib-K((2-[2-(2-Amino-ethoxy)-ethoxy]-acetyl)₂-CO-(CH₂)₁₂-CO₂H)FI-(D-Glu)-αMeY-LIEGGPSSGAPPPS-NH₂ | 265 | 5318.04 |
| 256 | Y-Aib-EGT-αMeF(2F)-TSD-2Nal-SI-αMeL-LD-Orn-K((2-[2-(2-Amino-ethoxy)-ethoxy]-acetyl)₂-CO-(CH₂)₁₂-CO₂H)AQ-Aib-EFI-(D-Glu)-αMeY-LIK((2-[2-(2-Amino-ethoxy)-ethoxy]-acetyl)₂-CO-(CH₂)₁₂-CO₂H)GGPSSGAPPPS-NH₂ | 266 | 5318.04 |
| 257 | Y-Aib-EGT-αMeF(2F)-TSD-2Nal-SI-αMeL-LD-Orn-K((2-[2-(2-Amino-ethoxy)-ethoxy]-acetyl)₂-CO-(CH₂)₁₂-CO₂H)AQ-Aib-EFI-(D-Glu)-αMeY-LIEGGPSSGAPPPSK((2-[2-(2-Amino-ethoxy)-ethoxy]-acetyl)₂-CO-(CH₂)₁₂-CO₂H)-NH₂ | 267 | 5302.08 |
| 258 | Y-Aib-EGT-αMeF(2F)-TSD-2Nal-SI-αMeL-LD-Orn-IAQ-Aib-K((2-[2-(2-Amino-ethoxy)-ethoxy]-acetyl)₂-CO-(CH₂)₁₂-CO₂H)Fl-(D-Glu)-αMeY-LIEGGPSSGAPPPSK((2-[2-(2-Amino-ethoxy)-ethoxy]-acetyl)₂-CO-(CH₂)₁₂-CO₂H)-NH₂ | 268 | 5431.2 |
| 259 | Y-Aib-EGT-αMeF(2F)-TSD-OMeY-SI-αMeL-LD-Orn-K((2-[2-(2-Amino-ethoxy)-ethoxy]-acetyl)₂-(γ-Glu)-CO-(CH₂)₁₂-CO₂H)AQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIEGGPSSGAPPPS-NH₂ | 269 | 5556.23 |
| 260 | Y-Aib-EGT-αMeF(2F)-TSD-OMeY-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 270 | 5556.23 |
| 261 | Y-Aib-EGT-αMeF(2F)-TSD-OMeY-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGPSSGAPPPSK((2-[2-(2-Amino-ethoxy)-ethoxy]-acetyl)₂-(γ-Glu)-CO-(CH₂)₁₂-CO₂H)-NH₂ | 271 | 5685.35 |
| 262 | Y-Aib-EGT-αMeF(2F)-TSD-OMeY-SI-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 272 | 5540.28 |
| 263 | Y-Aib-EGT-αMeF(2F)-TSD-OMeY-SI-αMeL-LD-Orn-IAQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 273 | 5540.28 |
| 264 | Y-Aib-EGT-αMeF(2F)-TSD-OMeY-SI-αMeL-LD-Orn-IAQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIEGGPSSGAPPPSK((2-[2-(2-Amino-ethoxy)-ethoxyl-acetyl)₂-(γ-Glu)-CO-(CH₂)₁₂-CO₂H)-NH₂ | 274 | 5669.39 |
| 265 | Y-Aib-EGT-αMeF(2F)-TSD-OMeY-SI-αMeL-LD-Orn-K((2-[2-(2-Amino-ethoxy)-ethoxy]-acetyl)₂-CO-(CH₂)₁₂-CO₂H)AQ-Aib-K((2-[2-(2-Amino-ethoxy)-ethoxy]-acetyl)₂-CO-(CH₂)₁₂-CO₂H)FI-(D-Glu)-αMeY-LIEGGPSSGAPPPS-NH₂ | 275 | 5298.01 |
| 266 | Y-Aib-EGT-αMeF(2F)-TSD-OMeY-SI-αMeL-LD-Orn-K((2-[2-(2-Amino-ethoxy)-ethoxy]-acetyl)₂-CO-(CH₂)₁₂-CO₂H)AQ-Aib-EFI-(D-Glu)-αMeY-LIK((2-[2-(2-Amino-ethoxy)-ethoxy]-acetyl)₂-CO-(CH₂)₁₂-CO₂H)GGPSSGAPPPS-NH₂ | 276 | 5298.01 |
| 267 | Y-Aib-EGT-αMeF(2F)-TSD-OMeY-SI-αMeL-LD-Orn-K((2-[2-(2-Amino-ethoxy)-ethoxy]-acetyl)₂-CO-(CH₂)₁₂-CO₂H)AQ-Aib-EFI-(D-Glu)-αMeY-LIEGGPSSGAPPPSK((2-[2-(2-Amino-ethoxy)-ethoxy]-acetyl)₂-CO-(CH₂)₁₂-CO₂H)-NH₂ | 277 | 5427.12 |
| 268 | Y-Aib-EGT-αMeF(2F)-TSD-OMeY-SI-αMeL-LD-Orn-IAQ-Aib-K((2-[2-(2-Amino-ethoxy)-ethoxy]-acetyl)₂-CO-(CH₂)₁₂-CO₂H)FI-(D-Glu)-αMeY-LIEGGPSSGAPPPSK((2-[2-(2-Amino-ethoxy)-ethoxy]-acetyl)₂-CO-(CH₂)₁₂-CO₂H)-NH₂ | 278 | 5411.16 |
| 269 | Y-Aib-EGT-αMeF(2F)-TSD-Nle-SI-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 279 | 5476.23 |
| 270 | Y-Aib-EGT-αMeF(2F)-TSD-hTyr-SI-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 280 | 5540.28 |
| 271 | Y-Aib-EGT-αMeF(2F)-TSD-Nle-SI-αMeL-LD-Orn-K((2-[2-(2-Amino-ethoxy)-ethoxy]-acetyl)₂-CO-(CH₂)₁₂-CO₂H)AQ-Aib-K((2-[2-(2-Amino-ethoxy)-ethoxy]-acetyl)₂-CO-(CH₂)₁₂-CO₂H)FI-(D-Glu)-αMeY-LIEGGPSSGAPPPS-NH₂ | 281 | 5233.96 |
| 272 | Y-Aib-EGT-αMeF(2F)-TSD-hTyr-SI-αMeL-LD-Orn-K((2-[2-(2-Amino-ethoxy)-ethoxy]-acetyl)₂-CO-(CH₂)₁₂-CO₂H)AQ-Aib-K((2-[2-(2-Amino-ethoxy)-ethoxy]-acetyl)₂-CO-(CH₂)₁₂-CO₂H)FI-(D-Glu)-αMeY-LIEGGPSSGAPPPS-NH₂ | 282 | 5298.01 |
| 273 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIEGGPSSGAPPPSK((2-[2-(2-Amino-ethoxy)-ethoxy]-acetyl)₂-(γ-Glu)-(10-(4-carboxyphenoxy)decanoyl))-NH₂ | 283 | 5756.34 |
| 274 | Y-Aib-EGT-αMeF(2F)-TSD-4Pa1-SI-αMeL-LD-Orn-IAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 284 | 5611.27 |
| 275 | Y-Aib-EGT-αMeF(2F)-TSD-4Pa1-SI-αMeL-LD-Orn-IAQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 285 | 5611.27 |
| 276 | Y-Aib-EGT-αMeF(2F)-TSD-4Pal-SI-αMeL-LD-Orn-IAQ-Aib-K((2-[2-(2-Amino-ethoxy)-ethoxy]-acetyl)₂-(y-Glu)-(10-(4-carboxyphenoxy)decanoyl))FI-(D-Glu)-αMeY-LIEGGPSSGAPPPSK((2-[2-(2-Amino-ethoxy)-ethoxy]-acetyl)₂-(γ-Glu)-(10-(4-carboxyphenoxy)decanoyl))-NH₂ | 286 | 5740.38 |
| 277 | Y-Aib-EGT-αMeF(2F)-TSD-OMeY-SI-αMeL-LD-Orn-K((2-[2-(2-Amino-ethoxy)-ethoxy]-acetyl)₂-(γ-Glu)-(10-(4-carboxyphenoxy)decanoyl))AQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIEGGPSSGAPPPS-NH₂ | 287 | 5656.26 |
| 278 | Y-Aib-EGT-αMeF(2F)-TSD-OMeY-SI-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 288 | 5656.26 |
| 279 | Y-Aib-EGT-αMeF(2F)-TSD-OMeY-SI-αMeL-LD-Orn-IAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 289 | 5640.31 |
| 280 | Y-Aib-EGT-αMeF(2F)-TSD-OMeY-SI-αMeL-LD-Orn-IAQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 290 | 5640.31 |
| 281 | Y-Aib-EGT-αMeF(2F)-TSD-2Nal-SI-αMeL-LD-Orn-K((2-[2-(2-Amino-ethoxy)-ethoxy]-acetyl)₂-(γ-Glu)-(10-(4-carboxyphenoxy)decanoyl))AQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIEGGPSSGAPPPS-NH₂ | 291 | 5676.3 |
| 282 | Y-Aib-EGT-αMeF(2F)-TSD-2Nal-SI-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 292 | 5676.3 |
| 283 | Y-Aib-EGT-αMeF(2F)-TSD-2Nal-SI-αMeL-LD-Orn-IAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 293 | 5660.34 |
| 284 | Y-Aib-EGT-αMeF(2F)-TSD-2Nal-SI-αMeL-LD-Orn-IAQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 294 | 5660.34 |
| 285 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-(11-(4-carboxyphenoxy)undecanoyl))AQ-Aib-EFI-(D-Glu)-αMeY-LIEGGPSSGAPPPSK((2-[2-(2-Amino-ethoxy)-ethoxy]-acetyl)₂-(γ-Glu)-(11-(4-carboxyphenoxy)undecanoyl))-NH2 | 295 | 5784.39 |
| 286 | Y-Aib-EGT-αMeF(2F)-TSD-4Pal-SI-αMeL-LD-Orn-IAQ-Aib-EFIK((2-[2-(2-Amino-ethoxy)-ethoxy]-acetyl)₂-(γ-Glu)-(11-(4-carboxyphenoxy)undecanoyl)-αMeY-LIK((2-[2-(2-Amino-ethoxy)-ethoxy]-acetyl)₂-(γ-Glu)-(11-(4-carboxyphenoxy)undecanoyl)GGPSSGAPPPS-NH₂ | 296 | 5639.32 |
| 287 | Y-Aib-EGT-αMeF(2F)-TSD-4Pal-SI-αMeL-LD-Orn-IAQ-Aib-K((2-[2-(2-Amino-ethoxy)-ethoxy]-acetyl)₂-(γ-Glu)-(11-(4-carboxyphenoxy)undecanoyl)FI-(D-Glu)-αMeY-LIK((2-[2-(2-Amino-ethoxy)-ethoxy]-acetyl)₂-(γ-Glu)-(11-(4-carboxyphenoxy)undecanoyl)GGPSSGAPPPS-NH₂ | 297 | 5639.32 |
| 288 | Y-Aib-EGT-αMeF(2F)-TSD-4Pal-SI-αMeL-LD-Orn-IAQ-Aib-K((2-[2-(2-Amino-ethoxy)-ethoxy]-acetyl)₂-(γ-Glu)-(11-(4-carboxyphenoxy)undecanoyl)FI-(D-Glu)-αMeY-LIEGGPSSGAPPPSK((2-[2-(2-Amino-ethoxy)-ethoxy]-acetyl)₂-(γ-Glu)-(11-(4-carboxyphenoxy)undecanoyl)-NH₂ | 298 | 5768.44 |
| 289 | Y-Aib-EGT-αMeF(2F)-TSD-OMeY-SI-αMeL-LD-Orn-K((2-[2-(2-Amino-ethoxy)-ethoxy]-acetyl)₂-(γ-Glu)-CO-(CH₂)₁₀-CH₃)AQ-Aib-K((2-[2-(2-Amino-ethoxy)-ethoxy]-acetyl)₂-(γ-Glu)-CO-(CH₂)₁₀-CH₃)FI-(D-Glu)-αMeY-LIEGGPSSGAPPPS-NH₂ | 299 | 5440.16 |
| 290 | Y-Aib-EGT-αMeF(2F)-TSD-OMeY-SI-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIK((2-[2-(2-Amino-ethoxy)-ethoxy]-acetyl)₂-(γ-Glu)-CO-(CH₂)₁₀-CH₃)GGPSSGAPPPS-NH₂ | 300 | 5440.16 |
| 291 | Y-Aib-EGT-αMeF(2F)-TSD-OMeY-SI-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGGPSSGAPPPSK((2-[2-(2-Amino-ethoxy)-ethoxy]-acetyl)₂-(γ-Glu)-CO-(CH₂)₁₀-CH₃)-NH₂ | 301 | 5569.27 |
| 292 | Y-Aib-EGT-αMeF(2F)-TSDK((2-[2-(2-Amino-ethoxy)-ethoxy]-acetyl)₂-(γ-Glu)-CO-(CH₂)₁₀-CH₃)-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGGPSSGAPPPS-NH₂ | 302 | 5390.1 |
| 293 | Y-Aib-EGT-αMeF(2F)-TSDK((2-[2-(2-Amino-ethoxy)-ethoxy]-acetyl)₂-(γ-Glu)-CO-(CH₂)₁₀-CH₃)SI-αMeL-LD-Orn-K((2-[2-(2-Amino-ethoxy)-ethoxy]-acetyl)₂-(γ-Glu)-CO-(CH₂)₁₀-CH₃)AQ-Aib-E-αMeF-I-(D-Glu)-αMeY-LIEGGPSSGAPPPS-NH₂ | 303 | 5406.1 |
| 294 | Y-Aib-EGT-αMeF(2F)-TSDK((2-[2-(2-Amino-ethoxy)-ethoxy]-acetyl)₂-(γ-Glu)-CO-(CH₂)₁₀-CH₃)SI-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGG-NH₂ | 304 | 4614.25 |
| 295 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-K((2-[2-(2-Amino-ethoxy)-ethoxy]-acetyl)₂-(γ-Glu)-CO-(CH₂)₁₀-CH₃)FI-(D-Glu)-αMeY-LIEGG-NH₂ | 305 | 4633.3 |
| 296 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIK((2-[2-(2-Amino-ethoxy)-ethoxy]-acetyl)₂-(γ-Glu)-CO-(CH₂)₁₀-CH₃)GG-NH₂ | 306 | 4633.3 |
| 297 | Y-Aib-EGT-αMeF(2F)-TSDK((2-[2-(2-Amino-ethoxy)-ethoxy]-acetyl)₂-(γ-Glu)-CO-(CH₂)₁₀-CH₃)SI-αMeL-LD-Orn-K((2-[2-(2-Amino-ethoxy)-ethoxy]-acetyl)₂-(γ-Glu)-CO-(CH₂)₁₀-CH₃)AQ-Aib-E-αMeF-I-(D-Glu)-αMeY-LIEGG-NH₂ | 307 | 4628.28 |
| 298 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-K((2-[2-(2-Amino-ethoxy)-ethoxy]-acetyl)₂-(γ-Glu)-CO-(CH₂)₁₀-CH₃)FI-(D-Glu)-αMeY-LI-Orn-GGPSSGAPPPS-NH₂ | 308 | 5396.15 |
| 299 | Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-Orn-FI-(D-Glu)-αMeY-LIK((2-[2-(2-Amino-ethoxy)-ethoxy]-acetyl)₂-(γ-Glu)-CO-(CH₂)₁₀-CH₃)GGPSSGAPPPS-NH₂ | 309 | 5396.15 |
| 300 | Y-Aib-EGT-αMeF(2F)-TSDY-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-K((2-[2-(2-Amino-ethoxy)-ethoxy]-acetyl)₂-(γ-Glu)-CO-(CH₂)₁₀-CH₃)FI-(D-Glu)-αMeY-LIEGGPSSGAPPPS-NH₂ | 310 | 5424.16 |
| 301 | Y-Aib-EGT-αMeF(2F)-TSDY-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIK((2-[2-(2-Amino-ethoxy)-ethoxy]-acetyl)₂-(γ-Glu)-CO-(CH₂)₁₀-CH₃)GGPSSGAPPPS-NH₂ | 311 | 5424.16 |
| 302 | Y-Aib-EGT-αMeF(2F)-TSDY-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGGPSSGAPPPSK((2-[2-(2-Amino-ethoxy)-ethoxy]-acetyl)₂-(γ-Glu)-CO-(CH₂)₁₀-CH₃)-NH₂ | 312 | 5553.28 |
| 303 | Y-Aib-EGT-αMeF(2F)-TSDY-αMeS-I-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 313 | 5540.28 |
| 304 | Y-Aib-EGT-αMeF(2F)-TSDV-Aib-I-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 314 | 5460.24 |
| 305 | Y-Aib-EGT-αMeF(2F)-TSDV-αMeS-I-αMeL-LD-Orn-IAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 315 | 5476.23 |
| 306 | Y-Aib-EGT-αMeF(2F)-TSD-4Pal-αMeS-I-αMeL-LD-Orn-IAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 316 | 5625.3 |
| 307 | Y-Aib-EGT-αMeF(2F)-TSDY-αMeS-I-αMeL-LD-Orn-IAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 317 | 5640.31 |
| 308 | Y-Aib-EGT-αMeF(2F)-TSDV-Aib-I-αMeL-LD-Orn-IAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 318 | 5560.27 |
| 309 | Y-Aib-EGT-αMeF(2F)-TSDV-αMeS-I-αMeL-LD-Orn-IAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 319 | 5576.27 |
| 310 | Y-Aib-EGT-αMeF(2F)-TSD-hTyr-SI-αMeL-LD-Orn-IAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 320 | 5640.31 |
| 311 | Y-Aib-EGT-αMeF(2F)-TSD-4Pal-Aib-I-αMeL-LD-Orn-IAQ-Aib-EFIK((2-[2-(2-Amino-ethoxy)-ethoxy]-acetyl)₂-(γ-Glu)-(11-(4-carboxyphenoxy)undecanoyl)-αMeY-LIK((2-[2-(2-Amino-ethoxy)-ethoxy]-acetyl)₂-(γ-Glu)-(11-(4-carboxyphenoxy)undecanoyl)GGPSSGAPPPS-NH₂ | 321 | 5637.35 |
| 312 | Y-Aib-EGT-αMeF(2F)-TSDY-Aib-I-αMeL-LD-Orn-IAQ-Aib-EFIK((2-[2-(2-Amino-ethoxy)-ethoxy]-acetyl)₂-(γ-Glu)-(11-(4-carboxyphenoxy)undecanoyl)-αMeY-LIK((2-[2-(2-Amino-ethoxy)-ethoxy]-acetyl)₂-(γ-Glu)-(11-(4-carboxyphenoxy)undecanoyl)GGPSSGAPPPS-NH₂ | 322 | 5652.36 |
| 313 | Y-Aib-EGT-αMeF(2F)-TSDV-Aib-I-αMeL-LD-Orn-IAQ-Aib-EFIK((2-[2-(2-Amino-ethoxy)-ethoxy]-acetyl)₂-(γ-Glu)-(11-(4-carboxyphenoxy)undecanoyl)-αMeY-LIK((2-[2-(2-Amino-ethoxy)-ethoxy]-acetyl)₂-(γ-Glu)-(11-(4-carboxyphenoxy)undecanoyl)GGPSSGAPPPS-NH₂ | 323 | 5588.32 |
| 314 | Y-Aib-EGT-αMeF(2F)-TSD-Nle-SI-αMeL-LD-Orn-IAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 324 | 5576.27 |
| 315 | Y-Aib-EGT-αMeF(2F)-TSD-4Pal-Aib-I-αMeL-LD-Orn-IAQ-Aib-Orn-FIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 325 | 5494.3 |
| 316 | Y-Aib-EGT-αMeF(2F)-TSDY-Aib-I-αMeL-LD-Orn-IAQ-Aib-Orn-FIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 326 | 5509.31 |
| 317 | Y-Aib-EGT-αMeF(2F)-TSDV-Aib-I-αMeL-LD-Orn-IAQ-Aib-Orn-FIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 327 | 5445.27 |
| 318 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIEGGPSSGAPPPS-NH₂ | 328 | 5476.19 |
| 319 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIEGGPSSGAPPPS-NH₂ | 329 | 5576.22 |
| 320 | Y-Aib-EGT-αMeF(2F)-TSD-hTyr-SI-αMeL-LD-Orn-K((2-[2-(2-Amino-ethoxy)-ethoxy]-acetyl)₂-(y-Glu)-(10-(4-carboxyphenoxy)decanoyl))AQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIEGGPSSGAPPPS-NH₂ | 330 | 5656.26 |
| 321 | Y-Aib-EGT-αMeF(2F)-TSD-Nle-SI-αMeL-LD-Orn-K((2-[2-(2-Amino-ethoxy)-ethoxy]-acetyl)₂-(γ-Glu)-(10-(4-carboxyphenoxy)decanoyl))AQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIEGGPSSGAPPPS-NH₂ | 331 | 5592.22 |
| 322 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-K((2-[2-(2-Amino-ethoxy)-ethoxy]-acetyl)₂-(γ-Glu)-CO-(CH₂)₁₀-CH₃)FI-(D-Glu)-αMeY-LIEGGPSSGAPPPS-NH2 | 332 | 5409.15 |
| 323 | Y-Aib-EGT-αMeF(2F)-TSD-hTyr-SI-αMeL-LD-Orn-K((2-[2-(2-Amino-ethoxy)-ethoxy]-acetyl)₂-(γ-Glu)-CO-(CH₂)₁₀-CH₃)AQ-Aib-K((2-[2-(2-Amino-ethoxy)-ethoxy]-acetyl)₂-(γ-Glu)-CO-(CH₂)₁₀-CH₃)FI-(D-Glu)-αMeY-LIEGGPSSGAPPPS-NH₂ | 333 | 5440.16 |
| 324 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGGPSSGAPPPSK((2-[2-(2-Amino-ethoxy)-ethoxy]-acetyl)₂-(γ-Glu)-CO-(CH₂)₁₀-CH₃)-NH₂ | 334 | 5538.26 |
| 325 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 335 | 5476.19 |
| 326 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 336 | 5576.22 |
| 327 | Y-Aib-EGT-αMeF(2F)-TSD-hTyr-SI-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 337 | 5656.26 |
| 328 | Y-Aib-EGT-αMeF(2F)-TSD-Nle-SI-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 338 | 5592.22 |
| 329 | Y-Aib-EGT-αMeF(2F)-TSDV-Aib-I-αMeL-LD-Orn-IAQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 339 | 5460.24 |
| 330 | Y-Aib-EGT-αMeF(2F)-TSDV-Aib-I-αMeL-LD-Orn-IAQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 340 | 5560.27 |
| 331 | Y-Aib-EGT-αMeF(2F)-TSD-4Pal-Aib-I-αMeL-LD-Orn-IAQ-Aib-K((2-[2-(2-Amino-ethoxy)-ethoxy]-acetyl)₂-(γ-Glu)-(11-(4-carboxyphenoxy)undecanoyl)FI-(D-Glu)-αMeY-LIK((2-[2-(2-Amino-ethoxy)-ethoxy]-acetyl)₂-(γ-Glu)-(11-(4-carboxyphenoxy)undecanoyl)GGPSSGAPPPS-NH₂ | 341 | 5637.35 |
| 332 | Y-Aib-EGT-αMeF(2F)-TSD-hTyr-SI-αMeL-LD-Orn-IAQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 342 | 5640.31 |
| 333 | Y-Aib-EGT-αMeF(2F)-TSD-Nle-SI-αMeL-LD-Orn-IAQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 343 | 5576.27 |
| 334 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIK((2-[2-(2-Amino-ethoxy)-ethoxy]-acetyl)₂-(γ-Glu)-CO-(CH₂)₁₀-CH₃)GGPSSGAPPPS-NH₂ | 344 | 5409.15 |
| 335 | Y-Aib-EGT-αMeF(2F)-TSD-hTyr-SI-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIK((2-[2-(2-Amino-ethoxy)-ethoxy]-acetyl)₂-(γ-Glu)-CO-(CH₂)₁₀-CH₃)GGPSSGAPPPS-NH₂ | 345 | 5440.16 |
| 336 | Y-Aib-EGT-αMeF(2F)-TSD-4Pal-Aib-I-αMeL-LD-Orn-K((2-[2-(2-Amino-ethoxy)-ethoxy]-acetyl)-(γ-Glu)-CO-(CH₂)₁₂-CO₂H)AQ-Aib-K((2-[2-(2-Amino-ethoxy)-ethoxy]-acetyl)-(γ-Glu)-CO-(CH₂)₁₂-CO₂H)FI-(D-Glu)-αMeY-LIEGGPSSGAPPPS-NH₂ | 346 | 5234.91 |
| 337 | Y-Aib-EGT-αMeF(2F)-TSD-4Pal-Aib-I-αMeL-LD-Orn-K((2-[2-(2-Amino-ethoxy)-ethoxy]-acetyl)-(γ-Glu)-CO-(CH₂)₁₂-CO₂H)AQ-Aib-EFI-(D-Glu)-αMeY-LIK((2-[2-(2-Amino-ethoxy)-ethoxy]-acetyl)-(γ-Glu)-CO-(CH₂)₁₂-CO₂H)GGPSSGAPPPS-NH₂ | 347 | 5234.91 |
| 338 | Y-Aib-EGT-αMeF(2F)-TSD-4Pal-Aib-I-αMeL-LD-Orn-K((2-[2-(2-Amino-ethoxy)-ethoxy]-acetyl)-(γ-Glu)-CO-(CH₂)₁₂-CO₂H)AQ-Aib-EFI-(D-Glu)-αMeY-LIEGGPSSGAPPPSK((2-[2-(2-Amino-ethoxy)-ethoxy]-acetyl)-(γ-Glu)-CO-(CH₂)₁₂-CO₂H)-NH₂ | 348 | 5364.02 |
| 339 | Y-Aib-EGT-αMeF(2F)-TSD-4Pal-Aib-I-αMeL-LD-Orn-IAQ-Aib-EFIK((2-[2-(2-Amino-ethoxy)-ethoxy]-acetyl)-(γ-Glu)-CO-(CH₂)₁₂-CO₂H)-αMeY-LIK((2-[2-(2-Amino-ethoxy)-ethoxy]-acetyl)-(γ-Glu)-CO-(CH₂)₁₂-CO₂H)GGPSSGAPPPS-NH₂ | 349 | 5218.95 |
| 340 | Y-Aib-EGT-αMeF(2F)-TSD-4Pal-Aib-I-αMeL-LD-Orn-IAQ-Aib-K((2-[2-(2-Amino-ethoxy)-ethoxy]-acetyl)-(γ-Glu)-CO-(CH₂)₁₂-CO₂H)FI-(D-Glu)-αMeY-LIK((2-[2-(2-Amino-ethoxy)-ethoxy]-acetyl)-(γ-Glu)-CO-(CH₂)₁₂-CO₂H)GGPSSGAPPPS-NH₂ | 350 | 5218.95 |
| 341 | Y-Aib-EGT-αMeF(2F)-TSD-4Pal-Aib-I-αMeL-LD-Orn-IAQ-Aib-K((2-[2-(2-Amino-ethoxy)-ethoxy]-acetyl)-(γ-Glu)-CO-(CH₂)₁₂-CO₂H)FI-(D-Glu)-αMeY-LIEGGPSSGAPPPSK((2-[2-(2-Amino-ethoxy)-ethoxy]-acetyl)-(γ-Glu)-CO-(CH₂)₁₂-CO₂H)-NH₂ | 351 | 5348.07 |
| 342 | Y-Aib-EGT-αMeF(2F)-TSD-4Pal-Aib-I-αMeL-LD-Orn-IAQ-Aib-EFIK((2-[2-(2-Amino-ethoxy)-ethoxy]-acetyl)₃-(γ-Glu)-CO-(CH₂)₁₂-CO₂H)-αMeY-LIK((2-[2-(2-Amino-ethoxy)-ethoxy]-acetyl)₃-(γ-Glu)-CO-(CH₂)₁₂-CO₂H)GGPSSGAPPPS-NH₂ | 352 | 5799.58 |
| 343 | Y-Aib-EGT-αMeF(2F)-TSDY-Aib-I-αMeL-LD-Orn-IAQ-Aib-EFIK((2-[2-(2-Amino-ethoxy)-ethoxy]-acetyl)₃-(γ-Glu)-CO-(CH₂)₁₂-CO₂H)-αMeY-LIK((2-[2-(2-Amino-ethoxy)-ethoxy]-acetyl)₃-(γ-Glu)-CO-(CH₂)₁₂-CO₂H)GGPSSGAPPPS-NH₂ | 353 | 5814.59 |
| 344 | Y-Aib-EGT-αMeF(2F)-TSDV-Aib-I-αMeL-LD-Orn-IAQ-Aib-EFIK((2-[2-(2-Amino-ethoxy)-ethoxy]-acetyl)₃-(γ-Glu)-CO-(CH₂)₁₂-CO₂H)-αMeY-LIK((2-[2-(2-Amino-ethoxy)-ethoxy]-acetyl)₃-(γ-Glu)-CO-(CH₂)₁₂-CO₂H)GGPSSGAPPPS-NH₂ | 354 | 5750.55 |
| 345 | Y-Aib-EGT-αMeF(2F)-TSD-4Pal-Aib-I-αMeL-LD-Orn-IAQ-Aib-EFIK((2-[2-(2-Amino-ethoxy)-ethoxy]-acetyl)₃-(γ-Glu)-(10-(4-carboxyphenoxy)decanoyl))-αMeY-LIK((2-[2-(2-Amino-ethoxy)-ethoxy]-acetyl)₃-(γ-Glu)-(10-(4-carboxyphenoxy)decanoyl))GGPSSGAPPPS-NH₂ | 355 | 5899.61 |
| 346 | Y-Aib-EGT-αMeF(2F)-TSDY-Aib-I-αMeL-LD-Orn-IAQ-Aib-EFIK((2-[2-(2-Amino-ethoxy)-ethoxy]-acetyl)₃-(γ-Glu)-(10-(4-carboxyphenoxy)decanoyl)-αMeY-LIK((2-[2-(2-Amino-ethoxy)-ethoxy]-acetyl)₃-(γ-Glu)-(10-(4-carboxyphenoxy)decanoyl)GGPSSGAPPPS-NH₂ | 356 | 5914.62 |
| 347 | Y-Aib-EGT-αMeF(2F)-TSDV-Aib-I-αMeL-LD-Orn-IAQ-Aib-EFIK((2-[2-(2-Amino-ethoxy)-ethoxy]-acetyl)₃-(γ-Glu)-(10-(4-carboxyphenoxy)decanoyl)-αMeY-LIK((2-[2-(2-Amino-ethoxy)-ethoxy]-acetyl)₃-(γ-Glu)-(10-(4-carboxyphenoxy)decanoyl)GGPSSGAPPPS-NH₂ | 357 | 5850.58 |
| 348 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-Orn-FI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 358 | 5510.25 |
| 349 | Y-Aib-EGT-γMeF(2F)-TSDY-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-Orn-FI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 359 | 5525.27 |
| 350 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-Orn-FI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 360 | 5461.22 |
| 351 | Y-Aib-EGT-αMeF(2F)-TSD-4Pal-Aib-I-αMeL-LD-Orn-K((2-[2-(2-Amino-ethoxy)-ethoxy]-acetyl)₂-(α-Glu)-(10-(4-carboxyphenoxy)decanoyl))AQ-Aib-Om-FI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 361 | 5610.29 |
| 352 | Y-Aib-EGT-αMeF(2F)-TSDY-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-Om-FI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 362 | 5625.3 |
| 353 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-Om-FI-(D-Glu)-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 363 | 5561.25 |
| 354 | Y-Aib-EGT-αMeF(2F)-TSD-4Pal-Aib-I-αMeL-LD-Orn-IAQ-Aib-Orn-FIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 364 | 5594.33 |
| 355 | Y-Aib-EGT-αMeF(2F)-TSDY-Aib-I-αMeL-LD-Orn-IAQ-Aib-Orn-FIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 365 | 5609.34 |
| 356 | Y-Aib-EGT-αMeF(2F)-TSDV-Aib-I-αMeL-LD-Orn-IAQ-Aib-Orn-FIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 366 | 5545.3 |
| 357 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIEGGPSSGAPPPS-NH₂ | 367 | 5525.22 |
| 358 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 368 | 5525.22 |
| 359 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGPSSGAPPPSK((2-[2-(2-Amino-ethoxy)-ethoxy]-acetyl)₂-(γ-Glu)-CO-(CH₂)₁₂-CO₂H)-NH₂ | 369 | 5654.34 |
| 360 | Y-Aib-EGT-αMeF(2F)-TSD-4Pal-Aib-I-αMeL-LD-Orn-K((2-[2-(2-Amino-ethoxy)-ethoxy]-acetyl)-(γ-Glu)-(10-(4-carboxyphenoxy)decanoyl))AQ-Aib-K((2-[2-(2-Amino-ethoxy)-ethoxy]-acetyl)-(γ-Glu)-(10-(4-carboxyphenoxy)decanoyl))FI-(D-Glu)-αMeY-LIEGGPSSGAPPPS-NH₂ | 370 | 5334.94 |
| 361 | Y-Aib-EGT-αMeF(2F)-TSD-4Pal-Aib-I-αMeL-LD-Orn-K((2-[2-(2-Amino-ethoxy)-ethoxy]-acetyl)-(γ-Glu)-(10-(4-carboxyphenoxy)decanoyl))AQ-Aib-EFIe-αMeY-LIK((2-[2-(2-Amino-ethoxy)-ethoxy]-acetyl)-(γ-Glu)-(10-(4-carboxyphenoxy)decanoyl))GGPSSGAPPPS-NH₂ | 371 | 5334.94 |
| 362 | Y-Aib-EGT-αMeF(2F)-TSD-4Pal-Aib-I-αMeL-LD-Orn-K((2-[2-(2-Amino-ethoxy)-ethoxy]-acetyl)-(y-Glu)-(10-(4-carboxyphenoxy)decanoyl))AQ-Aib-EFI-(D-Glu)-αMeY-LIEGGPSSGAPPPSK((2-[2-(2-Amino-ethoxy)-ethoxy]-acetyl)-(γ-Glu)-(10-(4-carboxyphenoxy)decanoyl))-NH₂ | 372 | 5464.05 |
| 363 | Y-Aib-EGT-αMeF(2F)-TSDY-Aib-I-αMeL-LD-Orn-IAQ-Aib-EFIK((2-[2-(2-Amino-ethoxy)-ethoxy]-acetyl)₂-(γ-Glu)-(2-[2-(2-Amino-ethoxy)-ethoxy]-acetyl)-CO-(CH₂)₁₂-CO₂H)-αMeY-LIK((2-[2-(2-Amino-ethoxy)-ethoxy]-acetyl)₂-(γ-Glu)-(2-[2-(2-Amino-ethoxy)-ethoxy]-acetyl)-CO-(CH₂)₁₂-CO₂H)GGPSSGAPPPS-NH₂ | 373 | 5814.59 |
| 364 | Y-Aib-EGT-αMeF(2F)-TSDY-Aib-I-αMeL-LD-Orn-IAQ-Aib-EFIK((2-[2-(2-Amino-ethoxy)-ethoxy]-acetyl)-(γ-Glu)-(2-[2-(2-Amino-ethoxy)-ethoxy]-acetyl)₂-CO-(CH₂)₁₂-CO₂H)-αMeY-LIK((2-[2-(2-Amino-ethoxy)-ethoxy]-acetyl)-(γ-Glu)-(2-[2-(2-Amino-ethoxy)-ethoxy]-acetyl)₂-CO-(CH₂)₁₂-CO₂H)GGPSSGAPPPS-NH₂ | 374 | 5814.59 |
| 365 | Y-Aib-EGT-αMeF(2F)-TSDY-Aib-I-αMeL-LD-Orn-IAQ-Aib-EFIK((2-[2-(2-Amino-ethoxy)-ethoxy]-acetyl)-(γ-Glu)-(2-[2-(2-Amino-ethoxy)-ethoxy]-acetyl)-CO-(CH₂)₁₂-CO₂H)-αMeY-LIK((2-[2-(2-Amino-ethoxy)-ethoxy]-acetyl)-(γ-Glu)-(2-[2-(2-Amino-ethoxy)-ethoxy]-acetyl)-CO-(CH₂)₁₂-CO₂H)GGPSSGAPPPS-NH₂ | 375 | 5524.28 |
| 366 | Y-Aib-EGT-αMeF(2F)-TSDY-Aib-I-αMeL-LD-Orn-K((2-[2-(2-Amino-ethoxy)-ethoxy]-acetyl)-(γ-Glu)-CO-(CH₂)₁₂-CO₂H)AQ-Aib-K((2-[2-(2-Amino-ethoxy)-ethoxy]-acetyl)-(γ-Glu)-CO-(CH₂)₁₂-CO₂H)FI-(D-Glu)-αMeY-LIEGGPSSGAPPPS-NH₂ | 376 | 5249.92 |
| 367 | Y-Aib-EGT-αMeF(2F)-TSDY-Aib-I-αMeL-LD-Orn-K((2-[2-(2-Amino-ethoxy)-ethoxy]-acetyl)-(γ-Glu)-CO-(CH₂)₁₂-CO₂H)AQ-Aib-EFI-(D-Glu)-αMeY-LIK((2-[2-(2-Amino-ethoxy)-ethoxy]-acetyl)-(γ-Glu)-CO-(CH₂)₁₂-CO₂H)GGPSSGAPPPS-NH₂ | 377 | 5249.92 |
| 368 | Y-Aib-EGT-αMeF(2F)-TSDY-Aib-I-αMeL-LD-Orn-K((2-[2-(2-Amino-ethoxy)-ethoxy]-acetyl)-(γ-Glu)-CO-(CH₂)₁₂-CO₂H)AQ-Aib-EFI-(D-Glu)-αMeY-LIEGGPSSGAPPPSK((2-[2-(2-Amino-ethoxy)-ethoxy]-acetyl)-(γ-Glu)-CO-(CH₂)₁₂-CO₂H)-NH₂ | 378 | 5379.03 |
| 369 | Y-Aib-EGT-αMeF(2F)-TSDY-Aib-I-αMeL-LD-Orn-IAQ-Aib-EFIK((2-[2-(2-Amino-ethoxy)-ethoxy]-acetyl)-(γ-Glu)-CO-(CH₂)₁₂-CO₂H)-αMeY-LIK((2-[2-(2-Amino-ethoxy)-ethoxy]-acetyl)-(γ-Glu)-CO-(CH₂)₁₂-CO₂H)GGPSSGAPPPS-NH₂ | 379 | 5233.96 |
| 370 | Y-Aib-EGT-αMeF(2F)-TSDY-Aib-I-αMeL-LD-Orn-IAQ-Aib-K((2-[2-(2-Amino-ethoxy)-ethoxy]-acetyl)-(γ-Glu)-CO-(CH₂)₁₂-CO₂H)FI-(D-Glu)-αMeY-LIK((2-[2-(2-Amino-ethoxy)-ethoxy]-acetyl)-(γ-Glu)-CO-(CH₂)₁₂-CO₂H)GGPSSGAPPPS-NH₂ | 380 | 5233.96 |
| 371 | Y-Aib-EGT-αMeF(2F)-TSDY-Aib-I-αMeL-LD-Orn-IAQ-Aib-K((2-[2-(2-Amino-ethoxy)-ethoxy]-acetyl)-(γ-Glu)-CO-(CH₂)₁₂-CO₂H)FI-(D-Glu)-αMeY-LIEGGPSSGAPPPSK((2-[2-(2-Amino-ethoxy)-ethoxy]-acetyl)-(γ-Glu)-CO-(CH₂)₁₂-CO₂H)-NH₂ | 381 | 5363.08 |
| 372 | Y-Aib-EGT-αMeF(2F)-TSDY-Aib-I-αMeL-LD-Orn-K((2-[2-(2-Amino-ethoxy)-ethoxy]-acetyl)-(γ-Glu)-(10-(4-carboxyphenoxy)decanoyl))AQ-Aib-K((2-[2-(2-Amino-ethoxy)-ethoxy]-acetyl)-(γ-Glu)-(10-(4-carboxyphenoxy)decanoyl))FI-(D-Glu)-αMeY-LIEGGPSSGAPPPS-NH₂ | 382 | 5349.95 |
| 373 | Y-Aib-EGT-αMeF(2F)-TSDY-Aib-I-αMeL-LD-Orn-K((2-[2-(2-Amino-ethoxy)-ethoxy]-acetyl)-(γ-Glu)-(10-(4-carboxyphenoxy)decanoyl))AQ-Aib-EFI-(D-Glu)-αMeY-LIK((2-[2-(2-Amino-ethoxy)-ethoxy]-acetyl)-(γ-Glu)-(10-(4-carboxyphenoxy)decanoyl))GGPSSGAPPPS-NH₂ | 383 | 5349.95 |
| 374 | Y-Aib-EGT-αMeF(2F)-TSDY-Aib-I-αMeL-LD-Orn-K((2-[2-(2-Amino-ethoxy)-ethoxy]-acetyl)-(γ-Glu)-(10-(4-carboxyphenoxy)decanoyl))AQ-Aib-EFI-(D-Glu)-αMeY-LIEGGPSSGAPPPSK((2-[2-(2-Amino-ethoxy)-ethoxy]-acetyl)-(γ-Glu)-(10-(4-carboxyphenoxy)decanoyl))-NH₂ | 384 | 5479.07 |
| 375 | Y-Aib-EGT-αMeF(2F)-TSDY-Aib-I-αMeL-LD-Orn-IAQ-Aib-EFIK((2-[2-(2-Amino-ethoxy)-ethoxy]-acetyl)-(γ-Glu)-(10-(4-carboxyphenoxy)decanoyl))-αMeY-LIK((2-[2-(2-Amino-ethoxy)-ethoxy]-acetyl)-(γ-Glu)-(10-(4-carboxyphenoxy)decanoyl))GGPSSGAPPPS-NH₂ | 385 | 5334 |
| 376 | Y-Aib-EGT-αMeF(2F)-TSDY-Aib-I-αMeL-LD-Orn-IAQ-Aib-K((2-[2-(2-Amino-ethoxy)-ethoxy]-acetyl)-(γ-Glu)-(10-(4-carboxyphenoxy)decanoyl))FI-(D-Glu)-αMeY-LIK((2-[2-(2-Amino-ethoxy)-ethoxy]-acetyl)-(γ-Glu)-(10-(4-carboxyphenoxy)decanoyl))GGPSSGAPPPS-NH₂ | 386 | 5334 |
| 377 | Y-Aib-EGT-αMeF(2F)-TSDY-Aib-I-αMeL-LD-Orn-IAQ-Aib-K((2-[2-(2-Amino-ethoxy)-ethoxy]-acetyl)-(γ-Glu)-(10-(4-carboxyphenoxy)decanoyl))FI-(D-Glu)-αMeY-LIEGGPSSGAPPPSK((2-[2-(2-Amino-ethoxy)-ethoxy]-acetyl)-(γ-Glu)-(10-(4-carboxyphenoxy)decanoyl))-NH₂ | 387 | 5463.11 |
| 378 | Y-Aib-EGT-αMeF(2F)-TSDV-Aib-I-αMeL-LD-Orn-K((2-[2-(2-Amino-ethoxy)-ethoxy]-acetyl)-(γ-Glu)-(10-(4-carboxyphenoxy)decanoyl))AQ-Aib-K((2-[2-(2-Amino-ethoxy)-ethoxy]-acetyl)-(γ-Glu)-(10-(4-carboxyphenoxy)decanoyl))FI-(D-Glu)-αMeY-LIEGGPSSGAPPPS-NH₂ | 388 | 5285.91 |
| 379 | Y-Aib-EGT-αMeF(2F)-TSDV-Aib-I-αMeL-LD-Orn-K((2-[2-(2-Amino-ethoxy)-ethoxy]-acetyl)-(γ-Glu)-(10-(4-carboxyphenoxy)decanoyl))AQ-Aib-EFI-(D-Glu)-αMeY-LIK((2-[2-(2-Amino-ethoxy)-ethoxy]-acetyl)-(γ-Glu)-(10-(4-carboxyphenoxy)decanoyl))GGPSSGAPPPS-NH₂ | 389 | 5285.91 |
| 380 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-αMeY-LIEGGPSSGAPPPSK((2-[2-(2-Amino-ethoxy)-ethoxy]-acetyl)₂-(γ-Glu)-(10-(4-carboxyphenoxy)decanoyl))-NH₂ | 390 | 5705.34 |
| 381 | Y-Aib-EGT-αMeF(2F)-TSDV-Aib-I-αMeL-LD-Orn-IAQ-Aib-EFIK((2-[2-(2-Amino-ethoxy)-ethoxy]-acetyl)-(γ-Glu)-(10-(4-carboxyphenoxy)decanoyl))-αMeY-LIK((2-[2-(2-Amino-ethoxy)-ethoxy]-acetyl)-(γ-Glu)-(10-(4-carboxyphenoxy)decanoyl))GGPSSGAPPPS-NH₂ | 391 | 5269.95 |
| 382 | Y-Aib-EGT-αMeF(2F)-TSDV-Aib-I-αMeL-LD-Orn-IAQ-Aib-K((2-[2-(2-Amino-ethoxy)-ethoxy]-acetyl)-(γ-Glu)-(10-(4-carboxyphenoxy)decanoyl))FI-(D-Glu)-αMeY-LIK((2-[2-(2-Amino-ethoxy)-ethoxy]-acetyl)-(γ-Glu)-(10-(4-carboxyphenoxy)decanoyl))GGPSSGAPPPS-NH₂ | 392 | 5269.95 |
| 383 | Y-Aib-EGT-αMeF(2F)-TSDV-Aib-I-αMeL-LD-Orn-IAQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LIEGGPSSGAPPPSK((2-[2-(2-Amino-ethoxy)-ethoxy]-acetyl)₂-(γ-Glu)-(10-(4-carboxyphenoxy)decanoyl))-NH₂ | 393 | 5689.38 |
| 384 | Y-Aib-EGT-αMeF(2F)-TSD-4Pal-Aib-I-αMeL-LD-Orn-K((2-[2-(2-Amino-ethoxy)-ethoxy]-acetyl)₂-CO-(CH₂)₁₂-CO₂H)AQ-Aib-K((2-[2-(2-Amino-ethoxy)-ethoxy]-acetyl)₂-CO-(CH₂)₁₂-CO₂H)FI-(D-Glu)-αMeY-LIEGGPSSGAPPPS-NH₂ | 394 | 5266.99 |
| 385 | Y-Aib-EGT-αMeF(2F)-TSDY-Aib-I-αMeL-LD-Orn-K((2-[2-(2-Amino-ethoxy)-ethoxy]-acetyl)₂-CO-(CH₂)₁₂-CO₂H)AQ-Aib-K((2-[2-(2-Amino-ethoxy)-ethoxy]-acetyl)₂-CO-(CH₂)₁₂-CO₂H)FI-(D-Glu)-αMeY-LIEGGPSSGAPPPS-NH₂ | 395 | 5282.01 |
| 386 | Y-Aib-EGT-αMeF(2F)-TSDV-Aib-I-αMeL-LD-Orn-K((2-[2-(2-Amino-ethoxy)-ethoxy]-acetyl)₂-CO-(CH₂)₁₂-CO₂H)AQ-Aib-K((2-[2-(2-Amino-ethoxy)-ethoxy]-acetyl)₂-CO-(CH₂)₁₂-CO₂H)FI-(D-Glu)-αMeY-LIEGGPSSGAPPPS-NH₂ | 396 | 5217.96 |
| 387 | Y-Aib-EGT-αMeF(2F)-TSD-4Pal-Aib-I-αMeL-LD-Orn-K((2-[2-(2-Amino-ethoxy)-ethoxy]-acetyl)₂-CO-(CH₂)₁₂-CO₂H)AQ-Aib-EFI-(D-Glu)-αMeY-LIK((2-[2-(2-Amino-ethoxy)-ethoxy]-acetyl)₂-CO-(CH₂)₁₂-CO₂H)GGPSSGAPPPS-NH₂ | 397 | 5266.99 |
| 388 | Y-Aib-EGT-αMeF(2F)-TSDY-Aib-I-αMeL-LD-Orn-K((2-[2-(2-Amino-ethoxy)-ethoxy]-acetyl)₂-CO-(CH₂)₁₂-CO₂H)AQ-Aib-EFI-(D-Glu)-αMeY-LIK((2-[2-(2-Amino-ethoxy)-ethoxy]-acetyl)₂-CO-(CH₂)₁₂-CO₂H)GGPSSGAPPPS-NH₂ | 398 | 5282.01 |
| 389 | Y-Aib-EGT-αMeF(2F)-TSDV-Aib-I-αMeL-LD-Orn-K((2-[2-(2-Amino-ethoxy)-ethoxy]-acetyl)₂-CO-(CH₂)₁₂-CO₂H)AQ-Aib-EFI-(D-Glu)-αMeY-LIK((2-[2-(2-Amino-ethoxy)-ethoxy]-acetyl)₂-CO-(CH₂)₁₂-CO₂H)GGPSSGAPPPS-NH₂ | 399 | 5217.96 |
| 390 | Y-Aib-EGT-αMeF(2F)-TSDV-Aib-I-αMeL-LD-Orn-QAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 400 | 5475.21 |
| 391 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 401 | 5509.27 |
| 392 | Y-Aib-EGT-αMeF(2F)-TSD-4Pal-Aib-I-αMeL-LD-Orn-K((2-[2-(2-Amino-ethoxy)-ethoxy]-acetyl)₂-(10-(4-carboxyphenoxy)decanoyl))AQ-Aib-K((2-[2-(2-Amino-ethoxy)-ethoxy]-acetyl)₂-(10-(4-carboxyphenoxy)decanoyl))FI-(D-Glu)-αMeY-LIEGGPSSGAPPPS-NH₂ | 402 | 5367.03 |
| 393 | Y-Aib-EGT-αMeF(2F)-TSDY-Aib-I-αMeL-LD-Orn-K((2-[2-(2-Amino-ethoxy)-ethoxy]-acetyl)₂-(10-(4-carboxyphenoxy)decanoyl))AQ-Aib-K((2-[2-(2-Amino-ethoxy)-ethoxy]-acetyl)₂-(10-(4-carboxyphenoxy)decanoyl))FI-(D-Glu)-αMeY-LIEGGPSSGAPPPS-NH₂ | 403 | 5382.04 |
| 394 | Y-Aib-EGT-αMeF(2F)-TSDV-Aib-I-αMeL-LD-Orn-K((2-[2-(2-Amino-ethoxy)-ethoxy]-acetyl)₂-(10-(4-carboxyphenoxy)decanoyl))AQ-Aib-K((2-[2-(2-Amino-ethoxy)-ethoxy]-acetyl)₂-(10-(4-carboxyphenoxy)decanoyl))FI-(D-Glu)-αMeY-LIEGGPSSGAPPPS-NH₂ | 404 | 5318 |
| 395 | Y-Aib-EGT-αMeF(2F)-TSD-4Pal-Aib-I-αMeL-LD-Orn-K((2-[2-(2-Amino-ethoxy)-ethoxy]-acetyl)₂-(10-(4-carboxyphenoxy)decanoyl))AQ-Aib-EFI-(D-Glu)-αMeY-LIK((2-[2-(2-Amino-ethoxy)-ethoxy]-acetyl)₂-(10-(4-carboxyphenoxy)decanoyl))GGPSSGAPPPS-NH₂ | 405 | 5367.03 |
| 396 | Y-Aib-EGT-αMeF(2F)-TSDY-Aib-I-αMeL-LD-Orn-K((2-[2-(2-Amino-ethoxy)-ethoxy]-acetyl)₂-(10-(4-carboxyphenoxy)decanoyl))AQ-Aib-EFI-(D-Glu)-αMeY-LIK((2-[2-(2-Amino-ethoxy)-ethoxy]-acetyl)₂-(10-(4-carboxyphenoxy)decanoyl))GGPSSGAPPPS-NH₂ | 406 | 5382.04 |
| 397 | Y-Aib-EGT-αMeF(2F)-TSDV-Aib-I-αMeL-LD-Orn-K((2-[2-(2-Amino-ethoxy)-ethoxy]-acetyl)₂-(10-(4-carboxyphenoxy)decanoyl))AQ-Aib-EFI-(D-Glu)-αMeY-LIK((2-[2-(2-Amino-ethoxy)-ethoxy]-acetyl)₂-(10-(4-carboxyphenoxy)decanoyl))GGPSSGAPPPS-NH₂ | 407 | 5318 |
| 398 | Y-Aib-EGT-αMeF(2F)-TSDV-Aib-I-αMeL-LD-Orn-K((2-[2-(2-Amino-ethoxy)-ethoxy]-acetyl)-(γ-Glu)-CO-(CH₂)₁₂-CO₂H)AQ-Aib-K((2-[2-(2-Amino-ethoxy)-ethoxy]-acetyl)-(γ-Glu)-CO-(CH₂)₁₂-CO₂H)FI-(D-Glu)-αMeY-LIEGGPSSGAPPPS-NH₂ | 408 | 5185.88 |
| 399 | Y-Aib-EGT-αMeF(2F)-TSDV-Aib-I-αMeL-LD-Orn-K((2-[2-(2-Amino-ethoxy)-ethoxy]-acetyl)-(γ-Glu)-CO-(CH₂)₁₂-CO₂H)AQ-Aib-EFI-(D-Glu)-αMeY-LIK((2-[2-(2-Amino-ethoxy)-ethoxy]-acetyl)-(γ-Glu)-CO-(CH₂)₁₂-CO₂H)GGPSSGAPPPS-NH₂ | 409 | 5185.88 |
| 400 | Y-Aib-EGT-αMeF(2F)-TSDV-Aib-I-αMeL-LD-Orn-IAQ-Aib-EFIK((2-[2-(2-Amino-ethoxy)-ethoxy]-acetyl)-(γ-Glu)-CO-(CH₂)₁₂-CO₂H)-αMeY-LIK((2-[2-(2-Amino-ethoxy)-ethoxy]-acetyl)-(γ-Glu)-CO-(CH₂)₁₂-CO₂H)GGPSSGAPPPS-NH₂ | 410 | 5169.92 |
| 401 | Y-Aib-EGT-αMeF(2F)-TSDY-Aib-I-αMeL-LD-Orn-QAQ-Aib-Orn-FIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 411 | 5524.28 |
| 402 | Y-Aib-EGT-αMeF(2F)-TSDY-Aib-I-αMeL-LD-Orn-QAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 412 | 5639.28 |
| 403 | Y-Aib-EGT-αMeF(2F)-TSD-4Pal-Aib-I-αMeL-LD-Orn-QAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 413 | 5624.27 |
| 404 | Y-Aib-EGT-αMeF(2F)-TSD-4Pal-Aib-I-αMeL-LD-Orn-K(PEG3-(γ-Glu)-CO-(CH₂)₁₂-CO₂H)AQ-Aib-K(PEG3-(γ-Glu)-CO-(CH₂)₁₂-CO₂H)FI-(D-Glu)-αMeY-LIEGGPSSGAPPPS-NH₂ | 414 | 5351.07 |
| 405 | Y-Aib-EGT-αMeF(2F)-TSD-4Pal-Aib-I-αMeL-LD-Orn-K(PEG4-(γ-Glu)-CO-(CH₂)₁₂-CO₂H)AQ-Aib-K(PEG4-(γ-Glu)-CO-(CH₂)₁₂-CO₂H)FI-(D-Glu)-αMeY-LIEGGPSSGAPPPS-NH₂ | 415 | 5439.17 |
| 406 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-K((2-[2-(2-Amino-ethoxy)-ethoxy]-acetyl)₂-(γ-Glu)-CO-(CH₂)₁₂-CO₂H)FI-(D-Glu)-αMeY-LI-Orn-GGPSSGAPPPS-NH₂ | 416 | 5510.25 |
| 407 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-CO-(CH₂)₁₂-CO₂H)AQ-Aib-K((2-[2-(2-Amino-ethoxy)-ethoxy]-acetyl)₂-(γ-Glu)-CO-(CH₂)₁₂-CO₂H)FI-(D-Glu)-αMeY-LI-Orn-GGPSSGAPPPS-NH₂ | 417 | 5461.22 |
| 408 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LI-Orn-GGPSSGAPPPS-NH₂ | 418 | 5610.29 |
| 409 | Y-Aib-EGT-αMeF(2F)-TSDY-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LI-Orn-GGPSSGAPPPS-NH₂ | 419 | 5625.3 |
| 410 | Y-Aib-EGT-αMeF(2F)-TSDV-Aib-I-αMeL-LD-Orn-K((2-[2-(2-Amino-ethoxy)-ethoxy]-acetyl)₂-(γ-Glu)-(10-(4-carboxyphenoxy)decanoyl))AQ-Aib-K((2-[2-(2-Amino-ethoxy)-ethoxy]-acetyl)₂-(γ-Glu)-(10-(4-carboxyphenoxy)decanoyl))FI-(D-Glu)-αMeY-LI-Orn-GGPSSGAPPPS-NH₂ | 420 | 5561.25 |
| 411 | Y-Aib-EGT-αMeF(2F)-TSD-4Pal-Aib-I-αMeL-LD-Orn-QAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 421 | 5524.24 |
| 412 | Y-Aib-EGT-αMeF(2F)-TSDY-Aib-I-αMeL-LD-Orn-QAQ-Aib-EFIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 422 | 5539.25 |
| 413 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-K((2-[2-(2-Amino-ethoxy)-ethoxy]-acetyl)₂-(γ-Glu)-CO-(CH₂)₁₀-CH₃)FI-(D-Glu)-αMeY-LI-Orn-GGPSSGAPPPS-NH₂ | 423 | 5394.18 |
| 414 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-Orn-FI-(D-Glu)-αMeY-LIK((2-[2-(2-Amino-ethoxy)-ethoxy]-acetyl)₂-(γ-Glu)-CO-(CH₂)₁₀-CH₃)GGPSSGAPPPS-NH₂ | 424 | 5394.18 |
| 415 | Y-Aib-EGT-αMeF(2F)-TSDK((2-[2-(2-Amino-ethoxy)-ethoxy]-acetyl)₂-(γ-Glu)-CO-(CH₂)₁₀-CH₃)-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-4Pal-FI-(D-Glu)-αMeY-LIEGGPSSGAPPPS-NH₂ | 425 | 5409.15 |
| 416 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-4Pal-FI-(D-Glu)-αMeY-LIK((2-[2-(2-Amino-ethoxy)-ethoxy]-acetyl)₂-(γ-Glu)-CO-(CH₂)₁₀-CH₃)GGPSSGAPPPS-NH₂ | 426 | 5428.2 |
| 417 | Y-Aib-EGT-αMeF(2F)-TSDK((2-[2-(2-Amino-ethoxy)-ethoxy]-acetyl)₂-(γ-Glu)-CO-(CH₂)₁₀-CH₃)-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-Orn-FI-(D-Glu)-αMeY-LIEGGPSSGAPPPS-NH₂ | 427 | 5375.13 |
| 418 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-Orn-FI-(D-Glu)-αMeY-LIEGGPSSGAPPPSK((2-[2-(2-Amino-ethoxy)-ethoxy]-acetyl)₂-(γ-Glu)-CO-(CH₂)₁₀-CH₃)-NH₂ | 428 | 5523.3 |
| 419 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-TFI-(D-Glu)-αMeY-LIK((2-[2-(2-Amino-ethoxy)-ethoxy]-acetyl)₂-(γ-Glu)-CO-(CH₂)₁₀-CH₃)GGPSSGAPPPS-NH₂ | 429 | 5381.14 |
| 420 | Y-Aib-EGT-αMeF(2F)-TSD-4Pal-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-4Pal-FI-(D-Glu)-αMeY-LIEGGPSSGAPPPSK((2-[2-(2-Amino-ethoxy)-ethoxyl-acetyl)₂-(γ-Glu)-CO-(CH₂)₁₀-CH₃)-NH₂ | 430 | 5557.31 |
| 421 | Y-Aib-EGT-αMeF(2F)-TSDK((2-[2-(2-Amino-ethoxy)-ethoxy]-acetyl)₃-(γ-Glu)-CO-(CH₂)₁₀-CH₃)-Aib-I-αMeL-LD-Orn-K((2-[2-(2-Amino-ethoxy)-ethoxy]-acetyl)₃-(γ-Glu)-CO-(CH₂)₁₀-CH₃)AQ-Aib-EFI-(D-Glu)-αMeY-LIEGGPSSGAPPPS-NH₂ | 431 | 5680.42 |
| 422 | Y-Aib-EGT-αMeF(2F)-TSD-4Pal-Aib-I-αMeL-LD-Orn-K((2-[2-(2-Amino-ethoxy)-ethoxy]-acetyl)₃-(γ-Glu)-CO-(CH₂)₁₀-CH₃)AQ-Aib-K((2-[2-(2-Amino-ethoxy)-ethoxy]-acetyl)₃-(γ-Glu)-CO-(CH₂)₁₀-CH₃)FI-(D-Glu)-αMeY-LIEGGPSSGAPPPS-NH₂ | 432 | 5699.46 |
| 423 | Y-Aib-EGT-αMeF(2F)-TSD-4Pal-Aib-I-αMeL-LD-Orn-K((2-[2-(2-Amino-ethoxy)-ethoxy]-acetyl)₃-(γ-Glu)-CO-(CH₂)₁₀-CH₃)AQ-Aib-EFI-(D-Glu)-αMeY-LIK((2-[2-(2-Amino-ethoxy)-ethoxy]-acetyl)₃-(γ-Glu)-CO-(CH₂)₁₀-CH₃)GGPSSGAPPPS-NH₂ | 433 | 5699.46 |
| 424 | Y-Aib-EGT-αMeF(2F)-TSD-4Pal-Aib-I-αMeL-LD-Orn-K((2-[2-(2-Amino-ethoxy)-ethoxy]-acetyl)₃-(γ-Glu)-CO-(CH₂)₁₀-CH₃)AQ-Aib-EFI-(D-Glu)-αMeY-LIEGGPSSGAPPPSK((2-[2-(2-Amino-ethoxy)-ethoxy]-acetyl)₃-(γ-Glu)-CO-(CH₂)₁₀-CH₃)-NH₂ | 434 | 5828.58 |
| 425 | Y-Aib-EGT-αMeF(2F)-TSDK((2-[2-(2-Amino-ethoxy)-ethoxy]-acetyl)-(γ-Glu)-CO-(CH₂)₁₀-CH₃)-Aib-I-αMeL-LD-Orn-K((2-[2-(2-Amino-ethoxy)-ethoxy]-acetyl)-(γ-Glu)-CO-(CH₂)₁₀-CH₃)AQ-Aib-EFI-(D-Glu)-αMeY-LIEGGPSSGAPPPS-NH₂ | 435 | 5099.79 |
| 426 | Y-Aib-EGT-αMeF(2F)-TSD-4Pal-Aib-I-αMeL-LD-Orn-K((2-[2-(2-Amino-ethoxy)-ethoxy]-acetyl)-(γ-Glu)-CO-(CH₂)₁₀-CH₃)AQ-Aib-K((2-[2-(2-Amino-ethoxy)-ethoxy]-acetyl)-(γ-Glu)-CO-(CH₂)₁₀-CH₃)FI-(D-Glu)-αMeY-LIEGGPSSGAPPPS-NH₂ | 436 | 5118.84 |
| 427 | Y-Aib-EGT-αMeF(2F)-TSD-4Pal-Aib-I-αMeL-LD-Orn-K((2-[2-(2-Amino-ethoxy)-ethoxy]-acetyl)-(γ-Glu)-CO-(CH₂)₁₀-CH₃)AQ-Aib-EFI-(D-Glu)-αMeY-LIK((2-[2-(2-Amino-ethoxy)-ethoxy]-acetyl)-(γ-Glu)-CO-(CH₂)₁₀-CH₃)GGPSSGAPPPS-NH₂ | 437 | 5118.84 |
| 428 | Y-Aib-EGT-αMeF(2F)-TSD-4Pal-Aib-I-αMeL-LD-Orn-K((2-[2-(2-Amino-ethoxy)-ethoxy]-acetyl)-(γ-Glu)-CO-(CH₂)₁₀-CH₃)AQ-Aib-EFI-(D-Glu)-αMeY-LIEGGPSSGAPPPSK((2-[2-(2-Amino-ethoxy)-ethoxy]-acetyl)-(γ-Glu)-CO-(CH₂)₁₀-CH₃)-NH₂ | 438 | 5247.95 |
| 429 | Y-Aib-EGT-αMeF(2F)-TSDK((2-[2-(2-Amino-ethoxy)-ethoxy]-acetyl)₂-(γ-Glu)-CO-(CH₂)₁₀-CH₃)-Aib-I-αMeL-LD-Orn-K((2-[2-(2-Amino-ethoxy)-ethoxy]-acetyl)₂-(γ-Glu)-CO-(CH₂)₁₀-CH₃)AQ-Aib-E-αMeF-I-(D-Glu)-αMeY-LIEGGPSSGAPPPS-NH₂ | 439 | 5404.13 |
| 430 | Y-Aib-EGT-αMeF(2F)-TSD-4Pal-Aib-I-αMeL-LD-Orn-K((2-[2-(2-Amino-ethoxy)-ethoxy]-acetyl)₂-(γ Glu)-CO-(CH₂)₁₀H₃)AQ-Aib-K((2-[2-(2-Amino-ethoxy)-ethoxy]-acetyl)₂-(γGlu)-CO-(CH₂)₁₀C₃)-αMeFI-(D-Glu)-αMeY-LIEGGPSSGAPPPS-NH₂ | 440 | 5423.18 |
| 431 | Y-Aib-EGT-αMeF(2F)-TSD-4Pal-Aib-I-αMeL-LD-Orn-K((2-[2-(2-Amino-ethoxy)-ethoxy]-acetyl)₂-(γ Glu)-CO-(CH₂)₁₀C₃)AQ-Aib-E-αMeF-I-(D-Glu)-αMeY-LIEGGPSSGAPPPSK((2-[2-(2-Amino-ethoxy)-ethoxy]-acetyl)₂-(γ-Glu)-CO-(CH₂)₁₀-CH₃)-NH₂ | 441 | 5552.29 |
| 432 | Y-Aib-EGT-αMeF(2F)-TSDK((2-[2-(2-Amino-ethoxy)-ethoxy]-acetyl)₃-(γ-Glu)-CO-(CH₂)₁₀-CH₃)-Aib-I-αMeL-LD-Orn-K((2-[2-(2-Amino-ethoxy)-ethoxy]-acetyl)₃-(γ-Glu)-CO-(CH₂)₁₀-CH₃)AQ-Aib-Orn-FI-(D-Glu)-αMeY-LIEGGPSSGAPPPS-NH₂ | 442 | 5665.45 |
| 433 | Y-Aib-EGT-αMeF(2F)-TSD-4Pal-Aib-I-αMeL-LD-Orn-K((2-[2-(2-Amino-ethoxy)-ethoxy]-acetyl)₃-(γ-Glu)-CO-(CH₂)₁₀-CH₃)AQ-Aib-K((2-[2-(2-Amino-ethoxy)-ethoxy]-acetyl)₃-(γ-Glu)-CO-(CH₂)₁₀-CH₃)FI-(D-Glu)-αMeY-LI-Orn-GGPSSGAPPPS-NH₂ | 443 | 5599.39 |
| 434 | Y-Aib-EGT-αMeF(2F)-TSD-4Pal-Aib-I-αMeL-LD-Orn-K((2-[2-(2-Amino-ethoxy)-ethoxy]-acetyl)₃-(γ-Glu)-CO-(CH₂)₁₀-CH₃)AQ-Aib-Orn-FI-(D-Glu)-αMeY-LIK((2-[2-(2-Amino-ethoxy)-ethoxy]-acetyl)₃-(γ-Glu)-CO-(CH₂)₁₀-CH₃)GGPSSGAPPPS-NH₂ | 444 | 5684.5 |
| 435 | Y-Aib-EGT-αMeF(2F)-TSD-4Pal-Aib-I-αMeL-LD-Orn-K((2-[2-(2-Amino-ethoxy)-ethoxy]-acetyl)₃-(γ Glu)-CO-(CH₂)₁₀CH₃)AQ-Aib-Orn-FI-(D-Glu)-αMeY-LIEGGPSSGAPPPSK((2-[2-(2-Amino-ethoxy)-ethoxy]-acetyl)₃-(γ-Glu)-CO-(CH₂)₁₀-CH₃)-NH₂ | 445 | 5813.61 |
| 436 | Y-Aib-EGT-αMeF(2F)-TSDK((2-[2-(2-Amino-ethoxy)-ethoxy]-acetyl)₃-(γ-Glu)-CO-(CH₂)₁₀-CH₃)-Aib-I-αMeL-LD-Orn-K((2-[2-(2-Amino-ethoxy)-ethoxy]-acetyl)₃-(γ-Glu)-CO-(CH₂)₁₀-CH₃)AQ-Aib-E-αMeF-I-(D-Glu)-αMeY-LIEGGPSSGAPPPS-NH₂ | 446 | 5694.44 |
| 437 | Y-Aib-EGT-αMeF(2F)-TSD-4Pal-Aib-I-αMeL-LD-Orn-K((2-[2-(2-Amino-ethoxy)-ethoxy]-acetyl)₃-(γ-Glu)-CO-(CH₂)₁₀-CH₃)AQ-Aib-K((2-[2-(2-Amino-ethoxy)-ethoxy]-acetyl)₃-(γ-Glu)-CO-(CH₂)₁₀-CH₃)-αMeF-I-(D-Glu)-αMeY-LIEGGPSSGAPPPS-NH₂ | 447 | 5729.53 |
| 438 | Y-Aib-EGT-αMeF(2F)-TSD-4Pal-Aib-I-αMeL-LD-Orn-K((2-[2-(2-Amino-ethoxy)-ethoxy]-acetyl)₃-(γ-Glu)-CO-(CH₂)₁₀-CH₃)AQ-Aib-E-αMeF-I-(D-Glu)-αMeY-LIK((2-[2-(2-Amino-ethoxy)-ethoxy]-acetyl)₃-(γ-Glu)-CO-(CH2)₁₀-CH₃)GGPSSGAPPPS-NH₂ | 448 | 5713.49 |
| 439 | Y-Aib-EGT-αMeF(2F)-TSD-4Pal-Aib-I-αMeL-LD-Orn-K((2-[2-(2-Amino-ethoxy)-ethoxy]-acetyl)₃-(_{Y}-Glu)-CO-(CH₂)₁₀-CH₃)AQ-Aib-E-αMeF-I-(D-Glu)-αMeY-LIEGGPSSGAPPPSK((2-[2-(2-Amino-ethoxy)-ethoxy]-acetyl)₃-(γ-Glu)-CO-(CH₂)₁₀-CH₃)-NH₂ | 449 | 5842.6 |
| 440 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 450 | 5609.3 |
| 441 | Y-Aib-EGT-αMeF(2F)-TSD-4Pal-Aib-G-αMeL-LD-Orn-QAQ-Aib-Orn-FIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 451 | 5453.16 |
| 442 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)₂-(γ-Glu)-(11-(4-carboxyphenoxy)undecanoyl)-αMeY-LIK((2-[2-(2-Amino-ethoxy)-ethoxy]-acetyl)₂-(γ-Glu)-(11-(4-carboxyphenoxy)undecanoyl)GGPSSGAPPPS-NH₂ | 452 | 5637.35 |
| 443 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)₂-(γ-Glu)-CO-(CH₂)₁₄-CO₂)-αMeY-LIK((2-[2-(2-Amino-ethoxy)-ethoxy]-acetyl)₂-(γ-Glu)-CO-(CH₂)₁₄-CO₂)GGPSSGAPPPS-NH₂ | 453 | 5565.38 |
| 444 | Y-Aib-EGT-αMeF(2F)-TSD-4Pal-Aib-I-αMeL-LD-Orn-QAQ-Aib-4Pal-FIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 454 | 5543.29 |
| 445 | Y-Aib-EGT-αMeF(2F)-TSDK((2-[2-(2-Amino-ethoxy)-ethoxy]-acetyl)₂-(γ-Glu)-CO-(CH₂)₁₀-CH₃)-Aib-I-αMeL-LDGK((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGGPSSGAPPPS-NH₂ | 455 | 5333.01 |
| 446 | Y-Aib-EGT-αMeF(2F)-TSDV-Aib-I-αMeL-LD-Orn QAQ-Aib-EFIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-γ-Glu)-(10-(4-carboxyphenoxy)decanoyl))GGPSSGAPPPS-NH₂ | 456 | 5575.24 |
| 447 | Y-Aib-EGT-αMeF(2F)-TSDV-Aib-I-αMeL-LD-Orn-QAQ-Aib-Orn-FIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 457 | 5560.27 |
| 448 | Y-Aib-EGT-αMeF(2F)-TSDV-Aib-I-αMeL-LD-Orn QAQ-Aib-Orn-FIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 458 | 5460.24 |
| 449 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)-Ahx-(γ-Glu)-CO-(CH₂)₁₂-CO₂)-αMeY-LIK((2-[2-(2-Amino-ethoxy)-ethoxy]-acetyl)-Ahx-(γ-Glu)-CO-(CH₂)₁₂-CO₂)GGPSSGAPPPS-NH₂ | 459 | 5445.27 |
| 450 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)-Aoc-(γ-Glu)-CO-(CH₂)₁₂-CO₂)-αMeY-LIK((2-[2-(2-Amino-ethoxy)-ethoxy]-acetyl)-Aoc-(γ-Glu)-CO-(CH₂)₁₂-CO₂)GGPSSGAPPPS-NH₂ | 460 | 5501.38 |
| 451 | Y-Aib-EGT-αMeF(2F)-TSD-4Pal-Aib-I-αMeL-LD-Orn-QAQ-Aib-Orn-FIK(Ahx-(2-[2-(2-Amino-ethoxy)-ethoxy]-acetyl)-(γ-Glu)-CO-(CH₂)₁₂-CO₂)-αMeY-LIK(Ahx-(2-[2-(2-Amino-ethoxy)-ethoxy]-acetyl)-(γ-Glu)-CO-(CH₂)₁₂-CO₂)GGPSSGAPPPS-NH₂ | 461 | 5445.27 |
| 452 | Y-Aib-EGT-αMeF(2F)-TSD-4Pal-Aib-I-αMeL-LD-Orn-QAQ-Aib-Orn-FIK(Aoc-(2-[2-(2-Amino-ethoxy)-ethoxy]-acetyl)-(γ-Glu)-CO-(CH₂)₁₂-CO₂)-αMeY-LIK(Aoc-(2-[2-(2-Amino-ethoxy)-ethoxy]-acetyl)-(γ-Glu)-CO-(CH₂)₁₂-CO₂)GGPSSGAPPPS-NH₂ | 462 | 5501.38 |
| 453 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)-Ahx-(γ-Glu)-(10-(4-carboxyphenoxy)decanoyl))-αMeY-LIK((2-[2-(2-Amino-ethoxy)-ethoxy]-acetyl)-Ahx-(γ-Glu)-(10-(4-carboxyphenoxy)decanoyl))GGPSSGAPPPS-NH₂ | 463 | 5545.3 |
| 454 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]10-(4-carboxyphenoxy)decanoyl))-αMeY-LIK((2-[2-(2-Amino-ethoxy)-ethoxy]-acetyl)-Aoc-(γ-Glu)-(10-(4-carboxyphenoxy)decanoyl))GGPSSGAPPPS-NH₂ | 464 | 5601.41 |
| 455 | Y-Aib-EGT-αMeF(2F)-TSD-4Pal-Aib-I-αMeL-LD-Orn-QAQ-Aib-Orn-FIK(Ahx-(2-[2-(2-Amino-ethoxy)-ethoxy]-acetyl)-(γ-Glu)-(10-(4-carboxyphenoxy)decanoyl))-αMeY-LIK(Ahx-(2-[2-(2-Amino-ethoxy)-ethoxy]-acetyl)-(γ-Glu)-(10-(4-carboxyphenoxy)decanoyl))GGPSSGAPPPS-NH₂ | 465 | 5545.3 |
| 456 | Y-Aib-EGT-αMeF(2F)-TSD-4Pal-Aib-I-αMeL-LD-Orn-QAQ-Aib-Orn-FIK(Aoc-(2-[2-(2-Amino-ethoxy)-ethoxy]-acetyl)-(γ-Glu)-(10-(4-carboxyphenoxy)decanoyl))-αMeY-LIK(Aoc-(2-[2-(2-Amino-ethoxy)-ethoxy]-acetyl)-(γ-Glu)-(10-(4-carboxyphenoxy)decanoyl))GGPSSGAPPPS-NH₂ | 466 | 5601.41 |
| 457 | Y-Aib-EGT-αMeF(2F)-TSD-4Pal-Aib-I-αMeL-LD-Orn-QAQ-Aib-Orn-FIK(PEG4-(γ-Glu)-CO-(CH₂)₁₂-CO₂H)-αMeY-LIK(PEG4-(γ-Glu)-CO-(CH₂)₁₂-CO₂H)GGPSSGAPPPS-NH₂ | 467 | 5423.22 |
| 458 | Y-Aib-EGT-αMeF(2F)-TSD-4Pal-Aib-I-αMeL-LD-Orn-QAQ-Aib-Orn-FIK(PEG3-(γ-Glu)-CO-(CH₂)₁₂-CO₂H)-αMeY-LIK(PEG3-(γ-Glu)-CO-(CH₂)₁₂-CO₂H)GGPSSGAPPPS-NH₂ | 468 | 5335.11 |
| 459 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)-(γ-Glu)-CO-(CH₂)₁₂-CO₂H)-αMeY-LIK((2-[2-(2-Amino-ethoxy)-ethoxy]-acetyl)-(γ-Glu)-CO-(CH₂)₁₂-CO₂H)GGPSSGAPPPS-NH₂ | 469 | 5218.96 |
| 460 | Y-Aib-EGT-αMeF(2F)-TSD-4Pal-Aib-I-αMeL-LD-Orn-QAQ-Aib-EFIK((2-[2-(2-Amino-ethoxy)-ethoxy]-acetyl)₃-(γ-Glu)-CO-(CH₂)₁₂-CO₂H)-αMeY-LIK((2-[2-(2-Amino-ethoxy)-ethoxy]-acetyl)₃-(γ-Glu)-CO-(CH₂)₁₂-CO₂H)GGPSSGAPPPS-NH₂ | 470 | 5814.55 |
| 461 | Y-Aib-EGT-αMeF(2F)-TSD-4Pal-Aib-I-αMeL-LD-Orn-QAQ-Aib-Orn-FIK(PEG4-(γ-Glu)-(10-(4-carboxyphenoxy)decanoyl))-αMeY-LIK(PEG4-(γ-Glu)-(10-(4-carboxyphenoxy)decanoyl))GGPSSGAPPPS-NH₂ | 471 | 5523.25 |
| 462 | Y-Aib-EGT-αMeF(2F)-TSD-4Pal-Aib-I-αMeL-LD-Orn-QAQ-Aib-Orn-FIK(PEG3-(γ-Glu)-(10-(4-carboxyphenoxy)decanoyl))-αMeY-LIK(PEG3-(γ-Glu)-(10-(4-carboxyphenoxy)decanoyl))GGPSSGAPPPS-NH₂ | 472 | 5435.15 |
| 463 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)-(γ-Glu)-(10-(4-carboxyphenoxy)decanoyl))-αMeY-LIK((2-[2-(2-Amino-ethoxy)-ethoxy]-acetyl)-(γ-Glu)-(10-(4-carboxyphenoxy)decanoyl))GGPSSGAPPPS-NH₂ | 473 | 5318.99 |
| 464 | Y-Aib-EGT-αMeF(2F)-TSD-4Pal-Aib-I-αMeL-LD-Orn-QAQ-Aib-EFIK((2-[2-(2-Amino-ethoxy)-ethoxy]-acetyl)₃-(γ-Glu)-(10-(4-carboxyphenoxy)decanoyl)-αMeY-LIK((2-[2-(2-Amino-ethoxy)-ethoxy]-acetyl)₃-(γ-Glu)-(10-(4-carboxyphenoxy)decanoyl)GGPSSGAPPPS-NH₂ | 474 | 5914.58 |
| 465 | Y-Aib-EGT-αMeF(2F)-TSD-4Pal-Aib-I-αMeL-LD-Orn-K(((2-[2-(2-Amino-ethoxy)-ethoxy]-acetyl)-Aoc-CO-(CH₂)₁₂-CO₂H)AQ-Aib-EFI-(D-Glu)-αMeY-LIK(((2-[2-(2-Amino-ethoxy)-ethoxy]-acetyl)-Aoc-CO-(CH₂)₁₂-CO₂H)GGPSSGAPPPS-NH₂ | 475 | 5259.1 |
| 466 | Y-Aib-EGT-αMeF(2F)-TSD-4Pal-Aib-I-αMeL-LD-Orn-QAQ-Aib-Orn-FIK(PEG6-(γ-Glu)-CO-(CH₂)₁₂-CO₂H)-αMeY-LIK(PEG6-(γ-Glu)-CO-(CH₂)₁₂-CO₂H)GGPSSGAPPPS-NH₂ | 476 | 5599.43 |
| 467 | Y-Aib-EGT-αMeF(2F)-TSD-4Pal-Aib-I-αMeL-LD-Orn-QAQ-Aib-Orn-FIK(PEG5-(γ-Glu)-CO-(CH₂)₁₂-CO₂H)-αMeY-LIK(PEG5-(γ-Glu)-CO-(CH₂)₁₂-CO₂H)GGPSSGAPPPS-NH₂ | 477 | 5511.33 |
| 468 | Y-Aib-EGT-αMeF(2F)-TSD-4Pal-Aib-I-αMeL-LD-Orn-QAQ-Aib-Orn-FIK(PEG6-(γ-Glu)-(10-(4-carboxyphenoxy)decanoyl))-αMeY-LIK(PEG6-(γ-Glu)-(10-(4-carboxyphenoxy)decanoyl))GGPSSGAPPPS-NH₂ | 478 | 5699.46 |
| 469 | Y-Aib-EGT-αMeF(2F)-TSD-4Pal-Aib-I-αMeL-LD-Orn-QAQ-Aib-Orn-FIK(PEG5-(γ-Glu)-(10-(4-carboxyphenoxy)decanoyl))-αMeY-LIK(PEG5-(γ-Glu)-(10-(4-carboxyphenoxy)decanoyl))GGPSSGAPPPS-NH₂ | 479 | 5611.36 |
| 470 | Y-Aib-EGT-αMeF(2F)-TSD-4Pal-Aib-I-αMeL-LD-Orn-QAQ-Aib-Orn-FIK(((2-[2-(2-Amino-ethoxy)-ethoxy]-acetyl)₂--Trx-(γ-Glu)-CO-(CH₂)₁₂-CO₂H)-αMeY-LIK(((2-[2-(2-Amino-ethoxy)-ethoxy]-acetyl)₂--Trx(γ-Glu)-CO-(CH₂)₁₂-CO₂H)GGPSSGAPPPS-NH₂ | 480 | 5787.66 |
| 471 | Y-Aib-EGT-αMeF(2F)-TSD-4Pal-Aib-I-αMeL-LD-Orn-QAQ-Aib-Orn-FIK(((2-[2-(2-Amino-ethoxy)-ethoxy]-acetyl)₂--Trx-(γ-Glu)-(10-(4-carboxyphenoxy)decanoyl))-αMeY-LIK(((2-[2-(2-Amino-ethoxy)-ethoxy]-acetyl)₂--Trx-(γ-Glu)-(10-(4-carboxyphenoxy)decanoyl))GGPSSGAPPPS-NH₂ | 481 | 5887.69 |
| 472 | Y-Aib-EGT-αMeF(2F)-TSD-4Pal-Aib-I-αMeL-LD-Orn-QAQ-Aib-Orn-FIK(((2-[2-(2-Amino-ethoxy)-ethoxy]-acetyl)-Trx-(γ-Glu)-CO-(CH₂)₁₂-CO₂H)-αMeY-LIK(((2-[2-(2-Amino-ethoxy)-ethoxy]-acetyl)-Trx-(γ-Glu)-CO-(CH₂)₁₂-CO₂H)GGPSSGAPPPS-NH₂ | 482 | 5497.35 |
| 473 | Y-Aib-EGT-αMeF(2F)-TSD-4Pal-Aib-I-αMeL-LD-Orn-QAQ-Aib-Orn-FIK(((2-[2-(2-Amino-ethoxy)-ethoxy]-acetyl)-Trx-(γ-Glu)-(10-(4-carboxyphenoxy)decanoyl))-αMeY-LIK(((2-[2-(2-Amino-ethoxy)-ethoxy]-acetyl)-Trx-(γ-Glu)-(10-(4-carboxyphenoxy)decanoyl))GGPSSGAPPPS-NH₂ | 483 | 5597.38 |
| 474 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)₂-(γ-Glu)₂-CO-(CH₂)₁₂-CO₂H)-αMeY-LIK((2-[2-(2-Amino-ethoxy)-ethoxy]-acetyl)₂-(γ-Glu)₂-CO-(CH₂)₁₂-CO₂H)GGPSSGAPPPS-NH₂ | 484 | 5767.5 |
| 475 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)₂-(γ-Glu)₂--(10-(4-carboxyphenoxy)decanoyl))-αMeY-LIK((2-[2-(2-Amino-ethoxy)-ethoxy]-acetyl)₂-(γ-Glu)₂--(10-(4-carboxyphenoxy)decanoyl))GGPSSGAPPPS-NH₂ | 485 | 5867.53 |
| 476 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)₃-(γ-Glu)-CO-(CH₂)₁₂-CO₂H)-αMeY-LIK((2-[2-(2-Amino-ethoxy)-ethoxy]-acetyl)₃-(γ-Glu)-CO-(CH₂)₁₂-CO₂H)GGPSSGAPPPS-NH₂ | 486 | 5799.58 |
| 477 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)₃-(γ-Glu)--(10-(4-carboxyphenoxy)decanoyl))-αMeY-LIK((2-[2-(2-Amino-ethoxy)-ethoxy]-acetyl)₃-(γ-Glu)--(10-(4-carboxyphenoxy)decanoyl))GGPSSGAPPPS-NH₂ | 487 | 5899.61 |
| 478 | Y-Aib-EGT-αMeF(2F)-TSDY-Aib-I-αMeL-LD-Orn-IAQ-Aib-Orn-FIK((2-[2-(2-Amino-ethoxy)-ethoxy]-acetyl)₃-(γ-Glu)-CO-(CH₂)₁₂-CO₂H)-αMeY-LIK((2-[2-(2-Amino-ethoxy)-ethoxy]-acetyl)₃-(γ-Glu)-CO-(CH₂)₁₂-CO₂H)GGPSSGAPPPS-NH₂ | 488 | 5799.62 |
| 479 | Y-Aib-EGT-αMeF(2F)-TSDY-Aib-I-αMeL-LD-Orn-QAQ-Aib-Orn-FIK((2-[2-(2-Amino-ethoxy)-ethoxy]-acetyl)₃-(γ-Glu)-CO-(CH₂)₁₂-CO₂H)-αMeY-LIK((2-[2-(2-Amino-ethoxy)-ethoxy]-acetyl)₃-(γ-Glu)-CO-(CH₂)₁₂-CO₂H)GGPSSGAPPPS-NH₂ | 489 | 5814.59 |
| 480 | Y-Aib-EGT-αMeF(2F)-TSDY-Aib-I-αMeL-LD-Orn-QAQ-Aib-Orn-FIK((2-[2-(2-Amino-ethoxy)-ethoxy]-acetyl)₂-(γ-Glu)₂-CO-(CH₂)₁₂-CO₂H)-αMeY-LIK((2-[2-(2-Amino-ethoxy)-ethoxy]-acetyl)₂-(γ-Glu)₂-CO-(CH₂)₁₂-CO₂H)GGPSSGAPPPS-NH₂ | 490 | 5782.51 |
| 481 | Y-Aib-EGT-αMeF(2F)-TSDY-Aib-I-αMeL-LD-Orn-IAQ-Aib-Orn-FIK((2-[2-(2-Amino-ethoxy)-ethoxy]-acetyl)₃-(γ-Glu)--(10-(4-carboxyphenoxy)decanoyl))-αMeY-LIK((2-[2-(2-Amino-ethoxy)-ethoxy]-acetyl)₃-(γ-Glu)--(10-(4-carboxyphenoxy)decanoyl))GGPSSGAPPPS-NH₂ | 491 | 5899.65 |
| 482 | Y-Aib-EGT-αMeF(2F)-TSD-4Pal-Aib-I-αMeL-LD-Orn-QAQ-Aib-Orn-FIK((γ-Glu)-(2-[2-(2-Amino-ethoxy)-ethoxy]-acetyl)₂-(γ-Glu)-CO-(CH₂)₁₂-CO₂H)-αMeY-LIK((γ-Glu)-(2-[2-(2-Amino-ethoxy)-ethoxy]-acetyl)₂-(γ-Glu)-CO-(CH₂)₁₂-CO₂H)GGPSSGAPPPS-NH₂ | 492 | 5767.5 |
| 483 | Y-Aib-EGT-αMeF(2F)-TSD-4Pal-Aib-I-αMeL-LD-Orn-QAQ-Aib-Orn-FIK((γ-Glu)-(2-[2-(2-Amino-ethoxy)-ethoxy]-acetyl)₂-(γ-Glu)-(10-(4-carboxyphenoxy)decanoyl))-αMeY-LIK((γ-Glu)-(2-[2-(2-Amino-ethoxy)-ethoxy]-acetyl)₂-(γ-Glu)-(10-(4-carboxyphenoxy)decanoyl))GGPSSGAPPPS-NH₂ | 493 | 5867.53 |
| 484 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)-(γ-Glu)-(2-[2-(2-Amino-ethoxy)-ethoxy]-acetyl)-(γ-Glu)-CO-(CH₂)₁₂-CO₂H)-αMeY-LIK((2-[2-(2-Amino-ethoxy)-ethoxy]-acetyl)-(γ-Glu)-(2-[2-(2-Amino-ethoxy)-ethoxy]-acetyl)-(γ-Glu)-CO-(CH₂)₁₂-CO₂H)GGPSSGAPPPS-NH₂ | 494 | 5767.5 |
| 485 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)-(γ-Glu)-(2-[2-(2-Amino-ethoxy)-ethoxy]-acetyl)-(γ-Glu)-(10-(4-carboxyphenoxy)decanoyl))-αMeY-LIK((2-[2-(2-Amino-ethoxy)-ethoxy]-acetyl)-(γ-Glu)-(2-[2-(2-Amino-ethoxy)-ethoxy]-acetyl)-(γ-Glu)-(10-(4-carboxyphenoxy)decanoyl))GGPSSGAPPPS-NH₂ | 495 | 5867.53 |
| 486 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)-(γ-Glu)₃-CO-(CH₂)₁₂-CO₂H)-αMeY-LIK((2-[2-(2-Amino-ethoxy)-ethoxy]-acetyl)-(γ-Glu)₃-CO-(CH₂)₁₂-CO₂H)GGPSSGAPPPS-NH₂ | 496 | 5735.41 |
| 487 | Y-Aib-EGT-αMeF(2F)-TSD-4Pal-Aib-I-αMeL-LD-Orn-QAQ-Aib-Orn-FIK((2-[2-(2-Amino-ethoxy)-ethoxy]-acetyl)-(γ-Glu)₃-(10-(4-carboxyphenoxy)decanoyl))-αMeY-LIK((2-[2-(2-Amino-ethoxy)-ethoxy]-acetyl)-(γ-Glu)₃--(10-(4-carboxyphenoxy)decanoyl))GGPSSGAPPPS-NH₂ | 497 | 5835.44 |
| 488 | Y-Aib-EGT-αMeF(2F)-TSD-4Pal-Aib-I-αMeL-LD-Orn-QAQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 498 | 5524.24 |
| 489 | Y-Aib-EGT-αMeF(2F)-TSD-4Pal-Aib-I-αMeL-LD-Orn-QAQ-Aib-K((2-[2-(2-Amino-ethoxy)-ethoxy]-acetyl)₂-(γ-Glu)-CO-(CH₂)₁₂-CO₂H)FI-(D-Glu)-αMeY-LIEGGPSSGAPPPSK((2-[2-(2-Amino-ethoxy)-ethoxy]-acetyl)₂-(γ-Glu)-CO-(CH₂)₁₂-CO₂H)-NH₂ | 499 | 5653.35 |
| 490 | Y-Aib-EGT-αMeF(2F)-TSD-Nle-SI-αMeL-LD-Orn-QAQ-Aib-Orn-FIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 500 | 5474.27 |
| 491 | Y-Aib-EGT-αMeF(2F)-TSD-hY-SI-αMeL-LD-Orn-QAQ-Aib-Orn-FIK((2-[2-(2-Amino-ethoxy)-ethoxy]-acetyl)₂-(γ-Glu)-CO-(CH₂)₁₂-CO₂H)-αMeY-LIK((2-[2-(2-Amino-ethoxy)-ethoxy]-acetyl)₂-(γ-Glu)-CO-(CH₂)₁₂-CO₂H)GGPSSGAPPPS-NH₂ | 501 | 5538.31 |
| 492 | Y-Aib-EGT-αMeF(2F)-TSDY-Aib-I-αMeL-LD-Orn-QAQ-Aib-Orn-FIK((2-[2-(2-Amino-ethoxy)-ethoxy]-acetyl)₂-(γ-Glu)--C10_4CP)-αMeY-LIK((2-[2-(2-Amino-ethoxy)-ethoxy]-acetyl)₂-(γ-Glu)--C10_4CP)GGPSSGAPPPS-NH₂ | 502 | 5624.31 |
| 493 | Y-Aib-EGT-αMeF(2F)-TSD-4Pal-Aib-I-αMeL-LD-Orn-QAQ-Aib-4Pal-FIK((2-[2-(2-Amino-ethoxy)-ethoxy]-acetyl)₂-(γ-Glu)-(10-(4-carboxyphenoxy)decanoyl))-αMeY-LIK((2-[2-(2-Amino-ethoxy)-ethoxy]-acetyl)₂-(γ-Glu)-(10-(4-carboxyphenoxy)decanoyl))GGPSSGAPPPS-NH₂ | 503 | 5643.32 |
| 494 | Y-Aib-EGT-aMeF(2F)-TSD-4Pal-Aib-I-aMeL-LD-Orn-K((2-[2-(2-Amino-ethoxy)-ethoxy]-acetyl)₂-(γ-Glu)-CO-(CH₂)₁₀-CH₃)AQ-Aib-GFIe-aMeY-LIK((2-[2-(2-Amino-ethoxy)-ethoxy]-acetyl)₂-(γ-Glu)-CO-(CH₂)₁₀-CH₃)GGPSSGAPPPS-NH2 | 504 | 5337.09 |

### Binding Assays

Glucagon (referred to as Gcg) is a Reference Standard prepared at Eli Lilly and Company. GLP-1, 7-36-NH₂ (referred to as GLP-1) is obtained from CPC Scientific (Sunnyvale, CA, 97.2% purity, 100 µM aliquots in 100% DMSO). GIP 1-42 (referred to as GIP) is prepared at Lilly Research Laboratories using peptide synthesis and HPLC chromatography as described above (>80% purity, 100 µM aliquots in 100% DMSO). [¹²⁵I]-radiolabeled Gcg, GLP-1, or GIP is prepared using [¹²⁵I]-lactoperoxidase and obtained from Perkin Elmer (Boston, MA).

Stably transfected cell lines are prepared by subcloning receptor cDNA into a pcDNA3 expression plasmid and transfected into human embryonic kidney (HEK) 293 (hGcgR and hGLP-1R) or Chinese Hamster Ovary (CHO) (hGIPR) cells followed by selection with Geneticin (hGLP-1R and hGIPR) or hygromycin B (hGcgR).

Two methods are used for the preparation of crude cell membranes.

Method 1: Frozen cell pellets are lysed on ice in hypotonic buffer containing 50 mM Tris HCl, pH 7.5, and Roche Complete™ Protease Inhibitors with EDTA. The cell suspension is disrupted using a glass Potter-Elvehjem homogenizer fitted with a Teflon® pestle for 25 strokes. The homogenate is centrifuged at 4° C. at 1100 × g for 10 minutes. The supernatant is collected and stored on ice while the pellets are resuspended in homogenization buffer and rehomogenized as described above. The homogenate is centrifuged at 1100 × g for 10 minutes. The second supernatant is combined with the first supernatant and centrifuged at 35000 × g for 1 hour at 4° C. The resulting membrane pellet is resuspended in homogenization buffer containing protease inhibitors at approximately 1 to 3 mg/mL, quick frozen in liquid nitrogen and stored as aliquots in a -80° C. freezer until use.

Method 2: Frozen cell pellets are lysed on ice in hypotonic buffer containing 50 mM Tris HCl, pH 7.5, 1 mM MgCh, Roche Complete™ EDTA-free Protease Inhibitors and 25 units/ml DNAse I (Invitrogen). The cell suspension is disrupted using a glass Potter-Elvehjem homogenizer fitted with a Teflon® pestle for 20 to 25 strokes. The homogenate is centrifuged at 4° C. at 1800 × g for 15 minutes. The supernatant is collected and stored on ice while the pellets are resuspended in homogenization buffer (without DNAse I) and rehomogenized as described above. The homogenate is centrifuged at 1800 × g for 15 minutes. The second supernatant is combined with the first supernatant and centrifuged an additional time at 1800 × g for 15 minutes. The overall supernatant is then centrifuged at 25000 × g for 30 minutes at 4° C. The resulting membrane pellet is resuspended in homogenization buffer (without DNAse I) containing protease inhibitors at approximately 1 to 3 mg/mL and stored as aliquots in a -80° C. freezer until use.

### Binding Determination Methods

The equilibrium binding dissociation constants (K_{d}) for the various receptor/radioligand interactions are determined from homologous competition binding analysis instead of saturation binding due to high propanol content in the [¹²⁵I] stock material. The K_{d} values determined for the receptor preparations were as follows: hGcgR (3.9 nM), hGLP-1R (1.2 nM) and hGIPR (0.14 nM).

### [¹²⁵I]-Glucagon Binding

The human Gcg receptor binding assays are performed using a Scintillation Proximity Assay (SPA) format with wheat germ agglutinin (WGA) beads (Perkin Elmer). The binding buffer contains 25 mM 4-(2-hydroxyethyl)-1-piperazineethanesulfonic acid (HEPES), pH 7.4, 2.5 mM CaCl₂, 1 mM MgCh, 0.1% (w/v) bacitracin (Research Products), 0.003% (w/v) Polyoxyethylenesorbitan monolaurate (TWEEN®-20), and Roche Complete™ Protease Inhibitors without EDTA. Peptides and Gcg are thawed and 3-fold serially diluted in 100% DMSO (10 point concentration response curves). Next, 5 µL serially diluted compound or DMSO is transferred into Corning® 3632 clear bottom assay plates containing 45 µL assay binding buffer or unlabeled Gcg control (non-specific binding or NSB, at 1 µM final). Then, 50 µL [¹²⁵I]-Gcg (0.15 nM final), 50 µL human GcgR membranes (1.5 µg/well) and 50 µL of WGA SPA beads (80 to 150 µg/well) are added with a Biotek Multiflo dispenser. Plates are sealed and mixed on a plate shaker (setting 6) for 1 minute and read with a PerkinElmer Trilux MicroBeta® scintillation counter after 12 hours of incubation/settling time at room temperature. Final assay concentration ranges for peptides tested in response curves is typically 1150 nM to 0.058 nM and for the control Gcg from 1000 nM to 0.05 nM.

### [¹²⁵I]-GLP-1 Binding

The human GLP-1 receptor binding assay is performed using an SPA format with WGA beads. The binding buffer contains 25 mM HEPES, pH 7.4, 2.5 mM CaCl₂, 1 mM MgCl₂, 0.1% (w/v) bacitracin, 0.003% (w/v) TWEEN®-20, and Roche Complete™ Protease Inhibitors without EDTA. Peptides and GLP-1 are thawed and 3-fold serially diluted in 100% DMSO (10 point concentration response curves). Next, 5 µL serially diluted compound or DMSO is transferred into Corning® 3632 clear bottom assay plates containing 45 µL assay binding buffer or unlabeled GLP-1 control (non-specific binding or NSB, at 0.25 µM final). Then, 50 µL [¹²⁵I]-GLP-1 (0.15 nM final), 50 µL human GLP-1R membranes (0.5 µg/well and 50 µL of WGA SPA beads (100 to 150 µg/well) are added with a Biotek Multiflo dispenser. Plates are sealed and mixed on a plate shaker (setting 6) for 1 minute and read with a PerkinElmer Trilux MicroBeta® scintillation counter after 5 to 12 hours of incubation/settling time at room temperature. Final assay concentration ranges for peptides tested in response curves are typically 1150 nM to 0.058 nM and for the control GLP-1, 250 nM to 0.013 nM.

### [125I]-GIP Binding

The human GIP receptor binding assay is performed using an SPA format with WGA beads. The binding buffer contains 25 mM HEPES, pH 7.4, 2.5 mM CaCl₂, 1 mM MgCl₂, 0.1% (w/v) bacitracin, 0.003% (w/v) TWEEN®-20, and Roche Complete™ Protease Inhibitors without EDTA. Peptides and GIP are thawed and 3 fold serially diluted in 100% DMSO (10 point concentration response curves). Next, 5 µL serially diluted compound or DMSO is transferred into Corning® 3632 clear bottom assay plates containing 45 µL assay binding buffer or unlabeled GIP control (non-specific binding or NSB, at 0.25 µM final). Then, 50 µL [¹²⁵I]-GIP (0.075-0.15 nM final), 50 µL human GIPR membranes (3 µg/well) and 50 µL of WGA SPA beads (100 to 150 µg/well) are added with a Biotek Multiflo dispenser. Plates are sealed and mixed on a plate shaker (setting 6) for 1 minute and read with a PerkinElmer Trilux MicroBeta® scintillation counter after 2.5 to 12 hours of incubation/settling time at room temperature. Final assay concentration ranges for peptides tested in response curves is typically 1150 to 0.058 nM or 115 nM to 0.0058 nM and for the control GIP, 250 nM to 0.013 nM.

### Binding Assay Data Analysis

Raw CPM data for concentration curves of peptides, Gcg, GLP-1, or GIP are converted to percent inhibition by subtracting nonspecific binding (binding in the presence of excess unlabeled Gcg, GLP-1, or GIP, respectively) from the individual CPM values and dividing by the total binding signal, also corrected by subtracting nonspecific binding. Data are analyzed using four-parameter (curve maximum, curve minimum, IC₅₀, Hill slope) nonlinear regression routines (Genedata Screener, version 12.0.4, Genedata AG, Basal, Switzerland). The affinity constant (Kᵢ) is calculated from the absolute IC₅₀ value based upon the equation Kᵢ = IC₅₀/(1 + D/K_{d}) where D is the concentration of radioligand used in the experiment, IC₅₀ is the concentration causing 50% inhibition of binding and K_{d} is the equilibrium binding dissociation constant of the radioligand (described above). Values for Kᵢ are reported as the geometric mean, with error expressed as the standard error of the mean (SEM) and n is equal to the number of independent replicates (determined in assays performed on different days). Geometric Means are calculated as follows:

$${\text{Geometric Mean = 10}}^{\left(\text{Arithmetic Mean of Log Ki Values)}\right)}$$

The Ki Ratio (Ki for native control peptide/Ki for test compound) at each receptor and each species is calculated. The Ki Ratio is a rapid indication of the apparent affinity of a peptide compared to the native control peptide. A Ki Ratio < 1 indicates that the test peptide has a lower affinity (higher Ki value) for the receptor than the native peptide, whereas a Ki Ratio >1 indicates that the test peptide has a higher affinity (lower Ki value) for the receptor than the native peptide.

n=⅟x means that only one value out of the total number of replicates (x) is used to express the mean. SEM is only calculated when n=2 or greater non-qualified results exist. Means are expressed as GeoMetric means with the standard error of the mean (SEM) and the number of replicates (n) indicated in parenthesis.

TABLE 1

In vitro Binding Affinity (Kᵢ) of indicated Examples and comparator molecules for human GLP-1R, GcgR and GIPR.
Binding Affinity
Ki, nM (SEM, n)

| Molecule or Example | hGIPR | hGLP-1R | hGCGR |
|---|---|---|---|
| hGIP(1-42)NH2 | 0.135 (0.00585, 378/380) | | |
| hGLP-1(7-36)NH2 | | 0.791 (0.0236, 545) | |
| hGCG | | | 3.07 (0.115, 510/511) |
| 1 | 0.0880 (0.0147, 3) | 2.33 (0.341, 3) | >969 (⅓) |
| 2 | 0.0446 | 1.04 | >958 |
| 3 | 0.0629 | 0.471 | 711 |
| 4 | 0.0528 (0.0149, 2) | 0.413 (0.0153, 2) | 637 (12.3, 2) |
| 5 | 0.0309 (0.00250, 3) | 0.154 (0.0391, 3) | 44.8 (53.4, 3) |
| 6 | 0.145 | 2.99 | >969 |
| 7 | 0.0501 (0.00636, 3) | 0.362 (0.101, 3) | 1.46 (0.930, 3) |
| 8 | 0.0240 (0.00569, 3) | 0.139 (0.0154, 3) | 4.29 (4.32, 3) |
| 9 | 0.0557 (0.00698, 3) | 1.05 (0.0625, 3) | 209 (⅓) |
| 10 | 0.0489 (0.00825, 2) | 0.681 (0.0423, 2) | 424 (214, 2) |
| 11 | 0.0508 (0.00450, 2) | 1.06 (0.222, 2) | 660 (24.3, 2) |
| 12 | 0.0575 (0.00224, 2) | 0.638 (0.249, 2) | 213 (27.9, 2) |
| 13 | 0.0575 (0.0351, 2) | 0.691 (0.304, 2) | 0.987 (0.0552, 2) |
| 14 | 0.0360 (0.00828, 2) | 0.286 (0.170, 2) | 17.6 (0.912, 2) |
| 15 | 0.0629 (0.00467, 3) | 2.11 (0.383, 3) | >966 (⅓) |
| 16 | 0.0540 (0.0165, 3) | 1.40 (0.275, 3) | 707 (53.3, ⅔) |
| 17 | 0.0649 (0.0248, 3) | 3.18 (0.127, 3) | >966 (⅓) |
| 18 | 0.0769 (0.0200, 2) | 8.41 (2.71, 2) | >958 (½) |
| 19 | 0.0807 (0.00750, 3) | 3.13 (0.185, 3) | >966 (⅓) |
| 20 | 0.0722 (0.00347, 4) | 1.96 (0.319, 4) | 139 (27.5, 4) |
| 21 | 0.0231 (0.00290, 3) | 0.246 (0.0413, 3) | 0.293 (0.0186, 3) |
| 22 | 0.0217 (0.00493, 3) | 0.301 (0.0914, 3) | 0.596 (0.0330, 3) |
| 23 | 0.0238 (0.00306, 3) | 0.251 (0.0187, 3) | 1.18 (0.130, 3) |
| 24 | 0.0361 (0.00975, 3) | 0.520 (0.0555, 3) | 0.646 (0.0169, 3) |
| 25 | 0.0293 (0.0105, 3) | 0.483 (0.0966, 3) | 0.283 (0.0162, 3) |
| 26 | 0.0863 (0.00997, 3) | 0.867 (0.300, 3) | 3.82 (0.560, 3) |
| 27 | 0.378 (0.140, 2) | 6.48 (1.80, 2) | >958 (½) |
| 28 | 0.0972 (0.00197, 2) | 3.01 (0.442, 2) | >966 (½) |
| 29 | 0.0505 | 0.412 | 0.923 |
| 30 | 0.0653 (0.0190, 2) | 2.66 (0.303, 2) | >966 (½) |
| 31 | 0.0308 | 0.315 | 0.62 |
| 33 | 0.117 | 1.34 | 264 |
| 34 | 0.131 | 2.81 | >957 |
| 35 | 0.077 | 1.38 | 868 |
| 36 | 0.103 | 1.47 | 611 |
| 37 | 0.171 | 2.22 | >957 |
| 38 | 0.0608 | 0.803 | 174 |
| 39 | 0.234 | 1.69 | 412 |
| 40 | 0.163 | 1.6 | 223 |
| 41 | 0.108 | 2.06 | >957 |
| 42 | 0.0759 | 2.02 | 458 |
| 43 | 0.092 | 2.5 | >957 |
| 44 | 0.0595 | 3.08 | >966 |
| 45 | 0.0891 | 6.9 | >966 |
| 46 | 0.0683 | 6.1 | >966 |
| 47 | 0.0761 | 29 | 217 |
| 48 | 0.0621 | 3.67 | >966 |
| 49 | 0.0573 | 1.21 | 196 |
| 50 | 0.0296 | 0.876 | 42 |
| 51 | 0.0741 | 1.64 | 51.7 |
| 52 | 0.0832 | 1.16 | >966 |
| 53 | 0.085 | 1.46 | 274 |
| 55 | 0.252 | 5.17 | 299 |
| 56 | 0.152 | 2.85 | 137 |
| 57 | 0.297 | 4.35 | 114 |
| 58 | 0.208 | 1.15 | 1920 |
| 59 | 0.171 | 0.789 | 2000 |
| 60 | 0.11 | 0.872 | 737 |
| 62 | 0.132 | 1.59 | >4780 |
| 64 | 0.161 | 1.52 | >4780 |
| 65 | 0.121 | 1.05 | 4290 |
| 66 | 0.263 | 1.55 | 4150 |
| 70 | 0.120 (0.0470, 2) | 1.67 (0.736, 4) | 151 |
| 73 | 0.0454 | 3.38 | 53.9 |
| 75 | 0.107 | 2.8 | 206 |
| 78 | 0.0627 | 0.881 (0.349, 4) | 625 |
| 91 | 0.126 | 1.26 (0.316, 4) | >955 |
| 128 | 0.0314 | 0.777 (0.202, 4) | >955 |
| 137 | 0.0403 | 0.250 (0.0518, 4) | 8.29 |
| 139 | 0.0415 (0.00520, 2) | 0.268 (0.0773, 4) | 109 |
| 141 | 0.0291 (0.00776, 2) | 0.194 (0.0837, 4) | 13.9 |
| 212 | 0.161 | 1.17 (0.333, 4) | >955 |
| 216 | 0.0491 | 0.863 (0.505, 4) | 328 |
| 219 | 0.114 | 0.939 (0.627, 4) | 135 |
| 246 | 0.0432 (0.00892, 2) | 0.557 | 44.6 |
| 292 | 0.0161 (0.00313, 5) | 0.289 (0.0849, 5) | 132 (25.5, 5) |
| 293 | 0.022 | 0.211 | 23.1 |
| 298 | 0.0131 | 0.119 | 14.3 |
| 299 | 0.0215 | 0.101 | 93.3 |
| 302 | 0.0228 | 0.137 | 2.84 |
| 322 | 0.0162 (0.00203, 5) | 0.234 (0.0542, 5) | 2.91 (0.693, 5) |
| 324 | 0.0163 (0.00117, 5) | 0.168 (0.0289, 5) | 6.28 (0.938, 5) |
| 334 | 0.0195 (0.00139, 5) | 0.325 (0.0497, 5) | 75.5 (12.7, 5) |
| 336 | 0.0782 | 1.85 | 230 |
| 337 | 0.0747 | 2.17 | >957 |
| 338 | 0.091 | 2.07 | >957 |
| 360 | 0.046 | 0.673 | 118 |
| 361 | 0.0578 | 1.22 | 238 |
| 362 | 0.053 | 0.765 | 360 |
| 391 | 0.0279 (0.00204, 4) | 0.346 (0.0663, 3) | 289 (41.4, 3) |
| 403 | 0.0375 | 0.424 | 134 |
| 411 | 0.0375 (0.00780, 2) | 1.33 | >957 |
| 414 | 0.0229 (0.000330, 3) | 0.194 (0.0265, 3) | 33.7 (5.31, 3) |
| 415 | 0.0152 (0.00797, 2) | 0.156 (0.109, 2) | 51.8 (18.2, 2) |
| 417 | 0.0214 (0.00497, 3) | 0.167 (0.0783, 3) | 58.4 (7.11, 3) |
| 418 | 0.0174 (0.00276, 2) | 0.0684 | 3.59 (1.87, 2) |
| 419 | 0.0166 | 0.265 | 60.6 |
| 421 | 0.0245 (0.00418, 3) | 0.198 (0.0413, 3) | 345 (101, 3) |
| 422 | 0.0218 (0.00310, 3) | 0.194 (0.0162, 3) | 12.2 (1.63, 3) |
| 423 | 0.0207 (0.00795, 3) | 0.275 (0.0192, 3) | 85.9 (10.6, 3) |
| 424 | 0.0206 (0.00786, 3) | 0.219 (0.0306, 3) | 16.0 (1.93, 3) |
| 432 | 0.0134 (0.00295, 2) | 0.134 (0.00717, 2) | 356 (72.8, 2) |
| 433 | 0.0175 | 0.187 | 28.3 |
| 434 | 0.00965 (0.00495, 2) | 0.150 (0.0253, 2) | 81.2 (12.2, 2) |
| 435 | 0.00910 (0.000766, 2) | 0.122 (0.00471, 2) | 14.4 (1.56, 2) |
| 436 | 0.0372 | 0.412 | 64.9 |
| 440 | 0.0312 (0.00264, 3) | 0.373 (0.118, 3) | 131 (27.8, 3) |
| 441 | 0.0539 (0.0323, 2) | 0.386 (0.0706, 2) | 592 (½) |
| 442 | 0.0507 | 0.164 | 130 |
| 446 | 0.0369 (0.00841, 2) | 0.382 (0.116, 2) | 109 (11.0, 2) |
| 448 | 0.0314 (0.00845, 2) | 0.367 (0.103, 2) | 116 (13.5, 2) |
| 449 | 0.0133 | 0.201 | 243 |
| 451 | 0.0239 | 0.234 | 258 |
| 453 | 0.013 | 0.123 | 71.8 |
| 455 | 0.0143 | 0.243 | 60.1 |
| 457 | 0.0419 (0.0217, 2) | 0.252 (0.0198, 2) | 351 (0.454, 2) |
| 458 | 0.0232 | 0.266 | 170 |
| 459 | 0.023 | 0.224 | 108 |
| 460 | 0.0528 (0.0151, 2) | 0.433 (0.0173, 2) | >959 (½) |
| 461 | 0.0100 (0.00141, 2) | 0.140 (0.0483, 2) | 101 (26.9, 2) |
| 462 | 0.0102 | 0.125 | 87.5 |
| 463 | 0.0183 | 0.143 | 54.5 |
| 464 | 0.0261 (0.00416, 2) | 0.352 (0.0418, 2) | 753 (58.6, 2) |
| 465 | 0.0464 | 1.29 | 304 |
| 466 | 0.0199 (0.00474, 2) | 0.200 (0.0377, 2) | 342 (223, 2) |
| 467 | 0.0486 | 0.381 | 208 |
| 468 | 0.0291 | 0.0943 | 269 |
| 469 | 0.0387 | 0.0847 | 144 |
| 470 | 0.0346 | 0.121 | 945 |
| 471 | 0.0493 | 0.197 | 452 |
| 472 | 0.0376 | 0.254 | 258 |
| 473 | 0.0319 | 0.169 | 162 |
| 474 | 0.0427 | 0.343 | 382 |
| 475 | 0.0294 | 0.189 | 139 |
| 476 | 0.0351 | 0.226 | 605 |
| 477 | 0.0279 | 0.143 | 268 |
| 478 | 0.0406 | 0.407 | 49.7 |
| 479 | 0.0249 | 0.246 | 213 |
| 480 | 0.0368 | 0.307 | 88.5 |
| 481 | 0.0266 | 0.326 | 20.1 |
| 482 | 0.0277 | 0.254 | 273 |
| 483 | 0.0236 | 0.263 | 151 |
| 484 | 0.0296 | 0.268 | 305 |
| 485 | 0.0548 | 0.3 | 121 |
| 486 | 0.046 | 0.299 | 115 |
| 487 | 0.0714 | 0.217 | 73.3 |
| 488 | 0.064 | 0.964 | >958 |
| 489 | 0.0912 | 0.947 | >957 |
| 490 | 0.0288 | 0.27 | 37.8 |
| 491 | 0.0301 | 0.182 | 94.4 |
| 492 | 0.0264 | 0.233 | 28.6 |
| 493 | 0.0345 | 0.25 | 70.1 |
| 494 | 0.0197 (0.00489, 2) | 0.188 (0.0462, 2) | 135 (26.5, 2) |

### Functional Activity

### Functional hGIP-R, hGLP-1R and hGCGR Assays

Methods: Functional activity is determined using cAMP formation in HEK-293 clonal cell lines expressing hGIPR, hGLP-1R or hGCGR. hGIPR, hGLP-1R or hGCGR receptor-expressing cells are treated with a control polypeptide or one of Examples 1 to 3 (20 point concentration-response curve in DMSO, 2.75-fold Labcyte Echo direct dilution, 384 well plate Corning Cat# 3570) in DMEM (Gibco Cat# 31053) supplemented with 1X GlutaMAX™ (Gibco Cat# 35050), 0.1% bovine casein (Sigma C4765-10ML), 250 µM IBMX (3-Isobutyl-1-methylxanthine, Acros Cat# 228420010) and 20 mM HEPES (Gibco Cat# 15630) in a 20 µL assay volume (final DMSO concentration was 0.5%). Experiments also are performed under identical assay conditions with the addition of 1.0 % fatty acid free, globulin free human serum albumin (Sigma Cat# A3782).

After a 30-min incubation at 37° C., the resulting increase in intracellular cAMP is quantitatively determined using a CisBio cAMP Dynamic 2 HTRF Assay Kit (62AM4PEJ). Briefly, cAMP levels within the cell are detected by adding the cAMP-d2 conjugate in cell lysis buffer (10 µL) followed by the antibody anti-cAMP-Eu³⁺-Cryptate, also in cell lysis buffer (10 µL). The resulting competitive assay is incubated for at least 60 min at room temperature, and then is detected using a PerkinElmer Envision® instrument with excitation at 320 nm and emission at 665 nm and 620 nm. Envision units (emission at 665 nm/620 nm* 10,000) are inversely proportional to the amount of cAMP present and are converted to nM cAMP per well us-ing a cAMP standard curve. The amount of cAMP generated (nM) in each well is converted to a percent of the maximal response observed with human GIP(1-42)NH₂, hGLP-1(7-36)NH₂ or hGCG. A relative EC₅₀ value and percent top (Eₘₐₓ) are derived by non-linear regression analysis using the percent maximal response vs. the concentration of peptide added, fitted to a four-parameter logistic equation.

Results: Functional data for hGIP(1-42)NH₂, hGLP-1(7-36)NH₂, hGCG and the Example compounds are provided below in Table 2 (0.1% bovine casein) and Table 3 (0.1% bovine casein, 1.0% human serum albumin).

TABLE 2

Functional cAMP Potency (EC₅₀) and Efficacy (Eₘₐₓ) for Peptides Incubated at 37° C. (in the presence of 0.1% bovine casein)

| | hGIPR ^{a} | | hGLP-1R ^{a} | | hGCGR^{a} | |
|---|---|---|---|---|---|---|
| Example | EC₅₀, nM (SEM, n) ^{b} | Emax, % ± SEM ^{c} | EC₅₀, nM (SEM, n) ^{b} | Emax, % ± SEM ^{c} | EC₅₀, nM (SEM, n) ^{b} | Emax, % ± SEM ^{c} |
| hGIP(1-42)NH2 | 0.988 (0.0675, 244/246) | 100 ± 0.863 | | | | |
| hGLP-1(7-36)NH2 | | | 0.315 (0.00999, 400) | 106 ± 0.687 | | |
| hGCG | | | | | 1.95 (0.0802, 301) | 101 (se=1.13, n=301) |
| 1 | 0.0145 (0.00159, 5) | 90.2 ± 2.82 | 0.925 (0.101, 5) | 97.0 ± 4.47 | >9950 (n=⅕) | ND |
| 6 | 0.0477 (0.00717, 3) | 101 ± 3.61 | 2.30 (0.212, 3) | 105 ± 1.05 | >9950 (n=⅓) | ND |
| 2 | 0.0154 (0.000643, 5) | 93.0 ± 0.813 | 1.14 (0.0827, 5) | 93.1 ± 5.83 | >9950 (n=⅕) | ND |
| 3 | 0.0133 (0.00186, 3) | 95.9 ± 2.95 | 0.223 (0.0583, 3) | 107 ± 2.51 | >9950 (n=⅓) | ND |
| 4 | 0.00962 (0.000822, 6) | 92.9 ± 0.381 | 0.137 (0.0117, 6) | 99.2 ± 1.24 | >9950 (n=⅙) | ND |
| 7 | 0.00327 (0.000331, 8) | 99.5 ± 2.88 | 0.00676 (0.00104, 8) | 95.4 ± 5.03 | >9950 (n=⅛) | ND |
| 8 | 0.00307 (0.000291, 5) | 103 ± 3.48 | 0.0157 (0.00381, 5) | 103 ± 2.63 | >9950 (n=⅕) | ND |
| 5 | 0.00585 (0.000683, 6) | 99.9 ± 0.649 | 0.0570 (0.0122, 6) | 103 ± 4.79 | >9150 (n=⅙) | ND |
| 9 | 0.0102 (0.000680, 5) | 93.6 ± 2.36 | 0.421 (0.0704, 5) | 98.0 ± 4.97 | >9950 (n=⅕) | ND |
| 10 | 0.0106 (0.00115, 4) | 97.9 ± 1.99 | 0.329 (0.0311, 4) | 99.1 ± 4.04 | >9950 (n=¼) | ND |
| 11 | 0.0135 (0.00157, 5) | 97.7 ± 2.82 | 0.511 (0.0355, 5) | 102 ± 2.11 | >9950 (n=⅕) | ND |
| 12 | 0.0127 (0.000647, 5) | 100.0 ± 3.09 | 0.423 (0.0365, 5) | 105 ± 5.32 | >9950 (n=⅕) | ND |
| 13 | 0.00597 (0.000599, 6) | 99.0 ± 1.77 | 0.0214 (0.00421, 6) | 104 ± 4.07 | >9950 (n=⅙) | ND |
| 14 | 0.00802 (0.00123, 6) | 95.0 ± 2.32 | 0.0884 (0.00658, 6) | 100 ± 3.78 | >9950 (n=⅙) | ND |
| 15 | 0.0133 (0.00177, 6) | 95.9 ± 2.34 | 0.995 (0.149, 6) | 94.3 ± 4.44 | >9950 (n=⅙) | ND |
| 16 | 0.0113 (0.00240, 6) | 101 ± 4.45 | 0.245 (0.0192, 6) | 104 ± 1.61 | >9950 (n=⅙) | ND |
| 17 | 0.0129 (0.00209, 5) | 97.5 ± 5.04 | 1.57 (0.314, 5) | 106 ± 4.38 | >9950 (n=⅕) | ND |
| 18 | 0.0226 (0.00161, 4) | 97.1 ± 3.28 | 5.06 (0.710, 4) | 104 ± 4.11 | >9950 (n=¼) | ND |
| 19 | 0.0158 (0.00198, 4) | 96.6 ± 2.70 | 1.34 (0.201, 4) | 104 ± 1.72 | >9950 (n=¼) | ND |
| 20 | 0.00985 (0.000468, 3) | 98.8 ± 3.19 | 0.552 (0.0748, 3) | 103 ± 3.37 | >9950 (n=⅓) | ND |
| 21 | 0.00347 (0.000301, 4) | 102 ± 2.44 | 0.00655 (0.000594, 4) | 106 ± 2.32 | >9950 (n=¼) | ND |
| 22 | 0.00656 (0.00118, 4) | 102 ± 3.06 | 0.0153 (0.00165, 4) | 98.6 ± 2.87 | >9950 (n=¼) | ND |
| 23 | 0.00299 (0.000153, 4) | 101 ± 3.70 | 0.00722 (0.00106, 4) | 102 ± 3.01 | >9950 (n=¼) | ND |
| 24 | 0.00266 (0.000185, 5) | 104 ± 2.75 | 0.00630 (0.000480, 5) | 104 ± 3.48 | >9950 (n=⅕) | ND |
| 25 | 0.00243 (0.000195, 5) | 101 ± 4.03 | 0.00426 (0.000485, 5) | 102 ± 5.06 | >9950 (n=⅕) | ND |
| 26 | 0.0218 (0.00198, 5) | 99.1 ± 0.793 | 0.0711 (0.00713, 5) | 106 ± 4.85 | >9950 (n=⅕) | ND |
| 27 | 0.116 | 88.8 | 2.77 | 102 | >9950 | ND |
| 28 | 0.0554 (0.0177, 3) | 95.4 ± 1.58 | 2.65 (0.180, 3) | 99.2 ± 4.11 | >9950 (n=⅓) | ND |
| 29 | 0.00252 (0.000214, 3) | 101 ± 3.24 | 0.00468 (0.000882, 3) | 100 ± 6.65 | >9950 (n=⅓) | ND |
| 30 | 0.0175 (0.00401, 3) | 99.3 ± 4.47 | 2.27 (0.367, 3) | 97.8 ± 10.7 | >9950 (n=⅓) | ND |
| 31 | 0.00157 (0.000662, 2) | 100.0 ± 5.25 | 0.00342 (0.00136, 2) | 91.3 ± 16.1 | >9950 (n=½) | ND |
| 32 | 0.002 | 98.4 | 0.0063 | 119 | >9950 | ND |
| 33 | 0.00957 (0.00110, 3) | 97.6 ± 2.45 | 0.370 (0.0515, 3) | 102 ± 4.78 | >9950 (n=⅓) | ND |
| 34 | 0.0148 (0.00105, 2) | 97.4 ± 2.34 | 1.00 (0.141, 2) | 99.6 ± 4.28 | >9950 (n=½) | ND |
| 35 | 0.0162 (0.00309, 3) | 97.0 ± 2.32 | 0.612 (0.125, 3) | 107 ± 4.76 | >9950 (n=⅓) | ND |
| 36 | 0.0185 (0.00551, 3) | 95.4 ± 2.02 | 0.536 (0.0920, 3) | 100 ± 3.79 | >9950 (n=⅓) | ND |
| 37 | 0.0211 (0.0115, 2) | 91.2 ± 3.67 | 0.909 (0.0795, 3) | 105 ± 3.13 | >9950 (n=⅓) | ND |
| 38 | 0.0155 (0.00104, 3) | 99.7 ± 1.92 | 0.225 (0.0142, 3) | 106 ± 1.38 | >9950 (n=⅓) | ND |
| 39 | 0.0238 (0.00641, 3) | 96.5 ± 1.44 | 0.358 (0.0311, 3) | 105 ± 6.57 | >9950 (n=⅓) | ND |
| 40 | 0.0116 (0.00653, 2) | 99.7 ± 2.94 | 0.279 (0.0725, 2) | 101 ± 5.30 | >9950 (n=½) | ND |
| 41 | 0.0108 (0.000456, 2) | 101 ± 3.16 | 0.691 (0.0736, 2) | 102 ± 3.70 | >9950 (n=½) | ND |
| 42 | 0.0103 (0.00133, 2) | 97.8 ± 7.18 | 0.429 (0.0612, 2) | 104 ± 1.38 | >9950 (n=½) | ND |
| 43 | 0.0123 (0.00510, 2) | 92.6 ± 5.56 | 0.821 (0.00670, 2) | 106 ± 2.02 | >9950 (n=½) | ND |
| 44 | 0.0637 (0.0744, 2) | 92.2 ± 6.17 | 4.77 (0.859, 2) | 90.9 ± 10.4 | >9950 (n=½) | ND |
| 45 | 0.0517 (0.0654, 2) | 91.3 ± 1.04 | 9.18 (5.30, 2) | 85.7 ± 8.37 | >9950 (n=½) | ND |
| 46 | 0.0801 (0.0992, 2) | 89.5 ± 2.64 | 3.29 (1.49, 2) | 79.2 ± 1.23 | >9950 (n=½) | ND |
| 47 | 0.0138 (0.00357, 2) | 89.8 ± 1.20 | 1.94 (4.03, 2) | 87.1 ± 14.1 | >9950 (n=½) | ND |
| 48 | 0.0182 (0.00262, 2) | 91.6 ± 3.38 | 1.000 (1.43, 2) | 87.8 ± 6.25 | >9950 (n=½) | ND |
| 49 | 0.0205 (0.00423, 5) | 91.0 ± 1.70 | 0.398 (0.0572, 5) | 97.3 ± 5.46 | >9950 (n=⅕) | ND |
| 50 | 0.0168 (0.00162, 4) | 94.7 ± 1.77 | 0.273 (0.0222, 4) | 101 ± 6.20 | >9950 (n=¼) | ND |
| 51 | 0.110 (0.0153, 2) | 101 ± 0.990 | 0.890 (0.0286, 2) | 85.7 ± 9.80 | >9950 (n=½) | ND |
| 52 | 0.258 (0.00726, 2) | 104 ± 1.25 | 0.384 (0.116, 2) | 84.3 ± 6.96 | >9950 (n=½) | ND |
| 53 | 0.0841 (0.0165, 4) | 99.6 ± 2.21 | 0.180 (0.0157, 4) | 89.9 ± 9.41 | >9950 (n=¼) | ND |
| 54 | 0.0675 | 113 | 0.538 | 101 | >9950 | ND |
| 55 | 0.0207 (0.00345, 4) | 97.1 ± 2.16 | 0.154 (0.0278, 4) | 108 ± 1.94 | >9950 (n=¼) | ND |
| 56 | 0.0138 (0.00215, 4) | 90.3 ± 2.57 | 0.205 (0.0208, 4) | 106 ± 4.02 | >9950 (n=¼) | ND |
| 57 | 0.0135 (0.00176, 3) | 91.5 ± 4.94 | 0.140 (0.0119, 3) | 102 ± 2.52 | >9950 (n=⅓) | ND |
| 58 | 0.0158 (0.00284, 3) | 89.2 ± 5.96 | 0.194 (0.0220, 3) | 101 ± 5.94 | >9950 (n=⅓) | ND |
| 59 | 0.0240 (0.00529, 3) | 89.2 ± 4.62 | 0.202 (0.0167, 3) | 107 ± 4.27 | >9950 (n=⅓) | ND |
| 60 | 0.0126 (0.00263, 3) | 94.0 ± 6.07 | 0.190 (0.0132, 3) | 97.2 ± 4.28 | >9950 (n=⅓) | ND |
| 61 | 0.012 | 88.2 | 1.35 | 82.9 | >9950 | ND |
| 62 | 0.0211 (0.00397, 3) | 96.1 ± 2.64 | 0.480 (0.0200, 3) | 99.4 ± 5.83 | >9950 (n=⅓) | ND |
| 63 | 0.0176 | 92.7 | 1.86 | 90.6 | >9950 | ND |
| 64 | 0.0158 (0.00140, 3) | 92.9 ± 3.06 | 0.557 (0.0434, 3) | 97.3 ± 4.13 | >9950 (n=⅓) | ND |
| 65 | 0.0139 (0.00208, 3) | 95.2 ± 2.93 | 0.300 (0.0590, 3) | 103 ± 0.751 | >9950 (n=⅓) | ND |
| 66 | 0.0168 (0.00351, 3) | 93.5 ± 1.39 | 0.355 (0.0103, 3) | 104 ± 1.78 | >9950 (n=⅓) | ND |
| 67 | 0.00795 | 95 | 2.63 | 91.7 | >9950 | ND |
| 68 | 0.0105 | 106 | 0.668 | 95.9 | >9950 | ND |
| 69 | 0.0265 (0.00500, 3) | 99.0 ± 2.43 | 0.276 (0.00951, 4) | 103 ± 1.65 | >9950 (n=¼) | ND |
| 70 | 0.0182 (0.00101, 4) | 89.1 ± 2.30 | 0.283 (0.0345, 5) | 100.0 ± 3.11 | >9950 (n=⅕) | ND |
| 71 | 0.0171 (0.00174, 3) | 93.8 ± 3.59 | 0.246 (0.0242, 4) | 106 ± 1.60 | >9950 (n=¼) | ND |
| 72 | 0.0160 (0.00172, 3) | 96.5 ± 3.71 | 0.245 (0.0271, 4) | 103 ± 3.06 | >9950 (n=¼) | ND |
| 73 | 0.0207 (0.00350, 4) | 97.1 ± 2.70 | 0.437 (0.0687, 4) | 99.2 ± 1.69 | >9950 (n=¼) | ND |
| 74 | 0.0156 (0.00216, 3) | 93.3 ± 2.89 | 0.334 (0.0383, 4) | 105 ± 3.28 | >9950 (n=¼) | ND |
| 75 | 0.0182 (0.00258, 4) | 92.3 ± 0.780 | 0.411 (0.0467, 5) | 104 ± 4.00 | >9950 (n=⅕) | ND |
| 76 | 0.0205 (0.00391, 4) | 102 ± 3.22 | 0.527 (0.0914, 4) | 93.5 ± 1.85 | >9950 (n=¼) | ND |
| 77 | 0.0288 (0.00467, 3) | 94.8 ± 2.69 | 3.03 (0.922, 3) | 87.0 ± 1.71 | >9950 (n=⅓) | ND |
| 78 | 0.0145 (0.00447, 4) | 101 ± 2.27 | 0.286 (0.0619, 4) | 100 ± 2.19 | >9950 (n=¼) | ND |
| 79 | 0.0130 (0.00567, 2) | 101 ± 0.395 | 0.223 (0.0242, 2) | 102 ± 1.09 | >9950 (n=½) | ND |
| 80 | 0.0245 (0.00347, 4) | 92.4 ± 1.14 | 0.718 (0.155, 4) | 102 ± 4.87 | >9950 (n=¼) | ND |
| 81 | 0.0114 (0.00273, 2) | 98.6 ± 6.19 | 0.176 (0.0351, 2) | 102 ± 3.94 | >9950 (n=½) | ND |
| 82 | 0.0206 (0.00375, 2) | 81.4 ± 6.09 | 4.78 (1.32, 2) | 103 ± 1.82 | >9950 (n=½) | ND |
| 83 | 0.0223 (0.00261, 2) | 89.6 ± 7.35 | 0.451 (0.0979, 2) | 103 ± 4.98 | >9950 (n=½) | ND |
| 84 | 0.0315 | 98.5 | 5.27 | 94 | >9950 | ND |
| 85 | 0.0312 | 91.7 | 7.69 | 95.3 | >9950 | ND |
| 86 | 0.0184 (0.00461, 2) | 94.2 ± 1.16 | 0.359 (0.0267, 2) | 103 ± 1.21 | >9950 (n=½) | ND |
| 87 | 0.029 | 84.7 | 1.34 | 106 | >9950 | ND |
| 88 | 0.00884 | 85.1 | 6.08 | 97.1 | >9950 | ND |
| 89 | 0.0301 (0.00853, 2) | 88.9 ± 3.76 | 1.03 (0.293, 2) | 105 ± 2.00 | >9950 (n=½) | ND |
| 90 | 0.0436 (0.0112, 3) | 93.0 ± 3.31 | 0.221 (0.0317, 3) | 96.2 ± 1.50 | >9950 (n=⅓) | ND |
| 91 | 0.0739 (0.0337, 3) | 92.8 ± 4.69 | 0.153 (0.0297, 3) | 94.0 ± 5.44 | >9950 (n=⅓) | ND |
| 92 | 0.0251 (0.0114, 2) | 104 ± 4.05 | 0.942 (0.0878, 2) | 105 ± 2.61 | >9950 (n=½) | ND |
| 93 | 0.0448 (0.0197, 2) | 99.4 ± 6.93 | 0.575 (0.0808, 2) | 106 ± 1.59 | >9950 (n=½) | ND |
| 94 | 0.0132 | 91 | 2.1 | 97.4 | >9950 | ND |
| 95 | 0.0748 | 97.3 | 0.181 | 86.9 | >9950 | ND |
| 96 | 0.0484 | 107 | 6.81 | 103 | >9950 | ND |
| 97 | 0.0276 (0.00391, 3) | 97.0 ± 1.53 | 0.683 (0.124, 3) | 96.6 ± 1.60 | >9950 (n=⅓) | ND |
| 98 | 0.0148 (0.00572, 3) | 97.7 ± 2.54 | 0.227 (0.0401, 3) | 98.6 ± 4.62 | >9950 (n=⅓) | ND |
| 99 | 0.0235 (0.00111, 2) | 93.4 ± 1.02 | 0.349 (0.114,3) | 99.1 ± 4.31 | >9950 (n=⅓) | ND |
| 100 | | | 11.4 | 111 | >9950 | ND |
| 101 | 0.0297 | 92.1 | 2.94 | 106 | >9950 | ND |
| 102 | | | 19.1 | 93.7 | >9950 | ND |
| 103 | 0.0305 | 92.8 | 2.10 (0.127, 2) | 106 ± 0.389 | >9950 (n=½) | ND |
| 104 | 0.0361 | 94.9 | 0.417 (0.00529, 2) | 107 ± 1.35 | >9950 (n=½) | ND |
| 105 | 0.0366 (0.00225, 3) | 103 ± 4.03 | 0.520 (0.0485, 3) | 105 ± 1.91 | >9950 (n=⅓) | ND |
| 106 | 0.0385 (0.00452, 3) | 98.7 ± 3.32 | 0.296 (0.0390, 3) | 103 ± 2.32 | >9950 (n=⅓) | ND |
| 107 | 0.0473 | 94.3 | 1.01 | 93.4 | >9950 | ND |
| 108 | 0.0249 | 85.2 | 0.701 | 97.6 | >9950 | ND |
| 109 | 0.0236 | 84.8 | 0.846 | 102 | >9950 | ND |
| 110 | 0.0417 | 106 | 1.43 | 98.8 | >9950 | ND |
| 111 | 0.0202 | 76.1 | 2.28 | 121 | >9950 | ND |
| 112 | 0.0229 | 93.6 | 2.26 | 107 | >9950 | ND |
| 113 | 0.0331 (0.00385, 3) | 97.9 ± 3.21 | 0.291 (0.0242, 3) | 101 ± 0.880 | >9950 (n=⅓) | ND |
| 114 | 0.0613 | 93.8 | 0.176 | 91.7 | >9950 | ND |
| 115 | 0.103 | 105 | 0.16 | 91.6 | >9950 | ND |
| 116 | 0.137 | 115 | 0.197 | 99.3 | >9950 | ND |
| 117 | 0.0352 | 96.4 | 5.6 | 98.2 | >9950 | ND |
| 118 | 0.0547 | 94.7 | 0.437 | 103 | >9950 | ND |
| 119 | 0.0863 | 103 | 0.351 | 99.4 | >9950 | ND |
| 120 | 0.0866 | 105 | 1.43 | 102 | >9950 | ND |
| 121 | 0.0271 | 92.8 | 8.95 | 106 | >9950 | ND |
| 122 | 0.064 | 105 | 0.41 | 94.3 | >9950 | ND |
| 123 | 0.101 | 94.9 | 0.508 | 96.3 | >9950 | ND |
| 124 | 0.1 | 102 | 1.24 | 93.3 | >9950 | ND |
| 125 | 0.0547 | 94.4 | 2.78 | 103 | >9950 | ND |
| 126 | 0.0472 | 91.8 | 1.69 | 101 | >9950 | ND |
| 127 | 0.027 | 99.3 | 0.762 | 98.5 | >9950 | ND |
| 128 | 0.0232 (0.000862, 3) | 98.5 ± 2.47 | 0.293 (0.0278, 3) | 98.4 ± 4.78 | >9950 (n=⅓) | ND |
| 129 | 0.0248 | 100 | 1.28 | 97.9 | >9950 | ND |
| 130 | 0.0266 (0.00252, 3) | 94.7 ± 4.51 | 0.204 (0.0158, 3) | 100 ± 1.69 | >9950 (n=⅓) | ND |
| 131 | 0.0156 (0.000742, 5) | 96.4 ± 1.95 | 0.190 (0.0152, 6) | 101 ± 2.01 | >9950 (n=⅙) | ND |
| 132 | 0.0208 (0.00119, 4) | 98.3 ± 1.68 | 0.182 (0.0131, 4) | 102 ± 3.32 | >9950 (n=¼) | ND |
| 133 | 0.0180 (0.00101, 5) | 92.7 ± 2.84 | 0.258 (0.0246, 6) | 101 ± 0.993 | >9950 (n=⅕) | ND |
| 134 | 0.0238 (0.00167, 3) | 99.2 ± 3.29 | 0.577 (0.0669, 3) | 106 ± 6.27 | >9950 (n=⅓) | ND |
| 135 | 0.0184 (0.00169, 4) | 92.3 ± 2.64 | 0.271 (0.00688, 5) | 98.4 ± 1.80 | >9950 (n=¼) | ND |
| 136 | 0.00690 (0.000974, 3) | 97.4 ± 4.23 | 0.0918 (0.0102, 3) | 107 ± 4.14 | >9950 (n=⅓) | ND |
| 137 | 0.0116 (0.00151, 3) | 105 ± 3.73 | 0.134 (0.0126, 4) | 99.0 ± 4.03 | >9950 (n=¼) | ND |
| 138 | 0.0197 (0.00311, 3) | 104 ± 2.12 | 0.267 (0.0474, 3) | 101 ± 2.93 | >9950 (n=⅓) | ND |
| 139 | 0.0108 | 99.3 | 0.141 | 111 | >9950 | ND |
| 140 | 0.0121 (0.000610, 3) | 98.5 ± 1.71 | 0.131 (0.00335, 3) | 106 ± 5.31 | >9950 (n=⅓) | ND |
| 141 | 0.00723 (0.000819, 3) | 98.8 ± 1.09 | 0.0693 (0.00248, 4) | 102 ± 2.84 | >9950 (n=¼) | ND |
| 142 | 0.0317 (0.00486, 2) | 98.6 ± 5.71 | 0.322 (0.0246, 2) | 103 ± 6.70 | >9950 (n=½) | ND |
| 143 | 0.022 | 95 | 0.512 | 103 | >9950 | ND |
| 144 | 0.0193 | 90 | 3.47 | 97 | >9950 | ND |
| 145 | 0.0286 | 74.8 | 0.555 | 95.1 | >9950 | ND |
| 146 | 0.0198 | 76.5 | 0.688 | 108 | >9950 | ND |
| 147 | 0.0229 | 98.6 | 0.338 | 93.5 | >9950 | ND |
| 148 | 0.0209 | 98.4 | 1.77 | 94.5 | >9950 | ND |
| 149 | 0.0791 (0.00155, 2) | 104 ± 0.229 | 1.50 (0.0754, 2) | 98.3 ± 0.579 | >9950 (n=½) | ND |
| 150 | 0.0268 (0.00212, 3) | 104 ± 6.96 | 0.455 (0.0582, 4) | 99.8 ± 3.10 | >9950 (n=¼) | ND |
| 151 | 0.0197 (0.00252, 3) | 99.9 ± 4.41 | 0.546 (0.0892, 4) | 102 ± 4.57 | >9950 (n=¼) | ND |
| 152 | 0.0416 (0.000338, 2) | 97.9 ± 3.25 | 3.91 (0.457, 2) | 102 ± 7.89 | >9950 (n=½) | ND |
| 153 | 0.0277 (0.00197, 2) | 97.8 ± 2.74 | 1.10 (0.248, 3) | 91.8 ± 4.63 | >9950 (n=⅓) | ND |
| 154 | 0.0299 (0.00189, 2) | 96.6 ± 3.26 | 1.69 (0.354, 3) | 102 ± 4.07 | >9950 (n=½) | ND |
| 155 | 0.0309 (0.00849, 2) | 96.6 ± 7.55 | 0.912 (0.163, 3) | 99.0 ± 5.16 | >9950 (n=½) | ND |
| 156 | 0.0422 | 89.9 | 4.6 | 110 | >9950 | ND |
| 157 | 0.0286 (0.000896, 2) | 98.8 ± 1.33 | 0.940 (0.206, 3) | 98.5 ± 4.73 | >9950 (n=⅓) | ND |
| 158 | 0.0204 | 93.4 | 0.448 | 87.2 | >9950 | ND |
| 159 | 0.0198 | 95.3 | 0.558 | 89.4 | >9950 | ND |
| 160 | 0.0197 | 101 | 0.228 | 93.6 | >9950 | ND |
| 161 | 0.0207 (0.00167, 3) | 94.6 ± 1.21 | 0.203 (0.00545, 3) | 107 ± 5.29 | >9950 (n=⅓) | ND |
| 162 | 0.022 | 89.7 | 0.327 | 99 | >9950 | ND |
| 163 | 0.0427 | 98.2 | 0.199 | 86.3 | >9950 | ND |
| 164 | 0.0464 (0.00253, 2) | 95.8 ± 5.48 | 0.236 (0.00998, 2) | 93.8 ± 0.298 | >9950 (n=½) | ND |
| 165 | 0.0442 | 96.1 | 0.291 | 94.3 | >9950 | ND |
| 166 | 0.0774 | 97 | 0.192 | 90.3 | >9950 | ND |
| 167 | 0.0534 (0.0163, 2) | 99.5 ± 6.17 | 0.217 (0.00420, 2) | 91.4 ± 3.21 | >9950 (n=½) | ND |
| 168 | 0.0482 | 93 | 0.312 | 93.3 | >9950 | ND |
| 169 | 0.0381 | 96.1 | 1.23 | 100 | >9950 | ND |
| 170 | 0.0244 | 93.9 | 1.03 | 104 | >9950 | ND |
| 171 | 0.0389 | 95.3 | 1.7 | 99.7 | >9950 | ND |
| 172 | 0.0149 (0.000516, 2) | 89.9 ± 1.05 | 0.141 (0.0138, 3) | 99.8 ± 2.93 | >9950 (n=⅓) | ND |
| 173 | 0.0155 (0.00731, 2) | 87.8 ± 6.22 | 0.343 (0.0897, 3) | 89.9 ± 2.72 | >9950 (n=⅓) | ND |
| 174 | 0.0284 | 92.2 | 0.33 | 98.2 | >9950 | ND |
| 175 | 0.0337 | 100 | 0.227 | 91.1 | >9950 | ND |
| 176 | 0.0485 | 89.3 | 0.298 | 98.9 | >9950 | ND |
| 177 | 0.0433 | 113 | 0.178 | 101 | >9950 | ND |
| 178 | 0.11 | 105 | 0.22 | 102 | >9950 | ND |
| 179 | 0.15 | 91 | 0.461 | 99 | >9950 | ND |
| 180 | 0.224 | 86.8 | 0.26 | 99.5 | >9950 | ND |
| 181 | 0.154 | 94.6 | 0.182 | 101 | >9950 | ND |
| 182 | 0.368 | 89 | 0.185 | 96.9 | >9950 | ND |
| 183 | 0.0326 (0.000430, 2) | 93.5 ± 1.46 | 1.37 (0.273, 2) | 93.9 ± 4.99 | >9950 (n=½) | ND |
| 184 | 0.0354 (0.00396, 2) | 95.2 ± 1.13 | 1.82 (0.135, 2) | 96.8 ± 12.0 | >9950 (n=½) | ND |
| 185 | 0.0180 (0.00136, 2) | 94.5 ± 0.288 | 0.419 (0.0758, 2) | 95.1 ± 1.74 | >9950 (n=½) | ND |
| 186 | 0.0274 (0.00339, 2) | 92.7 ± 0.689 | 0.371 (0.0267, 2) | 102 ± 5.53 | >9950 (n=½) | ND |
| 187 | 0.0260 (0.00396, 3) | 101 ± 2.77 | 0.386 (0.0467, 3) | 102 ± 7.10 | >9950 (n=⅓) | ND |
| 188 | 0.0228 (0.00116, 2) | 94.9 ± 1.49 | 0.332 (0.0557, 2) | 96.6 ± 4.28 | >9950 (n=½) | ND |
| 189 | 0.0202 (0.00407, 2) | 96.6 ± 4.38 | 0.280 (0.0253, 2) | 100 ± 3.68 | >9950 (n=½) | ND |
| 190 | 0.0240 (0.00181, 2) | 94.5 ± 0.611 | 0.303 (0.0624, 3) | 98.9 ± 4.64 | >9950 (n=⅓) | ND |
| 191 | 0.0601 (0.00698, 2) | 90.0 ± 3.29 | 0.926 (0.0435, 3) | 98.6 ± 5.09 | >9950 (n=⅓) | ND |
| 192 | 0.0375 (0.00284, 4) | 96.5 ± 4.86 | 0.518 (0.0340, 5) | 100 ± 2.34 | >9950 (n=⅕) | ND |
| 193 | 0.0201 (0.0000166, 2) | 97.2 ± 4.63 | 0.382 (0.0862, 3) | 104 ± 3.84 | >9950 (n=⅓) | ND |
| 194 | 0.0233 (0.00272, 2) | 93.1 ± 1.05 | 0.317 (0.0737, 3) | 103 ± 2.96 | >9950 (n=½) | ND |
| 195 | 0.0219 (0.000744, 2) | 88.5 ± 4.32 | 0.343 (0.0430, 3) | 101 ± 5.39 | >9950 (n=⅓) | ND |
| 196 | 0.0232 (0.00120, 4) | 96.0 ± 2.10 | 0.356 (0.0387, 5) | 96.8 ± 2.59 | >9950 (n=⅕) | ND |
| 197 | 0.0338 (0.00123, 3) | 102 ± 1.85 | 0.336 (0.0398, 4) | 103 ± 3.66 | >9950 (n=¼) | ND |
| 198 | 0.0269 (0.000614, 3) | 94.0 ± 1.17 | 0.362 (0.0292, 4) | 102 ± 2.25 | >9950 (n=¼) | ND |
| 199 | 0.0587 (0.00741, 2) | 96.9 ± 1.48 | 3.20 (1.31, 2) | 96.7 ± 6.69 | >9950 (n=½) | ND |
| 200 | 0.0503 (0.00678, 2) | 96.3 ± 2.76 | 0.834 (0.287, 2) | 93.7 ± 2.72 | >9950 (n=½) | ND |
| 201 | 0.188 (0.0191, 2) | 92.2 ± 3.26 | 0.487 (0.143, 2) | 105 ± 4.51 | >9950 (n=½) | ND |
| 202 | 0.057 | 86 | 0.944 | 92.2 | >9950 | ND |
| 203 | 0.0721 | 106 | 0.535 | 119 | >9950 | ND |
| 204 | 0.0304 | 99.3 | 0.47 | 126 | >9950 | ND |
| 205 | 0.0264 | 101 | 0.287 | 99.6 | >9950 | ND |
| 206 | 0.0204 | 96.4 | 0.299 | 100 | >9950 | ND |
| 207 | 0.019 | 96.2 | 0.209 | 95.1 | >9950 | ND |
| 208 | 0.018 | 94.9 | 0.238 | 101 | >9950 | ND |
| 209 | 0.0133 | 92.7 | 0.235 | 100 | >9950 | ND |
| 210 | 0.02 | 92.1 | 0.57 | 102 | >9950 | ND |
| 211 | 0.0465 | 91.6 | 3.55 | 93.1 | >9950 | ND |
| 212 | 0.0385 | 100 | 0.265 | 94.7 | >9950 | ND |
| 213 | 0.0275 | 92 | 2.14 | 94.8 | >9950 | ND |
| 214 | 0.0351 | 93.7 | 0.618 | 93.2 | >9950 | ND |
| 215 | 0.0437 | 85 | 0.44 | 89.8 | >9950 | ND |
| 216 | 0.0287 | 88.5 | 0.384 | 93.6 | >9950 | ND |
| 217 | 0.0309 | 95.5 | 0.436 | 88 | >9950 | ND |
| 218 | 0.0515 | 90.2 | 0.714 | 92.6 | >9950 | ND |
| 219 | 0.0227 (0.00409, 3) | 96.7 (2.65, 3) | 0.376 (0.0133, 3) | 103 (6.04, 3) | >9950 (n=⅓) | ND |
| 220 | 0.0237 | 91.6 | 0.535 | 87.6 | >9950 | ND |
| 221 | 0.0279 | 81.8 | 1.83 | 83 | >9950 | ND |
| 222 | 0.0195 | 98.7 | 0.518 | 91.6 | >9950 | ND |
| 223 | 0.0252 | 103 | 1.43 | 94 | >9950 | ND |
| 224 | 0.0374 | 109 | 7.51 | 88.4 | >9950 | ND |
| 225 | 0.0466 | 105 | 9.62 | 92.1 | >9950 | ND |
| 226 | 0.0232 | 101 | 0.611 | 101 | >9950 | ND |
| 227 | 0.0224 | 103 | 1.22 | 96.1 | >9950 | ND |
| 228 | 0.0232 | 110 | 1.34 | 91.1 | >9950 | ND |
| 229 | 0.0253 | 103 | 2.25 | 94.3 | >9950 | ND |
| 230 | 0.0199 (0.00457, 2) | 105 (4.33, 2) | 0.726 (0.336, 2) | 90.3 (7.27, 2) | >9950 (n=½) | ND |
| 231 | 0.0184 (0.000100, 2) | 102 (3.90, 2) | 1.22 (0.624, 2) | 107 (0.639, 2) | >9950 (n=½) | ND |
| 232 | 0.0192 | 101 | 0.565 | 103 | >9950 | ND |
| 233 | 0.0269 (0.00700, 2) | 105 (2.26, 2) | 0.604 (0.378, 2) | 111 (1.51, 2) | >9950 (n=½) | ND |
| 234 | 0.0182 | 107 | 0.319 | 112 | >9950 | ND |
| 235 | 0.0168 | 99.3 | 0.295 | 104 | >9950 | ND |
| 236 | 0.0154 (0.000744, 2) | 104 (0.0421, 2) | 0.336 (0.103, 2) | 96.5 (2.64, 2) | >9950 (n=½) | ND |
| 237 | 0.0186 (0.00115, 2) | 102 (0.313, 2) | 0.633 (0.0615, 2) | 102 (2.05, 2) | >9950 (n=½) | ND |
| 238 | 0.0214 (0.00134, 2) | 104 (0.833, 2) | 1.03 (0.378, 2) | 107 (1.17, 2) | >9950 (n=½) | ND |
| 239 | 0.0328 (0.00506, 2) | 104 (5.23, 2) | 0.546 (0.0853, 2) | 101 (4.97, 2) | >9950 (n=½) | ND |
| 240 | 0.0237 (0.000808, 2) | 99.1 (1.18, 2) | 0.508 (0.151, 2) | 98.5 (11.5, 2) | >9950 (n=½) | ND |
| 241 | 0.0213 (0.0000794, 2) | 105 (0.357, 2) | 0.362 (0.0106, 2) | 91.0 (16.3, 2) | >9950 (n=½) | ND |
| 242 | 0.0197 (0.00163, 2) | 100 (8.54, 2) | 0.584 (0.134, 2) | 96.3 (3.81, 2) | >9950 (n=½) | ND |
| 243 | 0.0154 (0.00300, 3) | 101 (6.26, 3) | 0.860 (0.294, 3) | 93.2 (4.52, 3) | >9950 (n=⅓) | ND |
| 244 | 0.0218 (0.00545, 3) | 101 (3.95, 3) | 2.25 (1.04, 3) | 104 (3.83, 3) | >9950 (n=⅓) | ND |
| 245 | 0.0195 (0.00407, 3) | 96.7 (3.74, 3) | 1.88 (0.824, 3) | 88.9 (8.09, 3) | >9950 (n=⅓) | ND |
| 246 | 0.0223 (0.00101, 4) | 96.9 (2.89, 4) | 0.441 (0.0598, 4) | 102 (1.52, 4) | >9950 (n=¼) | ND |
| 247 | 0.0220 (0.000585, 2) | 103 (1.70, 2) | 1.00 (0.641, 2) | 108 (0.981, 2) | >9950 (n=½) | ND |
| 248 | 0.0176 (0.00189, 2) | 99.2 (1.51,2) | 0.537 (0.130, 2) | 89.1 (7.08, 2) | >9950 (n=½) | ND |
| 249 | 0.0201 | 108 | 0.300 (0.0349, 2) | 101 (6.23, 2) | >9950 | ND |
| 250 | 0.021 | 102 | 0.414 (0.126, 2) | 114 (4.93, 2) | >9950 | ND |
| 251 | 0.0305 | 107 | 0.338 (0.0109, 2) | 99.0 (7.31, 2) | >9950 | ND |
| 252 | 0.049 | 109 | 0.555 (0.102, 2) | 115 (4.34, 2) | >9950 | ND |
| 253 | 0.0295 (0.00329, 2) | 101 (3.43, 2) | 0.430 (0.0422, 2) | 113 (3.67, 2) | >9950 (n=½) | ND |
| 254 | 0.0273 (0.000844, 2) | 98.2 (5.88, 2) | 0.507 (0.101, 2) | 103 (4.62, 2) | >9950 (n=½) | ND |
| 255 | 0.0213 | 103 | 0.357 | 103 | >9950 | ND |
| 256 | 0.0175 | 97.7 | 0.249 | 95.4 | >9950 | ND |
| 257 | 0.148 | 90.3 | 2.03 | 82 | >9950 | ND |
| 258 | 0.0473 | 90.3 | 0.653 | 94.9 | >9950 | ND |
| 259 | 0.0198 | 111 | 0.362 (0.0362, 2) | 99.5 (5.41, 2) | >9950 | ND |
| 260 | 0.0239 | 113 | 0.547 (0.0453, 2) | 108 (2.42, 2) | >9950 | ND |
| 261 | 0.025 | 105 | 0.531 (0.00408, 2) | 97.9 (1.27, 2) | >9950 | ND |
| 262 | 0.0327 | 89.8 | 0.472 (0.00490, 2) | 105 (5.10, 2) | >9950 | ND |
| 263 | 0.0214 | 88.6 | 0.294 (0.000357, 2) | 98.5 (5.68, 2) | >9950 | ND |
| 264 | 0.0208 | 114 | 0.413 (0.0548, 2) | 113 (1.32, 2) | >9950 | ND |
| 265 | 0.0145 (0.000696, 2) | 100 (1.69, 2) | 0.262 (0.0581, 2) | 99.8 (5.27, 2) | >9950 (n=½) | ND |
| 266 | 0.0136 (0.0000240, 2) | 93.2 (8.75, 2) | 0.282 (0.0352, 2) | 90.5 (17.9, 2) | >9950 (n=½) | ND |
| 267 | 0.0162 (0.000222, 2) | 104 (6.11,2) | 0.271 (0.0367, 2) | 103 (0.674, 2) | >9950 (n=½) | ND |
| 268 | 0.0265 | 102 | 0.613 | 112 | >9950 | ND |
| 269 | 0.0335 (0.00245, 2) | 95.3 (4.18, 2) | 1.72 (1.17, 2) | 96.5 (7.69, 2) | >9950 (n=½) | ND |
| 270 | 0.0567 | 91.7 | 0.551 | 97 | >9950 | ND |
| 271 | 0.0186 | 98.6 | 0.494 | 101 | >9950 | ND |
| 272 | 0.0264 | 86 | 0.388 | 99.9 | >9950 | ND |
| 273 | 0.0166 | 100 | 2.08 | 103 | >9950 | ND |
| 274 | 0.0226 | 103 | 0.406 | 114 | >9950 | ND |
| 275 | 0.0152 | 104 | 0.737 | 110 | >9950 | ND |
| 276 | 0.0134 | 102 | 0.49 | 103 | >9950 | ND |
| 277 | 0.0176 (0.000579, 2) | 107 (2.27, 2) | 0.225 (0.0241, 2) | 97.1 (0.819, 2) | >9950 (n=½) | ND |
| 278 | 0.0173 (0.000584, 2) | 105 (0.0609, 2) | 0.290 (0.0314, 2) | 109 (3.42, 2) | >9950 (n=½) | ND |
| 279 | 0.0305 | 103 | 0.406 | 112 | >9950 | ND |
| 280 | 0.0229 | 105 | 0.346 | 106 | >9950 | ND |
| 281 | 0.0187 | 110 | 0.252 | 100 | >9950 | ND |
| 282 | 0.0193 | 100 | 0.216 | 97.1 | >9950 | ND |
| 283 | 0.073 | 107 | 1.14 | 108 | >9950 | ND |
| 284 | 0.0389 | 98.3 | 0.505 | 110 | >9950 | ND |
| 285 | 0.0115 | 101 | 1.21 | 102 | >9950 | ND |
| 286 | 0.0344 | 108 | 0.678 | 110 | >9950 | ND |
| 287 | 0.0189 | 93 | 0.563 | 97.2 | >9950 | ND |
| 288 | 0.0219 | 110 | 0.354 | 103 | >9950 | ND |
| 289 | 0.017 | 111 | 0.731 | 96.7 | >9950 | ND |
| 290 | 0.0188 | 103 | 0.324 | 101 | >9950 | ND |
| 291 | 0.012 | 99.6 | 0.207 | 97.6 | >9950 | ND |
| 292 | 0.00620 (0.000221, 5) | 101 (2.00, 5) | 0.0758 (0.00345, 5) | 104 (1.25, 5) | >9950 (n=⅕) | ND |
| 293 | 0.00915 (0.00192, 3) | 102 (0.983, 3) | 0.102 (0.00688, 3) | 102 (1.78, 3) | >9950 (se=20.5, n=⅔) | ND |
| 294 | 0.00615 | 105 | 0.0665 | 106 | >9950 | ND |
| 295 | 0.0141 | 104 | 0.167 | 109 | >9950 | ND |
| 296 | 0.0121 | 100 | 0.108 | 109 | >9950 | ND |
| 297 | 0.0112 | 103 | 0.125 | 108 | >9950 | ND |
| 298 | 0.0107 (0.00103, 3) | 97.4 (2.75, 3) | 0.190 (0.0493, 3) | 106 (2.35, 3) | >9950 (n=⅓) | ND |
| 299 | 0.0104 (0.00202, 3) | 101 (4.84, 3) | 0.126 (0.0217, 3) | 103 (3.15, 3) | >9950 (n=⅓) | ND |
| 300 | 0.0128 (0.000753, 3) | 102 (1.14, 3) | 0.194 (0.00146, 3) | 107 (2.29, 3) | >9950 (n=⅓) | ND |
| 301 | 0.0128 (0.000550, 2) | 97.4 (2.71, 2) | 0.175 (0.0218, 2) | 99.2 (3.94, 2) | >9950 (n=½) | ND |
| 302 | 0.00916 (0.00192, 2) | 100 (2.39, 2) | 0.0917 (0.00850, 2) | 103 (0.561, 2) | >9950 (n=½) | ND |
| 303 | 0.0178 | 100 | 0.389 | 102 | >9950 | ND |
| 304 | 0.047 | 110 | 0.474 | 101 | >9950 | ND |
| 305 | 0.0289 | 105 | 0.629 | 92.7 | >9950 | ND |
| 306 | 0.0227 | 102 | 0.414 | 105 | >9950 | ND |
| 307 | 0.0292 | 105 | 0.376 | 101 | >9950 | ND |
| 308 | 0.0551 | 117 | 0.689 | 98 | >9950 | ND |
| 309 | 0.0212 | 107 | 0.527 | 104 | >9950 | ND |
| 310 | 0.0250 (0.00750, 2) | 99.9 (0.909, 2) | 0.347 (0.00203, 2) | 105 (1.16, 2) | >9950 (n=½) | ND |
| 311 | 0.0243 | 105 | 0.791 | 110 | >9950 | ND |
| 312 | 0.00757 | 105 | 0.663 | 104 | >9950 | ND |
| 313 | 0.00781 | 101 | 1.13 | 98.6 | >9950 | ND |
| 314 | 0.0216 | 100 | 0.673 | 104 | >9950 | ND |
| 315 | 0.0245 (0.000567, 2) | 108 (2.95, 2) | 0.376 (0.0331, 2) | 98.9 (6.60, 2) | >9950 (n=½) | ND |
| 316 | 0.0316 (0.00498, 2) | 103 (2.75, 2) | 0.571 (0.0402, 2) | 109 (0.715, 2) | >9950 (n=½) | ND |
| 317 | 0.0587 | 112 | 1.34 | 101 | >9950 (n=½) | ND |
| 318 | 0.0402 (0.0122, 3) | 106 (5.44, 3) | 0.219 (0.00541, 3) | 99.9 (2.22, 3) | >9950 (n=⅓) | ND |
| 319 | 0.0196 (0.00610, 3) | 98.1 (2.58, 3) | 0.180 (0.00678, 2) | 100 (4.36, 2) | >9950 (n=⅓) | ND |
| 320 | 0.0144 (0.00517, 2) | 94.2 (0.879, 2) | 0.246 (0.0335, 2) | 101 (5.01, 2) | >9950 (n=½) | ND |
| 321 | 0.0107 (0.00388, 2) | 108 (0.116, 2) | 0.295 (0.0332, 2) | 102 (1.81, 2) | >9950 (n=½) | ND |
| 322 | 0.00937 (0.00109, 6) | 101 (1.47, 6) | 0.144 (0.0108, 6) | 100 (1.87, 6) | >9950 (n=⅙) | ND |
| 323 | 0.00793 | 101 | 0.203 | 92.6 | >9950 | ND |
| 324 | 0.00632 (0.000630, 6) | 101 (2.01, 6) | 0.0647 (0.00427, 6) | 97.6 (3.15, 6) | >9950 (n=⅙) | ND |
| 325 | 0.0485 (0.0145, 3) | 104 (1.22, 3) | 0.225 (0.0687, 3) | 97.8 (1.80, 3) | >9950 (n=⅓) | ND |
| 326 | 0.0338 (0.0137, 3) | 102 (3.35, 3) | 0.186 (0.00851, 3) | 94.4 (4.49, 3) | >9950 (n=⅓) | ND |
| 327 | 0.0180 (0.00855, 2) | 105 (2.94, 2) | 0.211 (0.0578, 2) | 105 (1.05, 2) | >9950 (n=½) | ND |
| 328 | 0.00973 | 106 | 0.405 | 99.6 | >9950 | ND |
| 329 | 0.015 | 109 | 0.353 | 104 | >9950 | ND |
| 330 | 0.0185 | 112 | 0.477 | 103 | >9950 | ND |
| 331 | 0.0113 | 101 | 0.436 | 99.6 | >9950 | ND |
| 332 | 0.0109 | 107 | 0.263 | 114 | >9950 | ND |
| 333 | 0.0062 | 109 | 0.388 | 107 | >9950 | ND |
| 334 | 0.00987 (0.00114, 6) | 103 (2.09, 6) | 0.131 (0.0111, 6) | 104 (2.98, 6) | >9950 (n=⅙) | ND |
| 335 | 0.00871 | 107 | 0.175 | 113 | >9950 | ND |
| 336 | 0.0136 (0.00163, 4) | 109 (3.29, 4) | 0.338 (0.0360, 4) | 107 (4.12, 4) | >10100 (n=¼) | ND |
| 337 | 0.0151 (0.00142, 4) | 103 (4.79, 4) | 0.444 (0.0734, 4) | 107 (3.72, 4) | >10100 (n=¼) | ND |
| 338 | 0.0157 (0.000927, 4) | 104 (2.93, 4) | 0.468 (0.0280, 4) | 109 (4.09, 4) | >10100 (n=¼) | ND |
| 339 | 0.0190 (0.00238, 4) | 106 (3.70, 4) | 0.308 (0.0377, 4) | 113 (6.21, 4) | >10100 (n=¼) | ND |
| 340 | 0.0206 | 95.2 | 0.409 | 105 | >9950 | ND |
| 341 | 0.0127 (0.00156, 2) | 91.4 (2.36, 2) | 0.329 (0.00763, 2) | 99.9 (1.08, 2) | >9950 (n=½) | ND |
| 342 | 0.0105 (0.000707, 4) | 106 (4.47, 4) | 0.191 (0.0245, 4) | 113 (5.23, 4) | >10100 (n=¼) | ND |
| 343 | 0.0111 | 98.8 | 0.275 | 104 | >9950 | ND |
| 344 | 0.0174 | 97.9 | 0.336 | 100 | >9950 | ND |
| 345 | 0.0147 (0.000586, 2) | 98.9 (1.25, 2) | 0.285 (0.0205, 2) | 105 (3.05, 2) | >9950 (n=½) | ND |
| 346 | 0.0194 | 96.6 | 0.329 | 107 | >9950 | ND |
| 347 | 0.0259 | 98 | 0.356 | 103 | >9950 | ND |
| 348 | 0.0228 | 95.9 | 0.805 | 99.9 | >9950 | ND |
| 349 | 0.015 | 95.9 | 0.334 | 99 | >9950 | ND |
| 350 | 0.0288 | 101 | 0.202 | 95.9 | >9950 | ND |
| 351 | 0.0185 | 102 | 0.397 | 100 | >9950 | ND |
| 352 | 0.0106 | 98.3 | 0.262 | 99.5 | >9950 | ND |
| 353 | 0.0249 | 98.2 | 0.203 | 98 | >9950 | ND |
| 354 | 0.0249 | 95.4 | 0.505 | 99.9 | >9950 | ND |
| 355 | 0.0365 | 101 | 0.574 | 105 | >9950 | ND |
| 356 | 0.0488 | 96.2 | 0.936 | 101 | >9950 | ND |
| 357 | 0.0110 (0.00125, 4) | 99.9 (5.05, 4) | 0.649 (0.130, 4) | 107 (2.67, 4) | >10100 (n=¼) | ND |
| 358 | 0.0137 (0.000651, 4) | 99.6 (4.03, 4) | 0.773 (0.119, 4) | 107 (3.82, 4) | >10100 (n=¼) | ND |
| 359 | 0.0261 (0.00304, 4) | 103 (4.28, 4) | 1.18 (0.164, 4) | 104 (5.15, 4) | >10100 (n=¼) | ND |
| 360 | 0.0121 (0.00106, 2) | 106 (1.70, 2) | 0.301 (0.0234, 2) | 89.8 (0.316, 2) | >9950 (n=½) | ND |
| 361 | 0.0121 (0.00279, 2) | 107 (0.508, 2) | 0.580 (0.0801, 2) | 98.2 (2.23, 2) | >9950 (n=½) | ND |
| 362 | 0.0131 (0.000845, 4) | 107 (1.67, 4) | 0.340 (0.0419, 4) | 109 (2.00, 4) | >10100 (n=¼) | ND |
| 363 | 0.0183 | 111 | 0.34 | 108 | >9950 | ND |
| 364 | 0.0111 | 101 | 0.347 | 107 | >9950 | ND |
| 365 | 0.00809 | 105 | 0.362 | 98.5 | >9950 | ND |
| 366 | 0.0162 (0.00214, 2) | 108 (3.61, 2) | 0.279 (0.0415, 2) | 89.6 (8.49, 2) | >9950 (n=½) | ND |
| 367 | 0.019 | 112 | 0.366 | 93.9 | >9950 | ND |
| 368 | 0.0271 | 105 | 0.411 | 103 | >9950 | ND |
| 369 | 0.037 | 102 | 0.555 | 99.1 | >9950 | ND |
| 370 | 0.0282 | 105 | 0.402 | 112 | >9950 | ND |
| 371 | 0.0209 | 102 | 0.363 | 112 | >9950 | ND |
| 372 | 0.0112 | 97.3 | 0.291 | 108 | >9950 | ND |
| 373 | 0.0152 | 101 | 0.329 | 101 | >9950 | ND |
| 374 | 0.0135 | 96.2 | 0.269 | 106 | >9950 | ND |
| 375 | 0.0566 | 100 | 1.11 | 106 | >9950 | ND |
| 376 | 0.0281 | 99.8 | 0.539 | 104 | >9950 | ND |
| 377 | 0.0205 | 92.8 | 0.356 | 100 | >9950 | ND |
| 378 | 0.0132 (0.00169, 2) | 93.5 (2.41, 2) | 0.166 (0.00187, 2) | 98.9 (2.96, 2) | >9950 (n=½) | ND |
| 379 | 0.0167 (0.00194, 2) | 97.5 (1.50, 2) | 0.171 (0.0104, 2) | 104 (5.78, 2) | >9950 (n=½) | ND |
| 380 | 0.0209 (0.00237, 2) | 100 (3.53, 2) | 0.176 (0.0372, 2) | 96.2 (1.96, 2) | >9950 (n=½) | ND |
| 381 | 0.0784 | 101 | 1.1 | 105 | >9950 | ND |
| 382 | 0.0302 | 97.6 | 0.619 | 101 | >9950 | ND |
| 383 | 0.0147 (0.00111, 2) | 100 (1.51,2) | 0.306 (0.0408, 2) | 105 (4.16, 2) | >9950 (n=½) | ND |
| 384 | 0.0124 (0.000559, 2) | 94.5 (3.49, 2) | 0.308 (0.0612, 2) | 98.2 (1.53, 2) | >9950 (n=½) | ND |
| 385 | 0.0154 (0.00229, 2) | 95.2 (1.73, 2) | 0.189 (0.0226, 2) | 100 (4.42, 2) | >9950 (n=½) | ND |
| 386 | 0.0174 (0.00148, 2) | 95.7 (4.77, 2) | 0.194 (0.00360, 2) | 98.9 (5.82, 2) | >9950 (n=½) | ND |
| 387 | 0.0137 (0.0000708, 2) | 102 (3.23, 2) | 0.320 (0.125, 2) | 101 (1.61, 2) | >9950 (n=½) | ND |
| 388 | 0.0103 | 98.3 | 0.292 | 95.4 | >9950 | ND |
| 389 | 0.0178 (0.000953, 2) | 94.6 (0.800, 2) | 0.190 (0.00883, 2) | 92.4 (0.872, 2) | >9950 (n=½) | ND |
| 390 | 0.0187 (0.00223, 3) | 103 (1.73, 3) | 0.209 (0.00349, 3) | 97.7 (0.126, 3) | >9950 (n=⅓) | ND |
| 391 | 0.0101 (0.000893, 4) | 101 (2.26, 4) | 0.167 (0.0161, 4) | 109 (0.750, 4) | >9950 (n=¼) | ND |
| 392 | 0.0107 | 94.4 | 0.521 | 97.9 | >9950 | ND |
| 393 | 0.0115 | 94.3 | 0.372 | 96.8 | >9950 | ND |
| 394 | 0.0179 (0.00421, 2) | 97.7 (4.16, 2) | 0.223 (0.0106, 2) | 101 (2.99, 2) | >9950 (n=½) | ND |
| 395 | 0.0127 | 93.1 | 0.377 | 90.8 | >9950 | ND |
| 396 | 0.0113 | 96.2 | 0.291 | 107 | >9950 | ND |
| 397 | 0.0293 (0.0179, 2) | 94.6 (2.83, 2) | 0.166 (0.0101, 2) | 95.8 (4.65, 2) | >9950 (n=½) | ND |
| 398 | 0.0166 (0.000570, 2) | 97.4 (0.108, 2) | 0.215 (0.0107, 2) | 101 (5.97, 2) | >9950 (n=½) | ND |
| 399 | 0.0186 | 98.4 | 0.259 | 91.9 | >9950 | ND |
| 400 | 0.0821 | 98.1 | 0.708 | 108 | >9950 | ND |
| 401 | 0.0112 (0.00138, 3) | 98.9 (1.97, 3) | 0.235 (0.00554, 3) | 104 (7.95, 3) | >9950 (n=⅓) | ND |
| 402 | 0.0132 (0.00118, 3) | 98.9 (2.45, 3) | 0.221 (0.0238, 3) | 107 (4.31, 3) | >9950 (n=⅓) | ND |
| 403 | 0.00939 (0.000448, 3) | 98.1 (1.21, 3) | 0.175 (0.00248, 2) | 108 (0.362, 2) | >9950 (n=⅓) | ND |
| 404 | 0.014 | 101 | 0.632 | 94.3 | >9950 | ND |
| 405 | 0.0209 | 100 | 1.26 | 93.3 | >9950 | ND |
| 406 | 0.00896 (0.000684, 2) | 101 (2.15, 2) | 0.351 (0.0303, 2) | 98.5 (5.32, 2) | >9950 (n=½) | ND |
| 407 | 0.0114 (0.00120, 3) | 99.2 (1.86, 3) | 0.156 (0.00731, 3) | 100 (2.08, 3) | >9950 (n=⅓) | ND |
| 408 | 0.00888 (0.000279, 3) | 99.7 (1.81, 3) | 0.227 (0.0140, 3) | 102 (2.46, 3) | >9950 (n=⅓) | ND |
| 409 | 0.00928 (0.000716, 3) | 99.2 (0.111, 3) | 0.188 (0.0149, 3) | 103 (0.304, 3) | >9950 (n=⅓) | ND |
| 410 | 0.0118 (0.000492, 2) | 99.1 (1.74, 2) | 0.251 (0.147, 2) | 94.0 (4.37, 2) | >9950 (n=½) | ND |
| 411 | 0.0102 (0.00112, 3) | 101 (0.677, 3) | 0.167 (0.00866, 3) | 110 (5.40, 3) | >9950 (n=⅓) | ND |
| 412 | 0.0108 (0.00119, 3) | 95.4 (0.413, 3) | 0.160 (0.00954, 3) | 108 (2.23, 3) | >9950 (n=⅓) | ND |
| 413 | 0.0146 (0.00126, 3) | 101 (1.34, 3) | 0.234 (0.0297, 3) | 96.9 (5.28, 3) | >9950 (n=⅓) | ND |
| 414 | 0.00961 (0.000457, 4) | 98.4 (1.10, 4) | 0.119 (0.00178, 4) | 103 (1.77, 4) | >9950 (n=¼) | ND |
| 415 | 0.00991 (0.000319, 4) | 101 (0.864, 4) | 0.122 (0.00605, 4) | 109 (0.830, 4) | >9950 (n=¼) | ND |
| 416 | 0.0214 (0.000620, 3) | 99.0 (2.74, 3) | 0.332 (0.0140, 3) | 104 (1.88, 3) | >9950 (n=⅓) | ND |
| 417 | 0.00651 (0.000543, 4) | 101 (3.45, 4) | 0.0675 (0.00545, 4) | 109 (2.32, 4) | >9950 (n=¼) | ND |
| 418 | 0.00599 (0.000221, 3) | 91.0 (4.24, 3) | 0.0603 (0.00801, 3) | 93.7 (3.72, 3) | >9950 (n=⅓) | ND |
| 419 | 0.00958 (0.00102, 3) | 101 (2.60, 3) | 0.132 (0.0105, 3) | 113 (4.86, 3) | >9950 (n=⅓) | ND |
| 420 | 0.00964 (0.000804, 2) | 101 (0.422, 2) | 0.123 (0.0177, 2) | 99.7 (2.76, 2) | >9950 (n=½) | ND |
| 421 | 0.00618 (0.000456, 5) | 100 (4.89, 5) | 0.0570 (0.00402, 5) | 101 (4.19, 5) | >9950 (n=⅕) | ND |
| 422 | 0.00655 (0.000179, 5) | 104 (3.27, 5) | 0.0729 (0.00508, 5) | 106 (4.97, 5) | >9950 (n=⅕) | ND |
| 423 | 0.00658 (0.000673, 5) | 104 (2.28, 5) | 0.0721 (0.00530, 5) | 105 (3.34, 5) | >9950 (n=⅕) | ND |
| 424 | 0.00502 (0.000436, 5) | 101 (3.60, 5) | 0.0427 (0.00649, 5) | 102 (4.22, 5) | >9950 (n=⅕) | ND |
| 425 | 0.00943 (0.000670, 2) | 97.6 (2.09, 2) | 0.0965 (0.000509, 2) | 108 (1.41, 2) | >9950 (n=½) | ND |
| 426 | 0.0191 | 93.3 | 0.206 | 97.2 | >9950 (n=½) | ND |
| 427 | 0.0132 (0.000467, 2) | 95.9 (0.131, 2) | 0.206 (0.000352, 2) | 109 (1.69, 2) | >9950 (n=½) | ND |
| 428 | 0.00797 (0.000657, 2) | 95.5 (1.18, 2) | 0.102 (0.00942, 2) | 106 (2.33, 2) | >9950 (n=½) | ND |
| 429 | 0.00799 (0.000619, 2) | 108 (1.47, 2) | 0.116 (0.0211, 2) | 116 (8.54, 2) | >9950 (n=½) | ND |
| 430 | 0.0175 (0.00305, 2) | 107 (3.65, 2) | 0.342 (0.0582, 2) | 93.1 (9.40, 2) | >9950 (n=½) | ND |
| 431 | 0.00970 (0.000165, 2) | 104 (2.51, 2) | 0.134 (0.0264, 2) | 99.2 (5.72, 2) | >9950 (n=½) | ND |
| 432 | 0.00602 (0.000709, 5) | 103 (4.98, 5) | 0.0549 (0.00308, 5) | 106 (5.75, 5) | >9950 (n=⅕) | ND |
| 433 | 0.00972 (0.000248, 3) | 94.0 (6.04, 3) | 0.128 (0.00910, 3) | 102 (3.73, 3) | >9950 (n=⅓) | ND |
| 434 | 0.00704 (0.000555, 5) | 98.3 (4.47, 5) | 0.0837 (0.00431, 5) | 104 (4.56, 5) | >9950 (n=⅕) | ND |
| 435 | 0.00517 (0.000107, 5) | 104 (5.02, 5) | 0.0527 (0.00123, 5) | 111 (2.21, 5) | >9950 (n=⅕) | ND |
| 436 | 0.00714 (0.000579, 3) | 102 (0.413, 3) | 0.102 (0.00751, 3) | 98.9 (3.69, 3) | >9950 (n=⅓) | ND |
| 437 | 0.0146 (0.000707, 2) | 94.8 (7.99, 2) | 0.249 (0.0290, 2) | 89.2 (2.76, 2) | >9950 (n=½) | ND |
| 438 | 0.0157 (0.00257, 2) | 98.9 (3.64, 2) | 0.471 (0.0582, 2) | 88.5 (0.533, 2) | >9950 (n=½) | ND |
| 439 | 0.00799 (0.000558, 3) | 96.0 (3.26, 3) | 0.110 (0.0145, 3) | 95.7 (2.01, 3) | >9950 (n=⅓) | ND |
| 440 | 0.0104 (0.000447, 6) | 102 (1.92, 6) | 0.180 (0.0111, 6) | 107 (2.68, 6) | >10100 (n=⅙) | ND |
| 441 | 0.0174 (0.00153, 4) | 97.2 (0.594, 4) | 0.134 (0.0135, 4) | 110(4.43, 4) | >9950 (n=¼) | ND |
| 442 | 0.0129 (0.000387, 3) | 101 (1.37, 3) | 0.285 (0.0824, 3) | 113 (7.08, 3) | >9950 (n=⅓) | ND |
| 443 | 0.0178 (0.00241, 2) | 102 (1.04, 2) | 0.294 (0.00644, 2) | 114 (3.48, 2) | >9950 (n=½) | ND |
| 444 | 0.0157 (0.00115, 2) | 93.6 (7.04, 2) | 0.289 (0.00144, 2) | 106 (8.34, 2) | >9950 (n=½) | ND |
| 445 | 0.00728 (0.000110, 2) | 102 (1.47, 2) | 0.0781 (0.00635, 2) | 103 (1.23, 2) | >9950 (n=½) | ND |
| 446 | 0.0164 (0.000887, 3) | 105 (4.26, 3) | 0.162 (0.00890, 3) | 103 (5.00, 3) | >9950 (n=⅓) | ND |
| 447 | 0.0170 (0.00248, 3) | 102 (7.41, 3) | 0.219 (0.0213, 3) | 105 (4.10, 3) | >9950 (n=⅓) | ND |
| 448 | 0.0164 (0.00151, 3) | 105 (3.52, 3) | 0.157 (0.0114, 3) | 104 (1.69, 3) | >9950 (n=⅓) | ND |
| 449 | 0.0102 (0.000548, 5) | 101 (1.21, 5) | 0.160 (0.0159, 5) | 112 (2.67, 5) | >9950 (n=⅕) | ND |
| 450 | 0.0138 (0.000576, 5) | 103 (1.18, 5) | 0.201 (0.0102, 5) | 120 (3.81, 5) | >9950 (n=⅕) | ND |
| 451 | 0.0113 (0.000589, 5) | 101 (2.33, 5) | 0.172 (0.00777, 5) | 118 (3.25, 5) | >9950 (n=⅕) | ND |
| 452 | 0.0192 (0.00110, 3) | 102 (1.68, 3) | 0.259 (0.0104, 3) | 117 (1.07, 3) | >9950 (n=⅓) | ND |
| 453 | 0.0139 (0.00141, 5) | 101 (1.94, 5) | 0.200 (0.00941, 5) | 116 (3.41, 5) | >9950 (n=⅕) | ND |
| 454 | 0.0202 (0.00176, 4) | 101 (1.53, 4) | 0.266 (0.00975, 4) | 114 (3.68, 4) | >9950 (n=¼) | ND |
| 455 | 0.0146 (0.00112, 5) | 104 (1.34, 5) | 0.219 (0.0123, 5) | 117 (2.80, 5) | >9950 (n=⅕) | ND |
| 456 | 0.0236 (0.00188, 3) | 101 (2.59, 3) | 0.341 (0.0277, 3) | 116 (0.822, 3) | >9950 (n=⅓) | ND |
| 457 | 0.0106 (0.00217, 4) | 106 (3.22, 4) | 0.185 (0.0580, 4) | 119 (4.53, 4) | >9950 (n=¼) | ND |
| 458 | 0.0121 (0.000899, 4) | 101 (3.63, 4) | 0.179 (0.0147, 4) | 111 (4.25, 4) | >9950 (n=¼) | ND |
| 459 | 0.0130 (0.00287, 4) | 106 (3.71, 4) | 0.180 (0.0192, 4) | 120 (4.73, 4) | >9950 (n=¼) | ND |
| 460 | 0.0108 (0.00140, 4) | 102 (2.05, 4) | 0.187 (0.0213, 4) | 111 (2.78, 4) | >9950 (n=¼) | ND |
| 461 | 0.0103 (0.000757, 4) | 100 (3.07, 4) | 0.173 (0.0146, 4) | 108 (0.909, 4) | >10100 (n=¼) | ND |
| 462 | 0.0128 (0.00203, 3) | 99.3 (3.59, 3) | 0.189 (0.00876, 3) | 113 (7.43, 3) | >9950 (n=⅓) | ND |
| 463 | 0.0124 (0.00111, 3) | 91.4 (3.32, 3) | 0.267 (0.0917, 3) | 111 (4.12, 3) | >9950 (n=⅓) | ND |
| 464 | 0.00955 (0.000825, 4) | 99.7 (1.97, 4) | 0.161 (0.00664, 4) | 114 (4.40, 4) | >10100 (n=¼) | ND |
| 465 | 0.0174 (0.00309, 3) | 94.9 (2.15, 3) | 0.413 (0.0329, 3) | 98.6 (1.80, 3) | >9950 (n=⅓) | ND |
| 466 | 0.00971 (0.000114, 2) | 98.8 (0.994, 2) | 0.156 (0.00268, 2) | 105 (1.96, 2) | >10100 (n=½) | ND |
| 467 | 0.0113 (0.00170, 2) | 102 (4.24, 2) | 0.131 (0.00641, 2) | 110 (3.61, 2) | >10100 (n=½) | ND |
| 468 | 0.00991 (0.000823, 2) | 97.1 (2.96, 2) | 0.168 (0.00905, 2) | 109 (3.35, 2) | >10100 (n=½) | ND |
| 469 | 0.0121 (0.00130, 2) | 102 (1.70, 2) | 0.161 (0.00141, 2) | 111 (1.47, 2) | >9950 (n=½) | ND |
| 470 | 0.0103 (0.00167, 2) | 104 (7.15, 2) | 0.127 (0.00754, 2) | 112 (1.75, 2) | >9950 (n=½) | ND |
| 471 | 0.0107 (0.000902, 2) | 101 (5.62, 2) | 0.143 (0.0220, 2) | 112 (0.242, 2) | >9950 (n=½) | ND |
| 472 | 0.0131 (0.00159, 2) | 108 (3.90, 2) | 0.154 (0.0130, 2) | 118 (0.770, 2) | >9950 (n=½) | ND |
| 473 | 0.0163 (0.00190, 2) | 106 (3.37, 2) | 0.192 (0.0132, 2) | 118 (4.95, 2) | >9950 (n=½) | ND |
| 474 | 0.0106 (0.00162, 2) | 107 (1.06, 2) | 0.123 (0.0104, 2) | 113 (3.38, 2) | >9950 (n=½) | ND |
| 475 | 0.0120 (0.000617, 2) | 106 (2.22, 2) | 0.143 (0.0302, 2) | 116 (1.92, 2) | >9950 (n=½) | ND |
| 476 | 0.00886 (0.00204, 2) | 103 (0.456, 2) | 0.117 (0.0135, 2) | 114 (1.14, 2) | >9950 (n=½) | ND |
| 477 | 0.00861 (0.00132, 2) | 102 (1.19, 2) | 0.143 (0.0278, 2) | 110 (1.47, 2) | >9950 (n=½) | ND |
| 478 | 0.0139 | 93.9 | 0.322 | 109 | >9950 | ND |
| 479 | 0.00718 | 107 | 0.115 | 101 | >9950 | ND |
| 480 | 0.00955 | 102 | 0.124 | 120 | >9950 | ND |
| 481 | 0.0181 | 97.9 | 0.279 | 111 | >9950 | ND |
| 482 | 0.0158 (0.00283, 2) | 103 (2.04, 2) | 0.169 (0.00139, 2) | 118 (0.635, 2) | >9950 (n=½) | ND |
| 483 | 0.0138 (0.0000352, 2) | 97.4 (1.31, 2) | 0.169 (0.0278, 2) | 118 (4.57, 2) | >9950 (n=½) | ND |
| 484 | 0.0136 (0.00267, 2) | 102 (2.16, 2) | 0.170 (0.00710, 2) | 118 (0.624, 2) | >9950 (n=½) | ND |
| 485 | 0.0113 (0.000946, 2) | 106 (6.69, 2) | 0.153 (0.00444, 2) | 115 (0.806, 2) | >10100 (n=½) | ND |
| 486 | 0.0143 (0.00116, 2) | 94.1 (4.47, 2) | 0.166 (0.00560, 2) | 108 (0.513, 2) | >9950 (n=½) | ND |
| 487 | 0.0161 (0.00374, 2) | 103 (4.77, 2) | 0.199 (0.0396, 2) | 116 (5.51, 2) | >9950 (n=½) | ND |
| 488 | 0.0115 (0.00121, 5) | 104 (2.35, 5) | 0.207 (0.0233, 5) | 113 (2.17, 5) | >10100 (n=⅕) | ND |
| 489 | 0.0131 (0.000283, 3) | 103 (2.46, 3) | 0.257 (0.0330, 3) | 112 (5.59, 3) | >10100 (n=⅓) | ND |
| 490 | 0.0122 (0.00221, 2) | 110 (0.557, 2) | 0.143 (0.0153, 2) | 115 (3.29, 2) | >9950 (n=½) | ND |
| 491 | 0.0148 (0.00265, 2) | 100 (2.79, 2) | 0.148 (0.00935, 2) | 119 (2.42, 2) | >9950 (n=½) | ND |
| 492 | 0.0125 (0.00109, 4) | 101 (4.03, 4) | 0.143 (0.0174, 4) | 117 (2.36, 4) | >9950 (n=¼) | ND |
| 493 | 0.0155 (0.000342, 2) | 105 (1.97, 2) | 0.184 (0.0219, 2) | 126 (3.27, 2) | >9950 (n=½) | ND |
| 494 | 0.0123 (0.000792, 4) | 101 (2.14, 4) | 0.150 (0.00983, 4) | 104 (3.60, 4) | >9950 (n=¼) | ND |

ND = Not Determined
^{a} Expression density is determined using homologous competition binding of [¹²⁵I]GLP-1(7-36)NH₂ at hGLP-1R (112 fmol/mg protein), [¹²⁵I]GCG at hGCGR (98 fmol/mg protein) and [¹²⁵I]GIP(1-42) at hGIPR (124 fmol/mg protein).
^{b} EC₅₀, nM = the Geometric Mean, followed by the Standard Error of the Mean and the number of observations in parenthesis.
^{c} Eₘₐₓ, % = the Arithmetic Mean ± the Standard Error of the Mean for the percent of maximal response to GLP-1(7-36)NH₂ at hGLP-1R, GCG at hGCGR or GIP(1-42)NH₂ athGIPR.
All values shown are to three (3) significant digits.

TABLE 3

Functional cAMP Potency (EC₅₀) and Efficacy (Eₘₐₓ) for Peptides Incubated at 37° C. (in the presence of 0.1% bovine casein and 1.0% human serum albumin)

| | hGIPR ^{a} | | hGLP-1R ^{a} | | hGCGR^{a} | |
|---|---|---|---|---|---|---|
| Example | EC₅₀, nM (SEM, n) ^{b} | Emax, % ± SEM^{c} | EC₅₀, nM (SEM, n) ^{b} | Emax, % ± SEM^{c} | EC₅₀, nM (SEM, n) ^{b} | Emax, % ± SEM ^{c} |
| hGIP(1-42)NH2 | 0.597 (0.0522, 132) | 98.0 ± 1.16 | | | | |
| hGLP-1(7-36)NH2 | | | 0.340 (0.0195, 133) | 105 ± 1.39 | | |
| hGCG | | | | | 2.37 (0.153, 135) | 102 (se=1.49, n=135) |
| 1 | 0.696 (0.0329, 3) | 87.5 ± 3.24 | 30.7 (3.51, 3) | 94.3 ± 4.39 | >9950 (n=⅓) | ND |
| 6 | 0.0823 (0.00389, 3) | 89.4 ± 1.74 | 4.19 (0.479, 3) | 99.2 ± 5.54 | >9950 (n=⅓) | ND |
| 2 | 0.659 (0.150, 2) | 87.8 ± 5.11 | 32.1 (0.134, 2) | 105 ± 3.87 | >9950 (n=½) | ND |
| 3 | 0.0971 | 80 | 1.05 | 107 | >9950 | ND |
| 4 | 0.0296 (0.00275, 4) | 88.3 ± 2.17 | 0.584 (0.0543, 4) | 98.2 ± 1.60 | >9950 (n=¼) | ND |
| 7 | 0.566 (0.0700, 5) | 94.1 ± 2.10 | 2.74 (0.284, 5) | 105 ± 2.62 | >9950 (n=⅕) | ND |
| 8 | 0.635 (0.120, 3) | 91.3 ± 3.11 | 8.00 (1.46, 3) | 106 ± 3.25 | >9950 (n=⅓) | ND |
| 5 | 0.372 (0.0352, 5) | 91.2 ± 3.65 | 4.78 (0.559, 5) | 103 ± 4.77 | >9950 (n=⅕) | ND |
| 9 | 0.211 (0.0411, 3) | 91.6 ± 4.63 | 8.82 (1.34, 3) | 105 ± 3.71 | >9950 (n=⅓) | ND |
| 10 | 0.121 (0.0130, 4) | 84.9 ± 1.84 | 4.38 (0.399, 4) | 99.8 ± 1.37 | >9950 (n=¼) | ND |
| 11 | 1.22 (0.0617, 4) | 90.0 ± 2.71 | 34.5 (1.60, 4) | 102 ± 0.940 | >9950 (n=¼) | ND |
| 12 | 0.745 (0.0971, 4) | 93.0 ± 2.25 | 20.3 (2.83, 4) | 103 ± 5.98 | >9950 (n=¼) | ND |
| 13 | 1.45 (0.180, 4) | 95.1 ± 3.55 | 6.79 (0.450, 4) | 95.9 ± 1.76 | >9950 (n=¼) | ND |
| 14 | 0.769 (0.0465, 4) | 89.5 ± 1.02 | 9.19 (0.283, 4) | 101 ± 2.00 | >9950 (n=¼) | ND |
| 15 | 0.573 (0.0563, 4) | 94.4 ± 1.01 | 24.8 (0.964, 4) | 93.2 ± 3.68 | >9950 (n=¼) | ND |
| 16 | 0.625 (0.0549, 4) | 91.5 ± 1.01 | 9.06 (0.814, 4) | 98.3 ± 0.735 | >9950 (n=¼) | ND |
| 17 | 0.605 (0.0691, 3) | 88.9 ± 2.36 | 48.9 (6.33, 3) | 99.3 ± 0.621 | >9950 (n=⅓) | ND |
| 18 | 1.12 (0.150, 3) | 86.9 ± 3.59 | 164 (42.5, 3) | 103 ± 5.83 | >9950 (n=⅓) | ND |
| 19 | 0.438 (0.0168, 3) | 89.7 ± 2.13 | 24.4 (3.41, 3) | 102 ± 2.33 | >9950 (n=⅓) | ND |
| 20 | 1.66 (0.433, 2) | 90.6 ± 0.878 | 62.4 (20.7, 2) | 101 ± 4.72 | >9950 (n=½) | ND |
| 21 | 0.302 (0.0443, 3) | 94.2 ± 3.12 | 0.806 (0.119, 3) | 107 ± 4.52 | >9950 (n=⅓) | ND |
| 22 | 0.533 (0.0909, 3) | 93.4 ± 0.775 | 1.50 (0.0680, 3) | 101 ± 1.14 | >9950 (n=⅓) | ND |
| 23 | 0.623 (0.0883, 3) | 95.3 ± 0.863 | 1.98 (0.220, 3) | 106 ± 1.77 | >9950 (n=⅓) | ND |
| 24 | 0.444 (0.0309, 3) | 92.4 ± 5.97 | 2.04 (0.143, 3) | 103 ± 5.84 | >9950 (n=⅓) | ND |
| 25 | 0.274 (0.0482, 3) | 94.3 ± 1.73 | 1.11 (0.0768, 3) | 103 ± 1.65 | >9950 (n=⅓) | ND |
| 26 | 1.20 (0.0936, 3) | 95.4 ± 2.54 | 3.93 (0.133, 3) | 105 ± 3.59 | >9950 (n=⅓) | ND |
| 27 | 6.78 (1.82, 2) | 82.8 ± 1.14 | 70.2 (2.19, 2) | 110 ± 3.03 | >9950 (n=½) | ND |
| 28 | 2.08 (0.820, 2) | 93.0 ± 7.02 | 60.4 (15.0, 2) | 101 ± 2.12 | >9950 (n=½) | ND |
| 29 | 0.472 (0.0549, 2) | 96.9 ± 5.32 | 0.876 (0.104, 2) | 107 ± 2.92 | >9950 (n=½) | ND |
| 30 | 0.337 (0.252, 2) | 91.8 ± 0.797 | 18.3 (3.71, 2) | 96.5 ± 0.219 | >9950 (n=½) | ND |
| 31 | 0.147 | 88.9 | 0.545 (0.128, 2) | 109 ± 7.91 | >9950 (n=½) | ND |
| 32 | 0.148 | 98.2 | 1.73 | 102 | >9950 | ND |
| 33 | 0.0941 (0.0207, 2) | 92.0 ± 1.02 | 4.35 (0.0139, 2) | 109 ± 0.0920 | >9950 (n=½) | ND |
| 34 | 0.213 (0.0165, 2) | 89.1 ± 2.00 | 19.8 (3.06, 2) | 107 ± 1.76 | >9950 (n=½) | ND |
| 35 | 0.350 (0.00250, 2) | 91.7 ± 1.28 | 16.4 (2.30, 2) | 109 ± 0.873 | >9950 (n=½) | ND |
| 36 | 0.408 (0.0282, 2) | 91.4 ± 0.667 | 10.9 (1.38, 2) | 101 ± 0.511 | >9950 (n=½) | ND |
| 37 | 0.306 (0.0256, 2) | 88.5 ± 0.188 | 17.0 (2.86, 2) | 99.9 ± 4.41 | >9950 (n=½) | ND |
| 38 | 0.539 (0.0934, 2) | 88.8 ± 0.590 | 12.5 (1.43, 2) | 115 ± 2.53 | >9950 (n=½) | ND |
| 39 | 0.342 (0.00832, 2) | 86.8 ± 3.27 | 7.98 (1.61, 2) | 111 ± 1.02 | >9950 (n=½) | ND |
| 40 | 0.301 (0.00465, 2) | 92.2 ± 6.05 | 6.69 (2.15, 2) | 103 ± 2.62 | >9950 (n=½) | ND |
| 41 | 0.405 (0.0540, 2) | 88.5 ± 0.638 | 27.2 (1.30, 2) | 104 ± 3.41 | >9950 (n=½) | ND |
| 42 | 1.000 (0.00288, 2) | 91.1 ± 0.670 | 26.7 (4.92, 2) | 105 ± 10.9 | >9950 (n=½) | ND |
| 43 | 0.375 | 100 | 35.6 | 98.7 | >9950 | ND |
| 44 | 1.68 | 87 | 125 | 94.5 | >9950 | ND |
| 45 | 13.7 | 72.5 | 387 | 109 | >9950 | ND |
| 46 | 12.8 | 73.3 | 261 | 117 | >9950 | ND |
| 47 | 0.777 | 85.6 | 1670 | 134 | >9950 | ND |
| 48 | 2.12 | 90.8 | 118 | 111 | >9950 | ND |
| 49 | 1.51 (0.110, 3) | 92.4 ± 4.07 | 20.1 (2.77, 3) | 104 ± 0.273 | >9950 (n=⅓) | ND |
| 50 | 1.81 (0.416, 3) | 91.4 ± 4.20 | 27.8 (3.38, 3) | 106 ± 0.713 | >9950 (n=⅓) | ND |
| 51 | 15.8 | 90.7 | 100 | 92.9 | >9950 | ND |
| 52 | 18.6 | 87.6 | 37.8 | 101 | >9950 | ND |
| 53 | 3.53 (1.25, 3) | 91.7 ± 1.16 | 13.2 (2.18, 3) | 89.3 ± 6.12 | >9950 (n=⅓) | ND |
| 54 | 0.865 | 85.6 | 20.3 | 103 | >9950 | ND |
| 55 | 6.62 (0.645, 3) | 85.7 ± 0.885 | 70.5 (4.49, 3) | 112 ± 0.840 | >9950 (n=⅓) | ND |
| 56 | 6.75 (0.962, 3) | 91.4 ± 1.04 | 123 (8.75, 3) | 123 ± 6.57 | >9950 (n=⅓) | ND |
| 57 | 4.55 (0.383, 2) | 92.1 ± 5.54 | 39.3 (0.445, 2) | 114 ± 7.39 | >9950 (n=½) | ND |
| 58 | 1.12 (0.134, 2) | 92.2 ± 7.40 | 21.6 (2.74, 2) | 106 ± 3.99 | >9950 (n=½) | ND |
| 59 | 1.52 (0.323, 2) | 90.3 ± 3.64 | 16.2 (2.88, 2) | 108 ± 9.04 | >9950 (n=½) | ND |
| 60 | 1.20 (0.0303, 2) | 89.4 ± 4.57 | 28.5 (3.31, 2) | 111 ± 3.97 | >9950 (n=½) | ND |
| 61 | 0.744 | 92.8 | 36.9 | 89.7 | >9950 | ND |
| 62 | 1.27 (0.343, 2) | 94.3 ± 0.785 | 34.0 (10.3, 2) | 85.8 ± 3.31 | >9950 (n=½) | ND |
| 63 | 0.771 | 101 | 47.6 | 98 | >9950 | ND |
| 64 | 0.986 (0.230, 2) | 90.5 ± 3.44 | 27.0 (4.48, 2) | 87.8 ± 0.301 | >9950 (n=½) | ND |
| 65 | 1.05 (0.102, 2) | 93.8 ± 6.73 | 25.5 (5.32, 2) | 104 ± 2.15 | >9950 (n=½) | ND |
| 66 | 0.787 (0.0298, 2) | 90.6 ± 3.37 | 27.0 (8.48, 2) | 93.0 ± 2.36 | >9950 (n=½) | ND |
| 67 | 0.0417 | 91.9 | 12 | 104 | >9950 | ND |
| 68 | 0.0831 | 92.4 | 5.79 | 102 | >9950 | ND |
| 69 | 2.32 | 86.8 | 18.9 | 102 | >9950 | ND |
| 70 | 1.73 (0.0380, 2) | 89.9 ± 0.987 | 27.0 (3.69, 2) | 106 ± 4.82 | >9950 (n=½) | ND |
| 71 | 3.07 | 99.1 | 43.7 | 100 | >9950 | ND |
| 72 | 3.55 | 95.9 | 40.1 | 98.8 | >9950 | ND |
| 73 | 2.16 | 80.2 | 56.2 | 109 | >9950 | ND |
| 74 | 2.19 | 86.7 | 59.5 | 110 | >9950 | ND |
| 75 | 2.18 (0.153, 2) | 87.8 ± 3.33 | 41.3 (8.02, 2) | 97.0 ± 8.30 | >9950 (n=½) | ND |
| 76 | 1.40 (0.683, 2) | 89.9 ± 2.11 | 28.9 (1.46, 2) | 100 ± 13.5 | >9950 (n=½) | ND |
| 78 | 0.242 (0.0493, 2) | 89.7 ± 2.65 | 4.29 (0.505, 2) | 99.2 ± 0.361 | >9950 (n=½) | ND |
| 79 | 2.31 | 95 | 15.1 | 109 | >9950 | ND |
| 81 | 0.531 | 97.4 | 3.47 | 95 | >9950 | ND |
| 87 | 4.84 | 89.2 | 80 | 101 | >9950 | ND |
| 88 | 0.591 | 80.3 | 209 | 102 | >9950 | ND |
| 89 | 1.49 | 94.2 | 27.7 | 108 | >9950 | ND |
| 90 | 3.81 | 104 | 23.4 | 96.6 | >9950 | ND |
| 91 | 7.05 | 91.9 | 20.4 | 89.8 | >9950 | ND |
| 92 | 2.64 | 102 | 76.8 | 108 | >9950 | ND |
| 93 | 5.58 | 92 | 66 | 90.2 | >9950 | ND |
| 94 | 2.81 | 97.1 | 145 | 113 | >9950 | ND |
| 95 | 4.68 | 86.9 | 8.84 | 84 | >9950 | ND |
| 97 | 5.49 | 93.1 | 227 | 103 | >9950 | ND |
| 98 | 4.97 | 95 | 58.1 | 97.5 | >9950 | ND |
| 99 | 2.49 | 102 | 20 | 81.1 | >9950 | ND |
| 100 | 2.61 | 98.3 | 402 | 123 | >9950 | ND |
| 102 | 2.36 | 96.9 | 367 | 106 | >9950 | ND |
| 103 | 0.515 | 94.2 | 35.5 | 96.9 | >9950 | ND |
| 104 | 0.52 | 95.9 | 12.3 | 95.8 | >9950 | ND |
| 131 | 0.0581 (0.00374, 2) | 90.4 ± 1.62 | 1.04 (0.0207, 2) | 112 ± 1.76 | >9950 (n=½) | ND |
| 133 | 0.0695 (0.0139, 2) | 89.1 ± 4.64 | 1.17 (0.0663, 2) | 105 ± 5.52 | >9950 (n=½) | ND |
| 135 | 0.0609 (0.0105, 2) | 91.2 ± 4.78 | 0.936 (0.160, 2) | 101 ± 1.98 | >9950 (n=½) | ND |
| 137 | 0.728 (0.0218, 2) | 90.3 ± 0.387 | 5.02 (0.348, 2) | 103 ± 3.88 | >9950 (n=½) | ND |
| 141 | 0.379 (0.0143, 2) | 96.0 ± 7.42 | 5.78 (1.57, 2) | 115 ± 2.04 | >9950 (n=½) | ND |
| 150 | 0.350 (0.0449, 2) | 81.3 ± 3.18 | 11.0 (2.84, 2) | 100 ± 3.80 | >9950 (n=½) | ND |
| 151 | 0.306 (0.0354, 2) | 87.5 ± 3.78 | 17.3 (6.93, 2) | 112 ± 8.42 | >9950 (n=½) | ND |
| 153 | 1.15 (0.0744, 2) | 94.1 ± 3.21 | 40.8 (6.36, 2) | 98.2 ± 5.40 | >9950 (n=½) | ND |
| 154 | 1.34 (0.0132, 2) | 93.0 ± 4.96 | 70.5 (3.34, 2) | 103 ± 3.93 | >9950 (n=½) | ND |
| 155 | 1.25 (0.0855, 2) | 96.5 ± 1.04 | 34.6 (4.78, 2) | 102 ± 2.27 | >9950 (n=½) | ND |
| 157 | 0.718 (0.0184, 2) | 89.6 ± 3.39 | 36.0 (2.81, 2) | 101 ± 4.27 | >9950 (n=½) | ND |
| 164 | 3.62 | 93 | 26.6 | 95.4 | >9950 | ND |
| 172 | 0.385 (0.0503, 2) | 80.1 ± 2.06 | 7.70 (0.104,2) | 112 ± 10.3 | >9950 (n=½) | ND |
| 173 | 1.24 (0.0219, 2) | 92.9 ± 6.91 | 29.7 (4.49, 2) | 99.5 ± 1.80 | >9950 (n=½) | ND |
| 183 | 1.22 | 75.5 | 66.6 | 97.3 | >9950 | ND |
| 184 | 2.46 | 81.2 | 119 | 87.8 | >9950 | ND |
| 185 | 0.335 | 86.2 | 11.3 | 103 | >9950 | ND |
| 186 | 2.19 | 85.6 | 24.3 | 107 | >9950 | ND |
| 187 | 0.939 | 79.3 | 18 | 109 | >9950 | ND |
| 188 | 1.37 | 89 | 19.1 | 106 | >9950 | ND |
| 190 | 1.55 (0.169, 2) | 82.8 ± 3.36 | 28.9 (0.482, 2) | 112 ± 8.50 | >9950 (n=½) | ND |
| 191 | 0.866 (0.0257, 2) | 86.2 ± 5.50 | 13.1 (2.25, 2) | 105 ± 7.98 | >9950 (n=½) | ND |
| 192 | 0.832 (0.0527, 2) | 93.6 ± 3.98 | 11.2 (3.64, 2) | 121 ± 2.56 | >9950 (n=½) | ND |
| 193 | 1.47 (0.124, 2) | 92.2 ± 0.240 | 24.3 (4.18, 2) | 104 ± 8.64 | >9950 (n=½) | ND |
| 194 | 1.24 (0.0572, 2) | 80.7 ± 7.81 | 21.2 (4.53, 2) | 109 ± 10.5 | >9950 (n=½) | ND |
| 195 | 0.771 (0.268, 2) | 87.0 ± 12.2 | 14.0 (3.17, 2) | 106 ± 2.61 | >9950 (n=½) | ND |
| 196 | 0.712 (0.191, 2) | 80.8 ± 0.269 | 21.4 (1.40, 2) | 112 ± 4.62 | >9950 (n=½) | ND |
| 197 | 2.06 (0.255, 2) | 84.5 ± 4.08 | 26.2 (3.64, 2) | 102 ± 2.64 | >9950 (n=½) | ND |
| 198 | 1.06 (0.264, 2) | 83.1 ± 3.45 | 14.5 (3.33, 2) | 108 ± 0.518 | >9950 (n=½) | ND |
| 199 | 2.45 | 77.1 | 70 | 100 | >9950 | ND |
| 200 | 2.7 | 83.7 | 26.6 | 97.4 | >9950 | ND |
| 201 | 13.2 | 80.5 | 25.1 | 104 | >9950 | ND |
| 202 | 6.09 | 75.3 | 62.2 | 109 | >9950 | ND |
| 203 | 0.682 | 87.2 | 3.38 | 105 | >9950 | ND |
| 204 | 0.341 | 83.3 | 2.44 | 104 | >9950 | ND |
| 205 | 0.261 (0.0684, 2) | 72.1 (10.2, 2) | 2.18 (0.439, 2) | 106 (1.66, 2) | >9950 (n=½) | ND |
| 206 | 0.242 (0.107, 2) | 77.6 (6.91, 2) | 3.03 (0.792, 2) | 98.1 (0.421, 2) | >9950 (n=½) | ND |
| 207 | 0.591 (0.0545, 2) | 97.9 (9.30, 2) | 2.98 (0.814, 2) | 112 (10.5, 2) | >9950 (n=½) | ND |
| 208 | 0.126 (0.0370, 2) | 64.1 (0.979, 2) | 1.58 (0.664, 2) | 105 (8.90, 2) | >9950 (n=½) | ND |
| 209 | 0.173 (0.0236, 2) | 90.8 (2.77, 2) | 1.16 (0.312, 2) | 109 (4.47, 2) | >9950 (n=½) | ND |
| 210 | 0.440 (0.0195, 2) | 68.1 (7.17, 2) | 8.86 (0.573, 2) | 110 (7.68, 2) | >9950 (n=½) | ND |
| 211 | 2.16 | 64.5 | 94.2 | 99 | >9950 | ND |
| 212 | 16.6 | 81 | 33.7 | 91 | >9950 | ND |
| 213 | 4.38 | 95.6 | 40.3 | 102 | >9950 | ND |
| 214 | 2.03 | 80.4 | 17.9 | 108 | >9950 | ND |
| 215 | 0.925 | 91.3 | 14.8 | 107 | >9950 | ND |
| 216 | 5.31 | 63.4 | 32.3 | 117 | >9950 | ND |
| 217 | 7.84 | 62.7 | 87.8 | 118 | >9950 | ND |
| 218 | 3.27 | 95.9 | 36.6 | 121 | >9950 | ND |
| 219 | 2.86 (1.19, 2) | 82.9 (17.7, 2) | 25.9 (3.30, 2) | 101 (2.32, 2) | >9950 (n=½) | ND |
| 220 | 11.6 | 83.5 | 150 | 110 | >9950 | ND |
| 221 | 0.838 | 67.4 | 17.9 | 92.3 | >9950 | ND |
| 222 | 2.1 | 77.9 | 13.8 | 117 | >9950 | ND |
| 223 | 7.84 | 79.2 | 90.5 | 115 | >9950 | ND |
| 224 | 2.92 | 90.6 | 71 | 102 | >9950 | ND |
| 225 | 1.81 | 67.5 | 51.6 | 106 | >9950 | ND |
| 226 | 4.86 | 92.1 | 11.3 | 118 | >9950 | ND |
| 227 | 1.59 | 76.1 | 41.8 | 111 | >9950 | ND |
| 228 | 3.37 | 83.3 | 42 | 119 | >9950 | ND |
| 229 | 7.35 | 80.5 | 135 | 99 | >9950 | ND |
| 230 | 2.57 (0.207, 3) | 85.8 (2.02, 3) | 49.7 (3.88, 3) | 103 (3.29, 3) | >9950 (n=⅓) | ND |
| 231 | 2.46 (0.528, 2) | 85.4 (1.66, 2) | 51.8 (5.87, 2) | 109 (2.34, 2) | >9950 (n=½) | ND |
| 232 | 1.86 | 91.5 | 33.6 | 95.4 | >9950 | ND |
| 233 | 6.08 (0.278, 2) | 91.4 (1.15, 2) | 56.7 (19.2, 2) | 116 (15.0, 2) | >9950 (n=½) | ND |
| 234 | 7.58 | 84.4 | 156 | 102 | >9950 | ND |
| 235 | 10.1 | 92.2 | 598 | 152 | >9950 | ND |
| 236 | 0.641 (0.0940, 2) | 80.5 (3.87, 2) | 32.6 (4.16, 2) | 125 (2.87, 2) | >9950 (n=½) | ND |
| 237 | 0.889 (0.480, 2) | 93.1 (3.84, 2) | 38.1 (0.788, 2) | 113 (2.42, 2) | >9950 (n=½) | ND |
| 238 | 1.31 (0.644, 2) | 90.5 (11.3, 2) | 27.2(1.11, 2) | 103 (2.10, 2) | >9950 (n=½) | ND |
| 239 | 5.46 (2.04, 2) | 89.1 (0.575, 2) | 25.2 (9.76, 2) | 109 (1.64, 2) | >9950 (n=½) | ND |
| 240 | 11.7 (0.555, 2) | 87.2 (11.8, 2) | 150 (5.84, 2) | 107 (1.18, 2) | >9950 (n=½) | ND |
| 241 | 8.02 (3.37, 2) | 84.9 (9.74, 2) | 103 (8.35, 2) | 105 (4.79, 2) | >9950 (n=½) | ND |
| 242 | 11.0 (5.42, 2) | 83.9 (8.92, 2) | 214 (35.7, 2) | 102 (0.239, 2) | >9950 (n=½) | ND |
| 243 | 0.728 (0.185, 3) | 90.5 (0.658, 3) | 34.7 (7.58, 3) | 114 (12.1, 3) | >9950 (n=⅓) | ND |
| 244 | 0.983 (0.411, 3) | 89.5 (3.16, 3) | 30.8 (6.22, 3) | 96.3 (3.54, 3) | >9950 (n=⅓) | ND |
| 245 | 0.552 (0.0557, 3) | 81.9 (4.90, 3) | 27.0 (1.02, 3) | 93.8 (2.93, 3) | >9950 (n=⅓) | ND |
| 246 | 1.63 (0.495, 3) | 91.0 (3.43, 3) | 22.9 (6.17, 3) | 108 (9.08, 3) | >9950 (n=⅓) | ND |
| 247 | 4.24 (0.948, 2) | 90.2 (4.32, 2) | 77.7 (0.558, 2) | 109 (1.12, 2) | >9950 (n=½) | ND |
| 248 | 4.10 (1.68, 2) | 91.8 (0.258, 2) | 56.7 (2.29, 2) | 92.7 (4.61, 2) | >9950 (n=½) | ND |
| 249 | 5.88 | 88.9 | 51.6 | 105 | >9950 | ND |
| 250 | 38.8 | 101 | 63 | 105 | >9950 | ND |
| 251 | 14.3 | 71.2 | 51 | 104 | >9950 | ND |
| 252 | 29.9 | 87.2 | 131 | 115 | >9950 | ND |
| 253 | 21.7 (1.79, 3) | 78.9 (6.58, 3) | 192 (28.9, 3) | 114 (7.97, 3) | >9950 (n=⅓) | ND |
| 254 | 16.8 (1.47, 3) | 68.5 (4.32, 3) | 211 (49.2, 3) | 122 (16.8, 3) | >9950 (n=⅓) | ND |
| 255 | 2.27 | 93.4 | 13.7 | 106 | >9950 | ND |
| 256 | 1.5 | 75.3 | 12.6 | 95.5 | >9950 | ND |
| 257 | 5.61 | 72.2 | 46.3 | 102 | >9950 | ND |
| 258 | 5.28 | 84.3 | 41.5 | 103 | >9950 | ND |
| 259 | 2.34 | 66.4 | 28.9 | 99.2 | >9950 | ND |
| 260 | 3.27 | 83 | 32 | 107 | >9950 | ND |
| 261 | 2.28 | 75.7 | 15.6 | 104 | >9950 | ND |
| 262 | 8.15 | 79.7 | 69.8 | 115 | >9950 | ND |
| 263 | 5.91 | 72.7 | 56.7 | 95.8 | >9950 | ND |
| 264 | 7.12 | 84.4 | 75.2 | 105 | >9950 | ND |
| 265 | 0.775 (0.0602, 2) | 96.0 (0.960, 2) | 5.14 (1.17, 2) | 102 (2.46, 2) | >9950 (n=½) | ND |
| 266 | 0.575 | 82.7 | 5.83 | 97.6 | >9950 | ND |
| 267 | 0.808 | 88.1 | 8.46 | 104 | >9950 | ND |
| 268 | 5.06 | 92.3 | 26.5 | 104 | >9950 | ND |
| 269 | 2.65 (3.59, 3) | 87.3 (0.702, 3) | 46.5 (16.2, 3) | 105 (4.56, 3) | >9950 (n=⅓) | ND |
| 270 | 3.8 | 80.9 | 32.6 | 111 | >9950 | ND |
| 271 | 0.767 | 97.3 | 10.2 | 122 | >9950 | ND |
| 272 | 0.561 | 81.2 | 4.72 | 102 | >9950 | ND |
| 273 | 0.318 | 88.4 | 40.9 | 90.5 | >9950 | ND |
| 274 | 1.16 | 93.5 | 32.9 | 121 | >9950 | ND |
| 275 | 0.926 | 101 | 33.5 | 103 | >9950 | ND |
| 276 | 1.97 | 91.4 | 63.6 | 104 | >9950 | ND |
| 277 | 0.962 | 99.3 | 12.2 | 93.1 | >9950 | ND |
| 278 | 1.41 | 91.1 | 27 | 121 | >9950 | ND |
| 279 | 3.21 | 88.6 | 36.6 | 114 | >9950 | ND |
| 280 | 4.84 | 88.5 | 49.9 | 105 | >9950 | ND |
| 281 | 1.87 | 97.9 | 22.4 | 91.7 | >9950 | ND |
| 282 | 1.44 | 84.4 | 31.8 | 102 | >9950 | ND |
| 283 | 4.88 | 114 | 49.5 | 96.6 | >9950 | ND |
| 284 | 8.38 | 101 | 137 | 112 | >9950 | ND |
| 285 | 1.28 | 84.7 | 136 | 105 | >9950 | ND |
| 286 | 2.18 | 104 | 35 | 90.3 | >9950 | ND |
| 287 | 3.76 | 98.9 | 83.8 | 106 | >9950 | ND |
| 288 | 0.48 | 85.2 | 17.1 | 123 | >9950 | ND |
| 289 | 3.11 | 94.9 | 627 | 170 | >9950 | ND |
| 290 | 0.878 | 93.4 | 12.1 | 109 | >9950 | ND |
| 291 | 0.833 | 92.9 | 7.43 | 100 | >9950 | ND |
| 292 | 0.416 (0.0297, 5) | 88.4 (4.96, 5) | 4.35 (0.151, 5) | 101 (2.62, 5) | >9950 (n=⅕) | ND |
| 293 | 0.987 (0.168, 3) | 96.6 (2.52, 3) | 10.7 (0.553, 3) | 99.4 (3.13, 3) | >9950 (n=⅓) | ND |
| 294 | 0.833 | 107 | 6.26 | 104 | >9950 | ND |
| 295 | 1.09 | 99.6 | 10.4 | 101 | >9950 | ND |
| 296 | 0.823 | 91.9 | 13 | 109 | >9950 | ND |
| 297 | 1.54 | 93.7 | 19.1 | 108 | >9950 | ND |
| 298 | 0.237 (0.0133, 3) | 95.0 (3.55, 3) | 3.64 (1.18, 3) | 107 (10.3, 3) | >9950 (n=⅓) | ND |
| 299 | 0.204 (0.0143, 3) | 99.8 (4.40, 3) | 2.58 (0.211, 3) | 102 (1.94, 3) | >9950 (n=⅓) | ND |
| 300 | 1.25 (0.0915, 3) | 91.5 (6.45, 3) | 19.6 (2.89, 3) | 109 (2.14, 3) | >9950 (n=⅓) | ND |
| 301 | 0.919 (0.0935, 2) | 85.5 (3.43, 2) | 12.9 (0.666, 2) | 106 (7.59, 2) | >9950 (n=½) | ND |
| 302 | 0.453 (0.00185, 2) | 86.3 (0.816, 2) | 6.55 (0.397, 2) | 99.4 (1.04, 2) | >9950 (n=½) | ND |
| 303 | 2 | 89.8 | 50.4 | 111 | >9950 | ND |
| 304 | 4.83 | 99.3 | 64.2 | 98.3 | >9950 | ND |
| 305 | 4.34 | 95.4 | 80.3 | 125 | >9950 | ND |
| 306 | 1.12 | 102 | 18.2 | 102 | >9950 | ND |
| 307 | 1.66 | 81.8 | 33 | 109 | >9950 | ND |
| 308 | 10.5 | 98.4 | 67.3 | 92.2 | >9950 | ND |
| 309 | 6.08 | 95.3 | 64.6 | 106 | >9950 | ND |
| 310 | 2.4 | 83.9 | 22 | 106 | >9950 | ND |
| 311 | 2.16 | 98.3 | 33.8 | 104 | >9950 | ND |
| 312 | 4.48 | 89.5 | 50.8 | 112 | >9950 | ND |
| 313 | 21.9 | 101 | 98.8 | 95 | >9950 | ND |
| 314 | 3.51 | 101 | 38.6 | 102 | >9950 | ND |
| 315 | 0.393 | 89.2 | 5.38 | 93.5 | >9950 | ND |
| 316 | 1.11 | 102 | 19.2 | 113 | >9950 | ND |
| 317 | 2.03 (0.0584, 2) | 84.4 (8.11, 2) | 29.5 (4.64, 2) | 98.1 (5.04, 2) | >9950 (n=½) | ND |
| 318 | 5.42 (0.737, 2) | 91.4 (2.19, 2) | 28.7 (1.02, 2) | 101 (1.14, 2) | >9950 (n=½) | ND |
| 319 | 3.35 (1.04, 2) | 92.5 (5.28, 2) | 28.1 (3.97, 2) | 114 (17.1, 2) | >9950 (n=½) | ND |
| 320 | 0.338 | 84.1 | 6.22 | 99.2 | >9950 | ND |
| 321 | 0.631 | 94.5 | 13.9 | 96.6 | >9950 | ND |
| 322 | 0.617 (0.107, 5) | 89.7 (3.98, 5) | 8.09 (0.489, 5) | 94.5 (2.57, 5) | >9950 (n=⅕) | ND |
| 323 | 0.694 | 92.9 | 9.52 | 104 | >9950 | ND |
| 324 | 0.321 (0.0210, 5) | 85.4 (4.62, 5) | 4.38 (0.281, 5) | 99.4 (4.29, 5) | >9950 (n=⅕) | ND |
| 325 | 3.96 (0.456, 2) | 94.7 (5.70, 2) | 23.5 (6.01, 2) | 98.4 (2.48, 2) | >9950 (n=½) | ND |
| 326 | 4.62 (0.384, 2) | 88.7 (7.81, 2) | 26.1 (0.140, 2) | 96.1 (2.59, 2) | >9950 (n=½) | ND |
| 327 | 0.751 | 90.2 | 13.6 | 97.6 | >9950 | ND |
| 328 | 0.75 | 85.5 | 23.3 | 102 | >9950 | ND |
| 329 | 5.44 | 100 | 74.7 | 88.3 | >9950 | ND |
| 330 | 6.26 | 103 | 85.2 | 85 | >9950 | ND |
| 331 | 8.31 | 108 | 223 | 114 | >9950 | ND |
| 332 | 4.59 | 96.5 | 33.3 | 116 | >9950 | ND |
| 333 | 6.74 | 100 | 70.4 | 93.3 | >9950 | ND |
| 334 | 0.618 (0.0980, 5) | 89.0 (3.96, 5) | 8.67 (0.430, 5) | 105 (3.28, 5) | >9950 (n=⅕) | ND |
| 335 | 1.04 | 85.2 | 12.3 | 101 | >9950 | ND |
| 336 | 2.92 (0.0217, 2) | 94.6 (4.74, 2) | 47.0 (6.26, 2) | 103 (8.40, 2) | >10100 (n=½) | ND |
| 337 | 2.65 (0.235, 2) | 86.8 (4.14, 2) | 53.6 (8.71, 2) | 100 (1.63, 2) | >10100 (n=½) | ND |
| 338 | 1.24 (0.0430, 2) | 83.9 (2.56, 2) | 36.6 (6.57, 2) | 103 (1.43, 2) | >10100 (n=½) | ND |
| 339 | 4.35 (0.915, 2) | 87.3 (9.37, 2) | 68.2 (10.6, 2) | 115 (6.65, 2) | >10100 (n=½) | ND |
| 340 | 9.91 | 87.9 | 122 | 112 | >9950 | ND |
| 341 | 5.74 (0.563, 2) | 83.6 (3.95, 2) | 95.8 (1.69, 2) | 113 (8.64, 2) | >9950 (n=½) | ND |
| 342 | 1.04 (0.0681, 2) | 89.3 (1.18, 2) | 13.6 (0.635, 2) | 101 (2.94, 2) | >9950 (n=½) | ND |
| 343 | 2.38 | 90.2 | 20.2 | 98.2 | >9950 | ND |
| 344 | 6.78 | 87.3 | 48.3 | 96.5 | >9950 | ND |
| 345 | 0.731 (0.0103, 2) | 86.8 (8.74, 2) | 11.8 (1.13, 2) | 110 (2.77, 2) | >9950 (n=½) | ND |
| 346 | 1.83 | 83.9 | 19 | 97.7 | >9950 | ND |
| 347 | 7.27 | 84.6 | 51.7 | 99.5 | >9950 | ND |
| 348 | 1.79 | 79.3 | 8.95 | 98 | >9950 | ND |
| 349 | 0.606 | 79.3 | 5.15 | 94.3 | >9950 | ND |
| 350 | 5.82 | 73.5 | 6.4 | 91.4 | >9950 | ND |
| 351 | 0.722 | 67.9 | 5.8 | 91 | >9950 | ND |
| 352 | 0.277 | 71.3 | 5.46 | 93.5 | >9950 | ND |
| 353 | 5.35 | 84.8 | 7.12 | 92.5 | >9950 | ND |
| 354 | 0.857 | 67.5 | 7.25 | 95.9 | >9950 | ND |
| 355 | 1.81 | 86 | 11.3 | 109 | >9950 | ND |
| 356 | 2.47 | 82.7 | 23.4 | 93 | >9950 | ND |
| 357 | 1.19 (0.250, 2) | 78.2 (14.7, 2) | 36.2 (2.98, 2) | 92.8 (1.82, 2) | >9950 (n=½) | ND |
| 358 | 2.06 (0.735, 2) | 86.2 (0.397, 2) | 65.3 (2.90, 2) | 102 (4.28, 2) | >9950 (n=½) | ND |
| 359 | 1.58 (0.643, 2) | 81.0 (10.1, 2) | 40.5 (4.21, 2) | 95.1 (1.93, 2) | >9950 (n=½) | ND |
| 360 | 1.91 (0.0821, 2) | 84.3 (10.5, 2) | 28.2 (0.0380, 2) | 97.1 (5.05, 2) | >9950 (n=½) | ND |
| 361 | 1.44 (0.180, 2) | 88.8 (5.34, 2) | 43.0 (0.650, 2) | 98.8 (3.89, 2) | >9950 (n=½) | ND |
| 362 | 0.936 (0.0457, 2) | 86.7 (0.254, 2) | 29.9 (0.294, 2) | 107 (6.56, 2) | >9950 (n=½) | ND |
| 363 | 0.976 | 91.1 | 14.3 | 111 | >9950 | ND |
| 364 | 0.895 | 89.1 | 13 | 102 | >9950 | ND |
| 365 | 1.93 | 93.5 | 22.3 | 91 | >9950 | ND |
| 366 | 3.14 | 95.9 | 96.2 | 98.3 | >9950 | ND |
| 372 | 3.75 | 90.6 | 38 | 101 | >9950 | ND |
| 373 | 2.84 | 82.4 | 37.4 | 112 | >9950 | ND |
| 374 | 1.96 | 88.8 | 39.7 | 108 | >9950 | ND |
| 375 | 4.68 | 85.4 | 56.1 | 113 | >9950 | ND |
| 376 | 39.6 | 88.3 | 419 | 121 | >9950 | ND |
| 377 | 17.5 | 79.4 | 178 | 110 | >9950 | ND |
| 378 | 7.46 (0.401, 2) | 81.2 (0.297, 2) | 47.7 (5.86, 2) | 104 (2.97, 2) | >9950 (n=½) | ND |
| 379 | 11.6 (3.09, 2) | 90.0 (2.40, 2) | 48.5 (1.05, 2) | 101 (1.65, 2) | >9950 (n=½) | ND |
| 380 | 9.23 (1.41, 2) | 93.5 (4.71, 2) | 28.0 (2.79, 2) | 94.5 (0.549, 2) | >9950 (n=½) | ND |
| 381 | 28 | 90.3 | 153 | 106 | >9950 | ND |
| 382 | 18.7 | 87.2 | 204 | 118 | >9950 | ND |
| 383 | 10.7 (0.361, 2) | 82.8 (0.0145, 2) | 92.2 (7.22, 2) | 102 (1.72, 2) | >9950 (n=½) | ND |
| 384 | 0.457 (0.0717, 2) | 83.5 (7.50, 2) | 5.32 (0.0375, 2) | 99.5 (3.98, 2) | >9950 (n=½) | ND |
| 385 | 0.558 (0.0738, 2) | 83.5 (1.48, 2) | 10.6 (0.719, 2) | 112 (4.74, 2) | >9950 (n=½) | ND |
| 386 | 2.08 (0.338, 2) | 81.3 (1.47, 2) | 17.7 (0.911, 2) | 96.8 (3.81, 2) | >9950 (n=½) | ND |
| 387 | 0.278 (0.00115, 2) | 93.0 (6.92, 2) | 4.40 (1.07, 2) | 103 (1.08, 2) | >9950 (n=½) | ND |
| 388 | 0.463 | 82.1 | 6.31 | 104 | >9950 | ND |
| 389 | 1.50 (0.343, 2) | 82.0 (2.14, 2) | 9.58 (1.62, 2) | 106 (0.535, 2) | >9950 (n=½) | ND |
| 390 | 3.85 (0.361, 3) | 89.3 (3.58, 3) | 24.3 (1.05, 3) | 94.6 (2.35, 3) | >9950 (n=⅓) | ND |
| 391 | 0.266 (0.0311, 4) | 88.3 (1.76, 4) | 2.62 (0.437, 4) | 105 (2.45, 4) | >9950 (n=¼) | ND |
| 392 | 0.797 | 85.7 | 3.14 | 93.7 | >9950 | ND |
| 393 | 0.547 | 66.7 | 9.18 | 103 | >9950 | ND |
| 394 | 1.38 (0.437, 2) | 84.6 (11.0, 2) | 5.83 (1.31, 2) | 96.8 (7.02, 2) | >9950 (n=½) | ND |
| 395 | 0.327 | 57.8 | 6.26 | 96.1 | >9950 | ND |
| 396 | 0.595 | 67.1 | 6.49 | 94.4 | >9950 | ND |
| 397 | 1.92 (0.780, 2) | 84.5 (11.6, 2) | 7.17 (0.884, 2) | 103 (8.65, 2) | >9950 (n=½) | ND |
| 398 | 5.40 (1.52, 2) | 91.9 (0.357, 2) | 41.3 (4.18, 2) | 106 (4.28, 2) | >9950 (n=½) | ND |
| 399 | 16.2 | 86.8 | 43.6 | 89.7 | >9950 | ND |
| 400 | 16.7 | 83.2 | 107 | 109 | >9950 | ND |
| 401 | 0.434 (0.0341, 3) | 83.0 (7.25, 3) | 5.39 (0.0937, 3) | 107 (3.32, 3) | >9950 (n=⅓) | ND |
| 402 | 1.11 (0.0989, 3) | 84.5 (0.945, 3) | 13.5 (2.55, 3) | 110 (5.84, 3) | >9950 (n=⅓) | ND |
| 403 | 0.711 (0.103, 3) | 91.7 (6.87, 3) | 7.31 (0.446, 3) | 107 (2.08, 3) | >9950 (n=⅓) | ND |
| 404 | 1.74 | 77.3 | 49.1 | 99.3 | >9950 | ND |
| 405 | 1.23 | 74.3 | 37.5 | 78.1 | >9950 | ND |
| 406 | 0.527 (0.130, 2) | 87.3 (2.48, 2) | 9.21 (0.435, 2) | 96.0 (2.59, 2) | >9950 (n=½) | ND |
| 407 | 1.87 (0.139, 3) | 95.4 (4.35, 3) | 12.4(1.15, 3) | 97.2 (2.74, 3) | >9950 (n=⅓) | ND |
| 408 | 0.299 (0.0439, 3) | 84.4 (2.26, 3) | 5.92 (0.553, 3) | 99.5 (3.91, 3) | >9950 (n=⅓) | ND |
| 409 | 0.537 (0.0122, 3) | 84.0 (0.901, 3) | 8.05 (0.761, 3) | 97.7 (3.00, 3) | >9950 (n=⅓) | ND |
| 410 | 1.28 (0.255, 2) | 86.4 (5.77, 2) | 11.0 (0.428, 2) | 93.1 (0.997, 2) | >9950 (n=½) | ND |
| 411 | 1.09 (0.153, 3) | 84.6 (2.63, 3) | 11.4(1.88, 3) | 101 (2.54, 3) | >9950 (n=⅓) | ND |
| 412 | 1.57 (0.136, 3) | 87.5 (2.85, 3) | 18.3 (1.35, 3) | 106 (3.38, 3) | >9950 (n=⅓) | ND |
| 413 | 0.422 (0.0398, 3) | 91.8 (3.88, 3) | 5.47 (0.460, 3) | 107 (4.11, 3) | >9950 (n=⅓) | ND |
| 414 | 0.274 (0.0130, 5) | 92.2 (1.90, 5) | 3.24 (0.450, 5) | 103 (3.30, 5) | >9950 (n=⅕) | ND |
| 415 | 0.492 (0.0557, 4) | 93.9 (2.34, 4) | 5.72 (0.324, 4) | 104 (3.90, 4) | >9950 (n=¼) | ND |
| 416 | 0.723 (0.0458, 3) | 83.0 (5.25, 3) | 10.1 (0.689, 3) | 109 (2.55, 3) | >9950 (n=⅓) | ND |
| 417 | 0.201 (0.0212, 4) | 96.4 (1.41, 4) | 2.66 (0.254, 4) | 104 (2.88, 4) | >9950 (n=¼) | ND |
| 418 | 0.126 (0.0101, 3) | 87.2 (4.56, 3) | 1.72 (0.202, 3) | 98.8 (3.48, 3) | >9950 (n=⅓) | ND |
| 419 | 0.440 (0.0108, 3) | 88.2 (0.828, 3) | 7.19(1.28, 3) | 97.3 (3.90, 3) | >9950 (n=⅓) | ND |
| 420 | 0.351 (0.0281, 2) | 89.7 (1.93, 2) | 10.0 (1.52, 2) | 109 (14.4, 2) | >9950 (n=½) | ND |
| 421 | 0.164 (0.0176, 5) | 98.0 (2.76, 5) | 2.01 (0.326, 5) | 105 (3.71, 5) | >9950 (n=⅕) | ND |
| 422 | 0.199 (0.0226, 5) | 97.5 (1.55, 5) | 3.84 (0.645, 4) | 102 (6.38, 4) | >9950 (n=⅕) | ND |
| 423 | 0.294 (0.0208, 5) | 89.7 (3.15, 5) | 4.74 (0.644, 5) | 99.5 (3.90, 5) | >9950 (n=⅕) | ND |
| 424 | 0.159 (0.0110, 5) | 89.5 (1.11, 5) | 3.07 (0.179, 5) | 98.1 (3.03, 5) | >9950 (n=⅕) | ND |
| 425 | 0.875 (0.138, 2) | 89.9 (1.77, 2) | 10.3 (0.951, 2) | 103 (0.221, 2) | >9950 (n=½) | ND |
| 426 | 3.88 (0.200, 2) | 91.2 (3.07, 2) | 29.7 (2.34, 2) | 106 (11.5, 2) | >9950 (n=½) | ND |
| 427 | 2.04 (0.124, 2) | 92.4 (2.27, 2) | 25.1 (5.36, 2) | 109 (1.53, 2) | >9950 (n=½) | ND |
| 428 | 0.748 (0.0265, 2) | 93.6 (1.01, 2) | 10.2 (0.217, 2) | 105 (7.48, 2) | >9950 (n=½) | ND |
| 429 | 1.03 (0.0757, 2) | 93.3 (2.97, 2) | 17.3 (0.623, 2) | 101 (1.10, 2) | >9950 (n=½) | ND |
| 430 | 1.09 (0.108, 2) | 91.5 (4.62, 2) | 26.8 (3.10, 2) | 88.5 (3.34, 2) | >9950 (n=½) | ND |
| 431 | 0.645 (0.0819, 2) | 98.0 (6.44, 2) | 17.5 (0.336, 2) | 91.6 (8.16, 2) | >9950 (n=½) | ND |
| 432 | 0.0875 (0.00975, 5) | 96.7 (2.20, 5) | 1.28 (0.177, 5) | 103 (1.27, 5) | >9950 (n=⅕) | ND |
| 433 | 0.136 (0.0247, 3) | 100.0 (3.94, 3) | 2.63 (0.319, 2) | 101 (5.22, 2) | >9950 (n=⅓) | ND |
| 434 | 0.0994 (0.0113, 5) | 97.5 (2.66, 5) | 1.73 (0.292, 5) | 101 (2.09, 5) | >9950 (n=⅕) | ND |
| 435 | 0.0873 (0.00638, 5) | 98.7 (2.49, 5) | 1.63 (0.153, 5) | 97.8 (2.96, 5) | >9950 (n=⅕) | ND |
| 436 | 0.241 (0.0446, 3) | 96.0 (0.351, 3) | 4.98 (0.430, 3) | 100 (3.07, 3) | >9950 (n=⅓) | ND |
| 437 | 0.461 (0.0212, 2) | 96.5 (2.42, 2) | 9.65 (2.23, 2) | 91.2 (10.9, 2) | >9950 (n=½) | ND |
| 438 | 0.592 (0.100, 2) | 90.8 (1.84, 2) | 28.0 (1.64, 2) | 88.7 (20.6, 2) | >9950 (n=½) | ND |
| 439 | 0.396 (0.0386, 3) | 91.7 (2.17, 3) | 9.25 (2.51, 3) | 97.0 (5.24, 3) | >9950 (n=⅓) | ND |
| 440 | 0.209 (0.0138, 6) | 90.3 (1.85, 6) | 2.74 (0.115, 6) | 106 (1.98, 6) | >10100 (n=⅙) | ND |
| 441 | 1.38 (0.172, 4) | 83.8 (2.04, 4) | 5.75 (0.561, 4) | 111 (3.26, 4) | >9950 (n=¼) | ND |
| 442 | 0.687 (0.179, 3) | 92.1 (4.96, 3) | 7.63 (2.89, 3) | 106 (1.76, 3) | >9950 (n=⅓) | ND |
| 443 | 12.2(1.51, 2) | 91.1 (1.74, 2) | 133 (5.04, 2) | 99.3 (0.923, 2) | >9950 (n=½) | ND |
| 444 | 0.570 (0.0249, 2) | 83.6 (0.143, 2) | 7.51 (1.10, 2) | 98.8 (2.42, 2) | >9950 (n=½) | ND |
| 445 | 1.17 (0.102, 2) | 91.4 (0.322, 2) | 13.1 (0.359, 2) | 100 (0.430, 2) | >9950 (n=½) | ND |
| 446 | 5.14(1.45, 3) | 97.0 (4.46, 3) | 33.5 (2.16, 3) | 105 (1.85, 3) | >9950 (n=⅓) | ND |
| 447 | 1.46 (0.413, 3) | 101 (2.70, 3) | 7.85 (0.457, 3) | 98.4 (2.10, 3) | >9950 (n=⅓) | ND |
| 448 | 1.41 (0.531, 3) | 99.2 (0.884, 3) | 7.91 (0.258, 3) | 99.5 (1.45, 3) | >9950 (n=⅓) | ND |
| 449 | 0.595 (0.0397, 5) | 92.1 (4.58, 5) | 7.27 (0.337, 5) | 103 (5.19, 5) | >9950 (n=⅕) | ND |
| 450 | 2.44 (0.532, 5) | 91.4 (4.33, 5) | 32.3 (4.94, 5) | 102 (6.79, 5) | >9950 (n=⅕) | ND |
| 451 | 0.518 (0.103, 5) | 89.5 (2.84, 5) | 7.23 (0.798, 5) | 108 (5.40, 5) | >9950 (n=⅕) | ND |
| 452 | 1.21 (0.251, 3) | 100 (1.68, 3) | 16.8 (2.48, 3) | 108 (2.85, 3) | >9950 (n=⅓) | ND |
| 453 | 0.451 (0.0791, 5) | 92.0 (0.901, 5) | 5.47 (0.327, 5) | 108 (3.80, 5) | >9950 (n=⅕) | ND |
| 454 | 1.30 (0.201, 4) | 91.5 (0.474, 4) | 18.6(1.80, 4) | 103 (1.41, 4) | >9950 (n=¼) | ND |
| 455 | 0.362 (0.0529, 5) | 91.8 (1.84, 5) | 5.24 (0.295, 5) | 108 (2.00, 5) | >9950 (n=⅕) | ND |
| 456 | 0.878 (0.170, 3) | 94.7 (3.21, 3) | 14.7(1.55, 3) | 112 (5.14, 3) | >9950 (n=⅓) | ND |
| 457 | 0.334 (0.0519, 4) | 92.7 (3.24, 4) | 4.69 (1.37, 4) | 103 (3.76, 4) | >9950 (n=¼) | ND |
| 458 | 0.726 (0.0737, 4) | 93.1 (2.31, 4) | 7.61 (0.921, 4) | 106 (2.53, 4) | >9950 (n=¼) | ND |
| 459 | 0.323 (0.0736, 4) | 89.3 (1.16, 4) | 4.87 (0.929, 4) | 104 (2.02, 4) | >9950 (n=¼) | ND |
| 460 | 0.506 (0.0729, 4) | 89.5 (3.45, 4) | 6.31 (0.982, 4) | 99.5 (3.37, 4) | >9950 (n=¼) | ND |
| 461 | 0.247 (0.0520, 4) | 98.5 (0.822, 4) | 2.76 (0.361, 4) | 105 (2.29, 4) | >10100 (n=¼) | ND |
| 462 | 0.317 (0.0572, 3) | 98.4 (2.45, 3) | 3.34 (0.719, 3) | 104 (2.72, 3) | >9950 (n=⅓) | ND |
| 463 | 0.350 (0.0916, 3) | 93.7 (3.44, 3) | 4.09 (0.287, 2) | 103 (7.01, 2) | >9950 (n=⅓) | ND |
| 464 | 0.316 (0.0436, 4) | 95.5 (4.97, 4) | 4.48 (0.715, 4) | 104 (5.47, 4) | >10100 (n=¼) | ND |
| 465 | 3.63 (0.920, 3) | 94.4 (1.30, 3) | 73.2 (8.31, 3) | 93.3 (9.91, 3) | >9950 (n=⅓) | ND |
| 466 | 0.314 (0.0445, 2) | 93.4 (2.04, 2) | 3.32 (0.194, 2) | 99.8 (9.14, 2) | >10100 (n=½) | ND |
| 467 | 0.35 | 88.4 | 2.87 | 97.9 | >10100 (n=½) | ND |
| 468 | 0.222 | 85.9 | 1.84 | 102 | >10100 (n=½) | ND |
| 469 | 0.203 | 82.1 | 2.33 | 93.8 | >9950 (n=½) | ND |
| 470 | 0.719 | 93.9 | 4.54 | 113 | >9950 (n=½) | ND |
| 471 | 0.568 | 90.1 | 4.36 | 110 | >9950 (n=½) | ND |
| 472 | 0.663 | 76.3 | 7.41 | 103 | >9950 (n=½) | ND |
| 473 | 0.979 | 92.8 | 7.07 | 114 | >9950 (n=½) | ND |
| 474 | 0.156 | 85.6 | 1.1 | 104 | >9950 (n=½) | ND |
| 475 | 0.211 | 84.6 | 1.44 | 102 | >9950 (n=½) | ND |
| 476 | 0.154 | 81.8 | 1.5 | 107 | >9950 (n=½) | ND |
| 477 | 0.163 | 88.1 | 1.56 | 110 | >9950 (n=½) | ND |
| 478 | 0.517 | 84 | 6.16 | 108 | >9950 | ND |
| 479 | 0.196 | 89.5 | 2.55 | 95.3 | >9950 | ND |
| 480 | 0.29 | 99.7 | 2.65 | 104 | >9950 | ND |
| 481 | 0.467 | 82.7 | 6.16 | 112 | >9950 | ND |
| 482 | 0.89 | 104 | 4.85 | 110 | >9950 (n=½) | ND |
| 483 | 0.297 | 92.6 | 3.33 | 113 | >9950 (n=½) | ND |
| 484 | 0.453 | 91.3 | 3.66 | 103 | >9950 (n=½) | ND |
| 485 | 0.536 | 93.8 | 3.9 | 104 | >9950 | ND |
| 486 | 0.239 | 88.2 | 2 | 102 | >9950 (n=½) | ND |
| 487 | 0.429 | 89.1 | 3.16 | 104 | >9950 (n=½) | ND |
| 488 | 0.718 (0.161, 2) | 85.5 (1.49, 2) | 13.0 (0.709, 2) | 106 (4.29, 2) | >9950 (n=⅓) | ND |
| 489 | 0.578 | 79.4 | 13.5 | 98.7 | >9950 | ND |
| 490 | 0.976 | 97.1 | 7.68 | 101 | >9950 | ND |
| 491 | 1.08 | 87.5 | 5.51 | 105 | >9950 | ND |
| 492 | 0.371 | 88.7 | 4.31 | 104 | >9950 (n=½) | ND |
| 493 | 0.591 | 94.6 | 7.24 | 102 | >9950 | ND |
| 494 | 0.699 (0.0883, 4) | 92.6 (3.07, 4) | 9.17 (1.83, 4) | 106 (3.43, 4) | >9950 (n=¼) | ND |

ND = Not Determined
^{a} Expression density is determined using homologous competition binding of [¹²⁵I]GLP-1(7-36)NH₂ at hGLP-1R (112 fmol/mg protein), [¹²⁵I]GCG at hGCGR (98 fmol/mg protein) and [¹²⁵I]GIP(1-42) at hGIPR (124 fmol/mg protein).
^{b} EC₅₀, nM = the Geometric Mean, followed by the Standard Error of the Mean and the number of observations in parenthesis.
^{c} Eₘₐₓ, % = the Arithmetic Mean ± the Standard Error of the Mean for the percent of maximal response to GLP-1(7-36)NH₂ at hGLP-1R, GCG at hGCGR or GIP(1-42)NH₂ athGIPR.
All values shown are to three (3) significant digits.

### IN VIVO STUDIES

### Pharmacokinetics in Male CD-1 Mice

The pharmacokinetics of a test peptide is evaluated following a single subcutaneous administration of 200 nMol/kg to male CD-1 mice. Blood samples are collected over 168 hours and resulting individual plasma concentrations are used to calculate pharmacokinetic parameters. Plasma (K₃ EDTA) concentrations are determined using a qualified LC/MS method that measures the intact mass of the test peptide. Each test peptide and an analog as an internal standard are extracted from 100% mouse plasma using immunoaffinity based precipitation with anti-GIP/GLP1 antibodies. Instruments are combined for LC/MS detection. Mean pharmacokinetic parameters determine if the test peptide is consistent with an extended pharmacokinetic profile.

### Insulin Secretion in Male Wistar Rats

Male Wistar rats with femoral artery and femoral vein canulas (Envigo, Indianapolis, IN) (280- 320 grams) are single-housed in polycarbonate cages with filter tops. Rats maintained on a 12:12 h light-dark cycle (lights on at 6:00 A.M.) at 21° C. and receive food and deionized water ad libitum. Rats are randomized by body weight and dosed 1.5 ml/kg s.c. at test peptide doses of 0.04, 0.1, 0.3, 1, 3, and 10 nmol/kg 16 hours prior to glucose administration then fasted. Animals are weighed and anesthetized with sodium pentobarbital dosed i.p. (65 mg/kg, 30 mg/ml). A time 0 blood sample is collected into EDTA tubes after which glucose is administered i.v. (0.5 mg/kg, 5 ml/kg). Blood samples are collected for glucose and insulin levels at time 2, 4, 6, 10, 20 and 30 min post intravenous administration of glucose. Plasma glucose levels are determined using a clinical chemistry analyzer. Plasma insulin is determined using an electrochemiluminescence assay (Meso Scale, Gaithersburg, MD). Glucose and insulin AUC are examined compared to the vehicle control with n = 5 animals per group. Results are presented (SEM)(N). Results show the test peptide effect on insulin secretion during intravenous glucose tolerance test. Results show test peptide dose dependent effect on insulin secretion.

### Diet-Induced Obese C57/B16 Mice

C57/Bl6 diet-induced obese (DIO) male mice (Taconic, Germantown, NY) weighing 41-50 g are used. Animals are individually housed in a temperature-controlled (24° C.) facility with a 12 hour light/dark photoperiod (lights off at 10:00 AM and lights on at 10:00 PM), with free access to food and water. After 2 week acclimatization to the facility, mice are randomized to treatment groups (n=6/group) based on body weight so each group has similar starting mean body weight.

Mice are treated with either vehicle (40 mM Tris-HCl at pH 8.0) or a test peptide between the dose range of about 0.03 nmol/kg to about 10 nmol/kg. Treatments are subcutaneously administered to ad libitum fed DIO mice 30-90 minutes prior to the onset of the dark cycle daily (QD) for 14 days. Monitor body weight and food intake daily.

Data are expressed as mean ± SEM of 5-6 rats per group. Statistical analyses are assessed by one-way ANOVA followed by Dunnett’s multiple comparison test to compare treatment groups to vehicle group or each other. Significant differences are identified at p<0.05.

$$\begin{matrix}\text{Percent Body Weight} = \\ \frac{\text{Body weight after 14-day treatment}}{\text{Body weight before treatment started}} \times \text{100}\end{matrix}$$

“0” dose group represents the vehicle-treated mice during each study. All data are expressed as mean ± SEM of 5-6 mice per group. “Δ from vehicle” refers to difference between body weight at day 15 between test and vehicle groups. “% change” refers to percent decrease in body weight between days 1 and 15 in test groups. Record percent decrease in body weight for animals receiving vehicle. The Δ from vehicle and % change data are statistically significantly different (p<0.05) than control for a peptide testing positive in the assay.

### Amino Acid Sequences

SEQ ID NO: 1 GIP (Human)

YAEGTFISDYSIAMDKIHQQDFVNWLLAQKGKKNDWKHNITQ-NH₂

SEQ ID NO:2 Glucagon (Human)

HSQGTFTSDYSKYLDSRRAQDFVQWLMNT

SEQ ID NO:3 GLP-1 (7-36) (Human)

HAEGTFTSDVSSYLEGQAAKEFIAWLVKGR-NH₂

SEQ ID NO:4

X₁X₂EGTX₆TSDX₁₀X₁₁X₁₂X₁₃LDX₁₆X₁₇AQX₂₀X₂₁X₂₂IX₂₄X₂₅
LIX₂₈GX₃₀

SEQ ID NO:5

X₃₁SSG

SEQ ID NO:6

X₃₁SSG-R₃

SEQ ID NO:7

X₃₁SSGX₃₅PPPX₃₉

SEQ ID NO:8

X₃₁SSGX₃₅PPPX₃₉R₃

SEQ ID NO:9

X₃₁SSGX₃₅PPPX₃₉X₄₀

SEQ ID NO:10

X₃₁SSGX₃₅PPPX₃₉X₄₀R₃

SEQ ID NO:11

Y-Aib-EGT-αMeF(2F)-TSD-4Pal-SI-αMeL-LD-Orn-K((2-[2
-(2-Amino-ethoxy)-ethoxy]-acetyl)₂-(γ-Glu)-CO-(CH₂
)₁₂-CO₂H)AQ-Aib-EFI-(D-Glu)-αMeY-LIEGGK((2-[2-(2-A
mino-ethoxy)-ethoxy]-acetyl)₂-(γ-Glu)-CO-(CH₂)₁₂-C
O₂H)SSGAPPPS-NH₂

SEQ ID NO:12

Y-Aib-EGT-αMeF(2F)-TSD-4Pal-SI-αMeL-LD-Orn-K((2-[2
-(2-Amino-ethoxy)-ethoxy]-acetyl)₂-(γ-Glu)-(10-(4-
carboxyphenoxy)decanoyl))AQ-Aib-EFI-(D-Glu)-αMeY-L
IEGGK((2-[2-(2-Amino-ethoxy)-ethoxy]-acetyl)₂-(γ-G
lu)-(10-(4-carboxyphenoxy)decanoyl))SSGAPPPS-NH₂

SEQ ID NO:13

Y-Aib-EGT-αMeF(2F)-TSD-4Pal-SI-αMeL-LD-Orn-K((2-[2
-(2-Amino-ethoxy)-ethoxy]-acetyl)₂-(γ-Glu)-(4-(4-i
odophenyl)butanoyl))AQ-Aib-EFI-(D-Glu)-αMeY-LIEGGK
((2-[2-(2-Amino-ethoxy)-ethoxy]-acetyl)₂-(γ-Glu)-(
4-(4-iodophenyl)butanoyl))SSGAPPPS-NH₂

SEQ ID NO:14

Y-Aib-EGT-αMeF(2F)-TSD-4Pal-SI-αMeL-LD-Orn-K((2-[2
-(2-Amino-ethoxy)-ethoxy]-acetyl)₂-(γ-Glu)-(4-(4-t
ert-butylphenyl)butanoyl))AQ-Aib-EFI-(D-Glu)-αMeY-
LIEGGK((2-[2-(2-Amino-ethoxy)-ethoxy]-acetyl)₂-(γ-
Glu)-(4-(4-tert-butylphenyl)butanoyl))SSGAPPPS-NH₂

SEQ ID NO:15

Y-Aib-EGT-αMeF(2F)-TSD-4Pal-SI-αMeL-LD-Orn-K((2-[2
-(2-Amino-ethoxy)-ethoxy]-acetyl)₂-(γ-Glu)-CO-(CH₂
)₁₀-CH₃)AQ-Aib-EFI-(D-Glu)-αMeY-LIEGGK((2-[2-(2-Am
ino-ethoxy)-ethoxy]-acetyl)₂-(γ-Glu)-CO-(CH₂)₁₀-CH ₃)SSGAPPPS-NH₂

1. A compound of the formula:
X₁X₂EGTX₆TSDX₁₀X₁₁X₁₂X₁₃LDX₁₆X₁₇AQX₂₀X₂₁X₂₂IX₂₄X₂₅LIX₂₈GX₃₀ (SEQ ID NO:505) wherein
X₁ is selected from the group consisting of Y and R₁Y;
R₁ is an Ac modification of the N-terminal amino group;
X₂ is Aib;
X₆ is selected from the group consisting of αMeF and αMeF(2F);
X₁₀ is selected from the group consisting of 4Pal, Y, αMeF, αMeF(2F), αMeL, αMeV, Ac4c, Ac5c, Ac6c, Bip, 1Nal, 2Nal, OMeY, hTyr, Nle, V, 4CPhe, ChG, ChA, Bzt, 2FA, 4TAA, 2TA, 3TA, and KZ₁;
X₁₁ is selected from the group consisting of S, αMeS, Aib, G, Dap, Ac5c, and T1e;
X₁₂ is selected from the group consisting of I and KZ₁;
X₁₃ is selected from the group consisting of αMeL and αMeF;
X₁₆ is Orn;
X₁₇ is selected from the group consisting of Q, I, and KZ₁;
X₂₀ is selected from the group consisting of Aib, Orn, 4Pal, αMeF, Ac5c, and Ac6c;
X₂₁ is selected from the group consisting of E, KZ₁, G, Orn, and 4Pal;
X₂₂ is selected from the group consisting of F, 2ClPhe, 3ClPhe, 2FPhe, 3FPhe, 3,5FPhe, 1Nal, 2Nal, αMeF(2F), ChA, Bzt, and αMeF;
X₂₄ is selected from the group consisting of D-Glu, E, G, and KZ₁;
X₂₅ is selected from the group consisting of Y, αMeY, αMeF, and KZ₁;
X₂₈ is selected from the group consisting of E, Orn, and KZ₁;
X₃₀ is selected from the group consisting of G, Orn, KZ₁, K(Z₁)R₆, OrnR₂, and GR₂;
R₂ is selected from the group consisting of X₃₁, X₃₁SSG(SEQ ID NO:5), X₃₁SSG-R₃ (SEQ ID NO:6), X₃₁SSGX₃₅PPPX₃₉ (SEQ ID NO:7), X₃₁SSGX₃₅PPPX₃₉R₃ (SEQ ID NO:8), X₃₁SSGX₃₅PPPX₃₉X₄₀ (SEQ ID NO:9), X₃₁SSGX₃₅PPPX₃₉X₄₀R₃ (SEQ ID NO: 10), and a modification of the c-terminal group wherein the modification is NH₂; R₆ is selected from the group consisting of PSSG(SEQ ID NO:506), PSSG-R₃ (SEQ ID NO:507), PSSGX₃₅PPPX₃₉ (SEQ ID NO:508), PSSGX₃₅PPPX₃₉R₃ (SEQ ID NO:509), PSSGX₃₅PPPX₃₉X₄₀ (SEQ ID NO:510), PSSGX₃₅PPPX₃₉X₄₀R₃ (SEQ ID NO:511), and a modification of the c-terminal group wherein the modification is NH₂; X₃₁ is selected from the group consisting of P, and KZ₁;
X₃₅ is selected from the group consisting of A and Orn;
X₃₉ is selected from the group consisting of S and Orn;
X₄₀ is KZ₁,
R₃ is a modification of the C-terminal group, wherein the modification is NH₂;
wherein two, and only two, of X₁₀, X₁₂, X₁₇, X₂₁, X₂₄, X₂₅, X₂₈, X₃₀, X₃₁, and X₄₀ are KZ₁ or K(Z₁)R₆;
Z₁ is selected from the group consisting of R₅ and -R₄R₅;
R₄ is a linker; and
R₅ is a fatty acid; or
a pharmaceutically acceptable salt thereof.

2. A compound, or a pharmaceutically acceptable salt thereof, as claimed by claim 1 wherein
X₃₀ is selected from the group consisting of G, Orn, KZ₁, and GR₂; and
R₂ is selected from the group consisting of X₃₁SSG (SEQ ID NO:5), X₃₁SSG-R₃ (SEQ ID NO:6), X₃₁SSGX₃₅PPPX₃₉ (SEQ ID NO:7), X₃₁SSGX₃₅PPPX₃₉R₃ (SEQ ID NO:8), X₃₁SSGX₃₅PPPX₃₉X₄₀ (SEQ ID NO:9), X₃₁SSGX₃₅PPPX₃₉X₄₀R₃ (SEQ ID NO: 10), and a modification of the c-terminal group wherein the modification is NH₂.

3. A compound, or pharmaceutically acceptable salt thereof, as claimed by claim 2, wherein Z₁ is -R₄R₅.

4. A compound, or a pharmaceutically acceptable salt thereof, as claimed by claim 3 wherein R₅ is selected from the group consisting of -CO-(CH₂)₁₂-CO₂H, CO-(CH₂)₁₄-CO₂H, -CO-(CH₂)₁₀-CO₂H, -(10-(4-carboxyphenoxy)decanoyl), -(4-(4-iodophenyl)butanoyl), -(4-(4-tert-butylphenyl)butanoyl), -CO-(CH₂)₁₄-CH₃, -CO-(CH₂)₁₂-CH₃, -CO-(CH₂)₁₀-CH₃, -(7-(4-carboxyphenoxy)heptanoyl), -(11-(4-carboxyphenoxy)undecanoyl), -(12-(4-carboxyphenoxy)dodecanoyl), and -(8-(4-carboxyphenoxy)octanoyl).

5. A compound, or a pharmaceutically acceptable salt thereof, as claimed by claim 3 wherein R₅ is selected from the group consisting of
-CO-(CH₂)₁₂-CO₂H, -CO-(CH₂)₁₀-CO₂H, -(10-(4-carboxyphenoxy)decanoyl), -(4-(4-iodophenyl)butanoyl), -(4-(4-tert-butylphenyl)butanoyl), -CO-(CH₂)₁₄-CH₃, -CO-(CH₂)₁₂-CH₃, -CO-(CH₂)₁₀-CH₃, -(7-(4-carboxyphenoxy)heptanoyl), and -(8-(4-carboxyphenoxy)octanoyl).

6. A compound, or a pharmaceutically acceptable salt thereof, as claimed by claim 3 wherein R₅ is selected from the group consisting of
CO-(CH₂)₁₄-CO₂H, -(11-(4-carboxyphenoxy)undecanoyl), and -(12-(4-carboxyphenoxy)dodecanoyl).

7. A compound, or a pharmaceutically acceptable salt thereof, as claimed by claim 3 wherein R₄ is -(εK)ₐ₁-(γ-Glu)ₐ₂-(2-[2-(2-Amino-ethoxy)-ethoxy]-acetyl)ₐ₃-(εK)b1-(γ-Glu)_{b2}-;
a1 is selected from the group consisting of 0, 1, and 2;
a2 is selected from the group consisting of 0, 1, and 2;
a3 is selected from the group consisting of 0, 1, 2, and 3;
b1 is 0 or 1; and
b2 is 0 or 1.

8. A compound, or a pharmaceutically acceptable salt thereof, as claimed by claim 3 wherein R₄ is selected from the group consisting of
-(2-[2-(2-Amino-ethoxy)-ethoxy]-acetyl)₂-(γ-Glu)-,
-(2-[2-(2-Amino-ethoxy)-ethoxy]-acetyl)₂-,
-(2-[2-(2-Amino-ethoxy)-ethoxy]-acetyl)-(γ-Glu)-,
-(2-[2-(2-Amino-ethoxy)-ethoxy]-acetyl)₂-(γ-Glu)₂)-,
-(2-[2-(2-Amino-ethoxy)-ethoxy]-acetyl)₃-(γ-Glu)-,
-(2-[2-(2-Amino-ethoxy)-ethoxy]-acetyl)₂-(εK)-,
-(2-[2-(2-Amino-ethoxy)-ethoxy]-acetyl)₂-(εK)-(γ-Glu)-,
-(2-[2-(2-Amino-ethoxy)-ethoxy]-acetyl)-(εK)-(γ-Glu)-,
-(2-[2-(2-Amino-ethoxy)-ethoxy]-acetyl)-Trx-(γ-Glu)-,
-(2-[2-(2-Amino-ethoxy)-ethoxy]-acetyl)₂-Trx-(γ-Glu)-,
-(2-[2-(2-Amino-ethoxy)-ethoxy]-acetyl)-Aoc-,
-(2-[2-(2-Amino-ethoxy)-ethoxy]-acetyl)-Aoc-(γ-Glu)-,
-(2-[2-(2-Amino-ethoxy)-ethoxy]-acetyl)-Ahx-(γ-Glu)-,
-PEG3-(γ-Glu)-,
-PEG4-(γ-Glu)-,
-PEG5-(γ-Glu)-,
-PEG6-(γ-Glu)-,
-Ahx-(2-[2-(2-Amino-ethoxy)-ethoxy]-acetyl)-(γ-Glu)-,
-Aoc-(2-[2-(2-Amino-ethoxy)-ethoxy]-acetyl)-(γ-Glu)-,
-(εK)-(2-[2-(2-Amino-ethoxy)-ethoxy]-acetyl)-(γ-Glu)-,
-(εK)-(2-[2-(2-Amino-ethoxy)-ethoxy]-acetyl)₂-(γ-Glu)-,
-(εK)-(γ-Glu)-,
-(εK)-(γ-Glu)-(γ-Glu)-,
-(γ-Glu)-(2-[2-(2-Amino-ethoxy)-ethoxy]-acetyl)₂-,
-(εK)-(εK)-(γ-Glu)-,
-(γ-Glu)-(2-[2-(2-Amino-ethoxy)-ethoxy]-acetyl)-,
-(γ-Glu)-(2-[2-(2-Amino-ethoxy)-ethoxy]-acetyl)₂-(γ-Glu)-,
-(2-[2-(2-Amino-ethoxy)-ethoxy]-acetyl)-(γ-Glu)-(2-[2-(2-Amino-ethoxy)-ethoxy]-acetyl)-,
-(2-[2-(2-Amino-ethoxy)-ethoxy]-acetyl)₂-(γ-Glu)-(2-[2-(2-Amino-ethoxy)-ethoxy]-acetyl)-,
-(2-[2-(2-Amino-ethoxy)-ethoxy]-acetyl)-(y-Glu)-(2-[2-(2-Amino-ethoxy)-ethoxy]-acetyl)₂-, and
(2-[2-(2-Amino-ethoxy)-ethoxy]-acetyl)-(y-Glu)-(2-[2-(2-Amino-ethoxy)-ethoxy]-acetyl)-(γ-Glu)-.

9. A compound, or a pharmaceutically acceptable salt thereof, as claimed by claim 3 wherein R₄ is selected from the group consisting of
-(2-[2-(2-Amino-ethoxy)-ethoxy]-acetyl)₂-(γ-Glu)-,
-(2-[2-(2-Amino-ethoxy)-ethoxy]-acetyl)₂-,
-(2-[2-(2-Amino-ethoxy)-ethoxy]-acetyl)-(γ-Glu)-,
-(2-[2-(2-Amino-ethoxy)-ethoxy]-acetyl)₃-(γ-Glu)-,
-(2-[2-(2-Amino-ethoxy)-ethoxy]-acetyl)₂-(εK)-,
-(2-[2-(2-Amino-ethoxy)-ethoxy]-acetyl)₂-(εK)-(γ-Glu)-,
-(2-[2-(2-Amino-ethoxy)-ethoxy]-acetyl)-(εK)-(γ-Glu)-,
-(εK)-(2-[2-(2-Amino-ethoxy)-ethoxy]-acetyl)-(γ-Glu)-,
-(εK)-(2-[2-(2-Amino-ethoxy)-ethoxy]-acetyl)₂-(γ-Glu)-,
-(εK)-(γ-Glu)-,
-(εK)-(γ-Glu)-(γ-Glu)-,
-(γ-Glu)-(2-[2-(2-Amino-ethoxy)-ethoxy]-acetyl)₂-,
-(εK)-(εK)-(γ-Glu)-,
-(γ-Glu)-(2-[2-(2-Amino-ethoxy)-ethoxy]-acetyl)-, and
-(2-[2-(2-Amino-ethoxy)-ethoxy]-acetyl)-(γ-Glu)-(2-[2-(2-Amino-ethoxy)-ethoxy]-acetyl)-.

10. A compound, or pharmaceutically acceptable salt thereof, as claimed by claim 2, wherein Z₁ is selected from the group consisting of
-(2-[2-(2-Amino-ethoxy)-ethoxy]-acetyl)₂-(γ-Glu)-CO-(CH₂)₁₂-CO₂H,
-(2-[2-(2-Amino-ethoxy)-ethoxy]-acetyl)₂-(γ-Glu)-CO-(CH₂)₁₀-CO₂H,
-(2-[2-(2-Amino-ethoxy)-ethoxy]-acetyl)₂-(γ-Glu)-(10-(4-carboxyphenoxy)decanoyl),
-(2-[2-(2-Amino-ethoxy)-ethoxy]-acetyl)₂-(γ-Glu)-(4-(4-iodophenyl)butanoyl),
-(2-[2-(2-Amino-ethoxy)-ethoxy]-acetyl)₂-(γ-Glu)-(4-(4-tert-butylphenyl)butanoyl),
-(2-[2-(2-Amino-ethoxy)-ethoxy]-acetyl)₂-(γ-Glu)-CO-(CH₂)₁₄-CH₃,
-(2-[2-(2-Amino-ethoxy)-ethoxy]-acetyl)₂-(γ-Glu)-CO-(CH₂)₁₂-CH₃,
-(2-[2-(2-Amino-ethoxy)-ethoxy]-acetyl)₂-(γ-Glu)-CO-(CH₂)₁₀-CH₃,
-(2-[2-(2-Amino-ethoxy)-ethoxy]-acetyl)₂-CO-(CH₂)₁₂-CO₂H,
-(2-[2-(2-Amino-ethoxy)-ethoxy]-acetyl)₂-(10-(4-carboxyphenoxy)decanoyl),
-(2-[2-(2-Amino-ethoxy)-ethoxy]-acetyl)-(γ-Glu)-CO-(CH₂)₁₂-CO₂H,
-(2-[2-(2-Amino-ethoxy)-ethoxy]-acetyl)-(γ-Glu)-(10-(4-carboxyphenoxy)decanoyl),
-(2-[2-(2-Amino-ethoxy)-ethoxy]-acetyl)-(γ-Glu)-CO-(CH₂)₁₄-CH₃,
-(2-[2-(2-Amino-ethoxy)-ethoxy]-acetyl)-(γ-Glu)-CO-(CH₂)₁₀-CH₃,
-(2-[2-(2-Amino-ethoxy)-ethoxy]-acetyl)₃-(γ-Glu)-CO-(CH₂)₁₂-CO₂H,
-(2-[2-(2-Amino-ethoxy)-ethoxy]-acetyl)₃-(γ-Glu)-CO-(CH₂)₁₄-CH₃,
-(2-[2-(2-Amino-ethoxy)-ethoxy]-acetyl)₂-(εK)-CO-(CH₂)₁₂-CO₂H,
-(2-[2-(2-Amino-ethoxy)-ethoxy]-acetyl)₂-(εK)-(γ-Glu)-CO-(CH₂)₁₂-CO₂H,
-(2-[2-(2-Amino-ethoxy)-ethoxy]-acetyl)-(εK)-(γ-Glu)-CO-(CH₂)₁₂-CO₂H,
-(εK)-(2-[2-(2-Amino-ethoxy)-ethoxy]-acetyl)-(γ-Glu)-CO-(CH₂)₁₂-CO₂H,
-(εK)-(2-[2-(2-Amino-ethoxy)-ethoxy]-acetyl)₂-(γ-Glu)-CO-(CH₂)₁₂-CO₂H,
-(εK)-(γ-Glu)-CO-(CH₂)₁₂-CO₂H,
-(εK)-(γ-Glu)-(γ-Glu)-CO-(CH₂)₁₂-CO₂H,
-(γ-Glu)-(2-[2-(2-Amino-ethoxy)-ethoxy]-acetyl)₂-CO-(CH₂)₁₂-CO₂H,
-(εK)-(εK)-(γ-Glu)-CO-(CH₂)₁₂-CO₂H,
-(γ-Glu)-(2-[2-(2-Amino-ethoxy)-ethoxy]-acetyl)-CO-(CH₂)₁₂-CO₂H,
-(2-[2-(2-Amino-ethoxy)-ethoxy]-acetyl)-(γ-Glu)-(2-[2-(2-Amino-ethoxy)-ethoxy]-acetyl)-CO-(CH₂)₁₂-CO₂H,
-(2-[2-(2-Amino-ethoxy)-ethoxy]-acetyl)₂-(γ-Glu)-(7-(4-carboxyphenoxy)heptanoyl),
-(2-[2-(2-Amino-ethoxy)-ethoxy]-acetyl)₂-(γ-Glu)-(8-(4-carboxyphenoxy)octanoyl),
-(2-[2-(2-Amino-ethoxy)-ethoxy]-acetyl)-(γ-Glu)-(4-(4-tert-butylphenyl)butanoyl),
-(2-[2-(2-Amino-ethoxy)-ethoxy]-acetyl)₂-(γ-Glu)-(11-(4-carboxyphenoxy)undecanoyl),
-(2-[2-(2-Amino-ethoxy)-ethoxy]-acetyl)₃-(γ-Glu)-(10-(4-carboxyphenoxy)decanoyl),
-(2-[2-(2-Amino-ethoxy)-ethoxy]-acetyl)₂-(γ-Glu)-(2-[2-(2-Amino-ethoxy)-ethoxy]-acetyl)-CO-(CH₂)₁₂-CO₂H,
-(2-[2-(2-Amino-ethoxy)-ethoxy]-acetyl)-(γ-Glu)-(2-[2-(2-Amino-ethoxy)-ethoxy]-acetyl)₂-CO-(CH₂)₁₂-CO₂H,
-PEG3-(γ-Glu)-CO-(CH₂)₁₂-CO₂H,
-PEG4-(γ-Glu)-CO-(CH₂)₁₂-CO₂H,
-(2-[2-(2-Amino-ethoxy)-ethoxy]-acetyl)₃-(γ-Glu)-CO-(CH₂)₁₀-CH₃,
-(2-[2-(2-Amino-ethoxy)-ethoxy]-acetyl)₂-(γ-Glu)-(11-(4-carboxyphenoxy)undecanoyl,
-(2-[2-(2-Amino-ethoxy)-ethoxy]-acetyl)₂-(γ-Glu)-CO-(CH₂)₁₄-CO₂H,
-(2-[2-(2-Amino-ethoxy)-ethoxy]-acetyl)-Ahx-(γ-Glu)-CO-(CH₂)₁₂-CO₂H,
-(2-[2-(2-Amino-ethoxy)-ethoxy]-acetyl)-Aoc-(γ-Glu)-CO-(CH₂)₁₂-CO₂H,
-Ahx-(2-[2-(2-Amino-ethoxy)-ethoxy]-acetyl)-(γ-Glu)-CO-(CH₂)₁₂-CO₂H,
-Aoc-(2-[2-(2-Amino-ethoxy)-ethoxy]-acetyl)-(γ-Glu)-CO-(CH₂)₁₂-CO₂H,
-(2-[2-(2-Amino-ethoxy)-ethoxy]-acetyl)-Ahx-(γ-Glu)-(10-(4-carboxyphenoxy)decanoyl),
-(2-[2-(2-Amino-ethoxy)-ethoxy]-acetyl)-Aoc-(γ-Glu)-(10-(4-carboxyphenoxy)decanoyl),
-Ahx-(2-[2-(2-Amino-ethoxy)-ethoxy]-acetyl)-(γ-Glu)-(10-(4-carboxyphenoxy)decanoyl),
-Aoc-(2-[2-(2-Amino-ethoxy)-ethoxy]-acetyl)-(γ-Glu)-(10-(4-carboxyphenoxy)decanoyl),
-PEG4-(γ-Glu)-(10-(4-carboxyphenoxy)decanoyl),
-PEG3-(γ-Glu)-(10-(4-carboxyphenoxy)decanoyl),
-(2-[2-(2-Amino-ethoxy)-ethoxy]-acetyl)-Aoc-CO-(CH₂)₁₂-CO₂H,
-PEG6-(γ-Glu)-CO-(CH₂)₁₂-CO₂H,
-PEG5-(γ-Glu)-CO-(CH₂)₁₂-CO₂H,
-PEG6-(γ-Glu)-(10-(4-carboxyphenoxy)decanoyl),
-PEG5-(γ-Glu)-(10-(4-carboxyphenoxy)decanoyl),
-(2-[2-(2-Amino-ethoxy)-ethoxy]-acetyl)₂-Trx-(γ-Glu)-CO-(CH₂)₁₂-CO₂H,
-(2-[2-(2-Amino-ethoxy)-ethoxy]-acetyl)₂-Trx-(γ-Glu)-(10-(4-carboxyphenoxy)decanoyl),
-(2-[2-(2-Amino-ethoxy)-ethoxy]-acetyl)-Trx-(γ-Glu)-CO-(CH₂)₁₂-CO₂H,
-(2-[2-(2-Amino-ethoxy)-ethoxy]-acetyl)-Trx-(γ-Glu)-(10-(4-carboxyphenoxy)decanoyl),
-(2-[2-(2-Amino-ethoxy)-ethoxy]-acetyl)₂-(γ-Glu)₂-CO-(CH₂)₁₂-CO₂H,
-(2-[2-(2-Amino-ethoxy)-ethoxy]-acetyl)₂-(γ-Glu)₂-(10-(4-carboxyphenoxy)decanoyl),
-(γ-Glu)-(2-[2-(2-Amino-ethoxy)-ethoxy]-acetyl)₂-(γ-Glu)-CO-(CH₂)₁₂-CO₂H,
-(γ-Glu)-(2-[2-(2-Amino-ethoxy)-ethoxy]-acetyl)₂-(γ-Glu)-(10-(4-carboxyphenoxy)decanoyl),
-(2-[2-(2-Amino-ethoxy)-ethoxy]-acetyl)-(γ-Glu)-(2-[2-(2-Amino-ethoxy)-ethoxy]-acetyl)-(γ-Glu)-CO-(CH₂)₁₂-CO₂H, and
-(2-[2-(2-Amino-ethoxy)-ethoxy]-acetyl)-(γ-Glu)-(2-[2-(2-Amino-ethoxy)-ethoxy]-acetyl)-(γ-Glu)-(10-(4-carboxyphenoxy)decanoyl).

11. A compound, or a pharmaceutically acceptable salt thereof, as claimed by claim 10 wherein Z₁ is selected from the group consisting of:
-(2-[2-(2-Amino-ethoxy)-ethoxy]-acetyl)₂-(γ-Glu)-CO-(CH₂)₁₂-CO₂H,
-(2-[2-(2-Amino-ethoxy)-ethoxy]-acetyl)₂-(γ-Glu)-CO-(CH₂)₁₀-CO₂H,
-(2-[2-(2-Amino-ethoxy)-ethoxy]-acetyl)₂-(γ-Glu)-(10-(4-carboxyphenoxy)decanoyl),
-(2-[2-(2-Amino-ethoxy)-ethoxy]-acetyl)₂-(γ-Glu)-(4-(4-iodophenyl)butanoyl),
-(2-[2-(2-Amino-ethoxy)-ethoxy]-acetyl)₂-(γ-Glu)-(4-(4-tert-butylphenyl)butanoyl),
-(2-[2-(2-Amino-ethoxy)-ethoxy]-acetyl)₂-(γ-Glu)-CO-(CH₂)₁₄-CH₃,
-(2-[2-(2-Amino-ethoxy)-ethoxy]-acetyl)₂-(γ-Glu)-CO-(CH₂)₁₂-CH₃,
-(2-[2-(2-Amino-ethoxy)-ethoxy]-acetyl)₂-(γ-Glu)-CO-(CH₂)₁₀-CH₃,
-(2-[2-(2-Amino-ethoxy)-ethoxy]-acetyl)₂-CO-(CH₂)₁₂-CO₂H,
-(2-[2-(2-Amino-ethoxy)-ethoxy]-acetyl)₂-(10-(4-carboxyphenoxy)decanoyl),
-(2-[2-(2-Amino-ethoxy)-ethoxy]-acetyl)-(γ-Glu)-CO-(CH₂)₁₂-CO₂H,
-(2-[2-(2-Amino-ethoxy)-ethoxy]-acetyl)-(γ-Glu)-(10-(4-carboxyphenoxy)decanoyl),
-(2-[2-(2-Amino-ethoxy)-ethoxy]-acetyl)-(γ-Glu)-CO-(CH₂)₁₄-CH₃,
-(2-[2-(2-Amino-ethoxy)-ethoxy]-acetyl)-(γ-Glu)-CO-(CH₂)io-CH₃,
-(2-[2-(2-Amino-ethoxy)-ethoxy]-acetyl)₃-(γ-Glu)-CO-(CH₂)₁₂-CO₂H,
-(2-[2-(2-Amino-ethoxy)-ethoxy]-acetyl)₃-(γ-Glu)-CO-(CH₂)₁₄-CH₃,
-(2-[2-(2-Amino-ethoxy)-ethoxy]-acetyl)₂-(εK)-CO-(CH₂)₁₂-CO₂H,
-(2-[2-(2-Amino-ethoxy)-ethoxy]-acetyl)₂-(εK)-(γ-Glu)-CO-(CH₂)₁₂-CO₂H,
-(2-[2-(2-Amino-ethoxy)-ethoxy]-acetyl)-(εK)-(γ-Glu)-CO-(CH₂)₁₂-CO₂H,
-(εK)-(2-[2-(2-Amino-ethoxy)-ethoxy]-acetyl)-(γ-Glu)-CO-(CH₂)₁₂-CO₂H,
-(εK)-(2-[2-(2-Amino-ethoxy)-ethoxy]-acetyl)₂-(γ-Glu)-CO-(CH₂)₁₂-CO₂H,
-(εK)-(γ-Glu)-CO-(CH₂)₁₂-CO₂H,
-(εK)-(γ-Glu)-(γ-Glu)-CO-(CH₂)₁₂-CO₂H,
-(γ-Glu)-(2-[2-(2-Amino-ethoxy)-ethoxy]-acetyl)₂-CO-(CH₂)₁₂-CO₂H,
-(εK)-(εK)-(γ-Glu)-CO-(CH₂)₁₂-CO₂H,
-(γ-Glu)-(2-[2-(2-Amino-ethoxy)-ethoxy]-acetyl)-CO-(CH₂)₁₂-CO₂H,
-(2-[2-(2-Amino-ethoxy)-ethoxy]-acetyl)-(y-Glu)-(2-[2-(2-Amino-ethoxy)-ethoxy]-acetyl)-CO-(CH₂)₁₂-CO₂H,
-(2-[2-(2-Amino-ethoxy)-ethoxy]-acetyl)₂-(γ-Glu)-(7-(4-carboxyphenoxy)heptanoyl),
-(2-[2-(2-Amino-ethoxy)-ethoxy]-acetyl)₂-(γ-Glu)-(8-(4-carboxyphenoxy)octanoyl), and
-(2-[2-(2-Amino-ethoxy)-ethoxy]-acetyl)-(y-Glu)-(4-(4-tert-butylphenyl)butanoyl).

12. A compound, or a pharmaceutically acceptable salt thereof, as claimed by claim 10 wherein Z₁ is selected from the group consisting of:
-(2-[2-(2-Amino-ethoxy)-ethoxy]-acetyl)₂-(γ-Glu)-(11-(4-carboxyphenoxy)undecanoyl),
-(2-[2-(2-Amino-ethoxy)-ethoxy]-acetyl)₃-(γ-Glu)-(10-(4-carboxyphenoxy)decanoyl),
-(2-[2-(2-Amino-ethoxy)-ethoxy]-acetyl)₂-(γ-Glu)-(2-[2-(2-Amino-ethoxy)-ethoxy]-acetyl)-CO-(CH₂)₁₂-CO₂H,
-(2-[2-(2-Amino-ethoxy)-ethoxy]-acetyl)-(γ-Glu)-(2-[2-(2-Amino-ethoxy)-ethoxy]-acetyl)₂-CO-(CH₂)₁₂-CO₂H,
-PEG3-(γ-Glu)-CO-(CH₂)₁₂-CO₂H,
-PEG4-(γ-Glu)-CO-(CH₂)₁₂-CO₂H,
-(2-[2-(2-Amino-ethoxy)-ethoxy]-acetyl)₃-(γ-Glu)-CO-(CH₂)₁₀-CH₃,
-(2-[2-(2-Amino-ethoxy)-ethoxy]-acetyl)₂-(γ-Glu)-(11-(4-carboxyphenoxy)undecanoyl,
-(2-[2-(2-Amino-ethoxy)-ethoxy]-acetyl)₂-(γ-Glu)-CO-(CH₂)₁₄-CO₂H,
-(2-[2-(2-Amino-ethoxy)-ethoxy]-acetyl)-Ahx-(γ-Glu)-CO-(CH₂)₁₂-CO₂H,
-(2-[2-(2-Amino-ethoxy)-ethoxy]-acetyl)-Aoc-(γ-Glu)-CO-(CH₂)₁₂-CO₂H,
-Ahx-(2-[2-(2-Amino-ethoxy)-ethoxy]-acetyl)-(γ-Glu)-CO-(CH₂)₁₂-CO₂H,
-Aoc-(2-[2-(2-Amino-ethoxy)-ethoxy]-acetyl)-(γ-Glu)-CO-(CH₂)₁₂-CO₂H,
-(2-[2-(2-Amino-ethoxy)-ethoxy]-acetyl)-Ahx-(γ-Glu)-(10-(4-carboxyphenoxy)decanoyl),
-(2-[2-(2-Amino-ethoxy)-ethoxy]-acetyl)-Aoc-(γ-Glu)-(10-(4-carboxyphenoxy)decanoyl),
-Ahx-(2-[2-(2-Amino-ethoxy)-ethoxy]-acetyl)-(γ-Glu)-(10-(4-carboxyphenoxy)decanoyl),
-Aoc-(2-[2-(2-Amino-ethoxy)-ethoxy]-acetyl)-(γ-Glu)-(10-(4-carboxyphenoxy)decanoyl),
-PEG4-(γ-Glu)-(10-(4-carboxyphenoxy)decanoyl),
-PEG3-(γ-Glu)-(10-(4-carboxyphenoxy)decanoyl),
-(2-[2-(2-Amino-ethoxy)-ethoxy]-acetyl)-Aoc-CO-(CH₂)₁₂-CO₂H,
-PEG6-(γ-Glu)-CO-(CH₂)₁₂-CO₂H,
-PEG5-(γ-Glu)-CO-(CH₂)₁₂-CO₂H,
-PEG6-(γ-Glu)-(10-(4-carboxyphenoxy)decanoyl),
-PEG5-(γ-Glu)-(10-(4-carboxyphenoxy)decanoyl),
-(2-[2-(2-Amino-ethoxy)-ethoxy]-acetyl)₂-Trx-(γ-Glu)-CO-(CH₂)₁₂-CO₂H,
-(2-[2-(2-Amino-ethoxy)-ethoxy]-acetyl)₂-Trx-(γ-Glu)-(10-(4-carboxyphenoxy)decanoyl),
-(2-[2-(2-Amino-ethoxy)-ethoxy]-acetyl)-Trx-(γ-Glu)-CO-(CH₂)₁₂-CO₂H,
-(2-[2-(2-Amino-ethoxy)-ethoxy]-acetyl)-Trx-(γ-Glu)-(10-(4-carboxyphenoxy)decanoyl),
-(2-[2-(2-Amino-ethoxy)-ethoxy]-acetyl)₂-(γ-Glu)₂-CO-(CH₂)₁₂-CO₂H,
-(2-[2-(2-Amino-ethoxy)-ethoxy]-acetyl)₂-(γ-Glu)₂-(10-(4-carboxyphenoxy)decanoyl),
-(γ-Glu)-(2-[2-(2-Amino-ethoxy)-ethoxy]-acetyl)₂-(γ-Glu)-CO-(CH₂)₁₂-CO₂H,
-(γ-Glu)-(2-[2-(2-Amino-ethoxy)-ethoxy]-acetyl)₂-(γ-Glu)-(10-(4-carboxyphenoxy)decanoyl),
-(2-[2-(2-Amino-ethoxy)-ethoxy]-acetyl)-(γ-Glu)-(2-[2-(2-Amino-ethoxy)-ethoxy]-acetyl)-(γ-Glu)-CO-(CH₂)₁₂-CO₂H, and
-(2-[2-(2-Amino-ethoxy)-ethoxy]-acetyl)-(γ-Glu)-(2-[2-(2-Amino-ethoxy)-ethoxy]-acetyl)-(γ-Glu)-(10-(4-carboxyphenoxy)decanoyl).

13. A compound, or pharmaceutically acceptable salt thereof, as claimed by claim 1 wherein:
X₁ is Y;
X₆ is αMeF(2F);
X₁₀ is selected from the group consisting of 4Pal, Y, and KZ₁;
X₁₁ is selected from the group consisting of S, αMeS, and Aib;
X₁₂ is I;
X₁₃ is αMeL;
X₁₆ is Orn;
X₁₇ is selected from the group consisting of I and KZ₁;
X₂₀ is Aib;
X₂₁ is E or KZ₁;
X₂₂ is selected from the group consisting of F and αMeF;
X₂₄ is selected from the group consisting of D-Glu and KZ₁;
X₂₅ is αMeY;
X₂₈ is selected from the group consisting of E and KZ₁;
X₃₀ is selected from the group consisting of G and GR₂;
R₂ is selected from the group consisting of X₃₁SSGX₃₅PPPX₃₉ (SEQ ID NO:7),
X₃₁SSGX₃₅PPPX₃₉R₃ (SEQ ID NO:8), and X₃₁SSGX₃₅PPPX₃₉X₄₀ (SEQ ID NO:9), and a modification of the c-terminal group wherein the modification is NH₂;
or a pharmaceutically acceptable salt thereof.

14. A compound or pharmaceutically acceptable salt thereof, as claimed by claim 13 wherein
X₃₀ is GR₂; and
R₂ is selected from the group consisting of X₃₁SSGX₃₅PPPX₃₉ (SEQ ID NO:7), X₃₁SSGX₃₅PPPX₃₉R₃ (SEQ ID NO:8), and X₃₁SSGX₃₅PPPX₃₉X₄₀ (SEQ ID NO:9).

15. A compound, or pharmaceutically acceptable salt thereof, as claimed by claim 13 wherein X₁₇ and X₃₁ are each KZ₁.

16. A compound, or pharmaceutically acceptable salt thereof, as claimed by claim 13 wherein X₁₇ and X₂₄ are each KZ₁.

17. A compound, or pharmaceutically acceptable salt thereof, as claimed by claim 13 wherein X₁₇ and X₂₁ are each KZ₁.

18. A compound, or pharmaceutically acceptable salt thereof, as claimed by claim 13 wherein X₁₇ and X₂₈ are each KZ₁.

19. A compound, or pharmaceutically acceptable salt thereof, as claimed by claim 13 wherein X₂₁ and X₂₈ are each KZ₁.

20. A compound, or pharmaceutically acceptable salt thereof, as claimed by claim 13 wherein X₂₄ and X₂₈ are each KZ₁.

21. A compound, or pharmaceutically acceptable salt thereof, as claimed by claim 13 wherein X₁₇ and X₄₀ are each KZ₁.

22. A compound, or pharmaceutically acceptable salt thereof, as claimed by claim 13 wherein X₂₁ and X₄₀ are each KZ₁.

23. A compound, or pharmaceutically acceptable salt thereof, as claimed by claim 1 wherein the compound is selected from the group consisting of
Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂,
Y-Aib-EGT-aMeF(2F)-TSD-4Pal-SI-aMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-aMeY-LIEGGK((2-[2-(2-Amino-ethoxy)-ethoxy]-acetyl)₂-(γ-Glu)-(10-(4-carboxyphenoxy)decanoyl))SSGAPPPS-NH₂,
Y-Aib-EGT-aMeF(2F)-TSD-4Pal-SI-aMeL-LD-Orn-K((2-[2-(2-Amino-ethoxy)-ethoxy]-acetyl)₂-(γ-Glu)-(4-(4-iodophenyl)butanoyl))AQ-Aib-EFI-(D-Glu)-aMeY-LIEGGK((2-[2-(2-Amino-ethoxy)-ethoxy]-acetyl)₂-(γ-Glu)-(4-(4-iodophenyl)butanoyl))SSGAPPPS-NH₂,
Y-Aib-EGT-aMeF(2F)-TSD-4Pal-SI-aMeL-LD-Orn-K((2-[2-(2-Amino-ethoxy)-ethoxy]-acetyl)₂-(γ-Glu)-(4-(4-tert-butylphenyl)butanoyl))AQ-Aib-EFI-(D-Glu)-aMeY-LIEGGK((2-[2-(2-Amino-ethoxy)-ethoxy]-acetyl)₂-(γ-Glu)-(4-(4-tert-butylphenyl)butanoyl))SSGAPPPS-NH₂, and
Y-Aib-EGT-aMeF(2F)-TSD-4Pal-SI-aMeL-LD-Orn-K((2-[2-(2-Amino-ethoxy)-ethoxy]-acetyl)₂-(γ-Glu)-CO-(CH₂)₁₀-CH3)AQ-Aib-EFI-(D-Glu)-αMeY-LIEGGK((2-[2-(2-Amino-ethoxy)-ethoxy]-acetyl)₂-(γ-Glu)-CO-(CH₂)₁₀-CH₃)SSGAPPPS-NH₂.

24. A compound, or pharmaceutically acceptable salt thereof, as claimed by claim 23 wherein the compound is Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₀-CH₃)AQ-Aib-EFI-(D-Glu)-αMeY-LIEGGK((2-[2-(2-Amino-ethoxy)-ethoxy]-acetyl)₂-(γ-Glu)-CO-(CH₂)₁₀-CH₃)SSGAPPPS-NH₂.

25. A compound, or pharmaceutically acceptable salt thereof, as claimed by claim 23 wherein the compound is Y-Aib-EGT-aMeF(2F)-TSD-4Pal-SI-aMeL-LD-Orn-K((2-[2-(2-Amino-ethoxy)-ethoxy]-acetyl)₂-(γ-Glu)-(4-(4-tert-butylphenyl)butanoyl))AQ-Aib-EFI-(D-Glu)-aMeY-LIEGGK((2-[2-(2-Amino-ethoxy)-ethoxy]-acetyl)₂-(γ-Glu)-(4-(4-tert-butylphenyl)butanoyl))SSGAPPPS-NHz.

26. A compound, or pharmaceutically acceptable salt thereof, as claimed by claim 23 wherein the compound is Y-Aib-EGT-aMeF(2F)-TSD-4Pal-SI-aMeL-LD-Orn-K((2-[2-(2-Amino-ethoxy)-ethoxy]-acetyl)₂-(γ-Glu)-(4-(4-iodophenyl)butanoyl))AQ-Aib-EFI-(D-Glu)-aMeY-LIEGGK((2-[2-(2-Amino-ethoxy)-ethoxy]-acetyl)₂-(γ-Glu)-(4-(4-iodophenyl)butanoyl))SSGAPPPS-NH_{2.}

27. A compound, or pharmaceutically acceptable salt thereof, as claimed by claim 23 wherein the compound is Y-Aib-EGT-aMeF(2F)-TSD-4Pal-SI-aMeL-LD-Orn-K((2-[2-(2-Amino-ethoxy)-ethoxy]-acetyl)₂-(γ-Glu)-(10-(4-carboxyphenoxy)decanoyl))AQ-Aib-EFI-(D-Glu)-aMeY-LIEGGK((2-[2-(2-Amino-ethoxy)-ethoxy]-acetyl)₂-(γ-Glu)-(10-(4-carboxyphenoxy)decanoyl))SSGAPPPS-NH₂.

28. A compound, or pharmaceutically acceptable salt thereof, as claimed by claim 23 wherein the compound is Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO-(CH₂)₁₂-CO₂H)AQ-Aib-EFI-(D-Glu)-αMeY-LIEGGK((2-[2-(2-Amino-ethoxy)-ethoxy]-acetyl)₂-(γ-Glu)-CO-(CH₂)₁₂-CO₂H)SSGAPPPS-NH₂.

29. A method for treating a condition selected from the group consisting of type 2 diabetes mellitus, obesity, NAFLD, nonalcoholic steatohepatitis, dyslipidemia, and metabolic syndrome, comprising administering to a patient in need thereof, an effective amount of a compound, or a pharmaceutically acceptable salt thereof, as claimed by claim 1.

30. A method for treating obesity, comprising administering to a patient in need thereof, an effective amount of a compound, or a pharmaceutically acceptable salt thereof, as claimed by claim 1.

31. A method for providing therapeutic weight loss, comprising administering to a subject in need thereof, an effective amount of a compound, or a pharmaceutically acceptable salt thereof, as claimed by claim 1.

32. A method for treating type 2 diabetes mellitus comprising administering to a subject in need thereof, an effective amount of the compound, or a pharmaceutically acceptable salt thereof, as claimed by claim 1.

33. A pharmaceutical composition comprising the compound, or a pharmaceutically acceptable salt thereof, as claimed by claim 1, and at least one pharmaceutically acceptable carrier, diluent, or excipient.

34. A pharmaceutical composition as claimed by claim 1 wherein the composition is administered as a subcutaneous injection.

35. A pharmaceutical composition as claimed by claim 1 wherein the composition is administered orally.

36. A compound, or a pharmaceutically acceptable salt thereof, as claimed by claim 1 for use as a medicament.

37. A compound, or pharmaceutically acceptable salt thereof, as claimed by claim 1 for use in treating a condition selected from the group consisting of type 2 diabetes mellitus, obesity, NAFLD, nonalcoholic steatohepatitis, dyslipidemia, and metabolic syndrome.

38. A compound, or a pharmaceutically acceptable salt thereof, as claimed by claim 1 for use in treating type 2 diabetes mellitus.

39. The use of a compound, or a pharmaceutically acceptable salt thereof, as claimed by claim 1 in the manufacture of a medicament for treating a condition selected from the group consisting of type 2 diabetes mellitus, obesity, NAFLD, nonalcoholic steatohepatitis, dyslipidemia, and metabolic syndrome.

40. The use of a compound, or a pharmaceutically acceptable salt thereof, as claimed by claim 1 in the manufacture of a medicament for treating type 2 diabetes mellitus.
